(19) United States
(12) Patent Application Publication
(10) Pub. No.: US 2022/0281831 A1
(43) Pub. Date: Sep. 8, 2022

(54) FUSED-GLUTARIMIDE CRBN LIGANDS AND USES THEREOF

(71) Applicant: Kymera Therapeutics, Inc., Watertown, MA (US)

(72) Inventors: Nan JI, Arlington, MA (US); Michael d. SINTCHAK, Winchester, MA (US); Li ZHANG, Belmont, MA (US); Xiaozhang ZHENG, Lexington, MA (US)

(21) Appl. No.: 17/597,628

(22) PCT Filed: Jul. 15, 2020
(86) PCT No.: PCT/US2020/042105

§ 371 Date: Jan. 14, 2022

Related U.S. Application Data
(60) Provisional application No. 62/959,302, filed on Jan. 10, 2020.
(60) Provisional application No. 62/874,179, filed on Jul. 15, 2019.

(51) Int. Cl. C07D 265/26; C07D 401/06; C07D 217/24; C07D 413/06; C07D 471/04; C07D 498/04; C07D 239/96; C07D 471/10; C07D 209/96; C07D 223/16; C07D 487/04; C07D 519/00

(52) U.S. Cl. CPC C07D 265/26; C07D 401/06; C07D 217/24; C07D 413/06; C07D 471/04; C07D 519/00; C07D 239/96; C07D 471/10; C07D 209/96; C07D 223/16; C07D 487/04; C07D 498/04

(57) ABSTRACT

The present invention provides compounds, compositions thereof, and methods of using the same. The present invention also relates to compounds and methods useful for binding and modulating the activity of cereblon (CRBN), especially for the inhibition of CRBN, and the treatment of CRBN-mediated disorders.

### CROSS-REFERENCE TO RELATED APPLICATIONS

This application claims the benefit of U.S. Provisional App. No. 62/874,179, filed on Jul. 15, 2019, and U.S. Provisional App. No. 62/959,302, filed on Jan. 10, 2020, the content of each of which is hereby incorporated by reference.

### TECHNICAL FIELD OF THE INVENTION

The present invention relates to compounds and methods useful for binding and modulating the activity of cereblon (CRBN). The invention also provides pharmaceutically acceptable compositions comprising compounds of the present invention and methods of using said compositions in the treatment of various disorders.

### BACKGROUND OF THE INVENTION

The Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases. These ligases comprise over 500 different proteins and are categorized into multiple classes defined by the structural element of their E3 functional activity.

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (including cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e. abnormal or accelerated degradation of the protein target.

Cereblon (CRBN) interacts with damaged DNA binding protein 1 and forms an E3 ubiquitin ligase complex with Cullin 4 where it functions as a substrate receptor in which the proteins recognized by CRBN might be ubiquitinated and degraded by proteasomes.

Anew role for CRBN has been identified; i.e., the binding of immunomodulatory drugs (IMiDs), e.g. thalidomide, to CRBN has now been associated with teratogenicity and also the cytotoxicity of IMiDs, including lenalidomide, which are widely used to treat multiple myeloma patients. CRBN is likely a key player in the binding, ubiquitination and degradation of factors involved in maintaining function of myeloma cells. These new findings regarding the role of CRBN in IMiD action stimulated intense investigation of CRBN's downstream factors involved in maintaining regular function of a cell (Chang and Stewart, Int. J. Biochem. Mol. Biol. 2011 2(3):287-294).

Accordingly, there remains a need to find CRBN ligands useful as therapeutic agents.

### SUMMARY OF THE INVENTION

It has now been found that compounds of this invention, and pharmaceutically acceptable compositions thereof, are effective as CRBN ligands. Such compounds have the general formula I or I′:

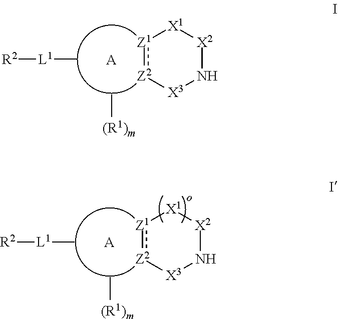

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with CRBN. Such diseases, disorders, or conditions include those described herein.

Compounds provided by this invention are also useful for the study of CRBN and associated proteins in biological and pathological phenomena; the study of CRBN occurring in bodily tissues; and the comparative evaluation of new CRBN ligands or other regulators of CRBN in vitro or in vivo.

### DETAILED DESCRIPTION OF CERTAIN EMBODIMENTS

### 1. General Description of Certain Embodiments of the Invention

Compounds of the present invention, and compositions thereof, are useful as CRBN ligands. As defined herein, the terms “binder,” “modulator,” and “ligand” are used interchangeably and describe a compound that binds to, modulates or is a ligand for CRBN.

In certain embodiments, the present invention provides a compound of formula I:

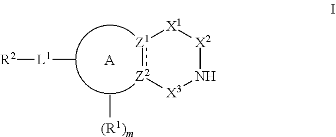

or a pharmaceutically acceptable salt thereof:
X¹ is a covalent bond, —CH₂—, —O—, —NR—, —CF₂—, or

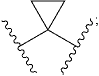 [SMILES: *C1(*)CC1]

X² and X³ are independently —CH₂—, —C(O)—, —C(S)—, or

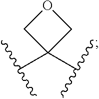 [SMILES: *C1(*)COC1]

Z¹ and Z² are independently a carbon atom or a nitrogen atom;
Ring A is a fused ring selected from benzo or a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

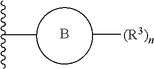

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4; and
n is 0, 1, 2, 3 or 4.

In certain embodiments, the present invention provides a compound of formula I′:

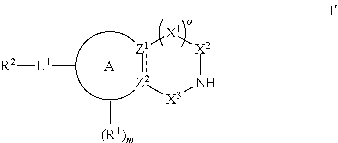

or a pharmaceutically acceptable salt thereof:
X¹ is —CR₂—, —O—, —NR—, —CF₂—,

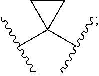 [SMILES: *C1(*)CC1]

### C(O)—, —C(S)—, or

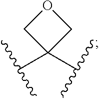 [SMILES: *C1(*)COC1] [0033] X² and X³ are independently —CR₂—, —C(O)—, —C(S)—, —CR₂C(O)—, or

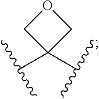 [SMILES: *C1(*)COC1]

Z¹ and Z² are independently a carbon atom or a nitrogen atom;
Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

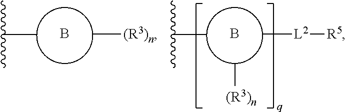

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
L² is a covalent bond or a bivalent, saturated or unsaturated, straight or branched C₁₋₂₀ hydrocarbon chain, wherein 0-6 methylene units of L are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

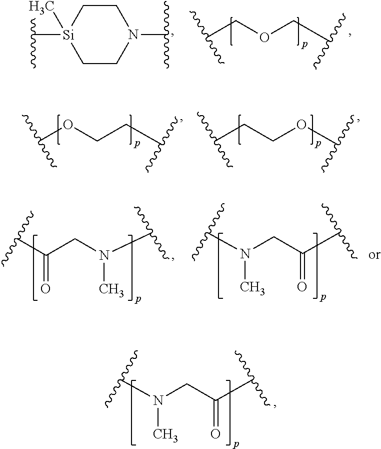

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
each p is independently 0, 1, 2, 3, 4, 5, 6, 7, 8, 9, or 10;
R⁵ is selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
[symbol] is a single or double bond;
m is 0, 1, 2, 3 or 4;
n is 0, 1, 2, 3 or 4;
o is 0, 1, or 2;
each p is independently 0, 1, 2, 3, 4, 5, 6, 7, 8, 9, or 10; and
q is 0 or 1.

### 2. Compounds and Definitions

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e. carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

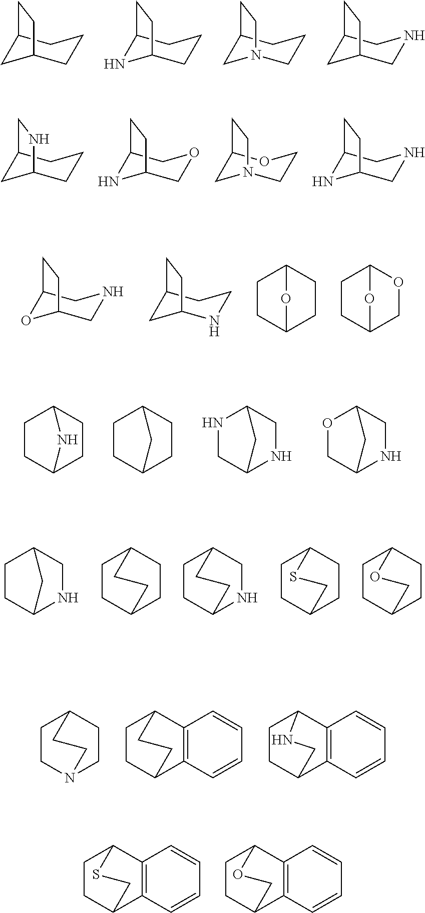

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

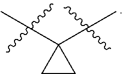 [SMILES: *C1(*)CC1]

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 9-membered monocyclic or 7- to 11-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, 2-oxa-6-azaspiro[3.3]heptane, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, —O—(CH₂)₀₋₄C(O)OR^{∘}; —(CH₂)₀₋₄CH(OR^{∘})₂; —(CH₂)₀₋₄SR^{∘}; —(CH₂)₀₋₄Ph, which may be substituted with R^{∘}; —(CH₂)₀₋₄O(CH₂)₀₋₁Ph which may be substituted with R^{∘}; —CH═CHPh, which may be substituted with R^{∘}; —(CH₂)₀₋₄O(CH₂)₀₋₁-pyridyl which may be substituted with R^{∘}; —NO₂; —CN; —N₃; —(CH₂)₀₋₄N(R^{∘})₂; —(CH₂)₀₋₄N(R^{∘})C(O)R^{∘}; —N(R^{∘})C(S)R^{∘}; —(CH₂)₀₋₄N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₂)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}; —SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —(CH₂)₀₋₄OC(O)NR^{∘}₂; —C(O)N(OR^{∘})R^{∘}; —C(O)C(O)R^{∘}; —C(O)CH₂C(O)R^{∘}; —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)₂NR^{∘}₂; —(CH₂)₀₋₄S(O)R^{∘}; —N(R^{∘})S(O)₂NR^{∘}₂; —N(R^{∘})S(O)₂R^{∘}; —N(OR^{∘})R^{∘}; —C(NH)NR^{∘}₂; —P(O)₂R^{∘}; —P(O)R^{∘}₂; —OP(O)R^{∘}₂; —OP(O)(OR^{∘})₂; —SiR^{∘}₃; —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₀₋₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂NHR^{●}, —(CH₂)₀₋₂NR^{●}₂, —NO₂, —SiR^{∘}3, —OSiR^{∘}3, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR_{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharm. Sci. 1977 66:1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools, as probes in biological assays, or as therapeutic agents in accordance with the present invention. In certain embodiments, a provided compound may be substituted with one or more deuterium atoms.

As used herein, the term “provided compound” refers to any genus, subgenus, and/or species set forth herein.

As used herein, the term “binder” or “ligand” is defined as a compound that binds to CRBN with measurable affinity. In certain embodiments, a compound has a binding constant of less than about 50 μM, less than about 1 μM, less than about 500 nM, less than about 100 nM, less than about 10 nM, or less than about 1 nM.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002 41:2596-99 and Sun et al., Bioconjugate Chem. 2006 17:52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably modulate,” as used herein, means a measurable change in a CRBN activity between a sample comprising a compound of the present invention, or composition thereof, and CRBN, and an equivalent sample comprising CRBN, in the absence of said compound, or composition thereof.

### 3. Description of Exemplary Embodiments

As described above, in certain embodiments, the present invention provides a compound of formula I:

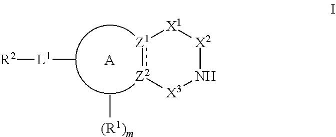

or a pharmaceutically acceptable salt thereof:
X¹ is a covalent bond, —CH₂—, —O—, —NR—, —CF₂—, or

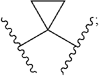 [SMILES: *C1(*)CC1]

X² and X³ are independently —CH₂—, —C(O)—, —C(S)—, or

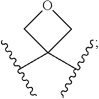 [SMILES: *C1(*)COC1]

Z¹ and Z² are independently a carbon atom or a nitrogen atom;
Ring A is a fused ring selected from benzo or a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

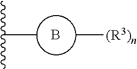

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
[symbol] is a single or double bond;
m is 0, 1, 2, 3 or 4; and
n is 0, 1, 2, 3 or 4.

As described above, in certain embodiments, the present invention provides a compound of formula I′:

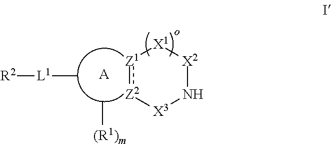

or a pharmaceutically acceptable salt thereof:
X¹ is —CR₂—, —O—, —NR—, —CF₂—,

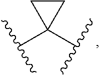 [SMILES: *C1(*)CC1]

### C(O)—, —C(S)—, or

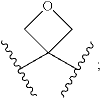 [SMILES: *C1(*)COC1] [0111] X² and X³ are independently —CR₂—, —C(O)—, —C(S)—, —CR₂C(O)—, or

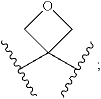 [SMILES: *C1(*)COC1]

Z¹ and Z² are independently a carbon atom or a nitrogen atom;
Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

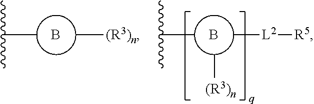

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
L² is a covalent bond or a bivalent, saturated or unsaturated, straight or branched C₁₋₂₀ hydrocarbon chain, wherein 0-6 methylene units of L are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

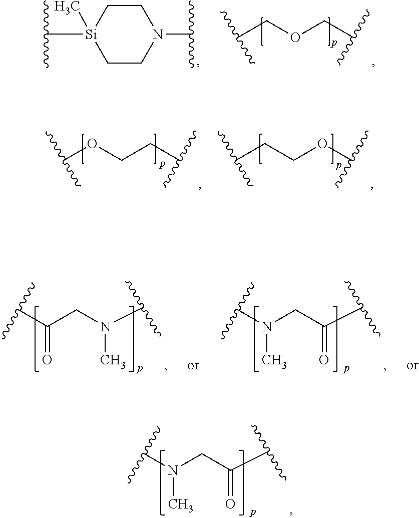

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
each p is independently 0, 1, 2, 3, 4, 5, 6, 7, 8, 9, or 10;
R⁵ is selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
[symbol] is a single or double bond;
m is 0, 1, 2, 3 or 4;
n is 0, 1, 2, 3 or 4;
o is 0, 1, or 2;
each p is independently 0, 1, 2, 3, 4, 5, 6, 7, 8, 9, or 10; and
q is 0 or 1.

As described above, in certain embodiments, the present invention provides a compound of formula II:

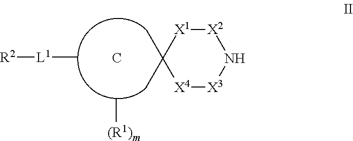

or a pharmaceutically acceptable salt thereof:
X¹ and X⁴ are independently a covalent bond, —CR₂—, —O—, —NR—, —C(O)—, —CF₂—, or

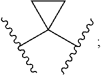 [SMILES: *C1(*)CC1]

X² and X³ are independently —CR₂—, —C(O)—, —C(S)—, or

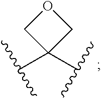 [SMILES: *C1(*)COC1]

Ring C is a spiro-fused ring selected from a 4-10 membered saturated or partially unsaturated mono- or bicyclic carbocyclic or heterocyclic ring having 1-3 heteroatoms independently selected from nitrogen, oxygen, and sulfur, wherein Ring C is optionally further substituted with 1-2 oxo groups;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
   two R¹ groups are optionally taken together to form an optionally substituted 5-8 membered partially unsaturated or aryl fused ring having 0-2 heteroatoms independently selected from nitrogen, oxygen, or sulfur;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected from

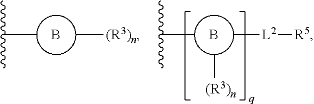

or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
L² is a covalent bond or a bivalent, saturated or unsaturated, straight or branched C₁₋₂₀ hydrocarbon chain, wherein 0-6 methylene units of L are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

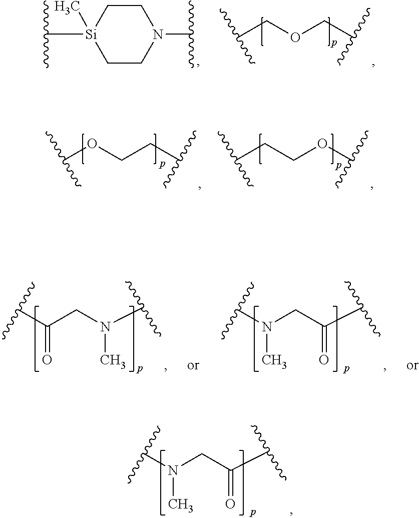

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
each p is independently 0, 1, 2, 3, 4, 5, 6, 7, 8, 9, or 10;
R⁵ is selected from hydrogen, deuterium, R⁴, halogen, —CN, —NO₂, —OR, —SR, —NR₂, —S(O)₂R, —S(O)₂NR₂, —S(O)R, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
m is 0, 1, 2, 3 or 4;
n is 0, 1, 2, 3 or 4;
each p is independently 0, 1, 2, 3, 4, 5, 6, 7, 8, 9, or 10; and
q is 0 or 1.

As defined above and described herein, X¹ is a covalent bond, —CH₂—, —CR₂—, —O—, —NR—, —CF₂—,

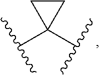 [SMILES: *C1(*)CC1]

### —C(O)—, —C(S)—, or

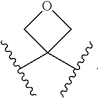 [SMILES: *C1(*)COC1]

In some embodiments, X¹ is a covalent bond. In some embodiments, X¹ is —CH₂—. In some embodiments, X¹ is —CR₂—. In some embodiments, X¹ is —O—. In some embodiments, X¹ is —NR—. In some embodiments, X¹ is —NH—. In some embodiments, X¹ is —NMe-. In some embodiments, X¹ is —CF₂—. In some embodiments, X¹ is

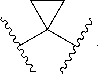 [SMILES: *C1(*)CC1]

In some embodiments, X¹ is —C(O)—. In some embodiments, X¹ is —C(S)—. In some embodiments, X¹ is

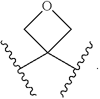 [SMILES: *C1(*)COC1]

In certain embodiments, X¹ is selected from those shown in the compounds of Table 1.

As defined above and described herein, X² and X³ are independently —CH₂—, —CR₂—, —C(O)—, —C(S)—, —CR₂C(O)—, or

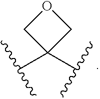 [SMILES: *C1(*)COC1]

In some embodiments, X² and X³ are independently —CH₂—. In some embodiments, X² and X³ are independently —CR₂—. In some embodiments, X² and X³ are independently —C(O)—. In some embodiments, X² and X³ are independently —C(S)—. In some embodiments, X² and X³ are independently —CR₂C(O)—. In some embodiments, X² and X³ are independently

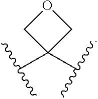 [SMILES: *C1(*)COC1]

In certain embodiments, X² and X³ are independently selected from those shown in the compounds of Table 1.

As defined above and described herein, X⁴ is a covalent bond, —CH₂—, —CR₂—, —O—, —NR—, —CF₂—,

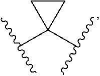 [SMILES: *C1(*)CC1]

### —C(O)—, —C(S)—, or

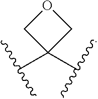 [SMILES: *C1(*)COC1]

In some embodiments, X⁴ is a covalent bond. In some embodiments, X⁴ is —CH₂—. In some embodiments, X⁴ is —CR₂—. In some embodiments, X⁴ is —O—. In some embodiments, X⁴ is —NR—. In some embodiments, X⁴ is —CF₂—. In some embodiments, X⁴ is

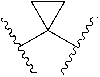 [SMILES: *C1(*)CC1]

In some embodiments, X⁴ is —C(O)—. In some embodiments, X⁴ is —C(S)—. In some embodiments, X⁴ is

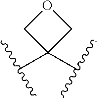 [SMILES: *C1(*)COC1]

In certain embodiments, X⁴ is selected from those shown in the compounds of Table 1.

As define above and described herein, Z¹ and Z² are independently a carbon atom or a nitrogen atom.

In some embodiments, Z¹ and Z² are independently a carbon atom. In some embodiments, Z¹ and Z² are independently a carbon atom.

In certain embodiments, Z¹ and Z² are independently selected from those shown in the compounds of Table 1.

As defined above and described herein, Ring A is a fused ring selected from benzo, a 4-6 membered saturated or partially unsaturated carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur.

In some embodiments, Ring A is benzo. In some embodiments, Ring A is a 4-6 membered saturated or partially unsaturated carbocyclic or heterocyclic ring having 1-3 heteroatoms independently selected from nitrogen, oxygen, and sulfur. In some embodiments, Ring A is a 5-6 membered heteroaryl ring having 1-4 heteroatoms independently selected from nitrogen, oxygen, and sulfur.

In some embodiments, Ring A is

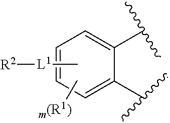

In some embodiments, Ring A is

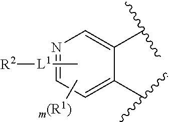

In some embodiments, Ring A is

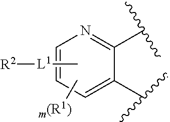

In some embodiments, Ring A is

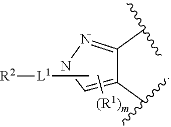

In some embodiments, Ring A is

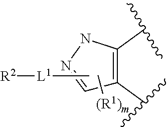

In some embodiments, Ring A is

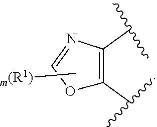

In some embodiments, Ring A is

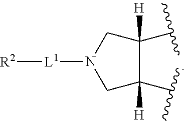

In certain embodiments, Ring A is selected from those shown in the compounds of Table 1.

As defined about and described herein, Ring C is a spiro-fused ring selected from a 4-10 membered saturated or partially unsaturated mono- or bicyclic carbocyclic or heterocyclic ring having 1-3 heteroatoms independently selected from nitrogen, oxygen, and sulfur, wherein Ring C is optionally further substituted with 1-2 oxo groups.

In some embodiments, Ring C is a spiro-fused ring selected from a 4-10 membered saturated or partially unsaturated mono- or bicyclic carbocyclic or heterocyclic ring having 1-3 heteroatoms independently selected from nitrogen, oxygen, and sulfur. In some embodiments, Ring C is optionally further substituted with 1-2 oxo groups.

In some embodiments, Ring C is

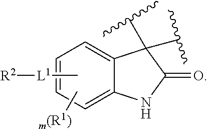

In some embodiments, Ring C is

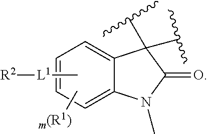

In some embodiments, Ring C is

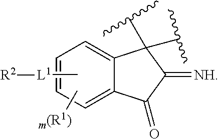

In some embodiments, Ring C is

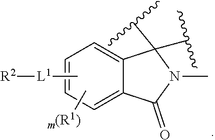

In some embodiments, Ring C is

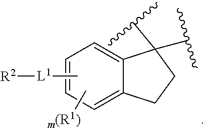

In some embodiments, Ring C is

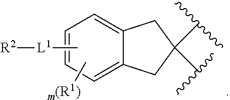

In some embodiments, Ring C is

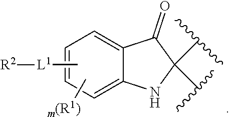

In some embodiments, Ring C is

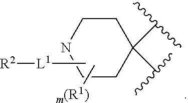

In certain embodiments, Ring C is selected from those shown in the compounds of Table 1.

As defined above and described herein, L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is a covalent bond. In some embodiments, L¹ is a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—.

In some embodiments, L¹ is —C(O)—. In some embodiments, L¹ is —CH₂—. In some embodiments, L¹ is —O—. In some embodiments, L¹ is —N(R)CH₂—.

In certain embodiments, L¹ is selected from those shown in the compounds of Table 1.

As defined above and described herein, each R¹ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃, or two R¹ groups are optionally taken together to form an optionally substituted 5-8 membered partially unsaturated or aryl fused ring having 0-2 heteroatoms independently selected from nitrogen, oxygen, or sulfur.

In some embodiments, R¹ is hydrogen. In some embodiments, R¹ is deuterium. In some embodiments, R¹ is R⁴. In some embodiments, R¹ is halogen. In some embodiments, R¹ is —CN. In some embodiments, R¹ is —NO₂. In some embodiments, R¹ is —OR. In some embodiments, R¹ is —SR. In some embodiments, R¹ is —NR₂. In some embodiments, R¹ is —S(O)₂R. In some embodiments, R¹ is —S(O)₂NR₂. In some embodiments, R¹ is —S(O)R. In some embodiments, R¹ is —CF₂R. In some embodiments, R¹ is —CF₃. In some embodiments, R¹ is —CR₂(OR). In some embodiments, R¹ is —CR₂(NR₂). In some embodiments, R¹ is —C(O)R. In some embodiments, R¹ is —C(O)OR. In some embodiments, R¹ is —C(O)NR₂. In some embodiments, R¹ is —C(O)N(R)OR. In some embodiments, R¹ is —OC(O)R. In some embodiments, R¹ is —OC(O)NR₂. In some embodiments, R¹ is —C(S)NR₂. In some embodiments, R¹ is —N(R)C(O)OR. In some embodiments, R¹ is —N(R)C(O)R. In some embodiments, R¹ is —N(R)C(O)NR₂. In some embodiments, R¹ is —N(R)S(O)₂R. In some embodiments, R¹ is —OP(O)R₂. In some embodiments, R¹ is —OP(O)(OR)₂. In some embodiments, R¹ is —OP(O)(OR)NR₂. In some embodiments, R¹ is —OP(O)(NR₂)₂. In some embodiments, R¹ is —Si(OR)R₂. In some embodiments, R¹ is —SiR₃. In some embodiments, two R¹ groups are optionally taken together to form an optionally substituted 5-8 membered partially unsaturated or aryl fused ring having 0-2 heteroatoms independently selected from nitrogen, oxygen, or sulfur.

In some embodiments, R¹ is fluoro. In some embodiments, R¹ is bromo. In some embodiments, R¹ is methyl. In some embodiments, R¹ is —OH. In some embodiments, R¹ is —NH₂. In some embodiments, R¹ is —NHCH₃. In some embodiments, R¹ is —N(CH₃)₂. In some embodiments, R¹ is —NHCH(CH₃)₂. In some embodiments, R¹ is —NHSO₂CH₃. In some embodiments, R¹ is —CH₂OH. In some embodiments, R¹ is —CH₂NH₂. In some embodiments, R¹ is —C(O)NH₂. In some embodiments, R¹ is —C(O)NBn₂. In some embodiments, R¹ is —C(O)NHCH₃. In some embodiments, R¹ is

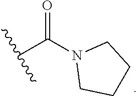 [SMILES: *C(=O)N1CCCC1]

In some embodiments, R¹ is

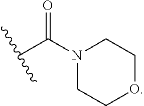 [SMILES: *C(=O)N1CCOCC1]

In some embodiments, R¹ is

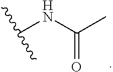 [SMILES: *NC(C)=O]

In some embodiments, R¹ is

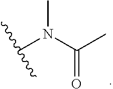 [SMILES: *N(C)C(C)=O]

In some embodiments, R¹ is

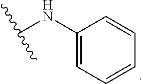 [SMILES: *Nc1ccccc1]

In some embodiments, R¹ is

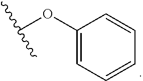 [SMILES: *Oc1ccccc1]

In some embodiments, R¹ is

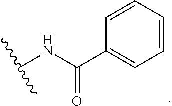 [SMILES: *NC(=O)c1ccccc1]

In some embodiments, R¹ is

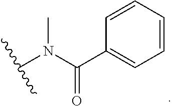 [SMILES: *N(C)C(=O)c1ccccc1]

In some embodiments, R¹ is

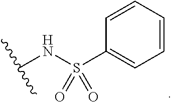 [SMILES: *NS(=O)(=O)c1ccccc1]

In certain embodiments, each R¹ is independently selected from those shown in the compounds of Table 1.

As defined above and described here, each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

In some embodiments, R is hydrogen. In some embodiments, R is an optionally substituted C₁₋₆ aliphatic. In some embodiments, R is an optionally substituted phenyl. In some embodiments, R is an optionally substituted 4-7 membered saturated or partially unsaturated heterocyclic having 1-2 heteroatoms independently selected from nitrogen, oxygen, and sulfur. In some embodiments, R is an optionally substituted a 5-6 membered heteroaryl ring having 1-4 heteroatoms independently selected from nitrogen, oxygen, and sulfur. In some embodiments, two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur.

As defined above and described herein, R² is selected from

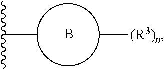

or hydrogen.

In some embodiment R² is

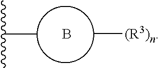

In some embodiment R² is

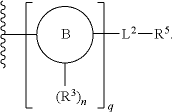

In some embodiments, R² is hydrogen.

In certain embodiments, R² is selected from those shown in the compounds of Table 1.

As defined above and described herein, Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups.

In some embodiments, Ring B is phenyl. In some embodiments, Ring B is a 4-10 membered saturated or partially unsaturated mono- or bicyclic carbocyclic or heterocyclic ring having 1-3 heteroatoms independently selected from nitrogen, oxygen, and sulfur In some embodiments, Ring B is a 5-6 membered heteroaryl ring having 1-4 heteroatoms independently selected from nitrogen, oxygen, and sulfur. In some embodiments, Ring B is further optionally substituted with 1-2 oxo groups.

In some embodiments, Ring B is

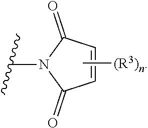

In some embodiments, Ring B is

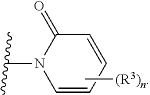

In some embodiments, Ring B is

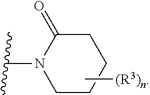

In some embodiments Ring B is

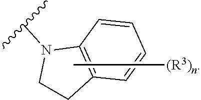

In some embodiments Ring B is

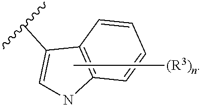

In some embodiments Ring B is

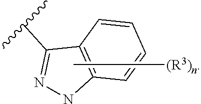

In some embodiments Ring B is

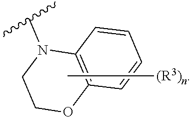

In some embodiments Ring B is

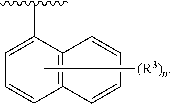

In some embodiments Ring B is

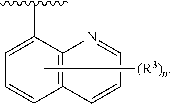

In some embodiments Ring B is

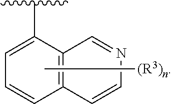

In some embodiments Ring B is

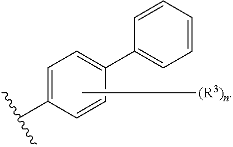

In some embodiments Ring B is

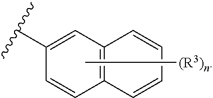

In some embodiments Ring B is

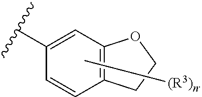

In some embodiments Ring B is

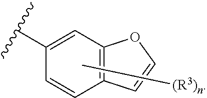

In some embodiments Ring B is

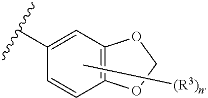

In some embodiments Ring B is

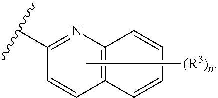

In some embodiments Ring B is

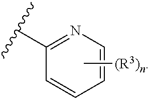

In some embodiments Ring B is

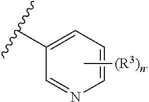

In some embodiments Ring B is

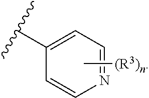

In some embodiments Ring B is

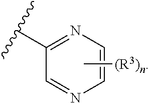

In some embodiments Ring B is

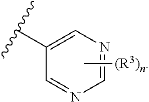

In some embodiments Ring B is

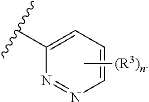

In some embodiments Ring B is

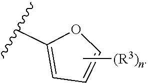

In some embodiments Ring B is

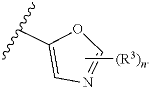

In some embodiments Ring B is

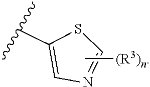

In some embodiments Ring B is

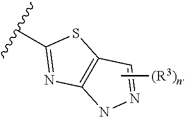

In some embodiments Ring B is

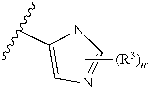

In some embodiments Ring B is

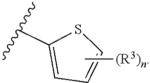

In some embodiments Ring B is

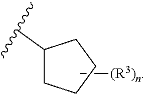

In some embodiments Ring B is

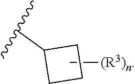

In certain embodiments, Ring B is selected from those shown in the compounds of Table 1.

As defined above and described herein, each R³ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R³ is hydrogen. In some embodiments, R³ is deuterium. In some embodiments, R³ is R⁴. In some embodiments, R³ is halogen. In some embodiments, R³ is —CN. In some embodiments, R³ is —NO₂. In some embodiments, R³ is —OR. In some embodiments, R³ is —SR. In some embodiments, R³ is —NR₂. In some embodiments, R³ is —S(O)₂R. In some embodiments, R³ is —S(O)₂NR₂. In some embodiments, R³ is —S(O)R. In some embodiments, R³ is —CF₂R. In some embodiments, R³ is —CF₃. In some embodiments, R³ is —CR₂(OR). In some embodiments, R³ is —CR₂(NR₂). In some embodiments, R³ is —C(O)R. In some embodiments, R³ is —C(O)OR. In some embodiments, R³ is —C(O)NR₂. In some embodiments, R³ is —C(O)N(R)OR. In some embodiments, R³ is —OC(O)R. In some embodiments, R³ is —OC(O)NR₂. In some embodiments, R³ is —N(R)C(O)OR. In some embodiments, R³ is —N(R)C(O)R. In some embodiments, R³ is —N(R)C(O)NR₂. In some embodiments, R³ is —N(R)S(O)₂R. In some embodiments, R³ is —OP(O)R₂. In some embodiments, R³ is —OP(O)(OR)₂. In some embodiments, R³ is —OP(O)(OR)NR₂. In some embodiments, R³ is —OP(O)(NR₂)₂. In some embodiments, R³ is —SiR₃.

In certain embodiments, R³ is selected from those shown in the compounds of Table 1.

As defined above and described herein, each R⁴ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁴ is an optionally substituted C₁₋₆ aliphatic. In some embodiments, R⁴ is an optionally substituted phenyl. In some embodiments, R⁴ is an optionally substituted 4-7 membered saturated or partially unsaturated heterocyclic ring having 1-2 heteroatoms independently selected from nitrogen, oxygen, and sulfur. In some embodiments, R⁴ is an optionally substituted 5-6 membered heteroaryl ring having 1-4 heteroatoms independently selected from nitrogen, oxygen, and sulfur.

In some embodiments, R⁴ is

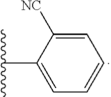 [SMILES: *c1ccccc1C#N]

In some embodiments, R⁴ is

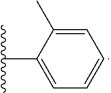 [SMILES: *c1ccccc1C]

In some embodiments, R⁴ is

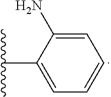 [SMILES: *c1ccccc1N]

In some embodiments, R⁴ is

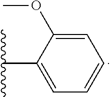 [SMILES: *c1ccccc1OC]

In some embodiments, R⁴ is

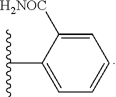 [SMILES: *c1ccccc1C(N)=O]

In some embodiments, R⁴ is

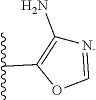 [SMILES: *c1ocnc1N]

In some embodiments, R⁴ is

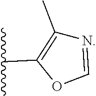 [SMILES: *c1ocnc1C]

In some embodiments, R⁴ is

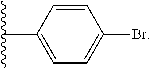 [SMILES: *c1ccc(Br)cc1]

In some embodiments, R⁴ is

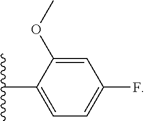 [SMILES: *c1ccc(F)cc1OC]

In some embodiments, R⁴ is

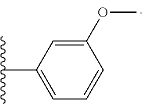 [SMILES: *c1cccc(OC)c1]

In some embodiments, R⁴ is

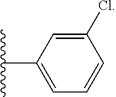 [SMILES: *c1cccc(Cl)c1]

In some embodiments, R is

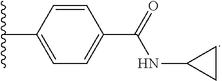 [SMILES: *c1ccc(C(=O)NC2CC2)cc1]

In some embodiments, R is

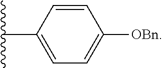 [SMILES: *c1ccc(OCc2ccccc2)cc1]

In some embodiments, R⁴ is

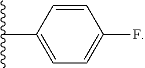 [SMILES: *c1ccc(F)cc1]

In some embodiments, R⁴ is

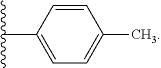 [SMILES: *c1ccc(C)cc1]

In some embodiments, R⁴ is

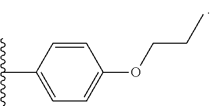 [SMILES: *c1ccc(OCCC)cc1]

In some embodiments, R⁴ is

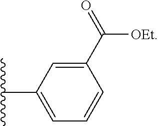 [SMILES: *c1cccc(C(=O)OCC)c1]

In some embodiments, R⁴ is

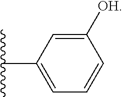 [SMILES: *c1cccc(O)c1]

In some embodiments, R⁴ is

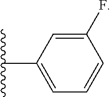 [SMILES: *c1cccc(F)c1]

In some embodiments, R⁴ is

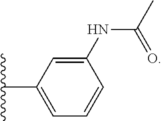 [SMILES: *c1cccc(NC(C)=O)c1]

In some embodiments, R⁴ is

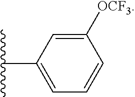 [SMILES: *c1cccc(OC(F)(F)F)c1]

In some embodiments, R⁴ is

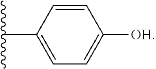 [SMILES: *c1ccc(O)cc1]

In some embodiments, R⁴ is

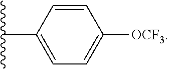 [SMILES: *c1ccc(OC(F)(F)F)cc1]

In some embodiments, R⁴ is

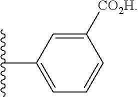 [SMILES: *c1cccc(C(=O)O)c1]

In some embodiments, R⁴ is

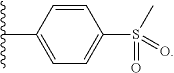 [SMILES: *c1ccc(S(C)(=O)=O)cc1]

In some embodiments, R⁴ is

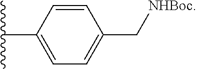 [SMILES: *c1ccc(CNC(=O)OC(C)(C)C)cc1]

In some embodiments, R⁴ is

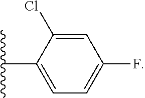 [SMILES: *c1ccc(F)cc1Cl]

In some embodiments, R⁴ is

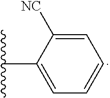 [SMILES: *c1ccccc1C#N]

In some embodiments, R⁴ is

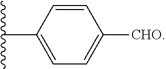 [SMILES: *c1ccc(C=O)cc1]

In some embodiments, R⁴ is

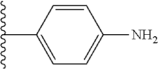 [SMILES: *c1ccc(N)cc1]

In some embodiments R⁴ is

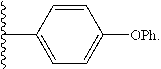 [SMILES: *c1ccc(Oc2ccccc2)cc1]

In some embodiments, R⁴ is

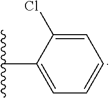 [SMILES: *c1ccccc1Cl]

In some embodiments, R⁴ is

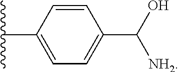 [SMILES: *c1ccc(C(N)O)cc1]

In some embodiments, R⁴ is

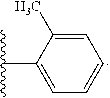 [SMILES: *c1ccccc1C]

In some embodiments, R⁴ is

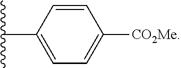 [SMILES: *c1ccc(C(=O)OC)cc1]

In some embodiments, R⁴ is

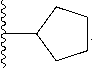 [SMILES: *C1CCCC1]

In some embodiments, R⁴ is

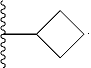 [SMILES: *C1CCC1]

In some embodiments, R⁴ is

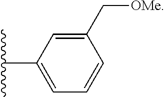 [SMILES: *c1cccc(COC)c1]

In some embodiments, R⁴ is

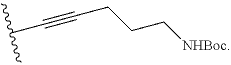 [SMILES: *C#CCCCNC(=O)OC(C)(C)C]

In certain embodiments, R⁴ is selected from those shown in the compounds of Table 1.

As defined above and described herein, L² is a covalent bond or a bivalent, saturated or unsaturated, straight or branched C₁₋₂₀ hydrocarbon chain, wherein 0-6 methylene units of L² are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—

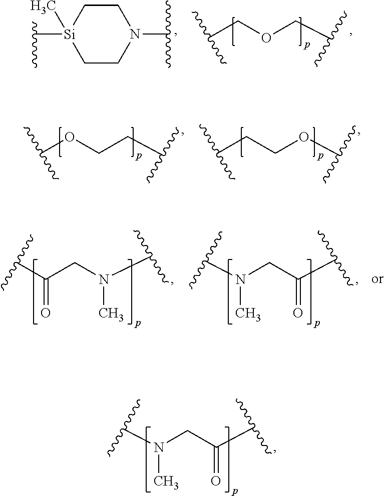

and wherein p is 0, 1, 2, 3, 4, 5, 6, 7, 8, 9, or 10.

In some embodiments, L² is a covalent bond. In some embodiments, L is a bivalent, saturated or unsaturated, straight or branched C₁₋₅₀ hydrocarbon chain, wherein 0-6 methylene units of L² are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—,

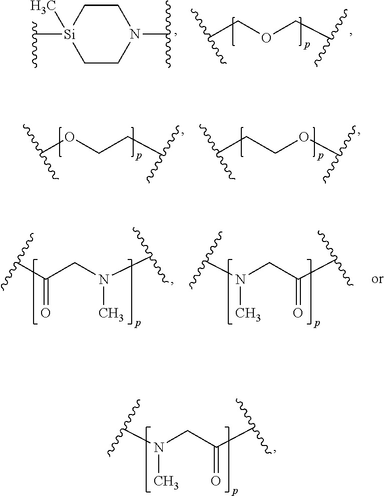

and wherein p is 0, 1, 2, 3, 4, 5, 6, 7, 8, 9, or 10.

As defined above and described herein, each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur.

In some embodiments, -Cy- is an optionally substituted phenylenyl. In some embodiments, -Cy- is an optionally substituted 8-10 membered bicyclic arylenyl. In some embodiments, -Cy- is an optionally substituted 4-7 membered saturated or partially unsaturated carbocyclylenyl. In some embodiments, -Cy- is an optionally substituted 4-11 membered saturated or partially unsaturated spiro carbocyclylenyl. In some embodiments, -Cy- is an optionally substituted 8-10 membered bicyclic saturated or partially unsaturated carbocyclylenyl. In some embodiments, -Cy- is an optionally substituted 4-7 membered saturated or partially unsaturated heterocyclylenyl having 1-2 heteroatoms independently selected from nitrogen, oxygen, and sulfur. In some embodiments, -Cy- is an optionally substituted 4-11 membered saturated or partially unsaturated spiro heterocyclylenyl having 1-2 heteroatoms independently selected from nitrogen, oxygen, and sulfur. In some embodiments, -Cy- is an optionally substituted 8-10 membered bicyclic saturated or partially unsaturated heterocyclylenyl having 1-2 heteroatoms independently selected from nitrogen, oxygen, and sulfur. In some embodiments, -Cy- is an optionally substituted 5-6 membered heteroarylenyl having 1-4 heteroatoms independently selected from nitrogen, oxygen, and sulfur. In some embodiments, -Cy- is an optionally substituted 8-10 membered bicyclic heteroarylenyl having 1-5 heteroatoms independently selected from nitrogen, oxygen, or sulfur.

In some embodiments, L² is

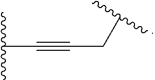 [SMILES: *C#CC*]

In some embodiments, L² is

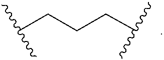 [SMILES: *CCC*]

In some embodiments, L² is

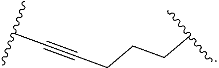 [SMILES: *C#CCCC*]

In some embodiments, L² is

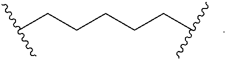 [SMILES: *CCCCC*]

In some embodiments, L² is

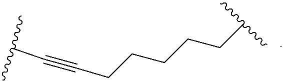 [SMILES: *C#CCCCCC*]

In some embodiments, L² is

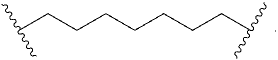 [SMILES: *CCCCCCC*]

In some embodiments, L² is

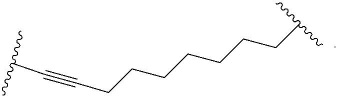 [SMILES: *C#CCCCCCCC*]

In some embodiments, L² is

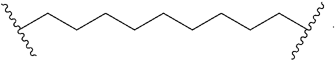 [SMILES: *CCCCCCCCC*]

In certain embodiments, L² is selected from those shown in the compounds of Table 1.

As defined above and described herein, R⁵ is selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃.

In some embodiments, R⁵ is hydrogen. In some embodiments, R⁵ is deuterium. In some embodiments, R⁵ is R⁴. In some embodiments, R⁵ is halogen. In some embodiments, R⁵ is —CN. In some embodiments, R⁵ is —NO₂. In some embodiments, R⁵ is —OR. In some embodiments, R⁵ is —SR. In some embodiments, R⁵ is —NR₂. In some embodiments, R⁵ is —S(O)₂R. In some embodiments, R⁵ is —S(O)₂NR₂. In some embodiments, R⁵ is —S(O)R. In some embodiments, R⁵ is —CF₂R. In some embodiments, R⁵ is —CF₃. In some embodiments, R⁵ is —CR₂(OR). In some embodiments, R⁵ is —CR₂(NR₂). In some embodiments, R⁵ is —C(O)R. In some embodiments, R⁵ is —C(O)OR. In some embodiments, R⁵ is —C(O)NR₂. In some embodiments, R⁵ is —C(O)N(R)OR. In some embodiments, R⁵ is —OC(O)R. In some embodiments, R⁵ is —OC(O)NR₂. In some embodiments, R⁵ is —N(R)C(O)OR. In some embodiments, R⁵ is —N(R)C(O)R. In some embodiments, R⁵ is —N(R)C(O)NR₂. In some embodiments, R⁵ is —N(R)S(O)₂R. In some embodiments, R⁵ is —OP(O)R₂. In some embodiments, R⁵ is —OP(O)(OR)₂. In some embodiments, R⁵ is —OP(O)(OR)NR₂. In some embodiments, R⁵ is —OP(O)(NR₂)₂. In some embodiments, R⁵ is —SiR₃.

In certain embodiments, R⁵ is selected from those shown in the compounds of Table 1.

As defined above and described herein, [symbol] is a single or double bond.

In some embodiments, [symbol] is a single bond. In some embodiments, [symbol] is a double bond.

In certain embodiments, [symbol] is selected from those shown in the compounds of Table 1.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In certain embodiments, m is selected from those shown in the compounds of Table 1.

As defined above and described herein, n is 0, 1, 2, 3 or 4.

In some embodiments, n is 0. In some embodiments, n is 1. In some embodiments, m is 2. In some embodiments, n is 3. In some embodiments, n is 4.

In certain embodiments, n is selected from those shown in the compounds of Table 1.

As defined above and described herein, o is 0, 1, or 2.

In some embodiments, o is 0. In some embodiments, o is 1. In some embodiments, o is 2.

In certain embodiments, o is selected from those shown in the compounds of Table 1.

As defined above and described herein, q is 0 or 1.

In some embodiments, q is 0. In some embodiments, q is 1.

In certain embodiments, q is selected from those shown in the compounds of Table 1.

In some embodiments, the present invention provides a compound of formula I′, wherein Ring A is benzo, o is 1, X¹ is —CH₂—, X² and X³ are —C(O)—, and Z¹ and Z² are carbon atoms as shown, to provide a compound of formula I-a:

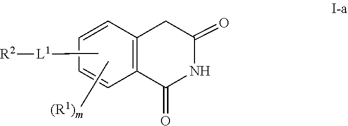

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is imidazolyl, o is 1, X¹ is —CH₂—, X² and X³ are —C(O)—, and Z¹ and Z² are carbon atoms as shown, to provide a compound of formula I-b:

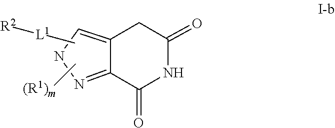

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is imidazolyl, o is 1, X¹ is —CH₂—, X² and X³ are —C(O)—, and Z¹ and Z² are carbon atoms as shown, to provide a compound of formula I-c:

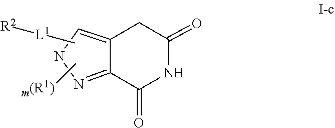

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is oxazolyl, o is 1, X¹ is —CH₂—, X² and X³ are —C(O)—, and Z¹ and Z² are carbon atoms as shown, to provide a compound of formula I-d:

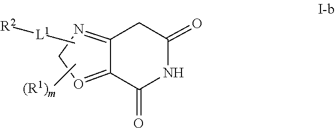

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is benzo, o is 0, X² and X³ are —C(O)—, and Z¹ and Z² are carbon atoms as shown, to provide a compound of formula I-e:

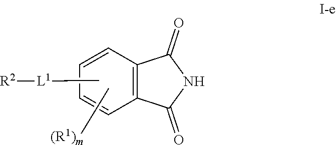

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is benzo, o is 1, X¹ is —O—, X² and X³ are —C(O)—, and Z¹ and Z² are carbon atoms as shown, to provide a compound of formula I-f:

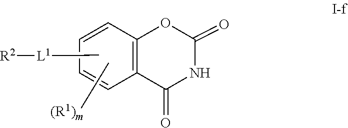

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is benzo, o is 1, X¹ is —NR—, X² and X³ are —C(O)—, and Z¹ and Z² are carbon atoms as shown, to provide a compound of formula I-g:

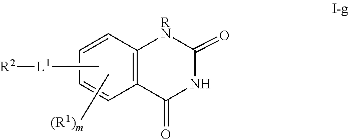

or a pharmaceutically acceptable salt thereof, wherein each of L¹, R, R¹, R², and m is as defined above and described in embodiments herein, both singly and in combination.

In some embodiments, the present invention provides a compound of formula I′, wherein Ring A is benzo, o is 1, X¹ is —CF₂—, X² and X³ are —C(O)—, and Z¹ and Z² are carbon atoms as shown, to provide a compound of formula I-h:

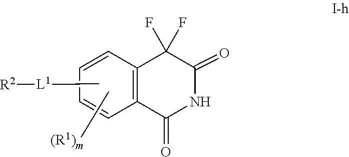

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is benzo, o is 1, X¹ is

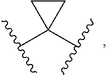 [SMILES: *C1(*)CC1]

X² and X³ are —C(O)—, and Z¹ and Z² are carbon atoms as shown, to provide a compound of formula I-i:

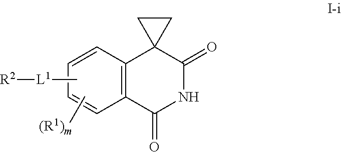

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is pyridyl, o is 1, X¹ is —CH₂—, X² and X³ are —C(O)—, and Z¹ and Z² are carbon atoms as shown, to provide a compound of formula I-j:

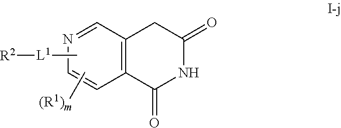

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is pyridyl, o is 1, X¹ is —CH₂—, X² and X³ are —C(O)—, and Z¹ and Z² are carbon atoms as shown, to provide a compound of formula I-k:

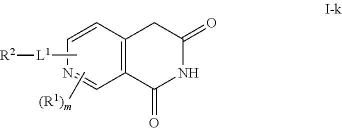

or a pharmaceutically acceptable salt thereof, wherein each of L¹, R¹, R², and m is as defined above and described in embodiments herein, both singly and in combination.

In some embodiments, the present invention provides a compound of formula I′, wherein Ring A is benzo, o is 1, X¹, X² and X³ are —C(O)—, and Z¹ and Z² are carbon atoms as shown, to provide a compound of formula I-1:

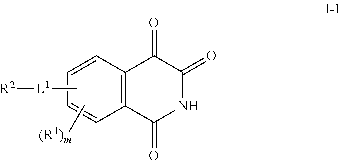

or a pharmaceutically acceptable salt thereof, wherein each of L¹, R¹, R², and m is as defined above and described in embodiments herein, both singly and in combination.

In some embodiments, the present invention provides a compound of formula I′, wherein Z¹ and Z² are carbon atoms and Ring A is

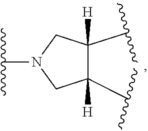 [SMILES: *[C@@H]1CN(*)C[C@@H]1*]

o is 0, and X² and X³ are —C(O)— as shown, to provide a compound of formula I-m:

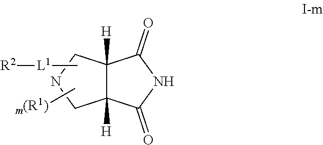

or a pharmaceutically acceptable salt thereof, wherein each of L¹, R¹, R², and m is as defined above and described in embodiments herein, both singly and in combination.

In some embodiments, the present invention provides a compound of formula II, wherein X¹ and X⁴ are —CH₂—, and X² and X³ are —C(O)— as shown, to provide a compound of formula II-a:

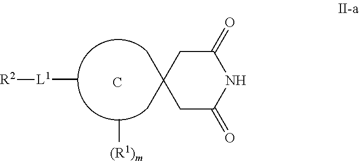

or a pharmaceutically acceptable salt thereof, wherein each of Ring C, L¹, R¹, R², and m is as defined above and described in embodiments herein, both singly and in combination.

In some embodiments, the present invention provides a compound of formula II, wherein X¹ is —CH₂—, X⁴ is a covalent bond, and X² and X³ are —C(O)— as shown, to provide a compound of formula II-b:

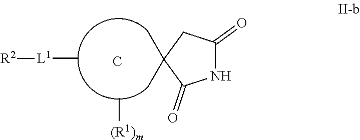

or a pharmaceutically acceptable salt thereof, wherein each of Ring C, L¹, R¹, R², and m is as defined above and described in embodiments herein, both singly and in combination.

Exemplary compounds of the invention are set forth in Table 1, below.

TABLE 1
Exemplary Compounds

| I-# | Structure |
|---|---|
| I-1 | 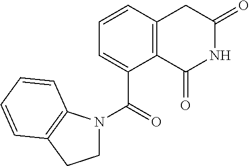 [SMILES: O=C1Cc2cccc(C(=O)N3CCc4ccccc43)c2C(=O)N1] |
| I-2 | 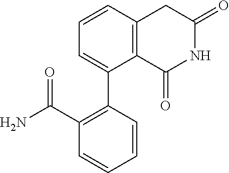 [SMILES: NC(=O)c1ccccc1-c1cccc2c1C(=O)NC(=O)C2] |
| I-3 | 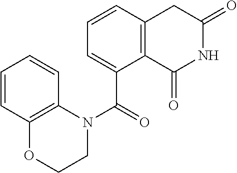 [SMILES: O=C1Cc2cccc(C(=O)N3CCOc4ccccc43)c2C(=O)N1] |
| I-4 | 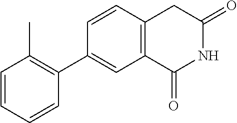 [SMILES: Cc1ccccc1-c1ccc2c(c1)C(=O)NC(=O)C2] |
| I-5 | 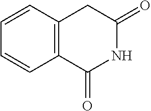 [SMILES: O=C1Cc2ccccc2C(=O)N1] |
| I-6 | 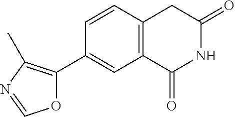 [SMILES: Cc1ncoc1-c1ccc2c(c1)C(=O)NC(=O)C2] |
| I-7 | 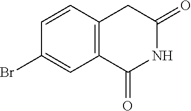 [SMILES: O=C1Cc2ccc(Br)cc2C(=O)N1] |
| I-8 | 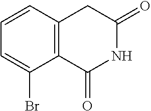 [SMILES: O=C1Cc2cccc(Br)c2C(=O)N1] |
| I-9 | 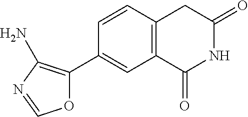 [SMILES: Nc1ncoc1-c1ccc2c(c1)C(=O)NC(=O)C2] |
| I-10 | 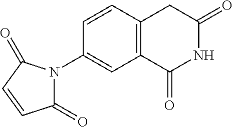 [SMILES: O=C1Cc2ccc(N3C(=O)C=CC3=O)cc2C(=O)N1] |
| I-11 | 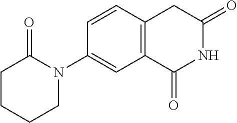 [SMILES: O=C1Cc2ccc(N3CCCCC3=O)cc2C(=O)N1] |
| I-12 | 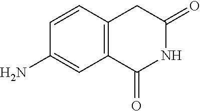 [SMILES: Nc1ccc2c(c1)C(=O)NC(=O)C2] |
| I-13 | 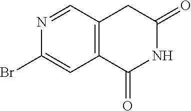 [SMILES: O=C1Cc2cnc(Br)cc2C(=O)N1] |
| I-14 | 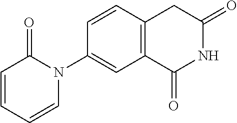 [SMILES: O=C1Cc2ccc(-n3ccccc3=O)cc2C(=O)N1] |
| I-15 | 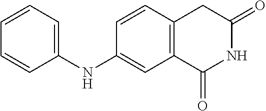 [SMILES: O=C1Cc2ccc(Nc3ccccc3)cc2C(=O)N1] |
| I-16 | 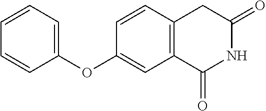 [SMILES: O=C1Cc2ccc(Oc3ccccc3)cc2C(=O)N1] |
| I-17 | 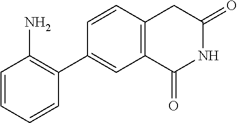 [SMILES: Nc1ccccc1-c1ccc2c(c1)C(=O)NC(=O)C2] |
| I-18 | 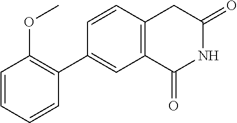 [SMILES: COc1ccccc1-c1ccc2c(c1)C(=O)NC(=O)C2] |
| I-19 | 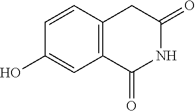 [SMILES: O=C1Cc2ccc(O)cc2C(=O)N1] |
| I-20 | 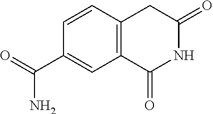 [SMILES: NC(=O)c1ccc2c(c1)C(=O)NC(=O)C2] |
| I-21 | 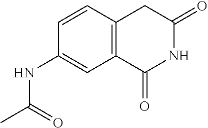 [SMILES: CC(=O)Nc1ccc2c(c1)C(=O)NC(=O)C2] |
| I-22 | 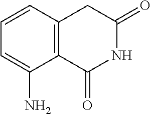 [SMILES: Nc1cccc2c1C(=O)NC(=O)C2] |
| I-23 | 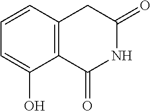 [SMILES: O=C1Cc2cccc(O)c2C(=O)N1] |
| I-24 | 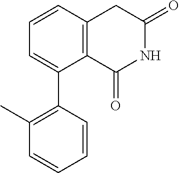 [SMILES: Cc1ccccc1-c1cccc2c1C(=O)NC(=O)C2] |
| I-25 | 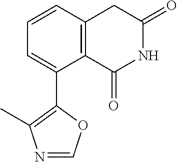 [SMILES: Cc1ncoc1-c1cccc2c1C(=O)NC(=O)C2] |
| I-26 | 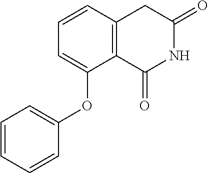 [SMILES: O=C1Cc2cccc(Oc3ccccc3)c2C(=O)N1] |
| I-27 | 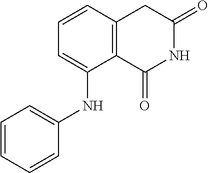 [SMILES: O=C1Cc2cccc(Nc3ccccc3)c2C(=O)N1] |
| I-28 | 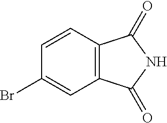 [SMILES: O=C1NC(=O)c2cc(Br)ccc21] |
| I-29 | 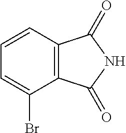 [SMILES: O=C1NC(=O)c2c(Br)cccc21] |
| I-30 | 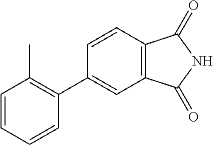 [SMILES: Cc1ccccc1-c1ccc2c(c1)C(=O)NC2=O] |
| I-31 | 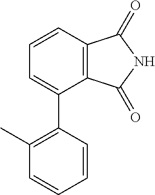 [SMILES: Cc1ccccc1-c1cccc2c1C(=O)NC2=O] |
| I-32 | 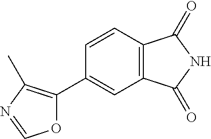 [SMILES: Cc1ncoc1-c1ccc2c(c1)C(=O)NC2=O] |
| I-33 | 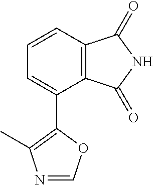 [SMILES: Cc1ncoc1-c1cccc2c1C(=O)NC2=O] |
| I-34 | 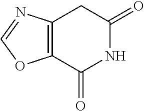 [SMILES: O=C1Cc2ncoc2C(=O)N1] |
| I-35 | 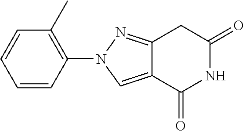 [SMILES: Cc1ccccc1-n1cc2c(n1)CC(=O)NC2=O] |
| I-36 | 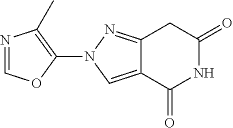 [SMILES: Cc1ncoc1-n1cc2c(n1)CC(=O)NC2=O] |
| I-37 | 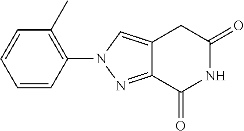 [SMILES: Cc1ccccc1-n1cc2c(n1)C(=O)NC(=O)C2] |
| I-38 | 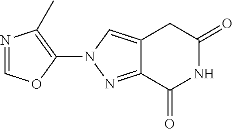 [SMILES: Cc1ncoc1-n1cc2c(n1)C(=O)NC(=O)C2] |
| I-39 | 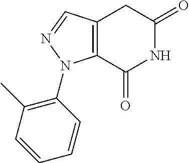 [SMILES: Cc1ccccc1-n1ncc2c1C(=O)NC(=O)C2] |
| I-40 | 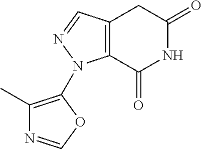 [SMILES: Cc1ncoc1-n1ncc2c1C(=O)NC(=O)C2] |
| I-41 | 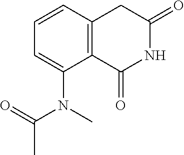 [SMILES: CC(=O)N(C)c1cccc2c1C(=O)NC(=O)C2] |
| I-42 | 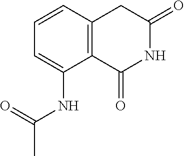 [SMILES: CC(=O)Nc1cccc2c1C(=O)NC(=O)C2] |
| I-43 | 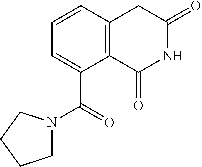 [SMILES: O=C1Cc2cccc(C(=O)N3CCCC3)c2C(=O)N1] |
| I-44 | 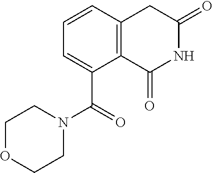 [SMILES: O=C1Cc2cccc(C(=O)N3CCOCC3)c2C(=O)N1] |
| I-45 | 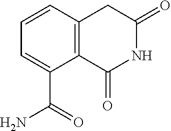 [SMILES: NC(=O)c1cccc2c1C(=O)NC(=O)C2] |
| I-46 | 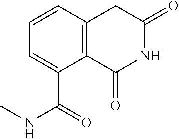 [SMILES: CNC(=O)c1cccc2c1C(=O)NC(=O)C2] |
| I-47 | 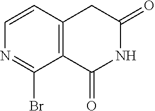 [SMILES: O=C1Cc2ccnc(Br)c2C(=O)N1] |
| I-48 | 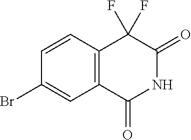 [SMILES: O=C1NC(=O)C(F)(F)c2ccc(Br)cc21] |
| I-49 | 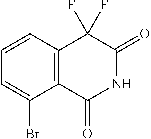 [SMILES: O=C1NC(=O)C(F)(F)c2cccc(Br)c21] |
| I-50 | 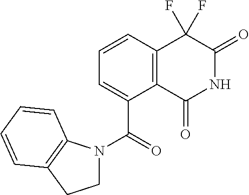 [SMILES: O=C1NC(=O)C(F)(F)c2cccc(C(=O)N3CCc4ccccc43)c21] |
| I-51 | 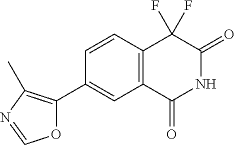 [SMILES: Cc1ncoc1-c1ccc2c(c1)C(=O)NC(=O)C2(F)F] |
| I-52 | 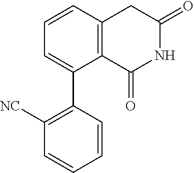 [SMILES: N#Cc1ccccc1-c1cccc2c1C(=O)NC(=O)C2] |
| I-53 | 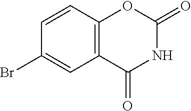 [SMILES: O=c1[nH]c(=O)c2cc(Br)ccc2o1] |
| I-54 | 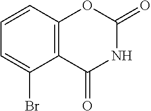 [SMILES: O=c1[nH]c(=O)c2c(Br)cccc2o1] |
| I-55 | 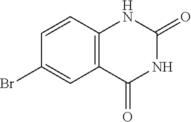 [SMILES: O=c1[nH]c(=O)c2cc(Br)ccc2[nH]1] |
| I-56 | 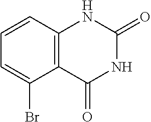 [SMILES: O=c1[nH]c(=O)c2c(Br)cccc2[nH]1] |
| I-57 | 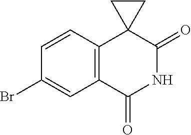 [SMILES: O=C1NC(=O)C2(CC2)c2ccc(Br)cc21] |
| I-58 | 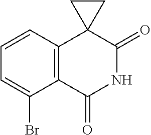 [SMILES: O=C1NC(=O)C2(CC2)c2cccc(Br)c21] |
| I-59 | 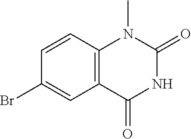 |
| I-60 | 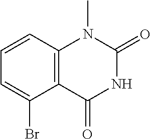 |
| I-61 | 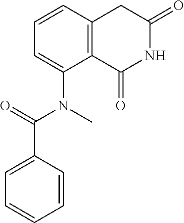 [SMILES: CN(C(=O)c1ccccc1)c1cccc2c1C(=O)NC(=O)C2] |
| I-62 | 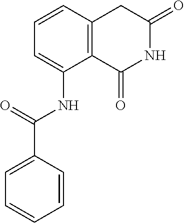 [SMILES: O=C1Cc2cccc(NC(=O)c3ccccc3)c2C(=O)N1] |
| I-63 | 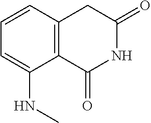 |
| I-64 | 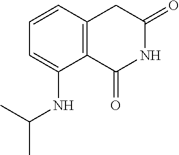 [SMILES: CC(C)Nc1cccc2c1C(=O)NC(=O)C2] |
| I-65 | 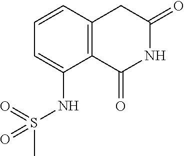 [SMILES: CS(=O)(=O)Nc1cccc2c1C(=O)NC(=O)C2] |
| I-66 | 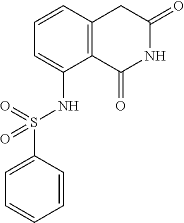 [SMILES: O=C1Cc2cccc(NS(=O)(=O)c3ccccc3)c2C(=O)N1] |
| I-67 | 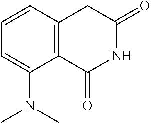 |
| I-68 | 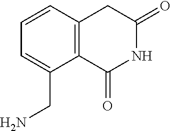 [SMILES: NCc1cccc2c1C(=O)NC(=O)C2] |
| I-69 | 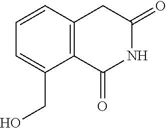 [SMILES: O=C1Cc2cccc(CO)c2C(=O)N1] |
| I-70 | 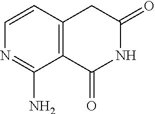 [SMILES: Nc1nccc2c1C(=O)NC(=O)C2] |
| I-71 | 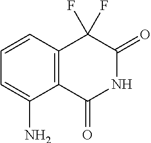 [SMILES: Nc1cccc2c1C(=O)NC(=O)C2(F)F] |
| I-72 | 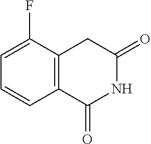 [SMILES: O=C1Cc2c(F)cccc2C(=O)N1] |
| I-73 | 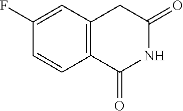 [SMILES: O=C1Cc2cc(F)ccc2C(=O)N1] |
| I-74 | 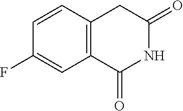 [SMILES: O=C1Cc2ccc(F)cc2C(=O)N1] |
| I-75 | 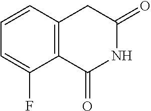 [SMILES: O=C1Cc2cccc(F)c2C(=O)N1] |
| I-76 | 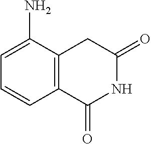 [SMILES: Nc1cccc2c1CC(=O)NC2=O] |
| I-77 | 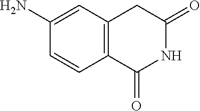 [SMILES: Nc1ccc2c(c1)CC(=O)NC2=O] |
| I-78 | 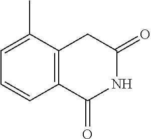 |
| I-79 | 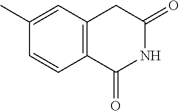 |
| I-80 | 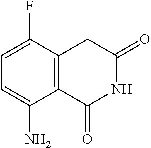 [SMILES: Nc1ccc(F)c2c1C(=O)NC(=O)C2] |
| I-81 | 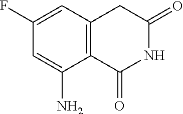 [SMILES: Nc1cc(F)cc2c1C(=O)NC(=O)C2] |
| I-82 | 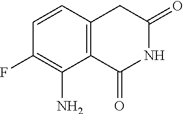 [SMILES: Nc1c(F)ccc2c1C(=O)NC(=O)C2] |
| I-83 | 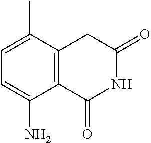 |
| I-84 | 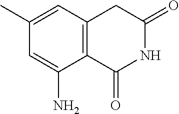 |
| I-85 | 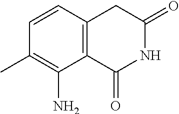 |
| I-86 | 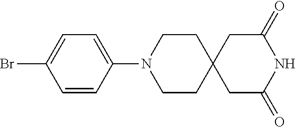 [SMILES: O=C1CC2(CCN(c3ccc(Br)cc3)CC2)CC(=O)N1] |
| I-87 | 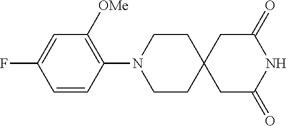 [SMILES: COc1cc(F)ccc1N1CCC2(CC1)CC(=O)NC(=O)C2] |
| I-88 | 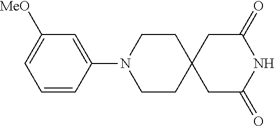 [SMILES: COc1cccc(N2CCC3(CC2)CC(=O)NC(=O)C3)c1] |
| I-89 | 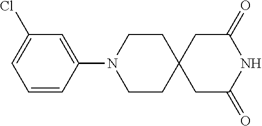 [SMILES: O=C1CC2(CCN(c3cccc(Cl)c3)CC2)CC(=O)N1] |
| I-90 | 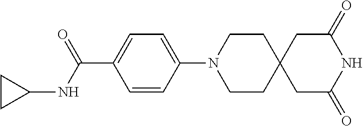 [SMILES: O=C1CC2(CCN(c3ccc(C(=O)NC4CC4)cc3)CC2)CC(=O)N1] |
| I-91 | 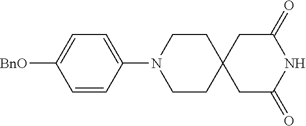 [SMILES: O=C1CC2(CCN(c3ccc(OCc4ccccc4)cc3)CC2)CC(=O)N1] |
| I-92 | 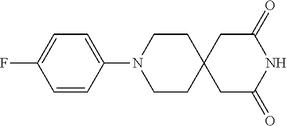 [SMILES: O=C1CC2(CCN(c3ccc(F)cc3)CC2)CC(=O)N1] |
| I-93 | 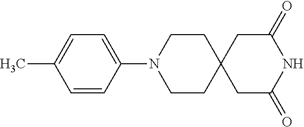 [SMILES: Cc1ccc(N2CCC3(CC2)CC(=O)NC(=O)C3)cc1] |
| I-94 | 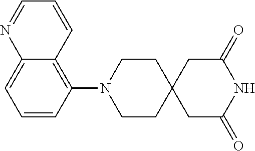 [SMILES: O=C1CC2(CCN(c3cccc4ncccc34)CC2)CC(=O)N1] |
| I-95 | 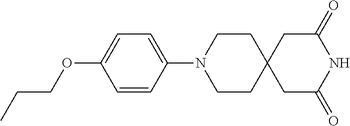 [SMILES: CCCOc1ccc(N2CCC3(CC2)CC(=O)NC(=O)C3)cc1] |
| I-96 | 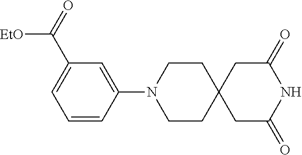 [SMILES: CCOC(=O)c1cccc(N2CCC3(CC2)CC(=O)NC(=O)C3)c1] |
| I-97 | 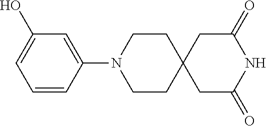 [SMILES: O=C1CC2(CCN(c3cccc(O)c3)CC2)CC(=O)N1] |
| I-98 | 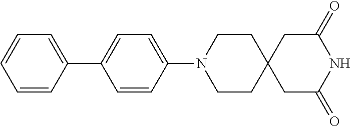 [SMILES: O=C1CC2(CCN(c3ccc(-c4ccccc4)cc3)CC2)CC(=O)N1] |
| I-99 | 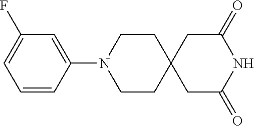 [SMILES: O=C1CC2(CCN(c3cccc(F)c3)CC2)CC(=O)N1] |
| I-100 | 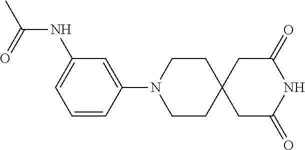 [SMILES: CC(=O)Nc1cccc(N2CCC3(CC2)CC(=O)NC(=O)C3)c1] |
| I-101 | 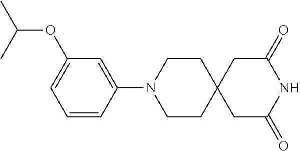 [SMILES: CC(C)Oc1cccc(N2CCC3(CC2)CC(=O)NC(=O)C3)c1] |
| I-102 | 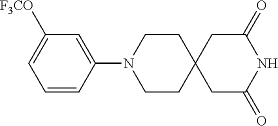 [SMILES: O=C1CC2(CCN(c3cccc(OC(F)(F)F)c3)CC2)CC(=O)N1] |
| I-103 | 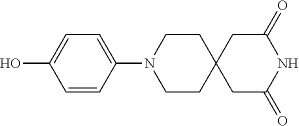 [SMILES: O=C1CC2(CCN(c3ccc(O)cc3)CC2)CC(=O)N1] |
| I-104 | 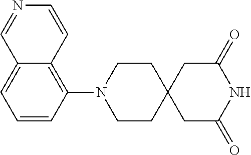 [SMILES: O=C1CC2(CCN(c3cccc4cnccc34)CC2)CC(=O)N1] |
| I-105 | 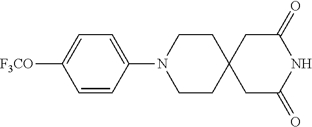 [SMILES: O=C1CC2(CCN(c3ccc(OC(F)(F)F)cc3)CC2)CC(=O)N1] |
| I-106 | 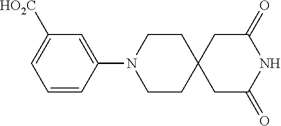 [SMILES: O=C1CC2(CCN(c3cccc(C(=O)O)c3)CC2)CC(=O)N1] |
| I-107 | 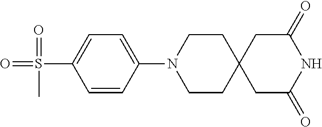 [SMILES: CS(=O)(=O)c1ccc(N2CCC3(CC2)CC(=O)NC(=O)C3)cc1] |
| I-108 | 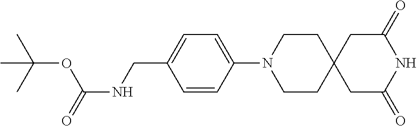 [SMILES: CC(C)(C)OC(=O)NCc1ccc(N2CCC3(CC2)CC(=O)NC(=O)C3)cc1] |
| I-109 | 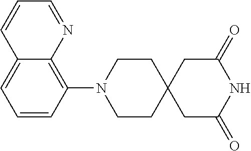 [SMILES: O=C1CC2(CCN(c3cccc4cccnc34)CC2)CC(=O)N1] |
| I-110 | 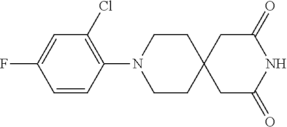 [SMILES: O=C1CC2(CCN(c3ccc(F)cc3Cl)CC2)CC(=O)N1] |
| I-111 | 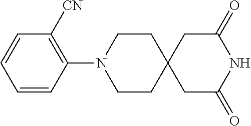 [SMILES: N#Cc1ccccc1N1CCC2(CC1)CC(=O)NC(=O)C2] |
| I-112 | 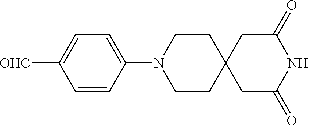 [SMILES: O=Cc1ccc(N2CCC3(CC2)CC(=O)NC(=O)C3)cc1] |
| I-113 | 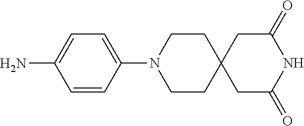 [SMILES: Nc1ccc(N2CCC3(CC2)CC(=O)NC(=O)C3)cc1] |
| I-114 | 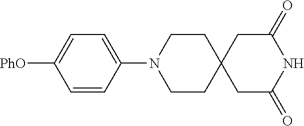 [SMILES: O=C1CC2(CCN(c3ccc(Oc4ccccc4)cc3)CC2)CC(=O)N1] |
| I-115 | 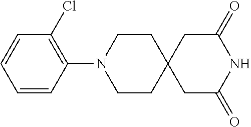 [SMILES: O=C1CC2(CCN(c3ccccc3Cl)CC2)CC(=O)N1] |
| I-116 | 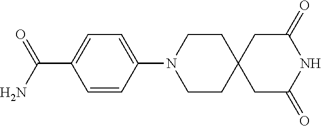 [SMILES: NC(=O)c1ccc(N2CCC3(CC2)CC(=O)NC(=O)C3)cc1] |
| I-117 | 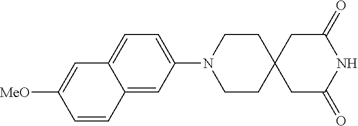 [SMILES: COc1ccc2cc(N3CCC4(CC3)CC(=O)NC(=O)C4)ccc2c1] |
| I-118 | 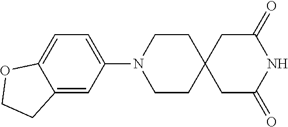 [SMILES: O=C1CC2(CCN(c3ccc4c(c3)CCO4)CC2)CC(=O)N1] |
| I-119 | 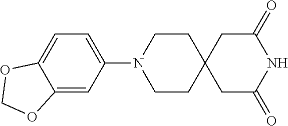 [SMILES: O=C1CC2(CCN(c3ccc4c(c3)OCO4)CC2)CC(=O)N1] |
| I-120 | 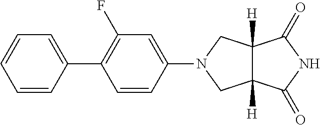 [SMILES: O=C1NC(=O)[C@@H]2CN(c3ccc(-c4ccccc4)c(F)c3)C[C@H]12] |
| I-121 | 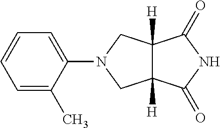 [SMILES: Cc1ccccc1N1C[C@@H]2C(=O)NC(=O)[C@@H]2C1] |
| I-122 | 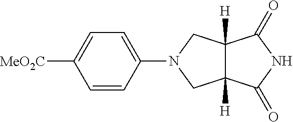 [SMILES: COC(=O)c1ccc(N2C[C@@H]3C(=O)NC(=O)[C@@H]3C2)cc1] |
| I-123 | 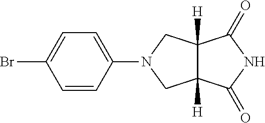 [SMILES: O=C1NC(=O)[C@@H]2CN(c3ccc(Br)cc3)C[C@H]12] |
| I-124 | 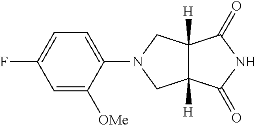 [SMILES: COc1cc(F)ccc1N1C[C@@H]2C(=O)NC(=O)[C@@H]2C1] |
| I-125 | 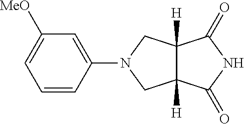 [SMILES: COc1cccc(N2C[C@@H]3C(=O)NC(=O)[C@@H]3C2)c1] |
| I-126 | 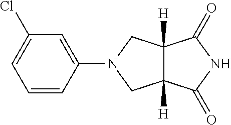 [SMILES: O=C1NC(=O)[C@@H]2CN(c3cccc(Cl)c3)C[C@H]12] |
| I-127 | 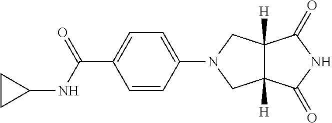 [SMILES: O=C(NC1CC1)c1ccc(N2C[C@@H]3C(=O)NC(=O)[C@@H]3C2)cc1] |
| I-128 | 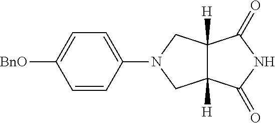 [SMILES: O=C1NC(=O)[C@@H]2CN(c3ccc(OCc4ccccc4)cc3)C[C@H]12] |
| I-129 | 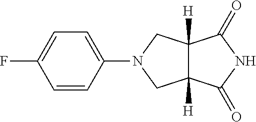 [SMILES: O=C1NC(=O)[C@@H]2CN(c3ccc(F)cc3)C[C@H]12] |
| I-130 | 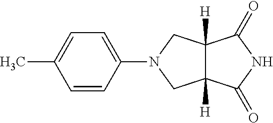 [SMILES: Cc1ccc(N2C[C@@H]3C(=O)NC(=O)[C@@H]3C2)cc1] |
| I-131 | 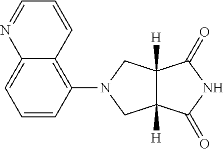 [SMILES: O=C1NC(=O)[C@@H]2CN(c3cccc4ncccc34)C[C@H]12] |
| I-132 | 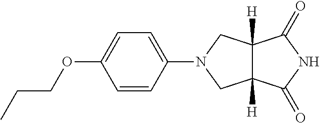 [SMILES: CCCOc1ccc(N2C[C@@H]3C(=O)NC(=O)[C@@H]3C2)cc1] |
| I-133 | 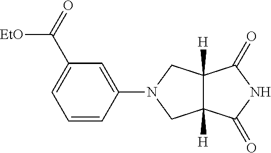 [SMILES: CCOC(=O)c1cccc(N2C[C@@H]3C(=O)NC(=O)[C@@H]3C2)c1] |
| I-134 | 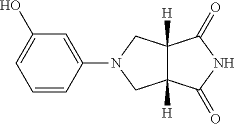 [SMILES: O=C1NC(=O)[C@@H]2CN(c3cccc(O)c3)C[C@H]12] |
| I-135 | 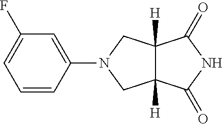 [SMILES: O=C1NC(=O)[C@@H]2CN(c3cccc(F)c3)C[C@H]12] |
| I-136 | 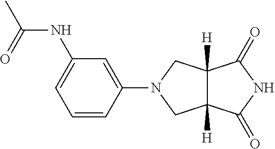 [SMILES: CC(=O)Nc1cccc(N2C[C@@H]3C(=O)NC(=O)[C@@H]3C2)c1] |
| I-137 | 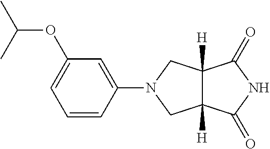 [SMILES: CC(C)Oc1cccc(N2C[C@@H]3C(=O)NC(=O)[C@@H]3C2)c1] |
| I-138 | 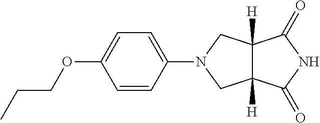 [SMILES: CCCOc1ccc(N2C[C@@H]3C(=O)NC(=O)[C@@H]3C2)cc1] |
| I-139 | 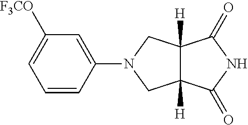 [SMILES: O=C1NC(=O)[C@@H]2CN(c3cccc(OC(F)(F)F)c3)C[C@H]12] |
| I-140 | 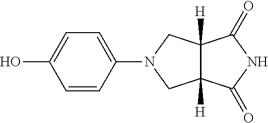 [SMILES: O=C1NC(=O)[C@@H]2CN(c3ccc(O)cc3)C[C@H]12] |
| I-141 | 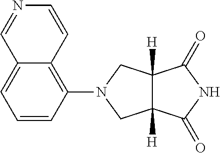 [SMILES: O=C1NC(=O)[C@@H]2CN(c3cccc4cnccc34)C[C@H]12] |
| I-142 | 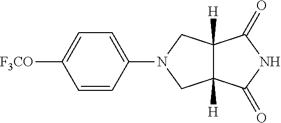 [SMILES: O=C1NC(=O)[C@@H]2CN(c3ccc(OC(F)(F)F)cc3)C[C@H]12] |
| I-143 | 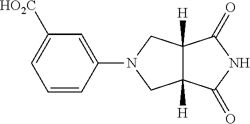 [SMILES: O=C(O)c1cccc(N2C[C@@H]3C(=O)NC(=O)[C@@H]3C2)c1] |
| I-144 | 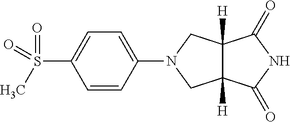 [SMILES: CS(=O)(=O)c1ccc(N2C[C@@H]3C(=O)NC(=O)[C@@H]3C2)cc1] |
| I-145 | 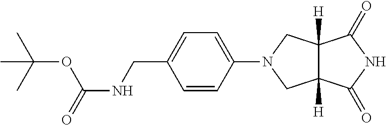 [SMILES: CC(C)(C)OC(=O)NCc1ccc(N2C[C@@H]3C(=O)NC(=O)[C@@H]3C2)cc1] |
| I-146 | 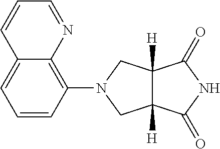 [SMILES: O=C1NC(=O)[C@@H]2CN(c3cccc4cccnc34)C[C@H]12] |
| I-147 | 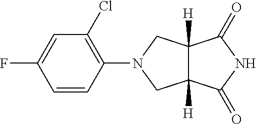 [SMILES: O=C1NC(=O)[C@@H]2CN(c3ccc(F)cc3Cl)C[C@H]12] |
| I-148 | 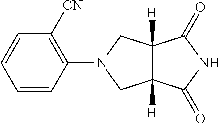 [SMILES: N#Cc1ccccc1N1C[C@@H]2C(=O)NC(=O)[C@@H]2C1] |
| I-149 | 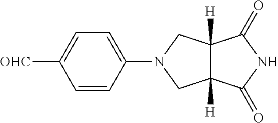 [SMILES: O=Cc1ccc(N2C[C@@H]3C(=O)NC(=O)[C@@H]3C2)cc1] |
| I-150 | 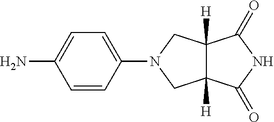 [SMILES: Nc1ccc(N2C[C@@H]3C(=O)NC(=O)[C@@H]3C2)cc1] |
| I-151 | 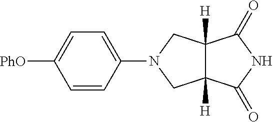 [SMILES: O=C1NC(=O)[C@@H]2CN(c3ccc(Oc4ccccc4)cc3)C[C@H]12] |
| I-152 | 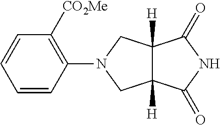 [SMILES: COC(=O)c1ccccc1N1C[C@@H]2C(=O)NC(=O)[C@@H]2C1] |
| I-153 | 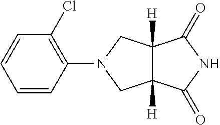 [SMILES: O=C1NC(=O)[C@@H]2CN(c3ccccc3Cl)C[C@H]12] |
| I-154 | 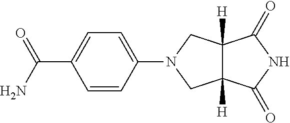 [SMILES: NC(=O)c1ccc(N2C[C@@H]3C(=O)NC(=O)[C@@H]3C2)cc1] |
| I-155 | 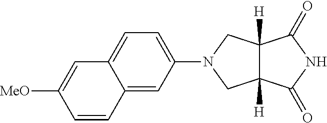 [SMILES: COc1ccc2cc(N3C[C@@H]4C(=O)NC(=O)[C@@H]4C3)ccc2c1] |
| I-156 | 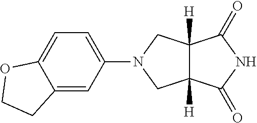 [SMILES: O=C1NC(=O)[C@@H]2CN(c3ccc4c(c3)CCO4)C[C@H]12] |
| I-157 | 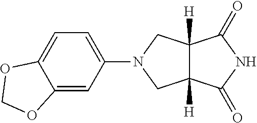 [SMILES: O=C1NC(=O)[C@@H]2CN(c3ccc4c(c3)OCO4)C[C@H]12] |
| I-158 | 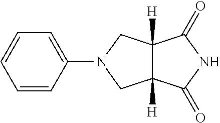 [SMILES: O=C1NC(=O)[C@@H]2CN(c3ccccc3)C[C@H]12] |
| I-159 | 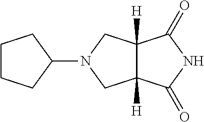 [SMILES: O=C1NC(=O)[C@@H]2CN(C3CCCC3)C[C@H]12] |
| I-160 | 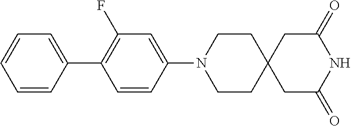 [SMILES: O=C1CC2(CCN(c3ccc(-c4ccccc4)c(F)c3)CC2)CC(=O)N1] |
| I-161 | 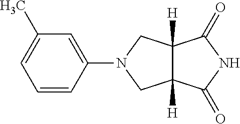 [SMILES: Cc1cccc(N2C[C@@H]3C(=O)NC(=O)[C@@H]3C2)c1] |
| I-162 | 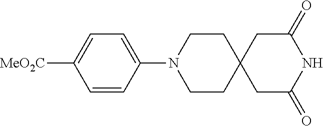 [SMILES: COC(=O)c1ccc(N2CCC3(CC2)CC(=O)NC(=O)C3)cc1] |
| I-163 | 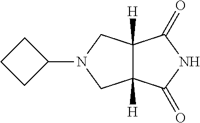 [SMILES: O=C1NC(=O)[C@@H]2CN(C3CCC3)C[C@H]12] |
| I-164 | 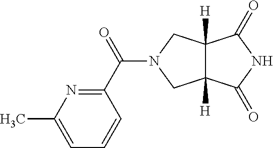 [SMILES: Cc1cccc(C(=O)N2C[C@@H]3C(=O)NC(=O)[C@@H]3C2)n1] |
| I-165 | 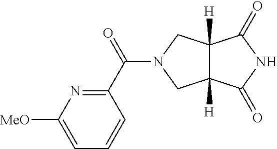 [SMILES: COc1cccc(C(=O)N2C[C@@H]3C(=O)NC(=O)[C@@H]3C2)n1] |
| I-166 | 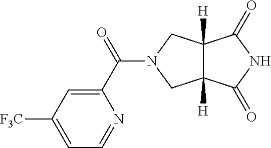 [SMILES: O=C1NC(=O)[C@@H]2CN(C(=O)c3cc(C(F)(F)F)ccn3)C[C@H]12] |
| I-167 | 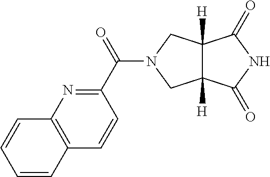 [SMILES: O=C1NC(=O)[C@@H]2CN(C(=O)c3ccc4ccccc4n3)C[C@H]12] |
| I-168 | 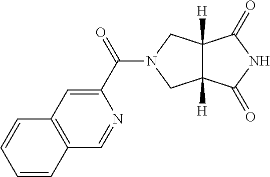 [SMILES: O=C1NC(=O)[C@@H]2CN(C(=O)c3cc4ccccc4cn3)C[C@H]12] |
| I-169 | 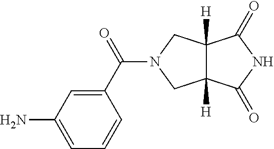 [SMILES: Nc1cccc(C(=O)N2C[C@@H]3C(=O)NC(=O)[C@@H]3C2)c1] |
| I-170 | 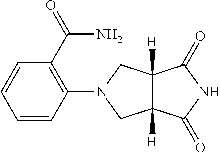 [SMILES: NC(=O)c1ccccc1N1C[C@@H]2C(=O)NC(=O)[C@@H]2C1] |
| I-171 | 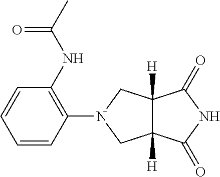 [SMILES: CC(=O)Nc1ccccc1N1C[C@@H]2C(=O)NC(=O)[C@@H]2C1] |
| I-172 | 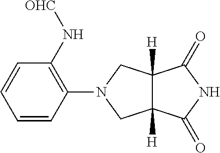 [SMILES: O=CNc1ccccc1N1C[C@@H]2C(=O)NC(=O)[C@@H]2C1] |
| I-173 | 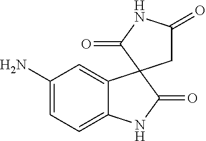 [SMILES: Nc1ccc2c(c1)C1(CC(=O)NC1=O)C(=O)N2] |
| I-174 | 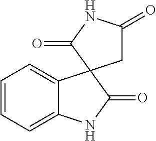 [SMILES: O=C1CC2(C(=O)N1)C(=O)Nc1ccccc12] |
| I-175 | 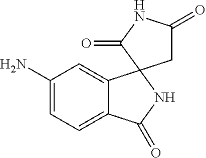 [SMILES: Nc1ccc2c(c1)C1(CC(=O)NC1=O)NC2=O] |
| I-176 | 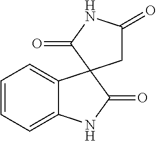 [SMILES: O=C1CC2(C(=O)N1)C(=O)Nc1ccccc12] |
| I-177 | 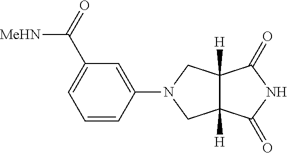 [SMILES: CNC(=O)c1cccc(N2C[C@@H]3C(=O)NC(=O)[C@@H]3C2)c1] |
| I-178 | 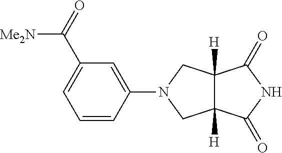 [SMILES: CN(C)C(=O)c1cccc(N2C[C@@H]3C(=O)NC(=O)[C@@H]3C2)c1] |
| I-179 | 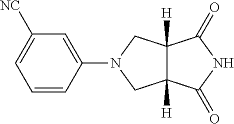 [SMILES: N#Cc1cccc(N2C[C@@H]3C(=O)NC(=O)[C@@H]3C2)c1] |
| I-180 | 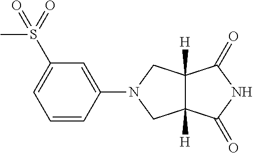 [SMILES: CS(=O)(=O)c1cccc(N2C[C@@H]3C(=O)NC(=O)[C@@H]3C2)c1] |
| I-181 | 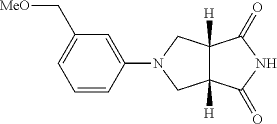 [SMILES: COCc1cccc(N2C[C@@H]3C(=O)NC(=O)[C@@H]3C2)c1] |
| I-182 | 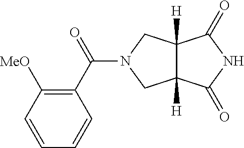 [SMILES: COc1ccccc1C(=O)N1C[C@@H]2C(=O)NC(=O)[C@@H]2C1] |
| I-183 | 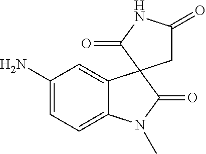 [SMILES: CN1C(=O)C2(CC(=O)NC2=O)c2cc(N)ccc21] |
| I-184 | 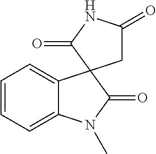 [SMILES: CN1C(=O)C2(CC(=O)NC2=O)c2ccccc21] |
| I-185 | 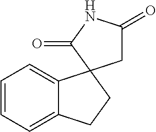 [SMILES: O=C1CC2(CCc3ccccc32)C(=O)N1] |
| I-186 | 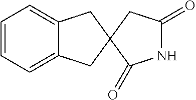 [SMILES: O=C1CC2(Cc3ccccc3C2)C(=O)N1] |
| I-187 | 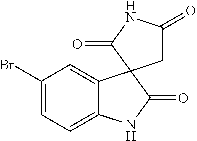 [SMILES: O=C1CC2(C(=O)N1)C(=O)Nc1ccc(Br)cc12] |
| I-188 | 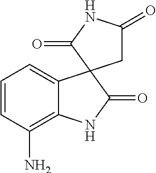 [SMILES: Nc1cccc2c1NC(=O)C21CC(=O)NC1=O] |
| I-189 | 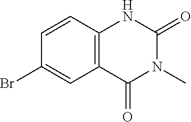 |
| I-190 | 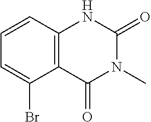 |
| I-191 | 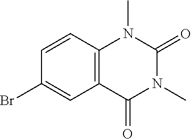 |
| I-192 | 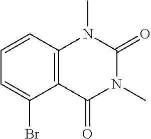 |
| I-193 | 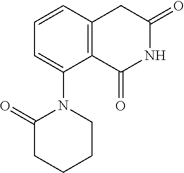 [SMILES: O=C1Cc2cccc(N3CCCCC3=O)c2C(=O)N1] |
| I-194 | 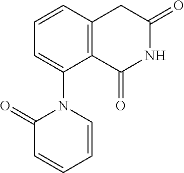 [SMILES: O=C1Cc2cccc(-n3ccccc3=O)c2C(=O)N1] |
| I-195 | 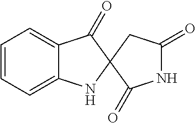 [SMILES: O=C1CC2(Nc3ccccc3C2=O)C(=O)N1] |
| I-196 | 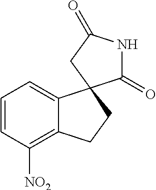 [SMILES: O=C1C[C@]2(CCc3c([N+](=O)[O-])cccc32)C(=O)N1] |
| I-197 | 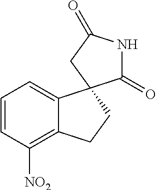 [SMILES: O=C1C[C@@]2(CCc3c([N+](=O)[O-])cccc32)C(=O)N1] |
| I-198 | 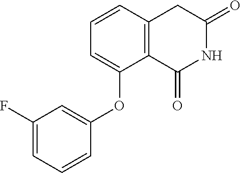 [SMILES: O=C1Cc2cccc(Oc3cccc(F)c3)c2C(=O)N1] |
| I-199 | 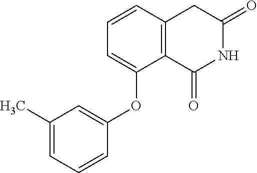 [SMILES: Cc1cccc(Oc2cccc3c2C(=O)NC(=O)C3)c1] |
| I-200 | 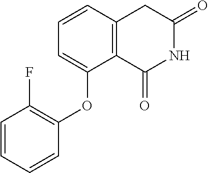 [SMILES: O=C1Cc2cccc(Oc3ccccc3F)c2C(=O)N1] |
| I-201 | 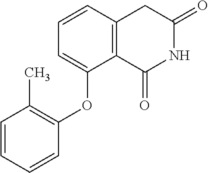 [SMILES: Cc1ccccc1Oc1cccc2c1C(=O)NC(=O)C2] |
| I-202 | 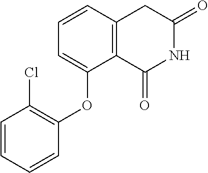 [SMILES: O=C1Cc2cccc(Oc3ccccc3Cl)c2C(=O)N1] |
| I-203 | 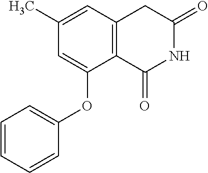 [SMILES: Cc1cc2c(c(Oc3ccccc3)c1)C(=O)NC(=O)C2] |
| I-204 | 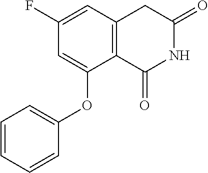 [SMILES: O=C1Cc2cc(F)cc(Oc3ccccc3)c2C(=O)N1] |
| I-205 | 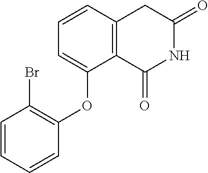 [SMILES: O=C1Cc2cccc(Oc3ccccc3Br)c2C(=O)N1] |
| I-206 | 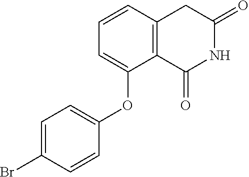 [SMILES: O=C1Cc2cccc(Oc3ccc(Br)cc3)c2C(=O)N1] |
| I-207 | 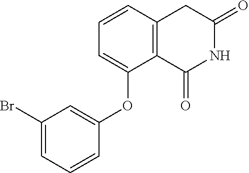 [SMILES: O=C1Cc2cccc(Oc3cccc(Br)c3)c2C(=O)N1] |
| I-208 | 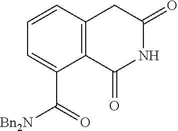 |
| I-209 | 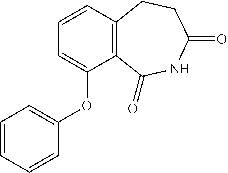 [SMILES: O=C1CCc2cccc(Oc3ccccc3)c2C(=O)N1] |
| I-210 | 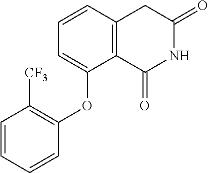 [SMILES: O=C1Cc2cccc(Oc3ccccc3C(F)(F)F)c2C(=O)N1] |
| I-211 | 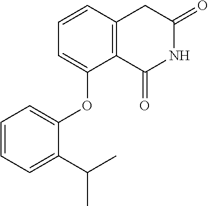 [SMILES: CC(C)c1ccccc1Oc1cccc2c1C(=O)NC(=O)C2] |
| I-212 | 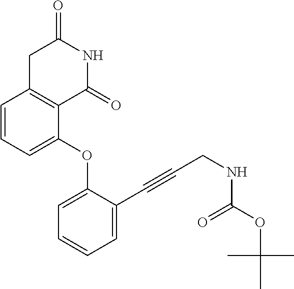 [SMILES: CC(C)(C)OC(=O)NCC#Cc1ccccc1Oc1cccc2c1C(=O)NC(=O)C2] |
| I-213 | 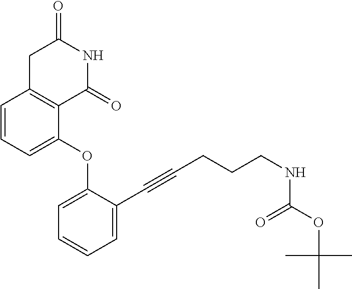 [SMILES: CC(C)(C)OC(=O)NCCCC#Cc1ccccc1Oc1cccc2c1C(=O)NC(=O)C2] |
| I-214 | 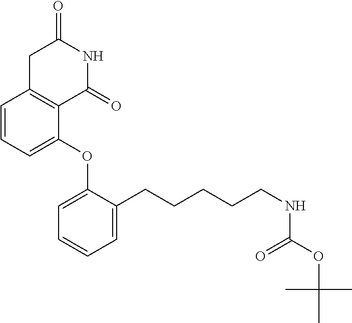 [SMILES: CC(C)(C)OC(=O)NCCCCCc1ccccc1Oc1cccc2c1C(=O)NC(=O)C2] |
| I-215 | 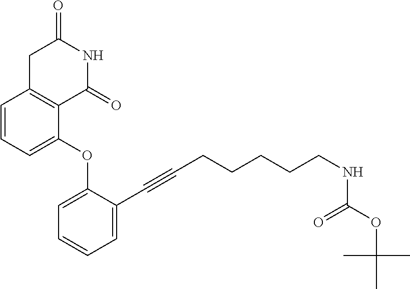 [SMILES: CC(C)(C)OC(=O)NCCCCCC#Cc1ccccc1Oc1cccc2c1C(=O)NC(=O)C2] |
| I-216 | 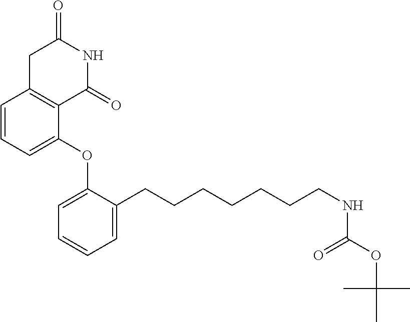 [SMILES: CC(C)(C)OC(=O)NCCCCCCCc1ccccc1Oc1cccc2c1C(=O)NC(=O)C2] |
| I-217 | 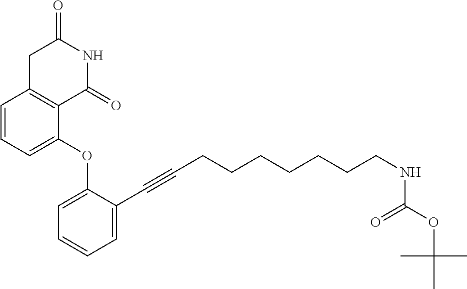 [SMILES: CC(C)(C)OC(=O)NCCCCCCCC#Cc1ccccc1Oc1cccc2c1C(=O)NC(=O)C2] |
| I-218 | 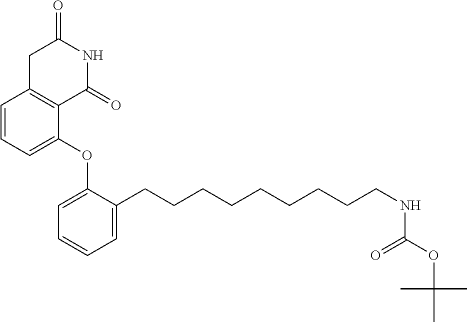 [SMILES: CC(C)(C)OC(=O)NCCCCCCCCCc1ccccc1Oc1cccc2c1C(=O)NC(=O)C2] |
| I-219 | 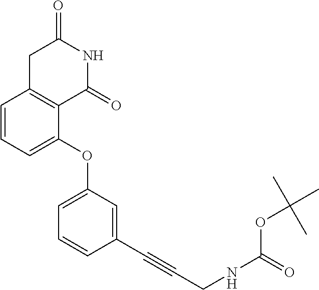 [SMILES: CC(C)(C)OC(=O)NCC#Cc1cccc(Oc2cccc3c2C(=O)NC(=O)C3)c1] |
| I-220 | 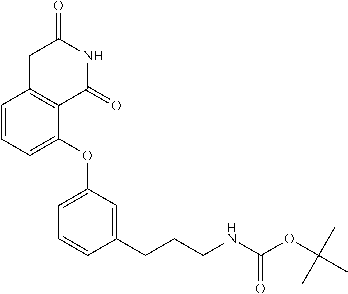 [SMILES: CC(C)(C)OC(=O)NCCCc1cccc(Oc2cccc3c2C(=O)NC(=O)C3)c1] |
| I-221 | 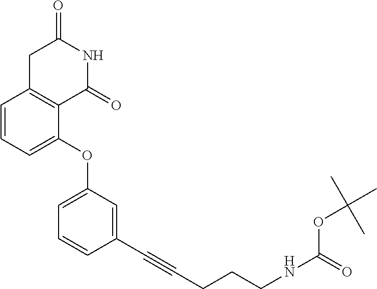 [SMILES: CC(C)(C)OC(=O)NCCCC#Cc1cccc(Oc2cccc3c2C(=O)NC(=O)C3)c1] |
| I-222 | 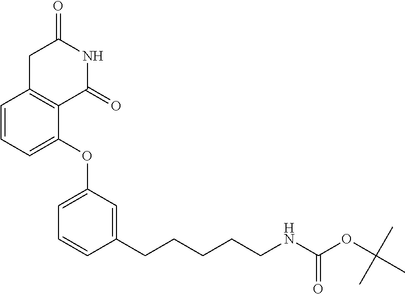 [SMILES: CC(C)(C)OC(=O)NCCCCCc1cccc(Oc2cccc3c2C(=O)NC(=O)C3)c1] |
| I-223 | 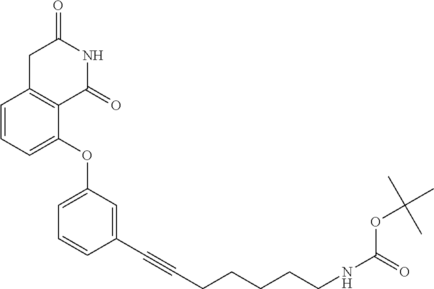 [SMILES: CC(C)(C)OC(=O)NCCCCCC#Cc1cccc(Oc2cccc3c2C(=O)NC(=O)C3)c1] |
| I-224 | 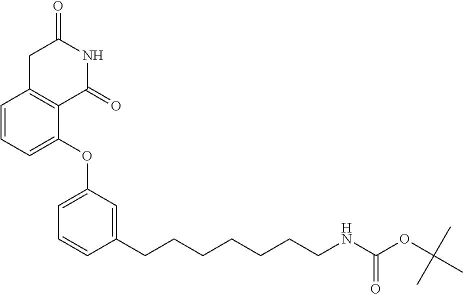 [SMILES: CC(C)(C)OC(=O)NCCCCCCCc1cccc(Oc2cccc3c2C(=O)NC(=O)C3)c1] |
| I-225 | 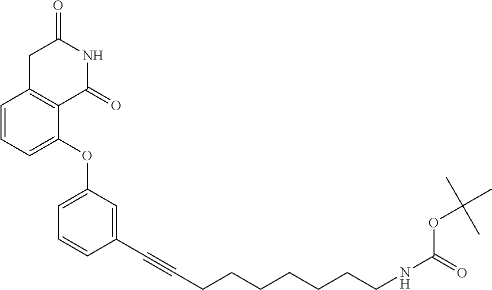 [SMILES: CC(C)(C)OC(=O)NCCCCCCCC#Cc1cccc(Oc2cccc3c2C(=O)NC(=O)C3)c1] |
| I-226 | 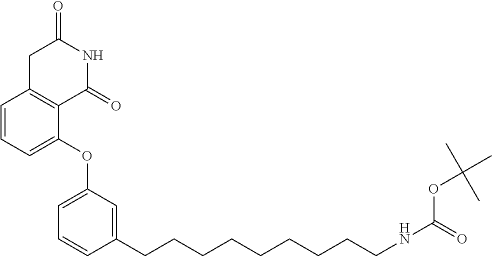 [SMILES: CC(C)(C)OC(=O)NCCCCCCCCCc1cccc(Oc2cccc3c2C(=O)NC(=O)C3)c1] |
| I-227 | 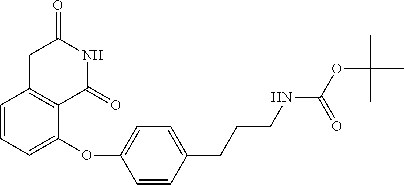 [SMILES: CC(C)(C)OC(=O)NCCCc1ccc(Oc2cccc3c2C(=O)NC(=O)C3)cc1] |
| I-228 | 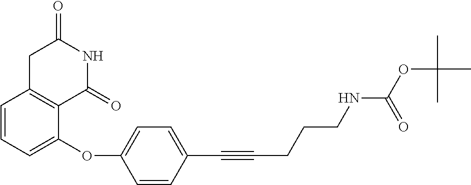 [SMILES: CC(C)(C)OC(=O)NCCCC#Cc1ccc(Oc2cccc3c2C(=O)NC(=O)C3)cc1] |
| I-229 | 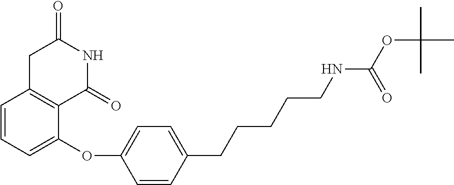 [SMILES: CC(C)(C)OC(=O)NCCCCCc1ccc(Oc2cccc3c2C(=O)NC(=O)C3)cc1] |
| I-230 | 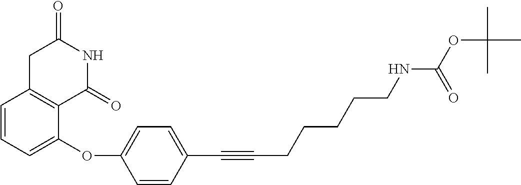 [SMILES: CC(C)(C)OC(=O)NCCCCCC#Cc1ccc(Oc2cccc3c2C(=O)NC(=O)C3)cc1] |
| I-231 | 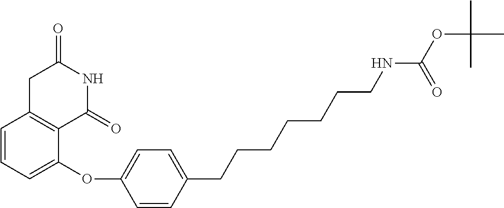 [SMILES: CC(C)(C)OC(=O)NCCCCCCCc1ccc(Oc2cccc3c2C(=O)NC(=O)C3)cc1] |
| I-232 | 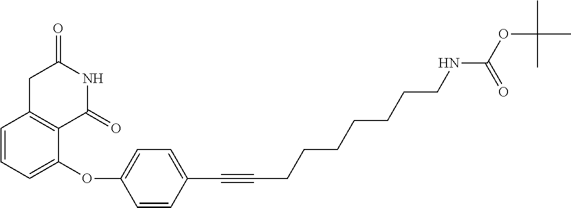 [SMILES: CC(C)(C)OC(=O)NCCCCCCCC#Cc1ccc(Oc2cccc3c2C(=O)NC(=O)C3)cc1] |
| I-233 | 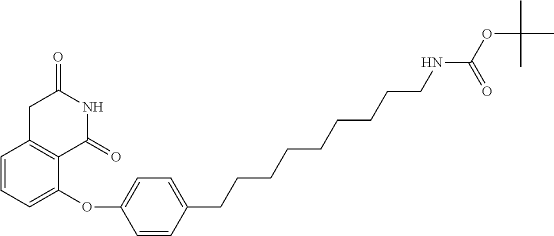 [SMILES: CC(C)(C)OC(=O)NCCCCCCCCCc1ccc(Oc2cccc3c2C(=O)NC(=O)C3)cc1] |
| I-234 | 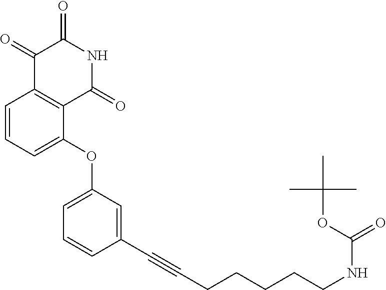 [SMILES: CC(C)(C)OC(=O)NCCCCCC#Cc1cccc(Oc2cccc3c2C(=O)NC(=O)C3=O)c1] |
| I-235 | 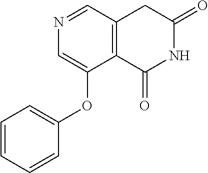 [SMILES: O=C1Cc2cncc(Oc3ccccc3)c2C(=O)N1] |
| I-236 | 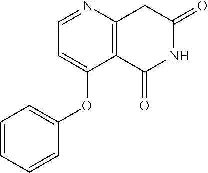 [SMILES: O=C1Cc2nccc(Oc3ccccc3)c2C(=O)N1] |
| I-237 | 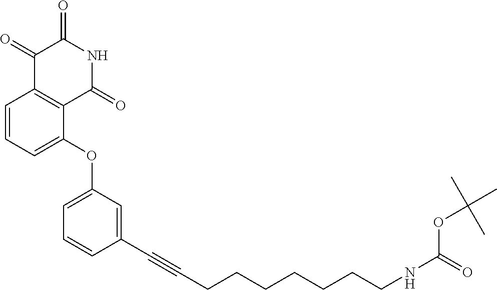 [SMILES: CC(C)(C)OC(=O)NCCCCCCCC#Cc1cccc(Oc2cccc3c2C(=O)NC(=O)C3=O)c1] |
| I-238 | 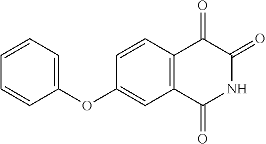 [SMILES: O=C1NC(=O)c2cc(Oc3ccccc3)ccc2C1=O] |
| I-239 | 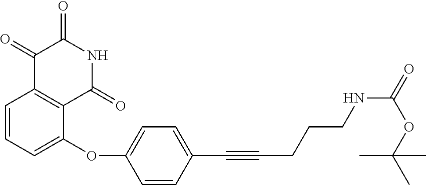 [SMILES: CC(C)(C)OC(=O)NCCCC#Cc1ccc(Oc2cccc3c2C(=O)NC(=O)C3=O)cc1] |
| I-240 | 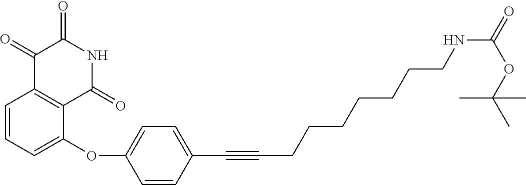 [SMILES: CC(C)(C)OC(=O)NCCCCCCCC#Cc1ccc(Oc2cccc3c2C(=O)NC(=O)C3=O)cc1] |
| I-241 | 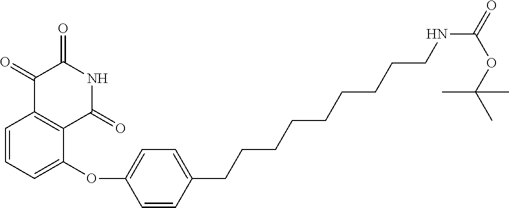 [SMILES: CC(C)(C)OC(=O)NCCCCCCCCCc1ccc(Oc2cccc3c2C(=O)NC(=O)C3=O)cc1] |
| I-242 | 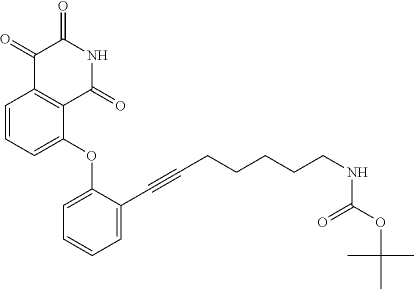 [SMILES: CC(C)(C)OC(=O)NCCCCCC#Cc1ccccc1Oc1cccc2c1C(=O)NC(=O)C2=O] |
| I-243 | 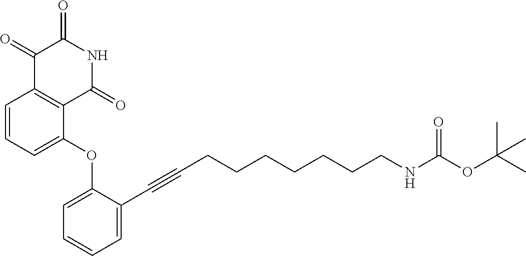 [SMILES: CC(C)(C)OC(=O)NCCCCCCCC#Cc1ccccc1Oc1cccc2c1C(=O)NC(=O)C2=O] |
| I-244 | 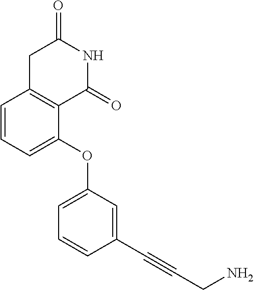 [SMILES: NCC#Cc1cccc(Oc2cccc3c2C(=O)NC(=O)C3)c1] |
| I-245 | 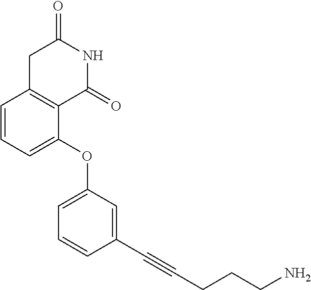 [SMILES: NCCCC#Cc1cccc(Oc2cccc3c2C(=O)NC(=O)C3)c1] |
| I-246 | 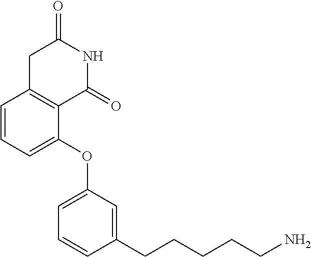 [SMILES: NCCCCCc1cccc(Oc2cccc3c2C(=O)NC(=O)C3)c1] |
| I-247 | 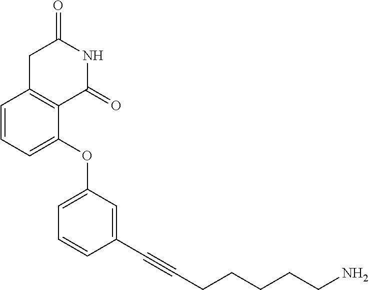 [SMILES: NCCCCCC#Cc1cccc(Oc2cccc3c2C(=O)NC(=O)C3)c1] |
| I-248 | 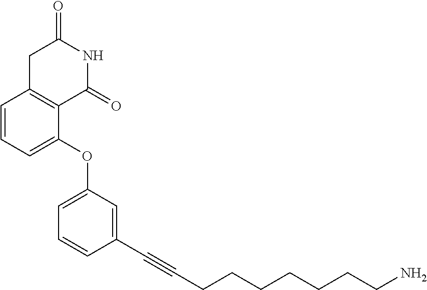 [SMILES: NCCCCCCCC#Cc1cccc(Oc2cccc3c2C(=O)NC(=O)C3)c1] |
| I-249 | 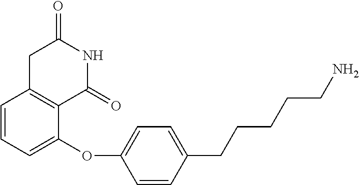 [SMILES: NCCCCCc1ccc(Oc2cccc3c2C(=O)NC(=O)C3)cc1] |
| I-250 | 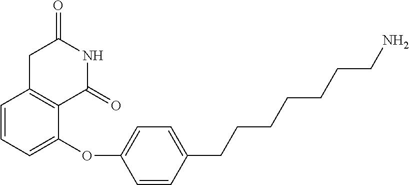 [SMILES: NCCCCCCCc1ccc(Oc2cccc3c2C(=O)NC(=O)C3)cc1] |
| I-251 | 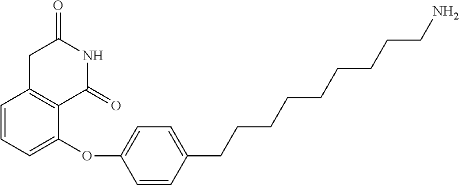 [SMILES: NCCCCCCCCCc1ccc(Oc2cccc3c2C(=O)NC(=O)C3)cc1] |
| I-252 | 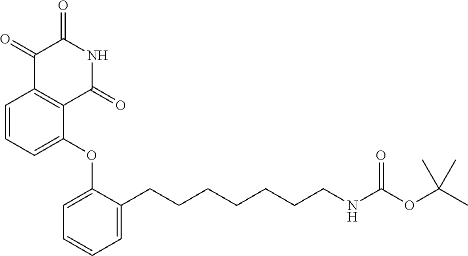 [SMILES: CC(C)(C)OC(=O)NCCCCCCCc1ccccc1Oc1cccc2c1C(=O)NC(=O)C2=O] |
| I-253 | 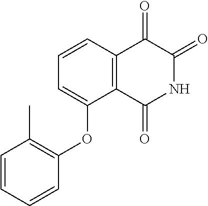 [SMILES: Cc1ccccc1Oc1cccc2c1C(=O)NC(=O)C2=O] |
| I-254 | 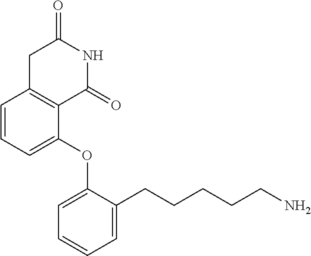 [SMILES: NCCCCCc1ccccc1Oc1cccc2c1C(=O)NC(=O)C2] |
| I-255 | 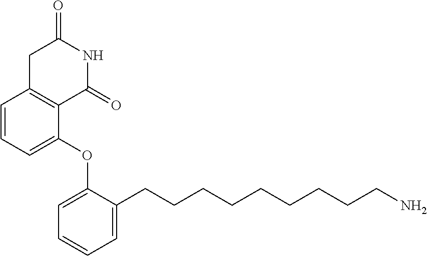 [SMILES: NCCCCCCCCCc1ccccc1Oc1cccc2c1C(=O)NC(=O)C2] |
| I-256 | 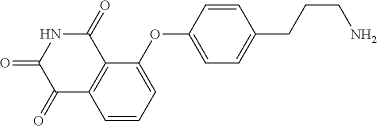 [SMILES: NCCCc1ccc(Oc2cccc3c2C(=O)NC(=O)C3=O)cc1] |
| I-257 | 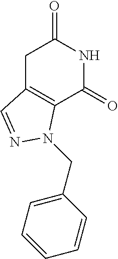 [SMILES: O=C1Cc2cnn(Cc3ccccc3)c2C(=O)N1] |
| I-258 | 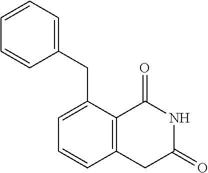 [SMILES: O=C1Cc2cccc(Cc3ccccc3)c2C(=O)N1] |
| I-259 | 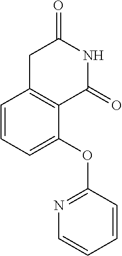 [SMILES: O=C1Cc2cccc(Oc3ccccn3)c2C(=O)N1] |
| I-260 | 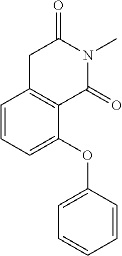 [SMILES: CN1C(=O)Cc2cccc(Oc3ccccc3)c2C1=O] |
| I-261 | 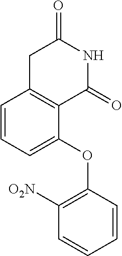 [SMILES: O=C1Cc2cccc(Oc3ccccc3[N+](=O)[O-])c2C(=O)N1] |
| I-262 | 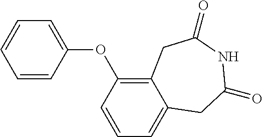 [SMILES: O=C1Cc2cccc(Oc3ccccc3)c2CC(=O)N1] |
| I-263 | 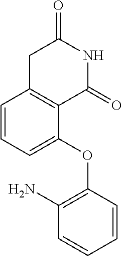 [SMILES: Nc1ccccc1Oc1cccc2c1C(=O)NC(=O)C2] |
| I-264 | 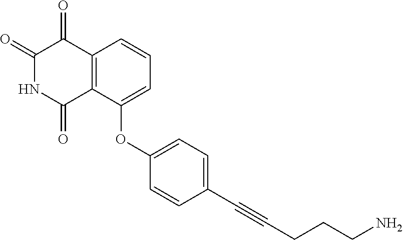 [SMILES: NCCCC#Cc1ccc(Oc2cccc3c2C(=O)NC(=O)C3=O)cc1] |
| I-265 | 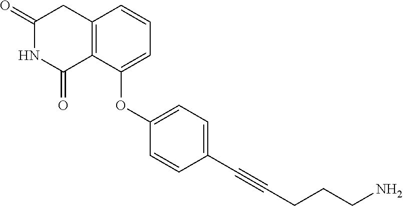 [SMILES: NCCCC#Cc1ccc(Oc2cccc3c2C(=O)NC(=O)C3)cc1] |
| I-266 | 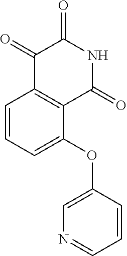 [SMILES: O=C1NC(=O)c2c(Oc3cccnc3)cccc2C1=O] |
| I-267 | 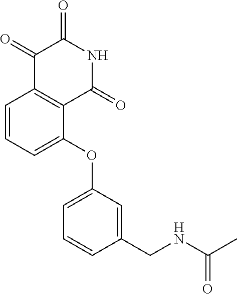 [SMILES: CC(=O)NCc1cccc(Oc2cccc3c2C(=O)NC(=O)C3=O)c1] |
| I-268 | 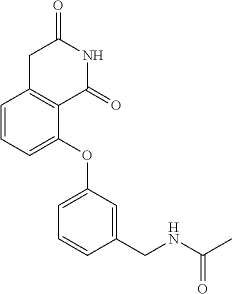 [SMILES: CC(=O)NCc1cccc(Oc2cccc3c2C(=O)NC(=O)C3)c1] |
| I-269 | 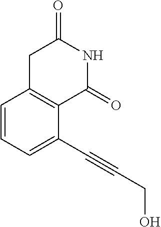 [SMILES: O=C1Cc2cccc(C#CCO)c2C(=O)N1] |
| I-270 | 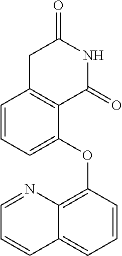 [SMILES: O=C1Cc2cccc(Oc3cccc4cccnc34)c2C(=O)N1] |
| I-271 | 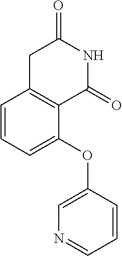 [SMILES: O=C1Cc2cccc(Oc3cccnc3)c2C(=O)N1] |
| I-272 | 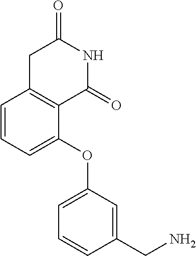 [SMILES: NCc1cccc(Oc2cccc3c2C(=O)NC(=O)C3)c1] |
| I-273 | 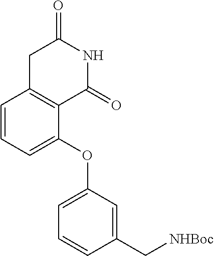 [SMILES: CC(C)(C)OC(=O)NCc1cccc(Oc2cccc3c2C(=O)NC(=O)C3)c1] |
| I-274 | 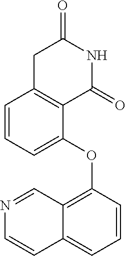 [SMILES: O=C1Cc2cccc(Oc3cccc4ccncc34)c2C(=O)N1] |
| I-275 | 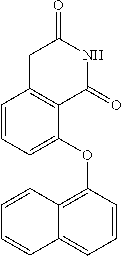 [SMILES: O=C1Cc2cccc(Oc3cccc4ccccc34)c2C(=O)N1] |
| I-276 | 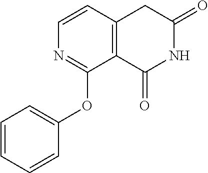 [SMILES: O=C1Cc2ccnc(Oc3ccccc3)c2C(=O)N1] |
| I-277 | 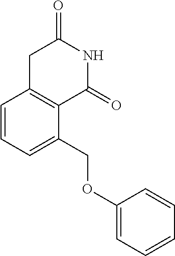 [SMILES: O=C1Cc2cccc(COc3ccccc3)c2C(=O)N1] |
| I-278 | 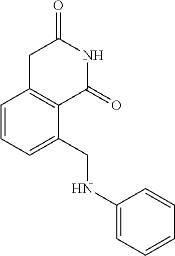 [SMILES: O=C1Cc2cccc(CNc3ccccc3)c2C(=O)N1] |
| I-279 | 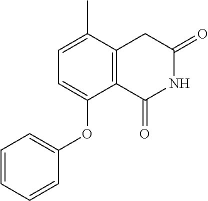 [SMILES: Cc1ccc(Oc2ccccc2)c2c1CC(=O)NC2=O] |
| I-280 | 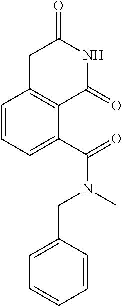 [SMILES: CN(Cc1ccccc1)C(=O)c1cccc2c1C(=O)NC(=O)C2] |
| I-281 | 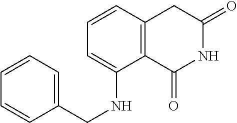 [SMILES: O=C1Cc2cccc(NCc3ccccc3)c2C(=O)N1] |
| I-282 | 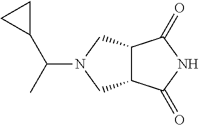 |
| I-283 | 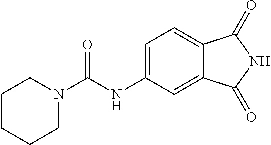 [SMILES: O=C1NC(=O)c2cc(NC(=O)N3CCCCC3)ccc21] |
| I-284 | 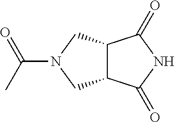 |
| I-285 | 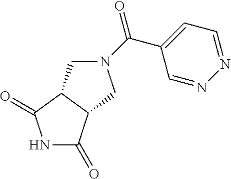 [SMILES: O=C1NC(=O)[C@@H]2CN(C(=O)c3ccnnc3)C[C@H]12] |
| I-286 | 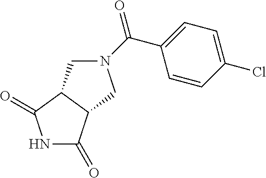 [SMILES: O=C1NC(=O)[C@@H]2CN(C(=O)c3ccc(Cl)cc3)C[C@H]12] |
| I-287 | 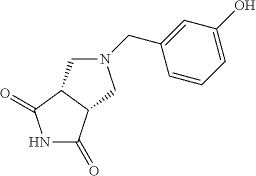 [SMILES: O=C1NC(=O)[C@@H]2CN(Cc3cccc(O)c3)C[C@H]12] |
| I-288 | 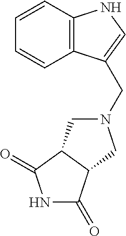 [SMILES: O=C1NC(=O)[C@@H]2CN(Cc3c[nH]c4ccccc34)C[C@H]12] |
| I-289 | 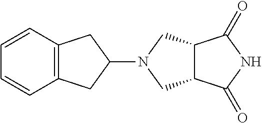 [SMILES: O=C1NC(=O)[C@@H]2CN(C3Cc4ccccc4C3)C[C@H]12] |
| I-290 | 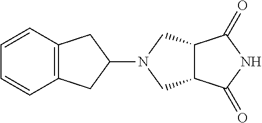 [SMILES: O=C1NC(=O)[C@@H]2CN(C3Cc4ccccc4C3)C[C@H]12] |
| I-291 | 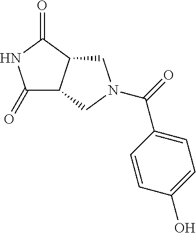 [SMILES: O=C1NC(=O)[C@@H]2CN(C(=O)c3ccc(O)cc3)C[C@H]12] |
| I-292 | 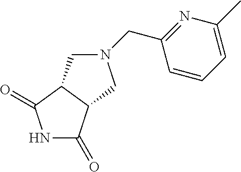 [SMILES: Cc1cccc(CN2C[C@@H]3C(=O)NC(=O)[C@@H]3C2)n1] |
| I-293 | 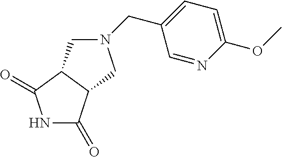 [SMILES: COc1ccc(CN2C[C@@H]3C(=O)NC(=O)[C@@H]3C2)cn1] |
| I-294 | 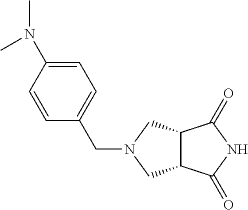 [SMILES: CN(C)c1ccc(CN2C[C@@H]3C(=O)NC(=O)[C@@H]3C2)cc1] |
| I-295 | 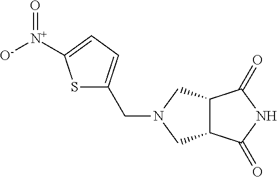 [SMILES: O=C1NC(=O)[C@@H]2CN(Cc3ccc([N+](=O)[O-])s3)C[C@H]12] |
| I-296 | 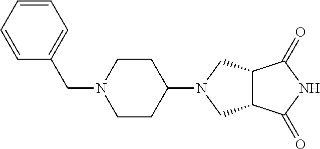 [SMILES: O=C1NC(=O)[C@@H]2CN(C3CCN(Cc4ccccc4)CC3)C[C@H]12] |
| I-297 | 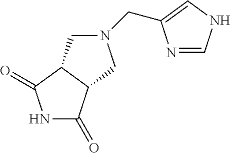 [SMILES: O=C1NC(=O)[C@@H]2CN(Cc3c[nH]cn3)C[C@H]12] |
| I-298 | 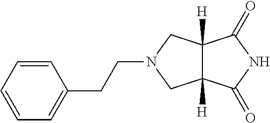 [SMILES: O=C1NC(=O)[C@@H]2CN(CCc3ccccc3)C[C@H]12] |
| I-299 | 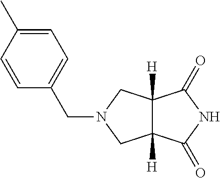 [SMILES: Cc1ccc(CN2C[C@@H]3C(=O)NC(=O)[C@@H]3C2)cc1] |
| I-300 | 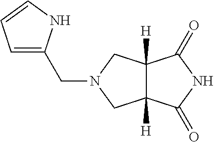 [SMILES: O=C1NC(=O)[C@@H]2CN(Cc3ccc[nH]3)C[C@H]12] |
| I-301 | 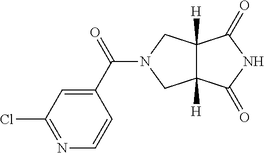 [SMILES: O=C1NC(=O)[C@@H]2CN(C(=O)c3ccnc(Cl)c3)C[C@H]12] |
| I-302 | 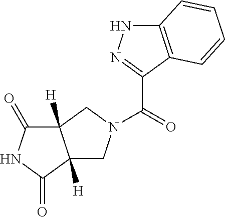 [SMILES: O=C1NC(=O)[C@@H]2CN(C(=O)c3n[nH]c4ccccc34)C[C@H]12] |
| I-303 | 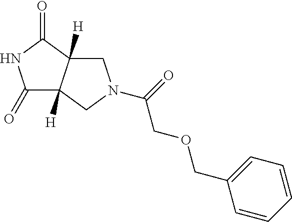 [SMILES: O=C1NC(=O)[C@@H]2CN(C(=O)COCc3ccccc3)C[C@H]12] |
| I-304 | 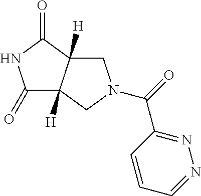 [SMILES: O=C1NC(=O)[C@@H]2CN(C(=O)c3cccnn3)C[C@H]12] |
| I-305 | 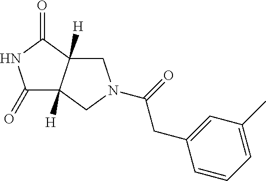 [SMILES: Cc1cccc(CC(=O)N2C[C@@H]3C(=O)NC(=O)[C@@H]3C2)c1] |
| I-306 | 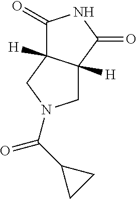 [SMILES: O=C1NC(=O)[C@@H]2CN(C(=O)C3CC3)C[C@H]12] |
| I-307 | 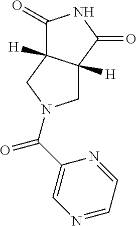 [SMILES: O=C1NC(=O)[C@@H]2CN(C(=O)c3cnccn3)C[C@H]12] |
| I-308 | 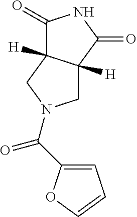 [SMILES: O=C1NC(=O)[C@@H]2CN(C(=O)c3ccco3)C[C@H]12] |
| I-309 | 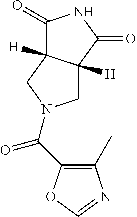 [SMILES: Cc1ncoc1C(=O)N1C[C@@H]2C(=O)NC(=O)[C@@H]2C1] |
| I-310 | 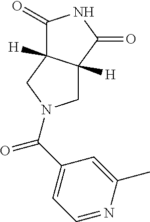 [SMILES: Cc1cc(C(=O)N2C[C@@H]3C(=O)NC(=O)[C@@H]3C2)ccn1] |
| I-311 | 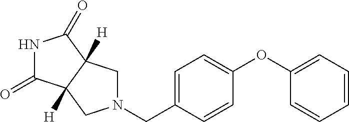 [SMILES: O=C1NC(=O)[C@@H]2CN(Cc3ccc(Oc4ccccc4)cc3)C[C@H]12] |
| I-312 | 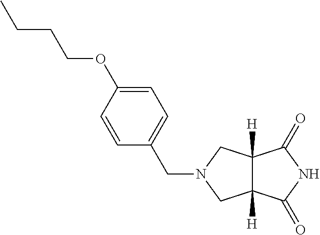 [SMILES: CCCCOc1ccc(CN2C[C@@H]3C(=O)NC(=O)[C@@H]3C2)cc1] |
| I-313 | 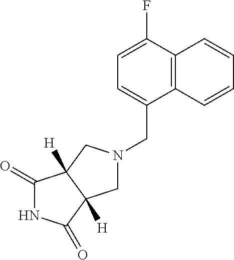 [SMILES: O=C1NC(=O)[C@@H]2CN(Cc3ccc(F)c4ccccc34)C[C@H]12] |
| I-314 | 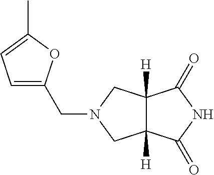 [SMILES: Cc1ccc(CN2C[C@@H]3C(=O)NC(=O)[C@@H]3C2)o1] |
| I-315 | 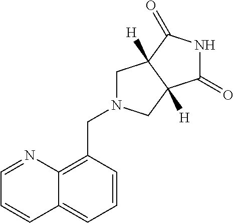 [SMILES: O=C1NC(=O)[C@@H]2CN(Cc3cccc4cccnc34)C[C@H]12] |
| I-316 | 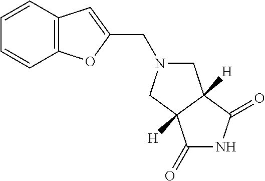 [SMILES: O=C1NC(=O)[C@@H]2CN(Cc3cc4ccccc4o3)C[C@H]12] |
| I-317 | 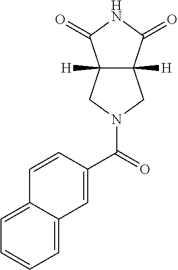 [SMILES: O=C1NC(=O)[C@@H]2CN(C(=O)c3ccc4ccccc4c3)C[C@H]12] |
| I-318 | 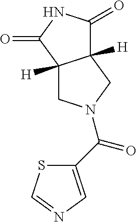 [SMILES: O=C1NC(=O)[C@@H]2CN(C(=O)c3cncs3)C[C@H]12] |
| I-319 | 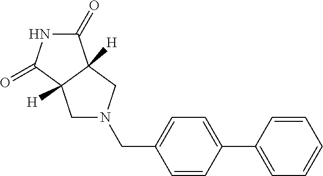 [SMILES: O=C1NC(=O)[C@@H]2CN(Cc3ccc(-c4ccccc4)cc3)C[C@H]12] |
| I-320 | 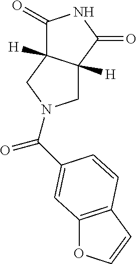 [SMILES: O=C1NC(=O)[C@@H]2CN(C(=O)c3ccc4ccoc4c3)C[C@H]12] |
| I-321 | 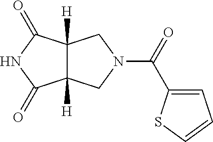 [SMILES: O=C1NC(=O)[C@@H]2CN(C(=O)c3cccs3)C[C@H]12] |
| I-322 | 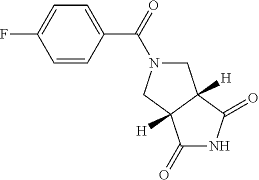 [SMILES: O=C1NC(=O)[C@@H]2CN(C(=O)c3ccc(F)cc3)C[C@H]12] |
| I-323 | 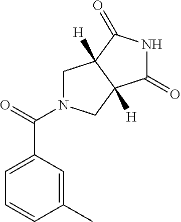 [SMILES: Cc1cccc(C(=O)N2C[C@@H]3C(=O)NC(=O)[C@@H]3C2)c1] |
| I-324 | 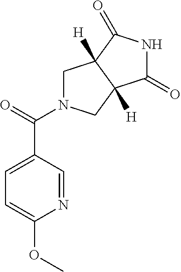 [SMILES: COc1ccc(C(=O)N2C[C@@H]3C(=O)NC(=O)[C@@H]3C2)cn1] |
| I-325 | 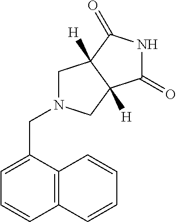 [SMILES: O=C1NC(=O)[C@@H]2CN(Cc3cccc4ccccc34)C[C@H]12] |
| I-326 | 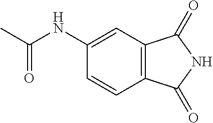 |
| I-327 | 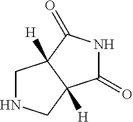 |
| I-328 | 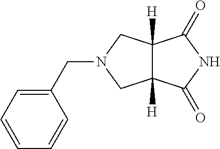 [SMILES: O=C1NC(=O)[C@@H]2CN(Cc3ccccc3)C[C@H]12] |
| I-329 | 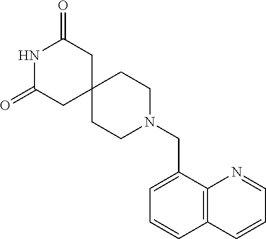 [SMILES: O=C1CC2(CCN(Cc3cccc4cccnc34)CC2)CC(=O)N1] |
| I-330 | 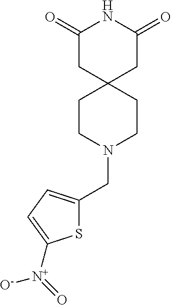 [SMILES: O=C1CC2(CCN(Cc3ccc([N+](=O)[O-])s3)CC2)CC(=O)N1] |
| I-331 | 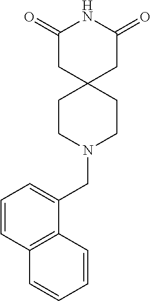 [SMILES: O=C1CC2(CCN(Cc3cccc4ccccc34)CC2)CC(=O)N1] |
| I-332 | 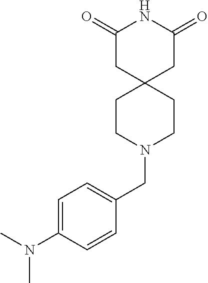 [SMILES: CN(C)c1ccc(CN2CCC3(CC2)CC(=O)NC(=O)C3)cc1] |
| I-333 | 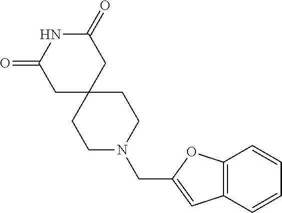 [SMILES: O=C1CC2(CCN(Cc3cc4ccccc4o3)CC2)CC(=O)N1] |
| I-334 | 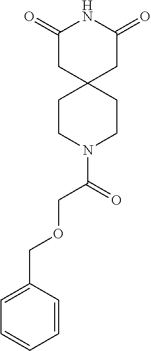 [SMILES: O=C1CC2(CCN(C(=O)COCc3ccccc3)CC2)CC(=O)N1] |
| I-335 | 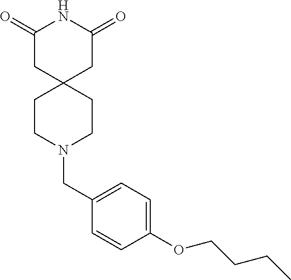 [SMILES: CCCCOc1ccc(CN2CCC3(CC2)CC(=O)NC(=O)C3)cc1] |
| I-336 | 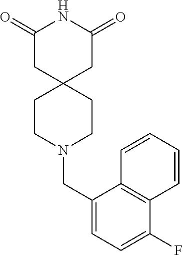 [SMILES: O=C1CC2(CCN(Cc3ccc(F)c4ccccc34)CC2)CC(=O)N1] |
| I-337 | 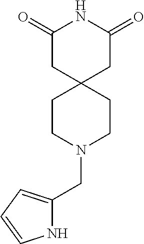 [SMILES: O=C1CC2(CCN(Cc3ccc[nH]3)CC2)CC(=O)N1] |
| I-338 | 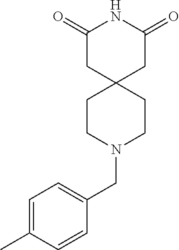 [SMILES: Cc1ccc(CN2CCC3(CC2)CC(=O)NC(=O)C3)cc1] |
| I-339 | 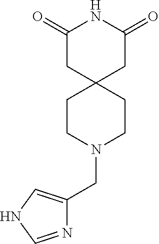 [SMILES: O=C1CC2(CCN(Cc3c[nH]cn3)CC2)CC(=O)N1] |
| I-340 | 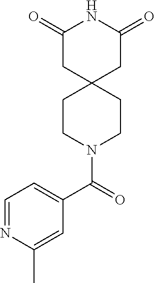 [SMILES: Cc1cc(C(=O)N2CCC3(CC2)CC(=O)NC(=O)C3)ccn1] |
| I-341 | 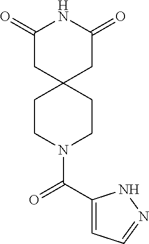 [SMILES: O=C1CC2(CCN(C(=O)c3ccn[nH]3)CC2)CC(=O)N1] |
| I-342 | 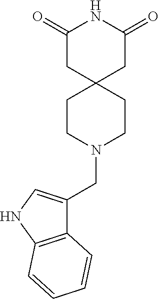 [SMILES: O=C1CC2(CCN(Cc3c[nH]c4ccccc34)CC2)CC(=O)N1] |
| I-343 | 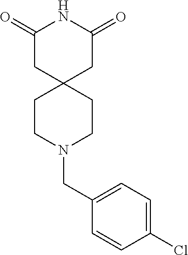 [SMILES: O=C1CC2(CCN(Cc3ccc(Cl)cc3)CC2)CC(=O)N1] |
| I-344 | 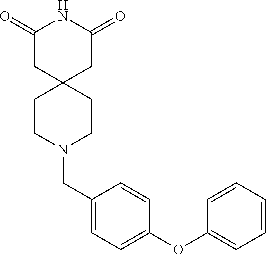 [SMILES: O=C1CC2(CCN(Cc3ccc(Oc4ccccc4)cc3)CC2)CC(=O)N1] |
| I-345 | 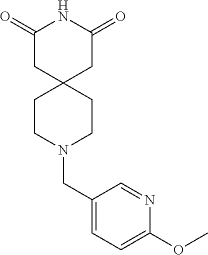 [SMILES: COc1ccc(CN2CCC3(CC2)CC(=O)NC(=O)C3)cn1] |
| I-346 | 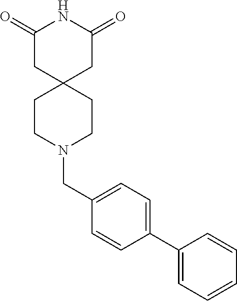 [SMILES: O=C1CC2(CCN(Cc3ccc(-c4ccccc4)cc3)CC2)CC(=O)N1] |
| I-347 | 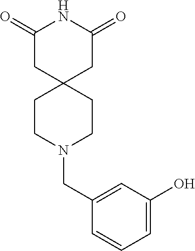 [SMILES: O=C1CC2(CCN(Cc3cccc(O)c3)CC2)CC(=O)N1] |
| I-348 | 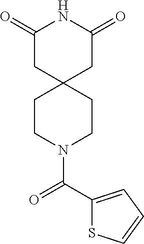 [SMILES: O=C1CC2(CCN(C(=O)c3cccs3)CC2)CC(=O)N1] |
| I-349 | 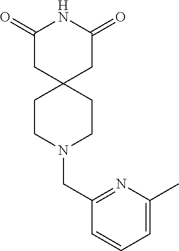 [SMILES: Cc1cccc(CN2CCC3(CC2)CC(=O)NC(=O)C3)n1] |
| I-350 | 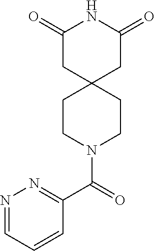 [SMILES: O=C1CC2(CCN(C(=O)c3cccnn3)CC2)CC(=O)N1] |
| I-351 | 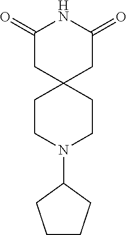 [SMILES: O=C1CC2(CCN(C3CCCC3)CC2)CC(=O)N1] |
| I-352 | 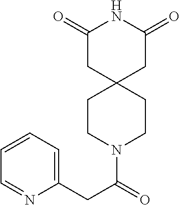 [SMILES: O=C1CC2(CCN(C(=O)Cc3ccccn3)CC2)CC(=O)N1] |
| I-353 | 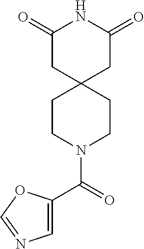 [SMILES: O=C1CC2(CCN(C(=O)c3cnco3)CC2)CC(=O)N1] |
| I-354 | 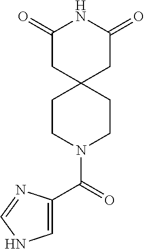 [SMILES: O=C1CC2(CCN(C(=O)c3c[nH]cn3)CC2)CC(=O)N1] |
| I-355 | 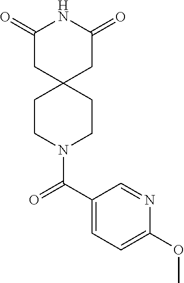 [SMILES: COc1ccc(C(=O)N2CCC3(CC2)CC(=O)NC(=O)C3)cn1] |
| I-356 | 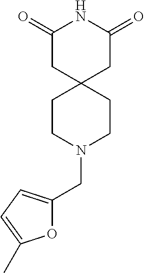 [SMILES: Cc1ccc(CN2CCC3(CC2)CC(=O)NC(=O)C3)o1] |
| I-357 | 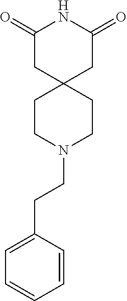 [SMILES: O=C1CC2(CCN(CCc3ccccc3)CC2)CC(=O)N1] |
| I-358 | 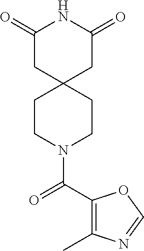 [SMILES: Cc1ncoc1C(=O)N1CCC2(CC1)CC(=O)NC(=O)C2] |
| I-359 | 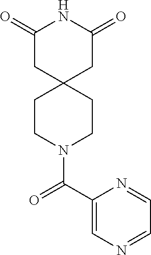 [SMILES: O=C1CC2(CCN(C(=O)c3cnccn3)CC2)CC(=O)N1] |
| I-360 | 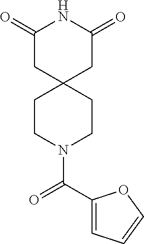 [SMILES: O=C1CC2(CCN(C(=O)c3ccco3)CC2)CC(=O)N1] |
| I-361 | 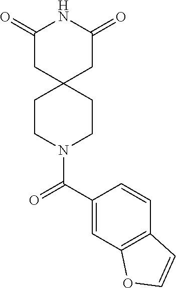 [SMILES: O=C1CC2(CCN(C(=O)c3ccc4ccoc4c3)CC2)CC(=O)N1] |
| I-362 | 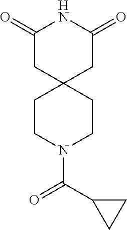 [SMILES: O=C1CC2(CCN(C(=O)C3CC3)CC2)CC(=O)N1] |
| I-363 | 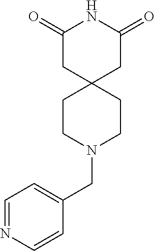 [SMILES: O=C1CC2(CCN(Cc3ccncc3)CC2)CC(=O)N1] |
| I-364 | 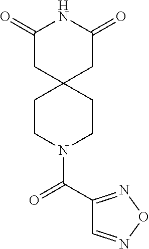 [SMILES: O=C1CC2(CCN(C(=O)c3cnon3)CC2)CC(=O)N1] |
| I-365 | 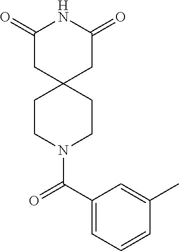 [SMILES: Cc1cccc(C(=O)N2CCC3(CC2)CC(=O)NC(=O)C3)c1] |
| I-366 | 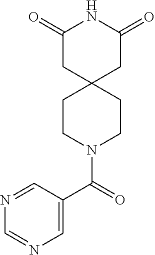 [SMILES: O=C1CC2(CCN(C(=O)c3cncnc3)CC2)CC(=O)N1] |
| I-367 | 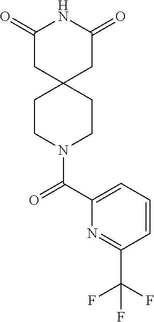 [SMILES: O=C1CC2(CCN(C(=O)c3cccc(C(F)(F)F)n3)CC2)CC(=O)N1] |
| I-368 | 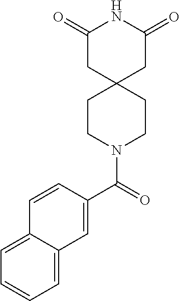 [SMILES: O=C1CC2(CCN(C(=O)c3ccc4ccccc4c3)CC2)CC(=O)N1] |
| I-369 | 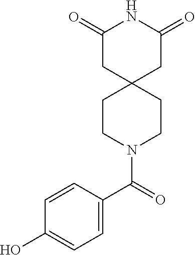 [SMILES: O=C1CC2(CCN(C(=O)c3ccc(O)cc3)CC2)CC(=O)N1] |
| I-370 | 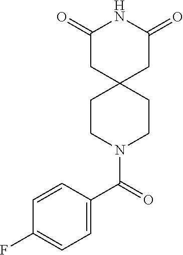 [SMILES: O=C1CC2(CCN(C(=O)c3ccc(F)cc3)CC2)CC(=O)N1] |
| I-370 | 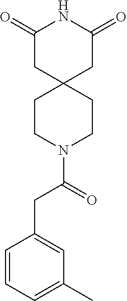 [SMILES: Cc1cccc(CC(=O)N2CCC3(CC2)CC(=O)NC(=O)C3)c1] |
| I-371 | 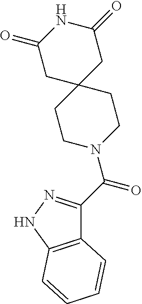 [SMILES: O=C1CC2(CCN(C(=O)c3n[nH]c4ccccc34)CC2)CC(=O)N1] |
| I-372 | 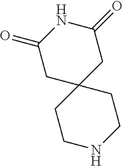 [SMILES: O=C1CC2(CCNCC2)CC(=O)N1] |
| I-373 | 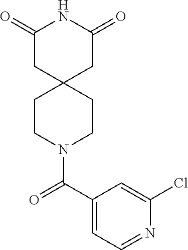 [SMILES: O=C1CC2(CCN(C(=O)c3ccnc(Cl)c3)CC2)CC(=O)N1] |
| I-374 | 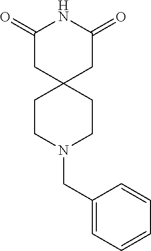 [SMILES: O=C1CC2(CCN(Cc3ccccc3)CC2)CC(=O)N1] |
| I-375 | 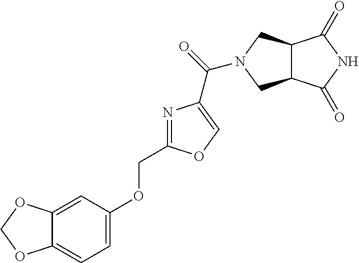 [SMILES: O=C1NC(=O)[C@@H]2CN(C(=O)c3coc(COc4ccc5c(c4)OCO5)n3)C[C@H]12] |
| I-376 | 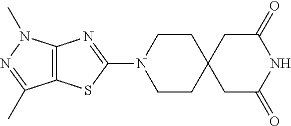 [SMILES: Cc1nn(C)c2nc(N3CCC4(CC3)CC(=O)NC(=O)C4)sc12] |
| I-377 | 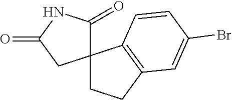 [SMILES: O=C1CC2(CCc3cc(Br)ccc32)C(=O)N1] |
| I-378 | 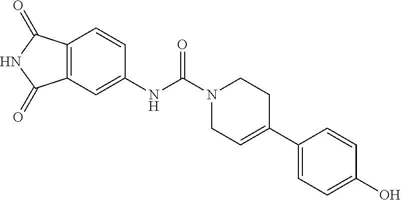 [SMILES: O=C1NC(=O)c2cc(NC(=O)N3CC=C(c4ccc(O)cc4)CC3)ccc21] |

In some embodiments, the present invention provides a compound set forth in Table 1, above, or a pharmaceutically acceptable salt thereof.

### 4. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably bind CRBN, or a mutant thereof, in a biological sample or in a patient. In certain embodiments, the amount of compound in compositions of this invention is such that is effective to measurably bind CRBN, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an active metabolite or residue thereof.

As used herein, the term “active metabolite or residue thereof” means that a metabolite or residue thereof is also a binder of CRBN, or a mutant thereof.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the modulation of CRBN. In some embodiments the protein complex bound by the compounds and methods of the invention comprises CRBN.

Cereblon is a protein that in humans is encoded by the CRBN gene. CRBN orthologs are highly conserved from plants to humans, which underscores its physiological importance. Cereblon forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, cereblon ubquitination of target proteins results in increased levels of fibroblast growth factor 8 (FGF8) and fibroblast growth factor 10 (FGF10). FGF8 in turn regulates a number of developmental processes, such as limb and auditory vesicle formation. The net result is that this ubiquitin ligase complex is important for limb outgrowth in embryos. In the absence of cereblon, DDB1 forms a complex with DDB2 that functions as a DNA damage-binding protein.

Accordingly, compounds that bind CRBN are beneficial, especially those with selectivity over other E3 ligases. Such compounds should deliver a pharmacological response that favorably treats one or more of the conditions described herein without the side-effects associated with the binding of other E3 ligases.

Even though CRBN ligands are known in the art, there is a continuing need to provide novel ligands having more effective or advantageous pharmaceutically relevant properties. For example, compounds with increased activity, selectivity over other E3 ligases, and ADMET (absorption, distribution, metabolism, excretion, and/or toxicity) properties. Thus, in some embodiments, the present invention provides binders of CRBN which show selectivity over other E3 ligases.

The activity of a compound utilized in this invention as an binder of CRBN, or a mutant thereof, may be assayed in vitro, in vivo or in a cell line. In vitro assays include assays that determine the subsequent functional consequences, or activity of activated CRBN, or a mutant thereof. Alternate in vitro assays quantitate the ability of the compound to bind to CRBN. Compound binding may be measured by radiolabeling the compound prior to binding, isolating the compound/CRBN complex and determining the amount of radiolabel bound. Alternatively, compound binding may be determined by running a competition experiment where new compounds are incubated with CRBN bound to known radioligands. Representative in vitro and in vivo assays useful in assaying a CRBN binder include those described and disclosed in, Boichenko et al. J. Med. Chem. 2016 59:770-774 and Iconomou and Saunders Biochemical Journal 2016 473:4083-4101, each of which is herein incorporated by reference in its entirety. Detailed conditions for assaying a compound utilized in this invention as a binder of CRBN, or a mutant thereof, are set forth in the Examples below.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are binders of CRBN and are therefore useful for treating one or more disorders associated with activity of CRBN or mutants thereof. Thus, in certain embodiments, the present invention provides a method for treating a CRBN-mediated disorder comprising the step of administering to a patient in need thereof a compound of the present invention, or pharmaceutically acceptable composition thereof.

As used herein, the term “CRBN-mediated” disorders, diseases, and/or conditions as used herein means any disease or other deleterious condition in which CRBN or a mutant thereof is known to play a role. Accordingly, another embodiment of the present invention relates to treating or lessening the severity of one or more diseases in which CRBN, or a mutant thereof, is known to play a role. Such CRBN-mediated disorders include but are not limited to proliferative disorders, neurological disorders and disorders associated with transplantation.

In some embodiments, the present invention provides a method for treating one or more disorders, wherein the disorders are selected from proliferative disorders, neurological disorders and disorders associated with transplantation, said method comprising administering to a patient in need thereof, a pharmaceutical composition comprising an effective amount of a compound of the present invention, or a pharmaceutically acceptable salt thereof.

In some embodiments, the disorder is a proliferative disorder. In some embodiments, the proliferative disorder is a hematological cancer. In some embodiments, the proliferative disorder is a leukemia. In some embodiments, the proliferative disorder is a leukemia selected from the group consisting of anemia, acute leukemia, acute lymphoblastic leukemia (ALL), chronic lymphocytic leukemia (CLL), acute myelogenous leukemia, acute myeloid leukemia (AML), myelodysplastic syndromes (MDS), adult acute basophilic leukemia, adult acute eosinophilic leukemia, adult acute megakaryoblastic leukemia, adult acute minimally differentiated myeloid leukemia, adult acute monoblastic leukemia, adult acute monocytic leukemia, adult acute myeloblastic leukemia with maturation, adult acute myeloblastic leukemia without maturation, adult acute myeloid leukemia with abnormalities, adult acute myelomonocytic leukemia, adult erythroleukemia, adult pure erythroid leukemia, secondary acute myeloid leukemia, untreated adult acute myeloid leukemia, adult acute myeloid leukemia in remission, adult acute promyelocytic leukemia with PML-RARA, alkylating agent-related acute myeloid leukemia, prolymphocytic leukemia, and chronic myelomonocytic leukemia.

In some embodiments, the proliferative disorder is a lymphoma. In some embodiments, the proliferative disorder is a lymphoma selected from the group consisting of adult grade III lymphomatoid granulomatosis, adult nasal type extranodal NK/T-cell lymphoma, anaplastic large cell lymphoma, angioimmunoblastic T-cell lymphoma, cutaneous B-Cell non-Hodgkin lymphoma, extranodal marginal zone lymphoma of mucosa-associated lymphoid tissue, hepatosplenic T-cell lymphoma, intraocular lymphoma, lymphomatous involvement of non-cutaneous extranodal site, mature T-cell and NK-cell non-Hodgkin lymphoma, nodal marginal zone lymphoma, post-transplant lymphoproliferative disorder, recurrent adult Burkitt lymphoma, recurrent adult diffuse large cell lymphoma, recurrent adult diffuse mixed cell lymphoma, recurrent adult diffuse small cleaved cell lymphoma, recurrent adult grade III lymphomatoid granulomatosis, recurrent adult immunoblastic lymphoma, recurrent adult lymphoblastic lymphoma, recurrent adult T-cell leukemia/lymphoma, recurrent cutaneous T-cell non-Hodgkin lymphoma, recurrent grade 1 follicular lymphoma, recurrent grade 2 follicular lymphoma, recurrent grade 3 follicular lymphoma, recurrent mantle cell lymphoma, recurrent marginal zone lymphoma, recurrent mycosis fungoides and Sezary syndrome, recurrent small lymphocytic lymphoma, refractory chronic lymphocytic leukemia, refractory hairy cell leukemia, Richter syndrome, small intestinal lymphoma, splenic marginal zone lymphoma, T-cell large granular lymphocyte leukemia, testicular lymphoma, Waldenstrom macroglobulinemia, adult T-cell leukemia-lymphoma, peripheral T-cell lymphoma, B-cell lymphoma, Hodgkin's disease, cutaneous T-cell lymphoma, diffuse large B-cell lymphoma, MALT lymphoma, mantle cell lymphoma, non-Hodgkins lymphoma, central nervous system lymphoma, refractory primary-cutaneous large B-cell lymphoma (Leg-type), relapsed or refractory chronic lymphocytic leukemia, refractory anemia, refractory anemia with excess blasts, refractory anemia with ringed sideroblasts, refractory cytopenia with multilineage dysplasia, and secondary myelodysplastic syndromes.

In some embodiments, the disorder is a neurological disorder. In some embodiments, the neurological disorder is Alzheimer's disease.

In some embodiments, the disorder is associated with transplantation. In some embodiments the disorder associated with transplantation is transplant rejection, or graft versus host disease.

In some embodiments, the proliferative disorder is a cancer or tumor. In some embodiments, the proliferative disorder is a cancer or tumor selected from the group consisting of head and neck cancer, liver cancer, hormone-refractory prostate cancer, kidney cancer, small intestine cancer, glioblastoma, non-small cell lung cancer, ovarian cancer, endometrial cancer, esophageal cancer, colon cancer, lung cancer, brain and central nervous system tumors, gastrointestinal carcinoid tumor, islet cell tumor, and childhood solid tumor.

In some embodiments, the proliferative disorder is a myeloma. In some embodiments, the proliferative disorder is a multiple myeloma.

In some embodiments, the proliferative disorder is a myeloma selected from the group consisting of refractory multiple myeloma, stage I multiple myeloma, stage II multiple myeloma, stage III multiple myeloma, smoldering plasma cell myeloma, and plasma cell myeloma.

In some embodiments, the proliferative disorder is selected from the group consisting of hepatocellular carcinoma, melanoma, malignant melanoma, thyroid neoplasms, urinary bladder neoplasms, amyotrophic lateral sclerosis (ALS), sickle cell anemia, ankylosing spondylitis, arachnoiditis, arterivenous malformation, and hereditary hemorrhagic telangiectasia.

In some embodiments, the disorder is selected from the group consisting of AIDS-related Kaposi sarcoma, amyloidosis, hematochezia, melena, autism, burning mouth syndrome associated with HIV infection, hepatocellular carcinoma, non-small-cell lung carcinoma, central nervous system neoplasms, medulloblastoma, chronic myeloproliferative disorders, secondary myelofibrosis, chronic pancreatitis, chronic prostatitis, complex regional pain syndrome (RSD), Type 1 complex regional pain syndrome, Crohn's disease, cutaneous lupus erythematosus (CLE), discoid lupus erythematosus, endometriosis, neoplastic syndrome, gastrointestinal hemorrhage, gastrointestinal vascular malformation, hepatitis C, high grade squamous intra-epithelial lesion (HSIL), HIV wasting syndrome, HIV-associated mycobacterium infections, HIV-associated tuberculosis, HIV-associated aphthous stomatitis, HIV-associated avium-intracellulare infection, idiopathic pulmonary fibrosis (IPF), Langerhans cell histiocytosis (LCH), histiocytosis, Erdheim-Chester disease, histiocytic light chain deposition disease, myelofibrosis, myeloproliferative neoplasms, neurofibromatosis type 1, recurrent central nervous system neoplasm, recurrent childhood brain stem glioma, recurrent childhood visual pathway glioma, refractory central nervous system neoplasm, nonmalignant monoclonal gammopathy of undetermined significance (MGUS), primary amyloidosis, primary myelofibrosis, primary sclerosing cholangitis, plaque-type psoriasis, pulmonary fibrosis, radiation injuries, radiculopathy, recurrent uterine corpus sarcoma, uterine carcinosarcoma, refractory epilepsy, sarcoidosis, systemic scleroderma, systemic sclerosis, Sjogren's Syndrome, xerostomia, soft tissue sarcoma, thalassemia, and uveitis.

In some embodiments, compounds of the present invention bind to CRBN, altering the specificity of the complex to induce the ubiquitination and degradation of Ikaros (IKZF1) and Aiolos (IKZF3), transcription factors essential for multiple myeloma growth.

In some embodiments, compounds of the present invention bind to CRBN, altering the specificity of the complex to induce the ubiquitination and degradation of a complex-associated protein selected from the group consisting of A1BG, A1CF, A2M, A2ML1, A3GALT2, A4GALT, A4GNT, AAAS, AACS, AADAC, AADACL2, AADACL3, AADACL4, AADAT, AAED1, AAGAB, AAK1, AAMDC, AAMP, AANAT, AAR2, AARD, AARS, AARS2, AARSD1, AASDH, AASDHPPT, AASS, AATF, AATK, AATK-AS1, ABAT, ABCA1, ABCA10, ABCA12, ABCA13, ABCA2, ABCA3, ABCA4, ABCA5, ABCA6, ABCA7, ABCA8, ABCA9, ABCB1, ABCB10, ABCB11, ABCB4, ABCB5, ABCB6, ABCB7, ABCB8, ABCB9, ABCC1, ABCC10, ABCC11, ABCC12, ABCC2, ABCC3, ABCC4, ABCC5, ABCC6, ABCC8, ABCC9, ABCD1, ABCD2, ABCD3, ABCD4, ABCE1, ABCF1, ABCF2, ABCF3, ABCG1, ABCG2, ABCG4, ABCG5, ABCG8, ABHD1, ABHD10, ABHD11, ABHD12, ABHD12B, ABHD13, ABHD14A, ABHD14A-ACY1, ABHD14B, ABHD15, ABHD16A, ABHD16B, ABHD17A, ABHD17B, ABHD17C, ABHD18, ABHD2, ABHD3, ABHD4, ABHD5, ABHD6, ABHD8, ABI1, ABI2, ABI3, ABI3BP, ABL1, ABL2, ABLIM1, ABLIM2, ABLIM3, ABO, ABR, ABRA, ABRACL, ABRAXAS1, ABRAXAS2, ABT1, ABTB1, ABTB2, AC001226.2, AC002094.3, AC002115.2, AC002310.4, AC002310.5, AC002429.2, AC002985.1, AC002996.1, AC003002.1, AC003002.2, AC003002.3, AC003002.4, AC003005.1, AC003006.1, AC003688.1, AC004076.1, AC004080.3, AC004223.3, AC004233.2, AC004556.1, AC004691.2, AC004706.4, AC004754.1, AC004805.1, AC004832.3, AC004922.1, AC004997.1, AC005020.2, AC005041.1, AC005154.6, AC005258.1, AC005324.3, AC005324.4, AC005520.1, AC005551.1, AC005670.2, AC005697.1, AC005702.2, AC005726.2, AC005779.2, AC005832.4, AC005833.1, AC005833.3, AC005837.2, AC005841.2, AC005885.1, AC005943.1, AC006030.1, AC006254.1, AC006269.1, AC006449.4, AC006486.1, AC006538.2, AC006978.2, AC007040.2, AC007192.1, AC007240.1, AC007325.1, AC007325.2, AC007325.4, AC007326.4, AC007375.2, AC007383.6, AC007537.5, AC007731.5, AC007906.2, AC007998.2, AC008073.3, AC008162.2, AC008393.2, AC008403.1, AC008481.3, AC008537.1, AC008560.1, AC008575.1, AC008575.2, AC008687.1, AC008687.4, AC008687.8, AC008695.1, AC008735.6, AC008750.8, AC008758.1, AC008758.4, AC008758.5, AC008758.6, AC008763.2, AC008763.3, AC008764.1, AC008764.4, AC008770.2, AC008770.3, AC008878.1, AC008878.2, AC008878.3, AC008982.1, AC008982.3, AC009014.1, AC009086.2, AC009119.2, AC009122.1, AC009133.6, AC009163.2, AC009163.4, AC009286.3, AC009336.2, AC009477.2, AC009690.1, AC009690.3, AC009779.3, AC010132.3, AC010255.3, AC010319.2, AC010323.1, AC010325.1, AC010326.2, AC010327.1, AC010422.3, AC010422.5, AC010422.6, AC010463.1, AC010487.3, AC010522.1, AC010531.1, AC010542.3, AC010547.4, AC010547.5, AC010615.4, AC010616.1, AC010619.1, AC010646.1, AC010724.2, AC011005.1, AC011043.1, AC011043.2, AC011195.2, AC011295.1, AC011346.1, AC011448.1, AC011452.1, AC011455.3, AC011455.4, AC011462.1, AC011473.4, AC011479.1, AC011498.4, AC011499.1, AC011511.1, AC011511.4, AC011530.1, AC011604.2, AC011841.1, AC012184.2, AC012254.2, AC012309.1, AC012314.1, AC012314.10, AC012314.11, AC012314.12, AC012314.4, AC012314.5, AC012314.6, AC012314.8, AC012531.3, AC012651.1, AC013269.1, AC013271.1, AC013394.1, AC013470.2, AC015688.5, AC015802.6, AC015813.2, AC017081.3, AC017081.4, AC017081.5, AC017083.4, AC018512.1, AC018523.2, AC018554.3, AC018630.6, AC018709.1, AC018755.2, AC018793.1, AC018793.2, AC018793.3, AC018793.4, AC018793.5, AC019117.3, AC020636.2, AC020909.1, AC020914.1, AC020915.1, AC020915.2, AC020915.6, AC020922.1, AC020934.3, AC021072.1, AC022016.2, AC022167.5, AC022335.1, AC022384.1, AC022400.6, AC022826.2, AC023055.1, AC023491.2, AC023509.3, AC024592.3, AC024940.1, AC024940.6, AC025165.3, AC025263.2, AC025283.2, AC025287.4, AC025594.2, AC026369.8, AC026398.1, AC026461.4, AC026464.1, AC026464.3, AC026464.4, AC026786.1, AC026954.2, AC027796.3, AC034102.2, AC036214.3, AC037459.1, AC037482.2, AC037482.3, AC040162.1, AC040162.4, AC044810.8, AC046185.1, AC048338.1, AC051649.2, AC053481.5, AC055811.2, AC058822.1, AC064853.2, AC064853.3, AC064853.4, AC064853.5, AC064853.6, AC067968.1, AC068234.1, AC068533.4, AC068547.1, AC068580.4, AC068631.2, AC068775.1, AC068775.2, AC068790.8, AC068896.1, AC068946.1, AC068987.5, AC069257.3, AC069368.1, AC069503.2, AC069544.2, AC072022.1, AC073082.1, AC073111.3, AC073111.5, AC073264.3, AC073508.2, AC073610.2, AC073610.3, AC073612.1, AC073896.1, AC074143.1, AC078927.1, AC079325.2, AC079447.1, AC079594.2, AC083800.1, AC083902.2, AC084337.2, AC087289.3, AC087498.1, AC087632.1, AC090004.1, AC090227.1, AC090360.1, AC090527.2, AC090958.3, AC091167.3, AC091167.7, AC091167.8, AC091304.7, AC091491.1, AC091551.1, AC091959.3, AC091980.2, AC092017.3, AC092042.3, AC092073.1, AC092111.3, AC092143.1, AC092329.3, AC092442.1, AC092587.1, AC092647.5, AC092718.3, AC092718.8, AC092821.1, AC092824.3, AC092835.1, AC093155.3, AC093227.3, AC093423.3, AC093525.1, AC093525.2, AC093668.1, AC093762.1, AC093762.2, AC093762.3, AC093899.2, AC096582.3, AC096887.1, AC097372.1, AC097495.1, AC097637.1, AC097662.2, AC098484.3, AC098650.1, AC098850.4, AC099329.3, AC099489.1, AC099518.3, AC099811.2, AC099850.2, AC100868.1, AC104109.3, AC104151.1, AC104304.1, AC104452.1, AC104532.1, AC104534.3, AC104581.1, AC104581.3, AC104662.2, AC104836.1, AC105001.2, AC105052.1, AC106774.10, AC106774.5, AC106774.6, AC106774.7, AC106774.8, AC106774.9, AC106782.1, AC106886.5, AC107871.1, AC108488.2, AC108750.1, AC108941.2, AC109583.3, AC110275.1, AC112229.3, AC112484.1, AC113189.6, AC113189.9, AC113331.2, AC113554.2, AC114296.1, AC114490.2, AC115220.1, AC116366.3, AC116565.1, AC117457.1, AC118470.1, AC118553.2, AC119396.1, AC119674.2, AC120057.3, AC120114.5, AC124312.1, AC126755.2, AC127537.5, AC127537.6, AC127537.8, AC129492.3, AC131097.2, AC131160.1, AC133551.1, AC133555.3, AC134669.2, AC134772.2, AC135050.2, AC135068.1, AC135068.2, AC135068.3, AC135068.8, AC135178.2, AC135586.2, AC136352.3, AC136352.4, AC136428.1, AC136612.1, AC136616.1, AC136616.2, AC136616.3, AC137834.1, AC138517.2, AC138647.1, AC138696.1, AC138811.2, AC138894.1, AC138969.1, AC139530.2, AC139677.1, AC139677.2, AC140504.1, AC141272.1, AC142391.1, AC142525.4, AC145029.2, AC145212.1, AC145212.2, AC171558.1, AC171558.3, AC171558.5, AC171558.6, AC187653.1, AC207056.1, AC209232.1, AC209539.2, AC210544.1, AC213203.1, AC229888.1, AC229888.10, AC229888.2, AC229888.3, AC229888.4, AC229888.5, AC229888.6, AC229888.7, AC229888.8, AC229888.9, AC233282.1, AC233282.2, AC233723.1, AC233724.12, AC233724.16, AC233724.17, AC233724.18, AC233724.19, AC233724.20, AC233724.21, AC233724.6, AC233755.1, AC233755.2, AC233992.2, AC234301.1, AC234301.3, AC234635.1, AC234635.3, AC234635.4, AC234635.5, AC236040.1, AC239612.1, AC239618.1, AC239618.2, AC239618.3, AC239618.4, AC239618.5, AC239618.6, AC239618.7, AC239618.9, AC239799.1, AC240274.1, AC241401.1, AC241409.2, AC241410.1, AC241556.3, AC241556.4, AC241640.1, AC241640.2, AC241640.4, AC242528.1, AC242528.2, AC243547.3, AC243733.1, AC243734.1, AC243756.1, AC243790.1, AC243967.1, AC244196.1, AC244196.2, AC244196.3, AC244196.4, AC244196.5, AC244197.3, AC244216.4, AC244216.5, AC244226.1, AC244226.2, AC244472.1, AC244472.2, AC244472.3, AC244472.4, AC244472.5, AC244489.1, AC244489.2, AC244517.10, AC244517.6, AC245033.1, AC245034.2, AC245078.1, AC245088.2, AC245088.3, AC245369.1, AC245369.2, AC245369.3, AC245369.4, AC245369.6, AC245427.1, AC245427.3, AC245427.4, AC245427.5, AC245427.6, AC245427.7, AC245427.8, AC245427.9, AC245748.1, AC247036.3, AC247036.4, AC247036.5, AC247036.6, AC254560.1, AC254788.1, AC254788.2, AC254952.1, AC255093.3, AC255093.5, AC256236.1, AC256236.2, AC256236.3, AC256300.2, AC256309.2, AC270107.1, AC270107.10, AC270107.12, AC270107.2, AC270107.3, AC270107.4, AC270107.5, AC270107.7, AC270107.8, AC270107.9, AC270227.1, AC270306.4, AC275455.2, ACAA1, ACAA2, ACACA, ACACB, ACAD10, ACAD11, ACAD8, ACAD9, ACADL, ACADM, ACADS, ACADSB, ACADVL, ACAN, ACAP1, ACAP2, ACAP3, ACAT1, ACAT2, ACBD3, ACBD4, ACBD5, ACBD6, ACBD7, ACCS, ACCSL, ACD, ACE, ACE2, ACER1, ACER2, ACER3, ACHE, ACIN1, ACKR1, ACKR2, ACKR3, ACKR4, ACLY, ACMSD, ACO1, ACO2, ACOD1, ACOT1, ACOT11, ACOT12, ACOT13, ACOT2, ACOT4, ACOT6, ACOT7, ACOT8, ACOT9, ACOX1, ACOX2, ACOX3, ACOXL, ACP1, ACP2, ACP4, ACP5, ACP6, ACP7, ACPP, ACR, ACRBP, ACRV1, ACSBG1, ACSBG2, ACSF2, ACSF3, ACSL1, ACSL3, ACSL4, ACSL5, ACSL6, ACSM1, ACSM2A, ACSM2B, ACSM3, ACSM4, ACSM5, ACSM6, ACSS1, ACSS2, ACSS3, ACTA1, ACTA2, ACTB, ACTBL2, ACTC1, ACTG1, ACTG2, ACTL10, ACTL6A, ACTL6B, ACTL7A, ACTL7B, ACTL8, ACTL9, ACTN1, ACTN2, ACTN3, ACTN4, ACTR10, ACTR1A, ACTR1B, ACTR2, ACTR3, ACTR3B, ACTR3C, ACTR5, ACTR6, ACTR8, ACTRT1, ACTRT2, ACTRT3, ACVR1, ACVR1B, ACVR1C, ACVR2A, ACVR2B, ACVRL1, ACY1, ACY3, ACYP1, ACYP2, AD000671.1, AD000671.2, ADA, ADA2, ADAD1, ADAD2, ADAL, ADAM10, ADAM11, ADAM12, ADAM15, ADAM17, ADAM18, ADAM19, ADAM2, ADAM20, ADAM21, ADAM22, ADAM23, ADAM28, ADAM29, ADAM30, ADAM32, ADAM33, ADAM7, ADAM8, ADAM9, ADAMDEC1, ADAMTS1, ADAMTS10, ADAMTS12, ADAMTS13, ADAMTS14, ADAMTS15, ADAMTS16, ADAMTS17, ADAMTS18, ADAMTS19, ADAMTS2, ADAMTS20, ADAMTS3, ADAMTS4, ADAMTS5, ADAMTS6, ADAMTS7, ADAMTS8, ADAMTS9, ADAMTSL1, ADAMTSL2, ADAMTSL3, ADAMTSL4, ADAMTSL5, ADAP1, ADAP2, ADAR, ADARB1, ADARB2, ADAT1, ADAT2, ADAT3, ADCK1, ADCK2, ADCK5, ADCY1, ADCY10, ADCY2, ADCY3, ADCY4, ADCY5, ADCY6, ADCY7, ADCY8, ADCY9, ADCYAP1, ADCYAP1R1, ADD1, ADD2, ADD3, ADGB, ADGRA1, ADGRA2, ADGRA3, ADGRB1, ADGRB2, ADGRB3, ADGRD1, ADGRD2, ADGRE1, ADGRE2, ADGRE3, ADGRE5, ADGRF1, ADGRF2, ADGRF3, ADGRF4, ADGRF5, ADGRG1, ADGRG2, ADGRG3, ADGRG4, ADGRG5, ADGRG6, ADGRG7, ADGRL1, ADGRL2, ADGRL3, ADGRL4, ADGRV1, ADH1A, ADH1B, ADH1C, ADH4, ADH5, ADH6, ADH7, ADHFE1, ADI1, ADIG, ADIPOQ, ADIPOR1, ADIPOR2, ADIRF, ADK, ADM, ADM2, ADM5, ADNP, ADNP2, ADO, ADORA1, ADORA2A, ADORA2B, ADORA3, ADPGK, ADPRH, ADPRHL1, ADPRHL2, ADPRM, ADRA1A, ADRA1B, ADRA1D, ADRA2A, ADRA2B, ADRA2C, ADRB1, ADRB2, ADRB3, ADRM1, ADSL, ADSS, ADSSL1, ADTRP, AEBP1, AEBP2, AEN, AES, AF130351.1, AF241726.2, AFAP1, AFAP1L1, AFAP1L2, AFDN, AFF1, AFF2, AFF3, AFF4, AFG1L, AFG3L2, AFM, AFMID, AFP, AFTPH, AGA, AGAP1, AGAP2, AGAP3, AGAP4, AGAP5, AGAP6, AGAP9, AGBL1, AGBL2, AGBL3, AGBL4, AGBL5, AGER, AGFG1, AGFG2, AGGF1, AGK, AGL, AGMAT, AGMO, AGO1, AGO2, AGO3, AGO4, AGPAT1, AGPAT2, AGPAT3, AGPAT4, AGPAT5, AGPS, AGR2, AGR3, AGRN, AGRP, AGT, AGTPBP1, AGTR1, AGTR2, AGTRAP, AGXT, AGXT2, AHCTF1, AHCY, AHCYL1, AHCYL2, AHDC1, AHI1, AHNAK, AHNAK2, AHR, AHRR, AHSA1, AHSA2, AHSG, AHSP, AICDA, AIDA, AIF1, AIF1L, AIFM1, AIFM2, AIFM3, AIG1, AIM2, AIMP1, AIMP2, AIP, AIPL1, AIRE, AJAP1, AJUBA, AK1, AK2, AK3, AK4, AK5, AK6, AK7, AK8, AK9, AKAIN1, AKAP1, AKAP10, AKAP11, AKAP12, AKAP13, AKAP14, AKAP17A, AKAP2, AKAP3, AKAP4, AKAP5, AKAP6, AKAP7, AKAP8, AKAP8L, AKAP9, AKIP1, AKIRIN1, AKIRIN2, AKNA, AKNAD1, AKR1A1, AKR1B1, AKR1B10, AKR1B15, AKR1C1, AKR1C2, AKR1C3, AKR1C4, AKR1D1, AKR1E2, AKR7A2, AKR7A3, AKR7L, AKT1, AKT1S1, AKT2, AKT3, AKTIP, AL020996.2, AL021154.3, AL021546.1, AL021997.3, AL022238.4, AL022318.4, AL024498.2, AL031708.1, AL032819.3, AL033529.1, AL035425.2, AL035460.1, AL049634.2, AL049650.1, AL049697.1, AL049779.1, AL049839.2, AL049844.1, AL049844.3, AL080251.1, AL096814.1, AL096870.1, AL109810.2, AL109811.4, AL109827.1, AL109936.3, AL109936.4, AL110118.2, AL110118.4, AL117258.1, AL117339.5, AL117348.2, AL121581.1, AL121594.3, AL121722.1, AL121753.1, AL121758.1, AL121845.2, AL121845.3, AL132671.2, AL132780.3, AL133352.1, AL133414.1, AL133414.2, AL136295.1, AL136295.3, AL136295.4, AL136295.5, AL136373.1, AL136531.2, AL138694.1, AL138752.2, AL138826.1, AL139011.2, AL139260.3, AL139300.1, AL139353.1, AL157392.5, AL159163.1, AL160275.1, AL160276.1, AL160396.2, AL161669.4, AL161911.1, AL162231.1, AL162231.3, AL163195.3, AL163636.2, AL353572.3, AL353588.1, AL354761.2, AL354822.1, AL355102.2, AL355315.1, AL355860.1, AL355916.3, AL355987.1, AL355987.3, AL356585.9, AL357673.1, AL358075.4, AL359736.1, AL359736.3, AL359922.1, AL360181.3, AL360181.5, AL365205.1, AL365214.3, AL365232.1, AL365273.2, AL391650.1, AL449266.1, AL451007.3, AL512428.1, AL512506.3, AL512785.2, AL513165.2, AL513523.10, AL513523.9, AL583836.1, AL589666.1, AL590132.1, AL590560.1, AL591806.3, AL592183.1, AL592490.1, AL593848.2, AL603832.3, AL645922.1, AL645941.2, AL662828.1, AL662852.6, AL662899.1, AL662899.2, AL662899.3, AL669918.1, AL672043.1, AL672142.1, AL691442.1, AL713999.1, AL772284.2, AL807752.6, AL807752.7, AL844853.2, AL845331.2, AL845464.1, AL928654.4, AL929554.1, AL929561.7, ALAD, ALAS1, ALAS2, ALB, ALCAM, ALDH16A1, ALDH18A1, ALDH1A1, ALDH1A2, ALDH1A3, ALDH1B1, ALDH1L1, ALDH1L2, ALDH2, ALDH3A1, ALDH3A2, ALDH3B1, ALDH3B2, ALDH4A1, ALDH5A1, ALDH6A1, ALDH7A1, ALDH8A1, ALDH9A1, ALDOA, ALDOB, ALDOC, ALG1, ALG10, ALG10B, ALG11, ALG12, ALG13, ALG14, ALG1L, ALG1L2, ALG2, ALG3, ALG5, ALG6, ALG8, ALG9, ALK, ALKAL1, ALKAL2, ALKBH1, ALKBH2, ALKBH3, ALKBH4, ALKBH5, ALKBH6, ALKBH7, ALKBH8, ALLC, ALMS1, ALOX12, ALOX12B, ALOX15, ALOX15B, ALOX5, ALOX5AP, ALOXE3, ALPI, ALPK1, ALPK2, ALPK3, ALPL, ALPP, ALPPL2, ALS2, ALS2CL, ALS2CR12, ALX1, ALX3, ALX4, ALYREF, AMACR, AMBN, AMBP, AMBRA1, AMD1, AMDHD1, AMDHD2, AMELX, AMELY, AMER1, AMER2, AMER3, AMFR, AMH, AMHR2, AMIGO1, AMIGO2, AMIGO3, AMMECR1, AMMECR1L, AMN, AMN1, AMOT, AMOTL1, AMOTL2, AMPD1, AMPD2, AMPD3, AMPH, AMT, AMTN, AMY1A, AMY1B, AMY1C, AMY2A, AMY2B, AMZ1, AMZ2, ANAPC1, ANAPC10, ANAPC11, ANAPC13, ANAPC15, ANAPC16, ANAPC2, ANAPC4, ANAPC5, ANAPC7, ANG, ANGEL1, ANGEL2, ANGPT1, ANGPT2, ANGPT4, ANGPTL1, ANGPTL2, ANGPTL3, ANGPTL4, ANGPTL5, ANGPTL6, ANGPTL7, ANGPTL8, ANHX, ANK1, ANK2, ANK3, ANKAR, ANKDD1A, ANKDD1B, ANKEF1, ANKFN1, ANKFY1, ANKH, ANKHD1, ANKHD1-EIF4EBP3, ANKIB1, ANKK1, ANKLE1, ANKLE2, ANKMY1, ANKMY2, ANKRA2, ANKRD1, ANKRD10, ANKRD11, ANKRD12, ANKRD13A, ANKRD13B, ANKRD13C, ANKRD13D, ANKRD16, ANKRD17, ANKRD18A, ANKRD18B, ANKRD2, ANKRD20A1, ANKRD20A2, ANKRD20A3, ANKRD20A4, ANKRD20A8P, ANKRD22, ANKRD23, ANKRD24, ANKRD26, ANKRD27, ANKRD28, ANKRD29, ANKRD30A, ANKRD30B, ANKRD30BL, ANKRD31, ANKRD33, ANKRD33B, ANKRD34A, ANKRD34B, ANKRD34C, ANKRD35, ANKRD36, ANKRD36B, ANKRD36C, ANKRD37, ANKRD39, ANKRD40, ANKRD42, ANKRD44, ANKRD45, ANKRD46, ANKRD49, ANKRD50, ANKRD52, ANKRD53, ANKRD54, ANKRD55, ANKRD6, ANKRD60, ANKRD61, ANKRD62, ANKRD63, ANKRD65, ANKRD66, ANKRD7, ANKRD9, ANKS1A, ANKS1B, ANKS3, ANKS4B, ANKS6, ANKUB1, ANKZF1, ANLN, ANO1, ANO10, ANO2, ANO3, ANO4, ANO5, ANO6, ANO7, ANO8, ANO9, ANOS1, ANP32A, ANP32B, ANP32D, ANP32E, ANPEP, ANTXR1, ANTXR2, ANTXRL, ANXA1, ANXA10, ANXA11, ANXA13, ANXA2, ANXA2R, ANXA3, ANXA4, ANXA5, ANXA6, ANXA7, ANXA8, ANXA8L1, ANXA9, AOAH, AOC1, AOC2, AOC3, AOX1, AP000275.2, AP000295.1, AP000311.1, AP000322.1, AP000349.1, AP000350.12, AP000350.4, AP000351.3, AP000351.7, AP000721.1, AP000781.2, AP001160.5, AP001273.2, AP001458.2, AP001781.3, AP001931.1, AP002360.1, AP002373.1, AP002495.1, AP002512.3, AP002512.4, AP002748.4, AP002990.1, AP003071.5, AP003108.2, AP003419.2, AP004243.1, AP006285.3, AP1AR, AP1B1, AP1G1, AP1G2, AP1M1, AP1M2, AP1S1, AP1S2, AP1S3, AP2A1, AP2A2, AP2B1, AP2M1, AP2S1, AP3B1, AP3B2, AP3D1, AP3M1, AP3M2, AP3S1, AP3S2, AP4B1, AP4E1, AP4M1, AP4S1, APSB1, AP5M1, AP5S1, APSZ1, APAF1, APBA1, APBA2, APBA3, APBB1, APBB1IP, APBB2, APBB3, APC, APC2, APCDD1, APCDD1L, APCS, APEH, APELA, APEX1, APEX2, APH1A, APH1B, APIS, APIP, APLF, APLN, APLNR, APLP1, APLP2, APMAP, APOA1, APOA2, APOA4, APOA5, APOB, APOBEC1, APOBEC2, APOBEC3A, APOBEC3B, APOBEC3C, APOBEC3D, APOBEC3F, APOBEC3G, APOBEC3H, APOBEC4, APOBR, APOC1, APOC2, APOC3, APOC4, APOC4-APOC2, APOD, APOE, APOF, APOH, APOL1, APOL2, APOL3, APOL4, APOL5, APOL6, APOLD1, APOM, APOO, APOOL, APOPT1, APP, APPBP2, APPL1, APPL2, APRT, APTX, AQP1, AQP10, AQP11, AQP12A, AQP12B, AQP2, AQP3, AQP4, AQP5, AQP6, AQP7, AQP8, AQP9, AQR, AR, ARAF, ARAP1, ARAP2, ARAP3, ARC, ARCN1, AREG, AREL1, ARF1, ARF3, ARF4, ARF5, ARF6, ARFGAP1, ARFGAP2, ARFGAP3, ARFGEF1, ARFGEF2, ARFGEF3, ARFIP1, ARFIP2, ARFRP1, ARG1, ARG2, ARGFX, ARGLU1, ARHGAP1, ARHGAP10, ARHGAP11A, ARHGAP11B, ARHGAP12, ARHGAP15, ARHGAP17, ARHGAP18, ARHGAP19, ARHGAP19-SLIT1, ARHGAP20, ARHGAP21, ARHGAP22, ARHGAP23, ARHGAP24, ARHGAP25, ARHGAP26, ARHGAP27, ARHGAP28, ARHGAP29, ARHGAP30, ARHGAP31, ARHGAP32, ARHGAP33, ARHGAP35, ARHGAP36, ARHGAP39, ARHGAP4, ARHGAP40, ARHGAP42, ARHGAP44, ARHGAP45, ARHGAP5, ARHGAP6, ARHGAP8, ARHGAP9, ARHGDIA, ARHGDIB, ARHGDIG, ARHGEF1, ARHGEF10, ARHGEF10L, ARHGEF11, ARHGEF12, ARHGEF15, ARHGEF16, ARHGEF17, ARHGEF18, ARHGEF19, ARHGEF2, ARHGEF25, ARHGEF26, ARHGEF28, ARHGEF3, ARHGEF33, ARHGEF35, ARHGEF37, ARHGEF38, ARHGEF39, ARHGEF4, ARHGEF40, ARHGEF5, ARHGEF6, ARHGEF7, ARHGEF9, ARID1A, ARID1B, ARID2, ARID3A, ARID3B, ARID3C, ARID4A, ARID4B, ARID5A, ARID5B, ARIH1, ARIH2, ARIH2OS, ARL1, ARL10, ARL11, ARL13A, ARL13B, ARL14, ARL14EP, ARL14EPL, ARL15, ARL16, ARL17A, ARL17B, ARL2, ARL2BP, ARL2-SNX15, ARL3, ARL4A, ARL4C, ARL4D, ARL5A, ARL5B, ARL5C, ARL6, ARL6IP1, ARL6IP4, ARL6IP5, ARL6IP6, ARL8A, ARL8B, ARL9, ARMC1, ARMC10, ARMC12, ARMC2, ARMC3, ARMC4, ARMC5, ARMC6, ARMC7, ARMC8, ARMC9, ARMCX1, ARMCX2, ARMCX3, ARMCX4, ARMCX5, ARMCX6, ARMS2, ARMT1, ARNT, ARNT2, ARNTL, ARNTL2, ARPC1A, ARPC1B, ARPC2, ARPC3, ARPC4, ARPC4-TTLL3, ARPC5, ARPC5L, ARPIN, ARPP19, ARPP21, ARR3, ARRB1, ARRB2, ARRDC1, ARRDC2, ARRDC3, ARRDC4, ARRDC5, ARSA, ARSB, ARSD, ARSE, ARSF, ARSG, ARSH, ARSI, ARSJ, ARSK, ART1, ART3, ART4, ART5, ARTN, ARV1, ARVCF, ARX, AS3MT, ASAH1, ASAH2, ASAH2B, ASAP1, ASAP2, ASAP3, ASB1, ASB10, ASB11, ASB12, ASB13, ASB14, ASB15, ASB16, ASB17, ASB18, ASB2, ASB3, ASB4, ASB5, ASB6, ASB7, ASB8, ASB9, ASCC1, ASCC2, ASCC3, ASCL1, ASCL2, ASCL3, ASCL4, ASCL5, ASF1A, ASF1B, ASGR1, ASGR2, ASH1L, ASH2L, ASIC1, ASIC2, ASIC3, ASIC4, ASIC5, ASIP, ASL, ASMT, ASMTL, ASNA1, ASNS, ASNSD1, ASPA, ASPDH, ASPG, ASPH, ASPHD1, ASPHD2, ASPM, ASPN, ASPRV1, ASPSCR1, ASRGL1, ASS1, ASTE1, ASTL, ASTN1, ASTN2, ASXL1, ASXL2, ASXL3, ASZ1, ATAD1, ATAD2, ATAD2B, ATAD3A, ATAD3B, ATAD3C, ATAD5, ATAT1, ATCAY, ATE1, ATF1, ATF2, ATF3, ATF4, ATF5, ATF6, ATF6B, ATF7, ATF7IP, ATF7IP2, ATG10, ATG101, ATG12, ATG13, ATG14, ATG16L1, ATG16L2, ATG2A, ATG2B, ATG3, ATG4A, ATG4B, ATG4C, ATG4D, ATG5, ATG7, ATG9A, ATG9B, ATIC, ATL1, ATL2, ATL3, ATM, ATMIN, ATN1, ATOH1, ATOH7, ATOH8, ATOX1, ATP10A, ATP10B, ATP10D, ATP11A, ATP11B, ATP11C, ATP12A, ATP13A1, ATP13A2, ATP13A3, ATP13A4, ATP13A5, ATP1A1, ATP1A2, ATP1A3, ATP1A4, ATP1B1, ATP1B2, ATP1B3, ATP1B4, ATP23, ATP2A1, ATP2A2, ATP2A3, ATP2B1, ATP2B2, ATP2B3, ATP2B4, ATP2C1, ATP2C2, ATP4A, ATP4B, ATP5A1, ATP5B, ATP5C1, ATP5D, ATP5E, ATP5EP2, ATP5F1, ATP5G1, ATP5G2, ATP5G3, ATP5H, ATP5I, ATP5J, ATP5J2, ATP5J2-PTCD1, ATP5L, ATP5L2, ATP50, ATP5S, ATP6AP1, ATP6AP1L, ATP6AP2, ATP6V0A1, ATP6V0A2, ATP6V0A4, ATP6V0B, ATP6V0C, ATP6V0D1, ATP6V0D2, ATP6V0E1, ATP6V0E2, ATP6V1A, ATP6V1B1, ATP6V1B2, ATP6V1C1, ATP6V1C2, ATP6V1D, ATP6V1E1, ATP6V1E2, ATP6V1F, ATP6V1G1, ATP6V1G2, ATP6V1G2-DDX39B, ATP6V1G3, ATP6V1H, ATP7A, ATP7B, ATP8A1, ATP8A2, ATP8B1, ATP8B2, ATP8B3, ATP8B4, ATP9A, ATP9B, ATPAF1, ATPAF2, ATPIF1, ATR, ATRAID, ATRIP, ATRN, ATRNL1, ATRX, ATXN1, ATXN10, ATXN1L, ATXN2, ATXN2L, ATXN3, ATXN3L, ATXN7, ATXN7L1, ATXN7L2, ATXN7L3, ATXN7L3B, AUH, AUNIP, AUP1, AURKA, AURKAIP1, AURKB, AURKC, AUTS2, AVEN, AVIL, AVL9, AVP, AVPI1, AVPR1A, AVPR1B, AVPR2, AWAT1, AWAT2, AXDND1, AXIN1, AXIN2, AXL, AZGP1, AZI2, AZIN1, AZIN2, AZU1, B2M, B3GALNT1, B3GALNT2, B3GALT1, B3GALT2, B3GALT4, B3GALT5, B3GALT6, B3GAT1, B3GAT2, B3GAT3, B3GLCT, B3GNT2, B3GNT3, B3GNT4, B3GNT5, B3GNT6, B3GNT7, B3GNT8, B3GNT9, B3GNTL1, B4GALNT1, B4GALNT2, B4GALNT3, B4GALNT4, B4GALT1, B4GALT2, B4GALT3, B4GALT4, B4GALT5, B4GALT6, B4GALT7, B4GAT1, B9D1, B9D2, BAALC, BAAT, BABAM1, BABAM2, BACE1, BACE2, BACH1, BACH2, BAD, BAG1, BAG2, BAG3, BAG4, BAG5, BAG6, BAGE3, BAHCC1, BAHD1, BAIAP2, BAIAP2L1, BAIAP2L2, BAIAP3, BAK1, BAMBI, BANF1, BANF2, BANK1, BANP, BAP1, BARD1, BARHL1, BARHL2, BARX1, BARX2, BASP1, BATF, BATF2, BATF3, BAX, BAZ1A, BAZ1B, BAZ2A, BAZ2B, BBC3, BBIP1, BBOF1, BBOX1, BBS1, BBS10, BBS12, BBS2, BBS4, BBS5, BBS7, BBS9, BBX, BCAM, BCAN, BCAP29, BCAP31, BCAR1, BCAR3, BCAS1, BCAS2, BCAS3, BCAS4, BCAT1, BCAT2, BCCIP, BCDIN3D, BCHE, BCKDHA, BCKDHB, BCKDK, BCL10, BCL11A, BCL11B, BCL2, BCL2A1, BCL2L1, BCL2L10, BCL2L11, BCL2L12, BCL2L13, BCL2L14, BCL2L15, BCL2L2, BCL2L2-PABPN1, BCL3, BCL6, BCL6B, BCL7A, BCL7B, BCL7C, BCL9, BCL9L, BCLAF1, BCLAF3, BCO1, BCO2, BCOR, BCORL1, BCR, BCS1L, BDH1, BDH2, BDKRB1, BDKRB2, BDNF, BDP1, BEAN1, BECN1, BECN2, BEGAIN, BEND2, BEND3, BEND4, BEND5, BEND6, BEND7, BEST1, BEST2, BEST3, BEST4, BET1, BET1L, BEX1, BEX2, BEX3, BEX4, BEX5, BFAR, BFSP1, BFSP2, BGLAP, BGN, BHLHA15, BHLHA9, BHLHB9, BHLHE22, BHLHE23, BHLHE40, BHLHE41, BHMG1, BHMT, BHMT2, BICC1, BICD1, BICD2, BICDL1, BICDL2, BICRA, BICRAL, BID, BIK, BIN1, BIN2, BIN3, BIRC2, BIRC3, BIRC5, BIRC6, BIRC7, BIRC8, BIVM, BIVM-ERCC5, BLACE, BLCAP, BLID, BLK, BLM, BLMH, BLNK, BLOC1S1, BLOC1S2, BLOC1S3, BLOC1S4, BLOC1S5, BLOC1S5-TXNDC5, BLOC1S6, BLVRA, BLVRB, BLZF1, BMF, BMI1, BMP1, BMP10, BMP15, BMP2, BMP2K, BMP3, BMP4, BMP5, BMP6, BMP7, BMP8A, BMP8B, BMPER, BMPR1A, BMPR1B, BMPR2, BMS1, BMT2, BMX, BNC1, BNC2, BNIP1, BNIP2, BNIP3, BNIP3L, BNIPL, BOC, BOD1, BOD1L1, BOD1L2, BOK, BOLA1, BOLA2, BOLA2B, BOLA2-SMG1P6, BOLA3, BOLL, BOP1, BORA, BORCS5, BORCS6, BORCS7, BORCS7-ASMT, BORCS8, BORCS8-MEF2B, BPGM, BPHL, BPI, BPIFA1, BPIFA2, BPIFA3, BPIFB1, BPIFB2, BPIFB3, BPIFB4, BPIFB6, BPIFC, BPNT1, BPTF, BPY2, BPY2B, BPY2C, BRAF, BRAP, BRAT1, BRCA1, BRCA2, BRCC3, BRD1, BRD2, BRD3, BRD4, BRD7, BRD8, BRD9, BRDT, BRF1, BRF2, BRI3, BRI3BP, BRICD5, BRINP1, BRINP2, BRINP3, BRIP1, BRIX1, BRK1, BRMS1, BRMS1L, BROX, BRPF1, BRPF3, BRS3, BRSK1, BRSK2, BRWD1, BRWD3, BSCL2, BSDC1, BSG, BSN, BSND, BSPH1, BSPRY, BST1, BST2, BSX, BTAF1, BTBD1, BTBD10, BTBD11, BTBD16, BTBD17, BTBD18, BTBD19, BTBD2, BTBD3, BTBD6, BTBD7, BTBD8, BTBD9, BTC, BTD, BTF3, BTF3L4, BTG1, BTG2, BTG3, BTG4, BTK, BTLA, BTN1A1, BTN2A1, BTN2A2, BTN3A1, BTN3A2, BTN3A3, BTNL2, BTNL3, BTNL8, BTNL9, BTRC, BUB1, BUB1B, BUB1B-PAK6, BUB3, BUD13, BUD23, BUD31, BVES, BX004987.1, BX072566.1, BX088645.1, BX248244.1, BX248413.4, BX248415.1, BX248516.1, BX276092.9, BYSL, BZW1, BZW2, C10orf10, C10orf105, C10orf107, C10orf113, C10orf120, C10orf126, C10orf128, C10orf142, C10orf35, C10orf53, C10orf55, C10orf62, C10orf67, C10orf71, C10orf76, C10orf82, C10orf88, C10orf90, C10orf95, C10orf99, C11orf1, C11orf16, C11orf21, C11orf24, C11orf40, C11orf42, C11orf45, C11orf49, C11orf52, C11orf53, C11orf54, C11orf57, C11orf58, C11orf63, C11orf65, C11orf68, C11orf70, C11orf71, C11orf74, C11orf80, C11orf84, C11orf86, C11orf87, C11orf88, C11orf91, C11orf94, C11orf95, C11orf96, C11orf97, C11orf98, C12orf10, C12orf29, C12orf4, C12orf40, C12orf42, C12orf43, C12orf45, C12orf49, C12orf50, C12orf54, C12orf56, C12orf57, C12orf60, C12orf65, C12orf66, C12orf71, C12orf73, C12orf74, C12orf75, C12orf76, C13orf42, C14orf105, C14orf119, C14orf132, C14orf159, C14orf166, C14orf177, C14orf178, C14orf180, C14orf2, C14orf28, C14orf37, C14orf39, C14orf79, C14orf80, C14orf93, C15orf38-AP3S2, C15orf39, C15orf40, C15orf41, C15orf48, C15orf52, C15orf53, C15orf59, C15orf61, C15orf62, C15orf65, C16orf45, C16orf46, C16orf52, C16orf54, C16orf58, C16orf59, C16orf62, C16orf70, C16orf71, C16orf72, C16orf74, C16orf78, C16orf82, C16orf86, C16orf87, C16orf89, C16orf90, C16orf91, C16orf92, C16orf95, C16orf96, C17orf100, C17orf105, C17orf107, C17orf113, C17orf47, C17orf49, C17orf50, C17orf51, C17orf53, C17orf58, C17orf62, C17orf64, C17orf67, C17orf74, C17orf75, C17orf78, C17orf80, C17orf97, C17orf98, C17orf99, C18orf21, C18orf25, C18orf32, C18orf54, C18orf63, C18orf8, C19orf12, C19orf18, C19orf24, C19orf25, C19orf33, C19orf35, C19orf38, C19orf44, C19orf47, C19orf48, C19orf53, C19orf54, C19orf57, C19orf60, C19orf66, C19orf67, C19orf68, C19orf70, C19orf71, C19orf73, C19orf81, C19orf84, C1D, C1GALT1, C1GALT1C1, C1GALT1C1L, C1orf100, C1orf105, C1orf109, C1orf112, C1orf115, C1orf116, C1orf122, C1orf123, C1orf127, C1orf131, C1orf141, C1orf146, C1orf158, C1orf159, C1orf162, C1orf167, C1orf174, C1orf185, C1orf186, C1orf189, C1orf194, C1orf198, C1orf21, C1orf210, C1orf216, C1orf226, C1orf228, C1orf232, C1orf27, C1orf35, C1orf43, C1orf50, C1orf52, C1orf53, C1orf54, C1orf56, C1orf61, C1orf64, C1orf68, C1orf74, C1orf87, C1orf94, C1QA, C1QB, C1QBP, C1QC, C1QL1, C1QL2, C1QL3, C1QL4, C1QTNF1, C1QTNF12, C1QTNF2, C1QTNF3, C1QTNF3-AMACR, C1QTNF4, C1QTNF5, C1QTNF6, C1QTNF7, C1QTNF8, C1QTNF9, C1QTNF9B, C1R, C1RL, CIS, C2, C20orf141, C20orf144, C20orf173, C20orf194, C20orf196, C20orf202, C20orf204, C20orf24, C20orf27, C20orf85, C20orf96, C21orf140, C21orf2, C21orf33, C21orf58, C21orf59, C21orf62, C21orf91, C22orf15, C22orf23, C22orf31, C22orf39, C22orf42, C22orf46, C2CD2, C2CD2L, C2CD3, C2CD4A, C2CD4B, C2CD4C, C2CD4D, C2CD5, C2CD6, C2orf15, C2orf16, C2orf40, C2orf42, C2orf49, C2orf50, C2orf54, C2orf66, C2orf68, C2orf69, C2orf70, C2orf71, C2orf72, C2orf73, C2orf74, C2orf76, C2orf78, C2orf80, C2orf81, C2orf82, C2orf83, C2orf88, C2orf91, C3, C3AR1, C3orf14, C3orf18, C3orf20, C3orf22, C3orf30, C3orf33, C3orf35, C3orf36, C3orf38, C3orf49, C3orf52, C3orf56, C3orf58, C3orf62, C3orf67, C3orf70, C3orf80, C3orf84, C3orf85, C4A, C4B, C4B_2, C4BPA, C4BPB, C4orf17, C4orf19, C4orf22, C4orf26, C4orf3, C4orf32, C4orf33, C4orf36, C4orf45, C4orf46, C4orf47, C4orf48, C4orf50, C4orf51, C5, C5AR1, C5AR2, C5orf15, C5orf22, C5orf24, C5orf30, C5orf34, C5orf38, C5orf42, C5orf46, C5orf47, C5orf49, C5orf51, C5orf52, C5orf56, C5orf58, C5orf60, C5orf63, C5orf67, C6, C6orf10, C6orf106, C6orf118, C6orf120, C6orf132, C6orf136, C6orf141, C6orf15, C6orf163, C6orf201, C6orf203, C6orf222, C6orf223, C6orf226, C6orf229, C6orf47, C6orf48, C6orf52, C6orf58, C6orf62, C6orf89, C7, C7orf25, C7orf26, C7orf31, C7orf33, C7orf34, C7orf43, C7orf49, C7orf50, C7orf55-LUC7L2, C7orf57, C7orf61, C7orf72, C7orf73, C7orf77, C8A, C8B, C8G, C8orf22, C8orf33, C8orf34, C8orf37, C8orf4, C8orf44, C8orf44-SGK3, C8orf46, C8orf48, C8orf58, C8orf59, C8orf74, C8orf76, C8orf82, C8orf86, C8orf88, C8orf89, C9, C9orf116, C9orf129, C9orf131, C9orf135, C9orf152, C9orf153, C9orf16, C9orf172, C9orf24, C9orf3, C9orf40, C9orf43, C9orf47, C9orf50, C9orf57, C9orf64, C9orf66, C9orf72, C9orf78, C9orf84, C9orf85, C9orf92, CA1, CA10, CA11, CA12, CA13, CA14, CA2, CA3, CA4, CA5A, CA5B, CA6, CA7, CA8, CA9, CAAP1, CAB39, CAB39L, CABIN1, CABLES1, CABLES2, CABP1, CABP2, CABP4, CABP5, CABP7, CABS1, CABYR, CACFD1, CACHD1, CACNA1A, CACNA1B, CACNA1C, CACNA1D, CACNA1E, CACNA1F, CACNA1G, CACNA1H, CACNA1I, CACNA1S, CACNA2D1, CACNA2D2, CACNA2D3, CACNA2D4, CACNB1, CACNB2, CACNB3, CACNB4, CACNG1, CACNG2, CACNG3, CACNG4, CACNG5, CACNG6, CACNG7, CACNG8, CACTIN, CACUL1, CACYBP, CAD, CADM1, CADM2, CADM3, CADM4, CADPS, CADPS2, CAGE1, CALB1, CALB2, CALCA, CALCB, CALCOCO1, CALCOCO2, CALCR, CALCRL, CALD1, CALHM1, CALHM2, CALHM3, CALM1, CALM2, CALM3, CALML3, CALML4, CALML5, CALML6, CALN1, CALR, CALR3, CALU, CALY, CAMK1, CAMK1D, CAMK1G, CAMK2A, CAMK2B, CAMK2D, CAMK2G, CAMK2N1, CAMK2N2, CAMK4, CAMKK1, CAMKK2, CAMKMT, CAMKV, CAMLG, CAMP, CAMSAP1, CAMSAP2, CAMSAP3, CAMTA1, CAMTA2, CAND1, CAND2, CANT1, CANX, CAP1, CAP2, CAPG, CAPN1, CAPN10, CAPN11, CAPN12, CAPN13, CAPN14, CAPN15, CAPN2, CAPN3, CAPN5, CAPN6, CAPN7, CAPN8, CAPN9, CAPNS1, CAPNS2, CAPRIN1, CAPRIN2, CAPS, CAPS2, CAPSL, CAPZA1, CAPZA2, CAPZA3, CAPZB, CARD10, CARD11, CARD14, CARD16, CARD17, CARD18, CARD19, CARD6, CARD8, CARD9, CARF, CARHSP1, CARM1, CARMIL1, CARMIL2, CARMIL3, CARNMT1, CARNS1, CARS, CARS2, CARTPT, CASC1, CASC10, CASC3, CASC4, CASD1, CASK, CASKIN1, CASKIN2, CASP1, CASP10, CASP12, CASP14, CASP2, CASP3, CASP4, CASP5, CASP6, CASP7, CASP8, CASP8AP2, CASP9, CASQ1, CASQ2, CASR, CASS4, CAST, CASTOR1, CASTOR2, CASZ1, CAT, CATIP, CATSPER1, CATSPER2, CATSPER3, CATSPER4, CATSPERB, CATSPERD, CATSPERE, CATSPERG, CATSPERZ, CAV1, CAV2, CAV3, CAVIN1, CAVIN2, CAVIN3, CAVIN4, CBARP, CBFA2T2, CBFA2T3, CBFB, CBL, CBLB, CBLC, CBLL1, CBLN1, CBLN2, CBLN3, CBLN4, CBR1, CBR3, CBR4, CBS, CBSL, CBWD1, CBWD2, CBWD3, CBWD5, CBWD6, CBX1, CBX2, CBX3, CBX4, CBX5, CBX6, CBX7, CBX8, CBY1, CBY3, CC2D1A, CC2D1B, CC2D2A, CC2D2B, CCAR1, CCAR2, CCBE1, CCDCl02A, CCDCl02B, CCDCl03, CCDCl05, CCDCl06, CCDCl07, CCDCl10, CCDCl12, CCDCl13, CCDCl14, CCDCl15, CCDCl16, CCDCl17, CCDCl2, CCDCl20, CCDCl21, CCDCl22, CCDCl24, CCDCl25, CCDCl26, CCDCl27, CCDCl29, CCDCl3, CCDCl30, CCDCl34, CCDCl36, CCDCl37, CCDCl38, CCDCl4, CCDCl40, CCDCl41, CCDCl42, CCDCl44A, CCDCl44NL, CCDCl46, CCDCl48, CCDCl49, CCDCl5, CCDCl50, CCDCl51, CCDCl52, CCDCl53, CCDCl54, CCDCl55, CCDCl57, CCDCl58, CCDCl59, CCDCl60, CCDCl63, CCDCl66, CCDCl67, CCDCl68, CCDCl69, CCDCl69-SOHLH2, CCDCl7, CCDCl70, CCDCl71, CCDCl72, CCDCl73, CCDCl74, CCDCl75, CCDCl77, CCDCl78, CCDCl79, CCDCl8, CCDCl80, CCDCl81, CCDCl82, CCDCl83, CCDCl84, CCDCl85, CCDCl86, CCDCl87, CCDCl88, CCDCl89, CCDCl90, CCDCl91, CCDCl92, CCDCl94, CCDCl95, CCDCl96, CCDCl97, CCDC22, CCDC24, CCDC25, CCDC27, CCDC28A, CCDC28B, CCDC3, CCDC30, CCDC32, CCDC33, CCDC34, CCDC36, CCDC38, CCDC39, CCDC40, CCDC42, CCDC43, CCDC47, CCDC50, CCDC51, CCDC54, CCDC57, CCDC58, CCDC59, CCDC6, CCDC60, CCDC61, CCDC62, CCDC63, CCDC65, CCDC66, CCDC68, CCDC69, CCDC7, CCDC70, CCDC71, CCDC71L, CCDC73, CCDC74A, CCDC74B, CCDC77, CCDC78, CCDC8, CCDC80, CCDC81, CCDC82, CCDC83, CCDC84, CCDC85A, CCDC85B, CCDC85C, CCDC86, CCDC87, CCDC88A, CCDC88B, CCDC88C, CCDC89, CCDC9, CCDC90B, CCDC91, CCDC92, CCDC93, CCDC94, CCDC96, CCDC97, CCER1, CCER2, CCHCR1, CCIN, CCK, CCKAR, CCKBR, CCL1, CCL11, CCL13, CCL14, CCL15, CCL15-CCL14, CCL16, CCL17, CCL18, CCL19, CCL2, CCL20, CCL21, CCL22, CCL23, CCL24, CCL25, CCL26, CCL27, CCL28, CCL3, CCL3L1, CCL3L3, CCL4, CCL4L2, CCL5, CCL7, CCL8, CCM2, CCM2L, CCNA1, CCNA2, CCNB1, CCNB1IP1, CCNB2, CCNB3, CCNC, CCND1, CCND2, CCND3, CCNDBP1, CCNE1, CCNE2, CCNF, CCNG1, CCNG2, CCNH, CCNI, CCNI2, CCNJ, CCNJL, CCNK, CCNL1, CCNL2, CCNO, CCNT1, CCNT2, CCNY, CCNYL1, CCP110, CCPG1, CCR1, CCR10, CCR2, CCR3, CCR4, CCR5, CCR6, CCR7, CCR8, CCR9, CCRL2, CCS, CCSAP, CCSER1, CCSER2, CCT2, CCT3, CCT4, CCT5, CCT6A, CCT6B, CCT7, CCT8, CCT8L2, CCZ1, CCZ1B, CD101, CD109, CD14, CD151, CD160, CD163, CD163L1, CD164, CD164L2, CD177, CD180, CD19, CD1A, CD1B, CD1C, CD1D, CD1E, CD2, CD200, CD200R1, CD200R1L, CD207, CD209, CD22, CD226, CD24, CD244, CD247, CD248, CD27, CD274, CD276, CD28, CD2AP, CD2BP2, CD300A, CD300C, CD300E, CD300LB, CD300LD, CD300LF, CD300LG, CD302, CD320, CD33, CD34, CD36, CD37, CD38, CD3D, CD3E, CD3EAP, CD3G, CD4, CD40, CD40LG, CD44, CD46, CD47, CD48, CD5, CD52, CD53, CD55, CD58, CD59, CD5L, CD6, CD63, CD68, CD69, CD7, CD70, CD72, CD74, CD79A, CD79B, CD80, CD81, CD82, CD83, CD84, CD86, CD8A, CD8B, CD9, CD93, CD96, CD99, CD99L2, CDA, CDADC1, CDAN1, CDC123, CDC14A, CDC14B, CDC16, CDC20, CDC20B, CDC23, CDC25A, CDC25B, CDC25C, CDC26, CDC27, CDC34, CDC37, CDC37L1, CDC40, CDC42, CDC42BPA, CDC42BPB, CDC42BPG, CDC42EP1, CDC42EP2, CDC42EP3, CDC42EP4, CDC42EP5, CDC42SE1, CDC42SE2, CDC45, CDC5L, CDC6, CDC7, CDC73, CDCA2, CDCA3, CDCA4, CDCA5, CDCA7, CDCA7L, CDCA8, CDCP1, CDCP2, CDH1, CDH10, CDH11, CDH12, CDH13, CDH15, CDH16, CDH17, CDH18, CDH19, CDH2, CDH20, CDH22, CDH23, CDH24, CDH26, CDH3, CDH4, CDH5, CDH6, CDH7, CDH8, CDH9, CDHR1, CDHR2, CDHR3, CDHR4, CDHR5, CDIP1, CDIPT, CDK1, CDK10, CDK11A, CDK11B, CDK12, CDK13, CDK14, CDK15, CDK16, CDK17, CDK18, CDK19, CDK2, CDK20, CDK2AP1, CDK2AP2, CDK3, CDK4, CDK5, CDK5R1, CDK5R2, CDK5RAP1, CDK5RAP2, CDK5RAP3, CDK6, CDK7, CDK8, CDK9, CDKAL1, CDKL1, CDKL2, CDKL3, CDKL4, CDKL5, CDKN1A, CDKN1B, CDKN1C, CDKN2A, CDKN2AIP, CDKN2AIPNL, CDKN2B, CDKN2C, CDKN2D, CDKN3, CDNF, CDO1, CDON, CDPF1, CDR1, CDR2, CDR2L, CDRT1, CDRT15, CDRT15L2, CDRT4, CDS1, CDS2, CDSN, CDT1, CDV3, CDX1, CDX2, CDX4, CDY1, CDY1B, CDY2A, CDY2B, CDYL, CDYL2, CEACAM1, CEACAM16, CEACAM19, CEACAM20, CEACAM21, CEACAM3, CEACAM4, CEACAM5, CEACAM6, CEACAM7, CEACAM8, CEBPA, CEBPB, CEBPD, CEBPE, CEBPG, CEBPZ, CEBPZOS, CECR2, CEL, CELA1, CELA2A, CELA2B, CELA3A, CELA3B, CELF1, CELF2, CELF3, CELF4, CELF5, CELF6, CELSR1, CELSR2, CELSR3, CEMIP, CEMP1, CEND1, CENPA, CENPB, CENPBD1, CENPC, CENPE, CENPF, CENPH, CENPI, CENPJ, CENPK, CENPL, CENPM, CENPN, CENPO, CENPP, CENPQ, CENPS, CENPS-CORT, CENPT, CENPU, CENPV, CENPVL1, CENPVL2, CENPVL3, CENPW, CENPX, CEP104, CEP112, CEP120, CEP126, CEP128, CEP131, CEP135, CEP152, CEP162, CEP164, CEP170, CEP170B, CEP19, CEP192, CEP250, CEP290, CEP295, CEP295NL, CEP350, CEP41, CEP44, CEP55, CEP57, CEP57L1, CEP63, CEP68, CEP70, CEP72, CEP76, CEP78, CEP83, CEP85, CEP85L, CEP89, CEP95, CEP97, CEPT1, CER1, CERCAM, CERK, CERKL, CERS1, CERS2, CERS3, CERS4, CERS5, CERS6, CES1, CES2, CES3, CES4A, CES5A, CETN1, CETN2, CETN3, CETP, CFAP100, CFAP126, CFAP157, CFAP161, CFAP20, CFAP206, CFAP221, CFAP36, CFAP43, CFAP44, CFAP45, CFAP46, CFAP47, CFAP52, CFAP53, CFAP54, CFAP57, CFAP58, CFAP61, CFAP65, CFAP69, CFAP70, CFAP73, CFAP74, CFAP77, CFAP97, CFAP99, CFB, CFC1, CFC1B, CFD, CFDP1, CFH, CFHR1, CFHR2, CFHR3, CFHR4, CFHR5, CFI, CFL1, CFL2, CFLAR, CFP, CFTR, CGA, CGB1, CGB2, CGB3, CGB5, CGB7, CGB8, CGGBP1, CGN, CGNL1, CGREF1, CGRRF1, CH25H, CHAC1, CHAC2, CHAD, CHADL, CHAFlA, CHAF1B, CHAMP1, CHAT, CHCHD1, CHCHD10, CHCHD2, CHCHD3, CHCHD4, CHCHD5, CHCHD6, CHCHD7, CHD1, CHD1L, CHD2, CHD3, CHD4, CHD5, CHD6, CHD7, CHD8, CHD9, CHDH, CHEK1, CHEK2, CHERP, CHFR, CHGA, CHGB, CHI3L1, CHI3L2, CHIA, CHIC1, CHIC2, CHID1, CHIT1, CHKA, CHKB, CHKB-CPT1B, CHL1, CHM, CHML, CHMP1A, CHMP1B, CHMP2A, CHMP2B, CHMP3, CHMP4A, CHMP4B, CHMP4C, CHMP5, CHMP6, CHMP7, CHN1, CHN2, CHODL, CHORDC1, CHP1, CHP2, CHPF, CHPF2, CHPT1, CHRAC1, CHRD, CHRDL1, CHRDL2, CHRFAM7A, CHRM1, CHRM2, CHRM3, CHRM4, CHRM5, CHRNA1, CHRNA10, CHRNA2, CHRNA3, CHRNA4, CHRNA5, CHRNA6, CHRNA7, CHRNA9, CHRNB1, CHRNB2, CHRNB3, CHRNB4, CHRND, CHRNE, CHRNG, CHST1, CHST10, CHST11, CHST12, CHST13, CHST14, CHST15, CHST2, CHST3, CHST4, CHST5, CHST6, CHST7, CHST8, CHST9, CHSY1, CHSY3, CHTF18, CHTF8, CHTOP, CHUK, CHURC1, CHURC1-FNTB, CIAO1, CIAPIN1, CIART, CIB1, CIB2, CIB3, CIB4, CIC, CIDEA, CIDEB, CIDEC, CIITA, CILP, CILP2, CINP, CIPC, CIR1, CIRBP, CISD1, CISD2, CISD3, CISH, CIT, CITED1, CITED2, CITED4, CIZ1, CKAP2, CKAP2L, CKAP4, CKAP5, CKB, CKLF, CKLF-CMTM1, CKM, CKMT1A, CKMT1B, CKMT2, CKS1B, CKS2, CLASP1, CLASP2, CLASRP, CLC, CLCA1, CLCA2, CLCA4, CLCC1, CLCF1, CLCN1, CLCN2, CLCN3, CLCN4, CLCN5, CLCN6, CLCN7, CLCNKA, CLCNKB, CLDN1, CLDN10, CLDN11, CLDN12, CLDN14, CLDN15, CLDN16, CLDN17, CLDN18, CLDN19, CLDN2, CLDN20, CLDN22, CLDN23, CLDN24, CLDN25, CLDN3, CLDN34, CLDN4, CLDN5, CLDN6, CLDN7, CLDN8, CLDN9, CLDND1, CLDND2, CLEC10A, CLEC11A, CLEC12A, CLEC12B, CLEC14A, CLEC16A, CLEC17A, CLEC18A, CLEC18B, CLEC18C, CLEC19A, CLEC1A, CLEC1B, CLEC20A, CLEC2A, CLEC2B, CLEC2D, CLEC2L, CLEC3A, CLEC3B, CLEC4A, CLEC4C, CLEC4D, CLEC4E, CLEC4F, CLEC4G, CLEC4M, CLEC5A, CLEC6A, CLEC7A, CLEC9A, CLECL1, CLGN, CLHC1, CLIC1, CLIC2, CLIC3, CLIC4, CLIC5, CLIC6, CLINT1, CLIP1, CLIP2, CLIP3, CLIP4, CLK1, CLK2, CLK3, CLK4, CLLU1, CLLU1OS, CLMN, CLMP, CLN3, CLN5, CLN6, CLN8, CLNK, CLNS1A, CLOCK, CLP1, CLPB, CLPP, CLPS, CLPSL1, CLPSL2, CLPTM1, CLPTM1L, CLPX, CLRN1, CLRN2, CLRN3, CLSPN, CLSTN1, CLSTN2, CLSTN3, CLTA, CLTB, CLTC, CLTCL1, CLU, CLUAP1, CLUH, CLUL1, CLVS1, CLVS2, CLYBL, CMA1, CMAS, CMBL, CMC1, CMC2, CMC4, CMIP, CMKLR1, CMPK1, CMPK2, CMSS1, CMTM1, CMTM2, CMTM3, CMTM4, CMTM5, CMTM6, CMTM7, CMTM8, CMTR1, CMTR2, CMYA5, CNBD1, CNBD2, CNBP, CNDP1, CNDP2, CNEP1R1, CNFN, CNGA1, CNGA2, CNGA3, CNGA4, CNGB1, CNGB3, CNIH1, CNIH2, CNIH3, CNIH4, CNKSR1, CNKSR2, CNKSR3, CNMD, CNN1, CNN2, CNN3, CNNM1, CNNM2, CNNM3, CNNM4, CNOT1, CNOT10, CNOT11, CNOT2, CNOT3, CNOT4, CNOT6, CNOT6L, CNOT7, CNOT8, CNOT9, CNP, CNPPD1, CNPY1, CNPY2, CNPY3, CNPY4, CNR1, CNR2, CNRIP1, CNST, CNTD1, CNTD2, CNTF, CNTFR, CNTLN, CNTN1, CNTN2, CNTN3, CNTN4, CNTN5, CNTN6, CNTNAP1, CNTNAP2, CNTNAP3, CNTNAP3B, CNTNAP4, CNTNAP5, CNTRL, CNTROB, COA1, COA3, COA4, COA5, COA6, COA7, COASY, COBL, COBLL1, COCH, COG1, COG2, COG3, COG4, COG5, COG6, COG7, COG8, COIL, COL10A1, COL11A1, COL11A2, COL12A1, COL13A1, COL14A1, COL15A1, COL16A1, COL17A1, COL18A1, COL19A1, COL1A1, COL1A2, COL20A1, COL21A1, COL22A1, COL23A1, COL24A1, COL25A1, COL26A1, COL27A1, COL28A1, COL2A1, COL3A1, COL4A1, COL4A2, COL4A3, COL4A3BP, COL4A4, COL4A5, COL4A6, COL5A1, COL5A2, COL5A3, COL6A1, COL6A2, COL6A3, COL6A5, COL6A6, COL7A1, COL8A1, COL8A2, COL9A1, COL9A2, COL9A3, COLCA2, COLEC10, COLEC11, COLEC12, COLGALT1, COLGALT2, COLQ, COMMD1, COMMD10, COMMD2, COMMD3, COMMD3-BMI1, COMMD4, COMMD5, COMMD6, COMMD7, COMMD8, COMMD9, COMP, COMT, COMTD1, COPA, COPB1, COPB2, COPE, COPG1, COPG2, COPRS, COPS2, COPS3, COPS4, COPS5, COPS6, COPS7A, COPS7B, COPS8, COPS9, COPZ1, COPZ2, COQ10A, COQ10B, COQ2, COQ3, COQ4, COQ5, COQ6, COQ7, COQ8A, COQ8B, COQ9, CORIN, CORO1A, CORO1B, CORO1C, CORO2A, CORO2B, CORO6, CORO7, CORO7-PAM16, COR^{†}, COTL1, COX10, COX11, COX14, COX15, COX16, COX17, COX18, COX19, COX20, COX4I1, COX4I2, COX5A, COX5B, COX6A1, COX6A2, COX6B1, COX6B2, COX6C, COX7A1, COX7A2, COX7A2L, COX7B, COX7B2, COX7C, COX8A, COX8C, CP, CPA1, CPA2, CPA3, CPA4, CPA5, CPA6, CPAMD8, CPB1, CPB2, CPD, CPE, CPEB1, CPEB2, CPEB3, CPEB4, CPED1, CPLX1, CPLX2, CPLX3, CPLX4, CPM, CPN1, CPN2, CPNE1, CPNE2, CPNE3, CPNE4, CPNE5, CPNE6, CPNE7, CPNE8, CPNE9, CPO, CPOX, CPPED1, CPQ, CPS1, CPSF1, CPSF2, CPSF3, CPSF4, CPSF4L, CPSF6, CPSF7, CPT1A, CPT1B, CPT1C, CPT2, CPTP, CPVL, CPXCR1, CPXM1, CPXM2, CPZ, CR1, CR1L, CR2, CR354443.1, CR354443.2, CR388407.3, CR547123.3, CR753842.1, CR753845.2, CR759815.2, CR788250.1, CR847794.2, CR854858.1, CR933783.3, CR936239.1, CRABP1, CRABP2, CRACR2A, CRACR2B, CRADD, CRAMP1, CRAT, CRB1, CRB2, CRB3, CRBN, CRCP, CRCT1, CREB1, CREB3, CREB3L1, CREB3L2, CREB3L3, CREB3L4, CREB5, CREBBP, CREBL2, CREBRF, CREBZF, CREG1, CREG2, CRELD1, CRELD2, CREM, CRH, CRHBP, CRHR1, CRHR2, CRIM1, CRIP1, CRIP2, CRIP3, CRIPT, CRISP1, CRISP2, CRISP3, CRISPLD1, CRISPLD2, CRK, CRKL, CRLF1, CRLF2, CRLF3, CRLS1, CRMP1, CRNKL1, CRNN, CROCC, CROCC2, CROT, CRP, CRTAC1, CRTAM, CRTAP, CRTC1, CRTC2, CRTC3, CRX, CRY1, CRY2, CRYAA, CRYAB, CRYBA1, CRYBA2, CRYBA4, CRYBB1, CRYBB2, CRYBB3, CRYBG1, CRYBG2, CRYBG3, CRYGA, CRYGB, CRYGC, CRYGD, CRYGN, CRYGS, CRYL1, CRYM, CRYZ, CRYZL1, CS, CSAD, CSAG1, CSAG2, CSAG3, CSDC2, CSDE1, CSE1L, CSF1, CSF1R, CSF2, CSF2RA, CSF2RB, CSF3, CSF3R, CSGALNACT1, CSGALNACT2, CSH1, CSH2, CSHL1, CSK, CSMD1, CSMD2, CSMD3, CSN1S1, CSN2, CSN3, CSNK1A1, CSNK1A1L, CSNKlD, CSNK1E, CSNK1G1, CSNK1G2, CSNK1G3, CSNK2A1, CSNK2A2, CSNK2A3, CSNK2B, CSPG4, CSPG5, CSPP1, CSRNP1, CSRNP2, CSRNP3, CSRP1, CSRP2, CSRP3, CST1, CST11, CST2, CST3, CST4, CST5, CST6, CST7, CST8, CST9, CST9L, CSTA, CSTB, CSTF1, CSTF2, CSTF2T, CSTF3, CSTL1, CT45A1, CT45A10, CT45A2, CT45A3, CT45A5, CT45A6, CT45A7, CT45A8, CT45A9, CT476828.1, CT476828.10, CT476828.11, CT476828.12, CT476828.13, CT476828.14, CT476828.15, CT476828.16, CT476828.17, CT476828.18, CT476828.19, CT476828.2, CT476828.20, CT476828.21, CT476828.22, CT476828.3, CT476828.4, CT476828.5, CT476828.6, CT476828.7, CT476828.8, CT476828.9, CT47A1, CT47A10, CT47A11, CT47A12, CT47A2, CT47A3, CT47A4, CT47A5, CT47A6, CT47A7, CT47A8, CT47A9, CT47B1, CT55, CT62, CT83, CTAG1A, CTAG1B, CTAG2, CTAGE1, CTAGE15, CTAGE4, CTAGE5, CTAGE6, CTAGE8, CTAGE9, CTBP1, CTBP2, CTBS, CTC1, CTCF, CTCFL, CTDNEP1, CTDP1, CTDSP1, CTDSP2, CTDSPL, CTDSPL2, CTF1, CTGF, CTH, CTHRC1, CTIF, CTLA4, CTNNA1, CTNNA2, CTNNA3, CTNNAL1, CTNNB1, CTNNBIP1, CTNNBL1, CTNND1, CTNND2, CTNS, CTPS1, CTPS2, CTR9, CTRB1, CTRB2, CTRC, CTRL, CTSA, CTSB, CTSC, CTSD, CTSE, CTSF, CTSG, CTSH, CTSK, CTSL, CTSO, CTSS, CTSV, CTSW, CTSZ, CTTN, CTTNBP2, CTTNBP2NL, CTU1, CTU2, CTXN1, CTXN2, CTXN3, CTXND1, CU464060.1, CU633846.1, CU633980.1, CU633980.2, CU639417.1, CU639417.2, CUBN, CUEDC1, CUEDC2, CUL1, CUL2, CUL3, CUL4A, CUL4B, CUL5, CUL7, CUL9, CUTA, CUTC, CUX1, CUX2, CUZD1, CWC15, CWC22, CWC25, CWC27, CWF19L1, CWF19L2, CWH43, CX3CL1, CX3CR1, CXADR, CXCL1, CXCL10, CXCL11, CXCL12, CXCL13, CXCL14, CXCL16, CXCL17, CXCL2, CXCL3, CXCL5, CXCL6, CXCL8, CXCL9, CXCR1, CXCR2, CXCR3, CXCR4, CXCR5, CXCR6, CXorf21, CXorf36, CXorf38, CXorf40A, CXorf40B, CXorf49, CXorf49B, CXorf51A, CXorf51B, CXorf56, CXorf57, CXorf58, CXorf65, CXorf66, CXorf67, CXXC1, CXXC4, CXXC5, CYB561, CYB561A3, CYB561D1, CYB561D2, CYB5A, CYB5B, CYB5D1, CYB5D2, CYB5R1, CYB5R2, CYB5R3, CYB5R4, CYB5RL, CYBA, CYBB, CYBRD1, CYC1, CYCS, CYFIP1, CYFIP2, CYGB, CYHR1, CYLC1, CYLC2, CYLD, CYP11A1, CYP11B1, CYP11B2, CYP17A1, CYP19A1, CYP1A1, CYP1A2, CYP1B1, CYP20A1, CYP21A2, CYP24A1, CYP26A1, CYP26B1, CYP26C1, CYP27A1, CYP27B1, CYP27C1, CYP2A13, CYP2A6, CYP2A7, CYP2B6, CYP2C18, CYP2C19, CYP2C8, CYP2C9, CYP2D6, CYP2D7, CYP2E1, CYP2F1, CYP2J2, CYP2R1, CYP2S1, CYP2U1, CYP2W1, CYP39A1, CYP3A4, CYP3A43, CYP3A5, CYP3A7, CYP3A7-CYP3A51P, CYP46A1, CYP4A11, CYP4A22, CYP4B1, CYP4F11, CYP4F12, CYP4F2, CYP4F22, CYP4F3, CYP4F8, CYP4V2, CYP4X1, CYP4Z1, CYP51A1, CYP7A1, CYP7B1, CYP8B1, CYR61, CYS1, CYSLTR1, CYSLTR2, CYSRT1, CYSTM1, CYTH1, CYTH2, CYTH3, CYTH4, CYTIP, CYTL1, CYYR1, D2HGDH, DAAM1, DAAM2, DAB1, DAB2, DAB2IP, DACH1, DACH2, DACT1, DACT2, DACT3, DAD1, DAG1, DAGLA, DAGLB, DALRD3, DAND5, DAO, DAOA, DAP, DAP3, DAPK1, DAPK2, DAPK3, DAPL1, DAPP1, DARS, DARS2, DAW1, DAXX, DAZ1, DAZ2, DAZ3, DAZ4, DAZAP1, DAZAP2, DAZL, DBF4, DBF4B, DBH, DBI, DBN1, DBNDD1, DBNDD2, DBNL, DBP, DBR1, DBT, DBX1, DBX2, DCAF1, DCAF10, DCAF11, DCAF12, DCAF12L1, DCAF12L2, DCAF13, DCAF15, DCAF16, DCAF17, DCAF4, DCAF4L1, DCAF4L2, DCAF5, DCAF6, DCAF7, DCAF8, DCAF8L1, DCAF8L2, DCAKD, DCANP1, DCBLD1, DCBLD2, DCC, DCD, DCDC1, DCDC2, DCDC2B, DCDC2C, DCHS1, DCHS2, DCK, DCLK1, DCLK2, DCLK3, DCLRE1A, DCLRE1B, DCLRE1C, DCN, DCP1A, DCP1B, DCP2, DCPS, DCST1, DCST2, DCSTAMP, DCT, DCTD, DCTN1, DCTN2, DCTN3, DCTN4, DCTN5, DCTN6, DCTPP1, DCUN1D1, DCUN1D2, DCUN1D3, DCUN1D4, DCUN1D5, DCX, DCXR, DDA1, DDAH1, DDAH2, DDB1, DDB2, DDC, DDHD1, DDHD2, DDI1, DDI2, DDIAS, DDIT3, DDIT4, DDIT4L, DDN, DDO, DDOST, DDR1, DDR2, DDRGK1, DDT, DDTL, DDX1, DDX10, DDX11, DDX17, DDX18, DDX19A, DDX19B, DDX20, DDX21, DDX23, DDX24, DDX25, DDX27, DDX28, DDX31, DDX39A, DDX39B, DDX3X, DDX3Y, DDX4, DDX41, DDX42, DDX43, DDX46, DDX47, DDX49, DDX5, DDX50, DDX51, DDX52, DDX53, DDX54, DDX55, DDX56, DDX58, DDX59, DDX6, DDX60, DDX60L, DEAF1, DEC 1, DECR1, DECR2, DEDD, DEDD2, DEF6, DEF8, DEFA1, DEFA1B, DEFA3, DEFA4, DEFA5, DEFA6, DEFB1, DEFB103A, DEFB103B, DEFB104A, DEFB104B, DEFB105A, DEFB105B, DEFB106A, DEFB106B, DEFB107A, DEFB107B, DEFB108B, DEFB110, DEFB112, DEFB113, DEFB114, DEFB115, DEFB116, DEFB118, DEFB119, DEFB121, DEFB123, DEFB124, DEFB125, DEFB126, DEFB127, DEFB128, DEFB129, DEFB130A, DEFB130B, DEFB131A, DEFB131B, DEFB132, DEFB133, DEFB134, DEFB135, DEFB136, DEFB4A, DEFB4B, DEGS1, DEGS2, DEK, DENND1A, DENND1B, DENNDl1C, DENND2A, DENND2C, DENND2D, DENND3, DENND4A, DENND4B, DENND4C, DENND5A, DENND5B, DENND6A, DENND6B, DENR, DEPDC1, DEPDC1B, DEPDC4, DEPDC5, DEPDC7, DEPTOR, DERA, DERL1, DERL2, DERL3, DES, DESII, DESI2, DET1, DEUP1, DEXI, DFFA, DFFB, DFNA5, DFNB59, DGAT1, DGAT2, DGAT2L6, DGCR2, DGCR6, DGCR6L, DGCR8, DGKA, DGKB, DGKD, DGKE, DGKG, DGKH, DGKI, DGKK, DGKQ, DGKZ, DGUOK, DHCR24, DHCR7, DHDDS, DHDH, DHFR, DHFR2, DHH, DHODH, DHPS, DHRS1, DHRS11, DHRS12, DHRS13, DHRS2, DHRS3, DHRS4, DHRS4L2, DHRS7, DHRS7B, DHRS7C, DHRS9, DHRSX, DHTKD1, DHX15, DHX16, DHX29, DHX30, DHX32, DHX33, DHX34, DHX35, DHX36, DHX37, DHX38, DHX40, DHX57, DHX58, DHX8, DHX9, DIABLO, DIAPH1, DIAPH2, DIAPH3, DICER1, DIDO1, DIEXF, DIMT1, DIO1, DIO2, DIO3, DIP2A, DIP2B, DIP2C, DIRAS1, DIRAS2, DIRAS3, DIRC1, DIRC2, DIRC3, DIS3, DIS3L, DIS3L2, DISC1, DISP1, DISP2, DISP3, DIXDC1, DKC1, DKK1, DKK2, DKK3, DKK4, DKKL1, DLAT, DLC1, DLD, DLEC1, DLEU7, DLG1, DLG2, DLG3, DLG4, DLG5, DLGAP1, DLGAP2, DLGAP3, DLGAP4, DLGAP5, DLK1, DLK2, DLL1, DLL3, DLL4, DLST, DLX1, DLX2, DLX3, DLX4, DLX5, DLX6, DMAC1, DMAC2, DMAP1, DMBT1, DMBX1, DMC1, DMD, DMGDH, DMKN, DMP1, DMPK, DMRT1, DMRT2, DMRT3, DMRTA1, DMRTA2, DMRTB1, DMRTC1, DMRTC1B, DMRTC2, DMTF1, DMTN, DMWD, DMXL1, DMXL2, DNA2, DNAAF1, DNAAF2, DNAAF3, DNAAF4, DNAAF5, DNAH1, DNAH10, DNAH10OS, DNAH11, DNAH12, DNAH14, DNAH17, DNAH2, DNAH3, DNAH5, DNAH6, DNAH7, DNAH8, DNAH9, DNAI1, DNAI2, DNAJA1, DNAJA2, DNAJA3, DNAJA4, DNAJB1, DNAJB11, DNAJB12, DNAJB13, DNAJB14, DNAJB2, DNAJB4, DNAJB5, DNAJB6, DNAJB7, DNAJB8, DNAJB9, DNAJC1, DNAJC10, DNAJC11, DNAJC12, DNAJC13, DNAJC14, DNAJC15, DNAJC16, DNAJC17, DNAJC18, DNAJC19, DNAJC2, DNAJC21, DNAJC22, DNAJC24, DNAJC25, DNAJC25-GNG10, DNAJC27, DNAJC28, DNAJC3, DNAJC30, DNAJC4, DNAJC5, DNAJC5B, DNAJC5G, DNAJC6, DNAJC7, DNAJC8, DNAJC9, DNAL1, DNAL4, DNALI1, DNASE1, DNASE1L1, DNASE1L2, DNASE1L3, DNASE2, DNASE2B, DND1, DNER, DNHD1, DNLZ, DNM1, DNM1L, DNM2, DNM3, DNMBP, DNMT1, DNMT3A, DNMT3B, DNMT3L, DNPEP, DNPH1, DNTT, DNTTIP1, DNTTIP2, DOC2A, DOC2B, DOCK1, DOCK10, DOCK11, DOCK2, DOCK3, DOCK4, DOCK5, DOCK6, DOCK7, DOCK8, DOCK9, DOHH, DOK1, DOK2, DOK3, DOK4, DOK5, DOK6, DOK7, DOLK, DOLPP1, DONSON, DOPEY1, DOPEY2, DOT1L, DPAGT1, DPCD, DPCR1, DPEP1, DPEP2, DPEP3, DPF1, DPF2, DPF3, DPH1, DPH2, DPH3, DPH5, DPH6, DPH7, DPM1, DPM2, DPM3, DPP10, DPP3, DPP4, DPP6, DPP7, DPP8, DPP9, DPPA2, DPPA3, DPPA4, DPPA5, DPRX, DPT, DPY19L1, DPY19L2, DPY19L3, DPY19L4, DPY30, DPYD, DPYS, DPYSL2, DPYSL3, DPYSL4, DPYSL5, DQX1, DR1, DRAM1, DRAM2, DRAP1, DRAXIN, DRC1, DRC3, DRC7, DRD1, DRD2, DRD3, DRD4, DRD5, DRG1, DRG2, DRGX, DRICH1, DROSHA, DRP2, DSC1, DSC2, DSC3, DSCAM, DSCAML1, DSCC1, DSCR3, DSCR4, DSCR8, DSE, DSEL, DSG1, DSG2, DSG3, DSG4, DSN1, DSP, DSPP, DST, DSTN, DSTYK, DTD1, DTD2, DTHD1, DTL, DTNA, DTNB, DTNBP1, DTWD1, DTWD2, DTX1, DTX2, DTX3, DTX3L, DTX4, DTYMK, DUOX1, DUOX2, DUOXA1, DUOXA2, DUPD1, DUS1L, DUS2, DUS3L, DUS4L, DUSP1, DUSP10, DUSP11, DUSP12, DUSP13, DUSP14, DUSP15, DUSP16, DUSP18, DUSP19, DUSP2, DUSP21, DUSP22, DUSP23, DUSP26, DUSP27, DUSP28, DUSP3, DUSP4, DUSP5, DUSP6, DUSP7, DUSP8, DUSP9, DUT, DUX4, DUXA, DUXB, DVL1, DVL2, DVL3, DWORF, DXO, DYDC1, DYDC2, DYM, DYNAP, DYNC1H1, DYNC1I1, DYNC1I2, DYNC1LI1, DYNC1LI2, DYNC2H1, DYNC2LI1, DYNLL1, DYNLL2, DYNLRB1, DYNLRB2, DYNLT1, DYNLT3, DYRK1A, DYRK1B, DYRK2, DYRK3, DYRK4, DYSF, DYTN, DZANK1, DZIP1, DZIP1L, DZIP3, E2F1, E2F2, E2F3, E2F4, E2F5, E2F6, E2F7, E2F8, E4F1, EAF1, EAF2, EAPP, EARS2, EBAG9, EBF1, EBF2, EBF3, EBF4, EBI3, EBLN1, EBLN2, EBNA1BP2, EBP, EBPL, ECD, ECE1, ECE2, ECEL1, ECH1, ECHDC1, ECHDC2, ECHDC3, ECHS1, ECI1, ECI2, ECM1, ECM2, ECSCR, ECS1T, ECT2, ECT2L, EDA, EDA2R, EDAR, EDARADD, EDC3, EDC4, EDDM13, EDDM3A, EDDM3B, EDEM1, EDEM2, EDEM3, EDF1, EDIL3, EDN1, EDN2, EDN3, EDNRA, EDNRB, EDRF1, EEA1, EED, EEF1A1, EEF1A2, EEF1AKMT1, EEF1AKMT2, EEF1AKMT3, EEF1B2, EEF1D, EEF1E1, EEF1E1-BLOC1S5, EEF1G, EEF2, EEF2K, EEF2KMT, EEFSEC, EEPD1, EFCAB1, EFCAB10, EFCAB11, EFCAB12, EFCAB13, EFCAB14, EFCAB2, EFCAB3, EFCAB5, EFCAB6, EFCAB7, EFCAB8, EFCAB9, EFCC1, EFEMP1, EFEMP2, EFHB, EFHC1, EFHC2, EFHD1, EFHD2, EFL1, EFNA1, EFNA2, EFNA3, EFNA4, EFNA5, EFNB1, EFNB2, EFNB3, EFR3A, EFR3B, EFS, EFTUD2, EGF, EGFL6, EGFL7, EGFL8, EGFLAM, EGFR, EGLN1, EGLN2, EGLN3, EGR1, EGR2, EGR3, EGR4, EHBP1, EHBP1L1, EHD1, EHD2, EHD3, EHD4, EHF, EHHADH, EHMT1, EHMT2, EI24, EID1, EID2, EID2B, EID3, EIF1, EIF1AD, EIF1AX, EIF1AY, EIF1B, EIF2A, EIF2AK1, EIF2AK2, EIF2AK3, EIF2AK4, EIF2B1, EIF2B2, EIF2B3, EIF2B4, EIF2B5, EIF2D, EIF2S1, EIF2S2, EIF2S3, EIF3A, EIF3B, EIF3C, EIF3CL, EIF3D, EIF3E, EIF3F, EIF3G, EIF3H, EIF3I, EIF3J, EIF3K, EIF3L, EIF3M, EIF4A1, EIF4A2, EIF4A3, EIF4B, EIF4E, EIF4E1B, EIF4E2, EIF4E3, EIF4EBP1, EIF4EBP2, EIF4EBP3, EIF4ENIF1, EIF4G1, EIF4G2, EIF4G3, EIF4H, EIF5, EIF5A, EIF5A2, EIF5AL1, EIF5B, EIF6, EIPR1, ELAC1, ELAC2, ELANE, ELAVL1, ELAVL2, ELAVL3, ELAVL4, ELF1, ELF2, ELF3, ELF4, ELF5, ELFN1, ELFN2, ELK1, ELK3, ELK4, ELL, ELL2, ELL3, ELMO1, ELMO2, ELMO3, ELMOD1, ELMOD2, ELMOD3, ELMSAN1, ELN, ELOA, ELOA2, ELOA3, ELOA3B, ELOA3C, ELOA3D, ELOB, ELOC, ELOF1, ELOVL1, ELOVL2, ELOVL3, ELOVL4, ELOVL5, ELOVL6, ELOVL7, ELP1, ELP2, ELP3, ELP4, ELP5, ELP6, ELSPBP1, EMB, EMC1, EMC10, EMC2, EMC3, EMC4, EMC6, EMC7, EMC8, EMC9, EMCN, EMD, EME1, EME2, EMG1, EMID1, EMILIN1, EMILIN2, EMILIN3, EML1, EML2, EML3, EML4, EML5, EML6, EMP1, EMP2, EMP3, EMSY, EMX1, EMX2, EN1, EN2, ENAH, ENAM, ENC1, ENDOD1, ENDOG, ENDOU, ENDOV, ENG, ENGASE, ENHO, ENKD1, ENKUR, ENO1, ENO2, ENO3, ENO4, ENOPH1, ENOSF1, ENOX1, ENOX2, ENPEP, ENPP1, ENPP2, ENPP3, ENPP4, ENPP5, ENPP6, ENPP7, ENSA, ENTHD1, ENTPD1, ENTPD2, ENTPD3, ENTPD4, ENTPD5, ENTPD6, ENTPD7, ENTPD8, ENY2, EOGT, EOMES, EP300, EP400, EPAS1, EPB41, EPB41L1, EPB41L2, EPB41L3, EPB41L4A, EPB41L4B, EPB41L5, EPB42, EPC1, EPC2, EPCAM, EPDR1, EPG5, EPGN, EPHA1, EPHA10, EPHA2, EPHA3, EPHA4, EPHA5, EPHA6, EPHA7, EPHA8, EPHB1, EPHB2, EPHB3, EPHB4, EPHB6, EPHX1, EPHX2, EPHX3, EPHX4, EPM2A, EPM2AIP1, EPN1, EPN2, EPN3, EPO, EPOP, EPOR, EPPIN, EPPIN-WFDC6, EPPK1, EPRS, EPS15, EPS15L1, EPS8, EPS8L1, EPS8L2, EPS8L3, EPSTI1, EPX, EPYC, EQTN, ERAL1, ERAP1, ERAP2, ERAS, ERBB2, ERBB3, ERBB4, ERBIN, ERC1, ERC2, ERCC1, ERCC2, ERCC3, ERCC4, ERCC5, ERCC6, ERCC6L, ERCC6L2, ERCC8, EREG, ERF, ERFE, ERG, ERG28, ERGIC1, ERGIC2, ERGIC3, ERH, ERI1, ERI2, ERI3, ERICH1, ERICH2, ERICH3, ERICH4, ERICH5, ERICH6, ERICH6B, ERLEC1, ERLIN1, ERLIN2, ERMAP, ERMARD, ERMN, ERMP1, ERN1, ERN2, ERO1A, ERO1B, ERP27, ERP29, ERP44, ERRFI1, ERV3-1, ERVFRD-1, ERVMER34-1, ERVV-1, ERVV-2, ERVW-1, ESAM, ESCO1, ESCO2, ESD, ESF1, ESM1, ESPL1, ESPN, ESPNL, ESR1, ESR2, ESRP1, ESRP2, ESRRA, ESRRB, ESRRG, ESS2, ESX1, ESYT1, ESYT2, ESYT3, ETAA1, ETDA, ETDB, ETDC, ETF1, ETFA, ETFB, ETFBKMT, ETFDH, ETFRF1, ETHE1, ETNK1, ETNK2, ETNPPL, ETS1, ETS2, ETV1, ETV2, ETV3, ETV3L, ETV4, ETV5, ETV6, ETV7, EVA1A, EVA1B, EVA1C, EVC, EVC2, EVI2A, EVI2B, EVI5, EVI5L, EVL, EVPL, EVPLL, EVX1, EVX2, EWSR1, EXD1, EXD2, EXD3, EXO1, EXO5, EXOC1, EXOC1L, EXOC2, EXOC3, EXOC3L1, EXOC3L2, EXOC3L4, EXOC4, EXOC5, EXOC6, EXOC6B, EXOC7, EXOC8, EXOG, EXOSC1, EXOSC10, EXOSC2, EXOSC3, EXOSC4, EXOSC5, EXOSC6, EXOSC7, EXOSC8, EXOSC9, EXPH5, EXT1, EXT2, EXTL1, EXTL2, EXTL3, EYA1, EYA2, EYA3, EYA4, EYS, EZH1, EZH2, EZR, F10, F11, F1R, F12, F13A1, F13B, F2, F2R, F2RL1, F2RL2, F2RL3, F3, F5, F7, F8, F8A1, F8A2, F8A3, F9, FA2H, FAAH, FAAH2, FAAP100, FAAP20, FAAP24, FABP1, FABP12, FABP2, FABP3, FABP4, FABP5, FABP6, FABP7, FABP9, FADD, FADS1, FADS2, FADS3, FADS6, FAF1, FAF2, FAH, FAHD1, FAHD2A, FAHD2B, FAIM, FAIM2, FAM102A, FAM102B, FAM103A1, FAM104A, FAM104B, FAM105A, FAM106A, FAM107A, FAM107B, FAM109A, FAM109B, FAM110A, FAM110B, FAM110C, FAM110D, FAM111A, FAM111B, FAM114A1, FAM114A2, FAM117A, FAM117B, FAM118A, FAM118B, FAM120A, FAM120AOS, FAM120B, FAM120C, FAM122A, FAM122B, FAM122C, FAM124A, FAM124B, FAM126A, FAM126B, FAM129A, FAM129B, FAM129C, FAM131A, FAM131B, FAM131C, FAM133A, FAM133B, FAM135A, FAM135B, FAM136A, FAM13A, FAM13B, FAM13C, FAM149A, FAM149B1, FAM151A, FAM151B, FAM153A, FAM153B, FAM153C, FAM155A, FAM155B, FAM156A, FAM156B, FAM159A, FAM159B, FAM160A1, FAM160A2, FAM160B1, FAM160B2, FAM161A, FAM161B, FAM162A, FAM162B, FAM163A, FAM163B, FAM166A, FAM166B, FAM167A, FAM167B, FAM168A, FAM168B, FAM169A, FAM169B, FAM170A, FAM170B, FAM171A1, FAM171A2, FAM171B, FAM172A, FAM173A, FAM173B, FAM174A, FAM174B, FAM177A1, FAM177B, FAM178B, FAM180A, FAM180B, FAM181A, FAM181B, FAM182B, FAM183A, FAM184A, FAM184B, FAM185A, FAM186A, FAM186B, FAM187A, FAM187B, FAM189A1, FAM189A2, FAM189B, FAM192A, FAM193A, FAM193B, FAM196A, FAM196B, FAM198A, FAM198B, FAM199X, FAM19A1, FAM19A2, FAM19A3, FAM19A4, FAM19A5, FAM200A, FAM200B, FAM204A, FAM205A, FAM205C, FAM206A, FAM207A, FAM208A, FAM208B, FAM209A, FAM209B, FAM20A, FAM20B, FAM20C, FAM210A, FAM210B, FAM212A, FAM212B, FAM213A, FAM213B, FAM214A, FAM214B, FAM216A, FAM216B, FAM217A, FAM217B, FAM218A, FAM219A, FAM219B, FAM220A, FAM221A, FAM221B, FAM222A, FAM222B, FAM227A, FAM227B, FAM228A, FAM228B, FAM229A, FAM229B, FAM230A, FAM231A, FAM231B, FAM231C, FAM231D, FAM234A, FAM234B, FAM236A, FAM236B, FAM236C, FAM236D, FAM237A, FAM237B, FAM240A, FAM240B, FAM24A, FAM24B, FAM25A, FAM25C, FAM25G, FAM26D, FAM26E, FAM26F, FAM32A, FAM35A, FAM3A, FAM3B, FAM3C, FAM3D, FAM43A, FAM43B, FAM45A, FAM46A, FAM46B, FAM46C, FAM46D, FAM47A, FAM47B, FAM47C, FAM47E, FAM47E-STBD1, FAM49A, FAM49B, FAM50A, FAM50B, FAM53A, FAM53B, FAM53C, FAM57A, FAM57B, FAM58A, FAM60A, FAM69A, FAM69B, FAM69C, FAM71A, FAM71B, FAM71C, FAM71D, FAM71E1, FAM71E2, FAM71F1, FAM71F2, FAM72A, FAM72B, FAM72C, FAM72D, FAM76A, FAM76B, FAM78A, FAM78B, FAM81A, FAM81B, FAM83A, FAM83B, FAM83C, FAM83D, FAM83E, FAM83F, FAM83G, FAM83H, FAM84A, FAM84B, FAM86B1, FAM86B2, FAM86C1, FAM89A, FAM89B, FAM8A1, FAM90A1, FAM90A26, FAM91A1, FAM92A, FAM92B, FAM95C, FAM96A, FAM96B, FAM98A, FAM98B, FAM98C, FAM9A, FAM9B, FAM9C, FAN1, FANCA, FANCB, FANCC, FANCD2, FANCD2OS, FANCE, FANCF, FANCG, FANCI, FANCL, FANCM, FANK1, FAP, FAR1, FAR2, FARP1, FARP2, FARS2, FARSA, FARSB, FAS, FASLG, FASN, FASTK, FASTKD1, FASTKD2, FASTKD3, FASTKD5, FAT1, FAT2, FAT3, FAT4, FATE1, FAU, FAXC, FAXDC2, FBF1, FBL, FBLIM1, FBLL1, FBLN1, FBLN2, FBLN5, FBLN7, FBN1, FBN2, FBN3, FBP1, FBP2, FBRS, FBRSL1, FBXL12, FBXL13, FBXL14, FBXL15, FBXL16, FBXL17, FBXL18, FBXL19, FBXL2, FBXL20, FBXL22, FBXL3, FBXL4, FBXL5, FBXL6, FBXL7, FBXL8, FBXO10, FBXO11, FBX015, FBX016, FBX017, FBX018, FBXO2, FBXO21, FBXO22, FBXO24, FBXO25, FBXO27, FBXO28, FBXO3, FBXO30, FBXO31, FBXO32, FBXO33, FBXO34, FBXO36, FBXO38, FBXO39, FBXO4, FBXO40, FBXO41, FBXO42, FBXO43, FBXO44, FBXO45, FBXO46, FBXO47, FBXO48, FBX05, FBXO6, FBXO7, FBXO8, FBXO9, FBXW10, FBXW11, FBXW12, FBXW2, FBXW4, FBXW5, FBXW7, FBXW8, FBXW9, FCAMR, FCAR, FCER1A, FCER1G, FCER2, FCF1, FCGBP, FCGR1A, FCGR1B, FCGR2A, FCGR2B, FCGR2C, FCGR3A, FCGR3B, FCGRT, FCHO1, FCHO2, FCHSD1, FCHSD2, FCMR, FCN1, FCN2, FCN3, FCRL1, FCRL2, FCRL3, FCRL4, FCRL5, FCRL6, FCRLA, FCRLB, FDCSP, FDFT1, FDPS, FDX1, FDX2, FDXACB1, FDXR, FECH, FEM1A, FEM1B, FEM1C, FEN1, FER, FER1L5, FER1L6, FERD3L, FERMT1, FERMT2, FERMT3, FES, FETUB, FEV, FEZ1, FEZ2, FEZF1, FEZF2, FFAR1, FFAR2, FFAR3, FFAR4, FGA, FGB, FGD1, FGD2, FGD3, FGD4, FGD5, FGD6, FGF1, FGF10, FGF11, FGF12, FGF13, FGF14, FGF16, FGF17, FGF18, FGF19, FGF2, FGF20, FGF21, FGF22, FGF23, FGF3, FGF4, FGF5, FGF6, FGF7, FGF8, FGF9, FGFBP1, FGFBP2, FGFBP3, FGFR1, FGFR1OP, FGFR10P2, FGFR2, FGFR3, FGFR4, FGFRL1, FGG, FGGY, FGL1, FGL2, FGR, FH, FHAD1, FHDC1, FHIT, FHL1, FHL2, FHL3, FHL5, FHOD1, FHOD3, FIBCD1, FIBIN, FIBP, FICD, FIG4, FIGLA, FIGN, FIGNL1, FIGNL2, FILIP1, FILIP1L, FIP1L1, FIS1, FITM1, FITM2, FIZ1, FJX1, FKBP10, FKBP11, FKBP14, FKBP15, FKBP1A, FKBP1B, FKBP1C, FKBP2, FKBP3, FKBP4, FKBP5, FKBP6, FKBP7, FKBP8, FKBP9, FKBPL, FKRP, FKTN, FLAD1, FLCN, FLG, FLG2, FLI1, FLII, FLNA, FLNB, FLNC, FLOT1, FLOT2, FLRT1, FLRT2, FLRT3, FLT1, FLT3, FLT3LG, FLT4, FLVCR1, FLVCR2, FLYWCH1, FLYWCH2, FMC1, FMN1, FMN2, FMNL1, FMNL2, FMNL3, FMO1, FMO2, FMO3, FMO4, FMO5, FMOD, FMR1, FMR1NB, FN1, FN3K, FN3KRP, FNBP1, FNBP1L, FNBP4, FNDC1, FNDC10, FNDC11, FNDC3A, FNDC3B, FNDC4, FNDC5, FNDC7, FNDC8, FNDC9, FNIP1, FNIP2, FNTA, FNTB, F0681492.1, F0681542.1, FOCAD, FOLH1, FOLR1, FOLR2, FOLR3, FOPNL, FOS, FOSB, FOSL1, FOSL2, FOXA1, FOXA2, FOXA3, FOXB1, FOXB2, FOXC1, FOXC2, FOXD1, FOXD2, FOXD3, FOXD4, FOXD4L1, FOXD4L3, FOXD4L4, FOXD4L5, FOXD4L6, FOXE1, FOXE3, FOXF1, FOXF2, FOXG1, FOXH1, FOXI1, FOXI2, FOXI3, FOXJ1, FOXJ2, FOXJ3, FOXK1, FOXK2, FOXL1, FOXL2, FOXL2NB, FOXM1, FOXN1, FOXN2, FOXN3, FOXN4, FOXO1, FOXO3, FOXO4, FOXO6, FOXP1, FOXP2, FOXP3, FOXP4, FOXQ1, FOXR1, FOXR2, FOXRED1, FOXRED2, FOXS1, FP236240.1, FP565260.1, FP565260.2, FP565260.3, FP565260.4, FP565260.6, FP565260.7, FP565324.1, FP565324.2, FPGS, FPGT, FPGT-TNNI3K, FPR1, FPR2, FPR3, FRA10AC1, FRAS1, FRAT1, FRAT2, FREM1, FREM2, FREM3, FRG1, FRG2, FRG2B, FRG2C, FRK, FRMD1, FRMD3, FRMD4A, FRMD4B, FRMD5, FRMD6, FRMD7, FRMD8, FRMPD1, FRMPD2, FRMPD3, FRMPD4, FRRS1, FRRS1L, FRS2, FRS3, FRY, FRYL, FRZB, FSBP, FSCB, FSCN1, FSCN2, FSCN3, FSD1, FSD1L, FSD2, FSHB, FSHR, FSIP1, FSIP2, FST, FSTL1, FSTL3, FSTL4, FSTL5, FTCD, FTCDNL1, FTH1, FTHL17, FTL, FTMT, FTO, FTSJ1, FTSJ3, FUBP1, FUBP3, FUCA1, FUCA2, FUK, FUNDC1, FUNDC2, FUOM, FURIN, FUS, FUT1, FUT10, FUT11, FUT2, FUT3, FUT4, FUT5, FUT6, FUT7, FUT8, FUT9, FUZ, FXN, FXR1, FXR2, FXYD1, FXYD2, FXYD3, FXYD4, FXYD5, FXYD6, FXYD6-FXYD2, FXYD7, FYB1, FYB2, FYCO1, FYN, FYTTD1, FZD1, FZD10, FZD2, FZD3, FZD4, FZD5, FZD6, FZD7, FZD8, FZD9, FZR1, G0S2, G2E3, G3BP1, G3BP2, G6PC, G6PC2, G6PC3, G6PD, GAA, GAB1, GAB2, GAB3, GAB4, GABARAP, GABARAPL1, GABARAPL2, GABBR1, GABBR2, GABPA, GABPB1, GABPB2, GABRA1, GABRA2, GABRA3, GABRA4, GABRA5, GABRA6, GABRB1, GABRB2, GABRB3, GABRD, GABRE, GABRG1, GABRG2, GABRG3, GABRP, GABRQ, GABRR1, GABRR2, GABRR3, GAD1, GAD2, GADD45A, GADD45B, GADD45G, GADD45GIP1, GADL1, GAGE1, GAGE10, GAGE12B, GAGE12C, GAGE12D, GAGE12E, GAGE12F, GAGE12G, GAGE12H, GAGE12J, GAGE13, GAGE2A, GAGE2E, GAK, GAL, GAL3ST1, GAL3ST2, GAL3ST3, GAL3ST4, GALC, GALE, GALK1, GALK2, GALM, GALNS, GALNT1, GALNT10, GALNT11, GALNT12, GALNT13, GALNT14, GALNT15, GALNT16, GALNT17, GALNT18, GALNT2, GALNT3, GALNT4, GALNT5, GALNT6, GALNT7, GALNT8, GALNT9, GALNTL5, GALNTL6, GALP, GALR1, GALR2, GALR3, GALT, GAMT, GAN, GANAB, GANC, GAP43, GAPDH, GAPDHS, GAPT, GAPVD1, GAR1, GAREM1, GAREM2, GARNL3, GARS, GART, GAS1, GAS2, GAS2L1, GAS2L2, GAS2L3, GAS6, GAS7, GAS8, GAST, GATA1, GATA2, GATA3, GATA4, GATA5, GATA6, GATAD1, GATAD2A, GATAD2B, GATB, GATC, GATD1, GATM, GATS, GBA, GBA2, GBA3, GBE1, GBF1, GBGT1, GBP1, GBP2, GBP3, GBP4, GBP5, GBP6, GBP7, GBX1, GBX2, GC, GCA, GCAT, GCC1, GCC2, GCDH, GCFC2, GCG, GCGR, GCH1, GCHFR, GCK, GCKR, GCLC, GCLM, GCM1, GCM2, GCN1, GCNA, GCNT1, GCNT2, GCNT3, GCNT4, GCNT7, GCOM1, GCSAM, GCSAML, GCSH, GDA, GDAP1, GDAP1L1, GDAP2, GDE1, GDF1, GDF10, GDF11, GDF15, GDF2, GDF3, GDF5, GDF5OS, GDF6, GDF7, GDF9, GDI1, GDI2, GDNF, GDPD1, GDPD2, GDPD3, GDPD4, GDPD5, GDPGP1, GEM, GEMIN2, GEMIN4, GEMIN5, GEMIN6, GEMIN7, GEMIN8, GEN1, GET4, GFAP, GFER, GFI1, GFI1B, GFM1, GFM2, GFOD1, GFOD2, GFPT1, GFPT2, GFRA1, GFRA2, GFRA3, GFRA4, GFRAL, GFY, GGA1, GGA2, GGA3, GGACT, GGCT, GGCX, GGH, GGN, GGNBP2, GGPS1, GGT1, GGT2, GGT5, GGT6, GGT7, GGTLC1, GGTLC2, GGTLC3, GH1, GH2, GHDC, GHITM, GHR, GHRH, GHRHR, GHRL, GHSR, GID4, GID8, GIF, GIGYF1, GIGYF2, GIMAP1, GIMAP1-GIMAP5, GIMAP2, GIMAP4, GIMAP5, GIMAP6, GIMAP7, GIMAP8, GIMD1, GIN1, GINM1, GINS1, GINS2, GINS3, GINS4, GIP, GIPC1, GIPC2, GIPC3, GIPR, GIT1, GIT2, GJA1, GJA10, GJA3, GJA4, GJA5, GJA8, GJA9, GJB1, GJB2, GJB3, GJB4, GJB5, GJB6, GJB7, GJC1, GJC2, GJC3, GJD2, GJD3, GJD4, GJE1, GK, GK2, GK3P, GK5, GKAP1, GKN1, GKN2, GLA, GLB1, GLB1L, GLB1L2, GLB1L3, GLCCI1, GLCE, GLDC, GLDN, GLE1, GLG1, GLI1, GLI2, GLI3, GLI4, GLIPR1, GLIPR1L1, GLIPR1L2, GLIPR2, GLIS1, GLIS2, GLIS3, GLMN, GLMP, GLO1, GLOD4, GLOD5, GLP1R, GLP2R, GLRA1, GLRA2, GLRA3, GLRA4, GLRB, GLRX, GLRX2, GLRX3, GLRX5, GLS, GLS2, GLT1D1, GLT6D1, GLT8D1, GLT8D2, GLTP, GLTPD2, GLUD1, GLUD2, GLUL, GLYAT, GLYATL1, GLYATL1P3, GLYATL2, GLYATL3, GLYCTK, GLYR1, GM2A, GMCL1, GMDS, GMEB1, GMEB2, GMFB, GMFG, GMIP, GML, GMNC, GMNN, GMPPA, GMPPB, GMPR, GMPR2, GMPS, GNA11, GNA12, GNA13, GNA14, GNA15, GNAI1, GNAI2, GNAI3, GNAL, GNAO1, GNAQ, GNAS, GNAT1, GNAT2, GNAT3, GNAZ, GNB1, GNB1L, GNB2, GNB3, GNB4, GNB5, GNE, GNG10, GNG11, GNG12, GNG13, GNG14, GNG2, GNG3, GNG4, GNG5, GNG7, GNG8, GNGT1, GNGT2, GNL1, GNL2, GNL3, GNL3L, GNLY, GNMT, GNPAT, GNPDA1, GNPDA2, GNPNAT1, GNPTAB, GNPTG, GNRH1, GNRH2, GNRHR, GNS, GOLGA1, GOLGA2, GOLGA3, GOLGA4, GOLGA5, GOLGA6A, GOLGA6B, GOLGA6C, GOLGA6D, GOLGA6L1, GOLGA6L10, GOLGA6L2, GOLGA6L22, GOLGA6L4, GOLGA6L6, GOLGA6L7P, GOLGA6L9, GOLGA7, GOLGA7B, GOLGA8A, GOLGA8B, GOLGA8F, GOLGA8G, GOLGA8H, GOLGA8J, GOLGA8K, GOLGA8M, GOLGA8N, GOLGA80, GOLGA8Q, GOLGA8R, GOLGA8S, GOLGA8T, GOLGB1, GOLIM4, GOLM1, GOLPH3, GOLPH3L, GOLT1A, GOLT1B, GON4L, GON7, GOPC, GORAB, GORASP1, GORASP2, GOSR1, GOSR2, GOT1, GOT1L1, GOT2, GP1BA, GP1BB, GP2, GP5, GP6, GP9, GPA33, GPAA1, GPALPP1, GPAM, GPANK1, GPAT2, GPAT3, GPAT4, GPATCH1, GPATCH11, GPATCH2, GPATCH2L, GPATCH3, GPATCH4, GPATCH8, GPBAR1, GPBP1, GPBP1L1, GPC1, GPC2, GPC3, GPC4, GPC5, GPC6, GPCPD1, GPD1, GPD1L, GPD2, GPER1, GPHA2, GPHB5, GPHN, GPI, GPIBP1, GPKOW, GPLD1, GPM6A, GPM6B, GPN1, GPN2, GPN3, GPNMB, GPR1, GPR101, GPR107, GPR108, GPR119, GPR12, GPR132, GPR135, GPR137, GPR137B, GPR137C, GPR139, GPR141, GPR142, GPR143, GPR146, GPR148, GPR149, GPR15, GPR150, GPR151, GPR152, GPR153, GPR155, GPR156, GPR157, GPR158, GPR160, GPR161, GPR162, GPR17, GPR171, GPR173, GPR174, GPR176, GPR179, GPR18, GPR180, GPR182, GPR183, GPR19, GPR20, GPR21, GPR22, GPR25, GPR26, GPR27, GPR3, GPR31, GPR32, GPR33, GPR34, GPR35, GPR37, GPR37L1, GPR39, GPR4, GPR42, GPR45, GPR50, GPR52, GPR55, GPR6, GPR61, GPR62, GPR63, GPR65, GPR68, GPR75, GPR75-ASB3, GPR78, GPR82, GPR83, GPR84, GPR85, GPR87, GPR88, GPR89A, GPR89B, GPRASP1, GPRASP2, GPRC5A, GPRC5B, GPRC5C, GPRC5D, GPRC6A, GPRIN1, GPRIN2, GPRIN3, GPS1, GPS2, GPSM1, GPSM2, GPSM3, GPT, GPT2, GPX1, GPX2, GPX3, GPX4, GPX5, GPX6, GPX7, GPX8, GRAMD1A, GRAMD1B, GRAMD1C, GRAMD2A, GRAMD2B, GRAMD4, GRAP, GRAP2, GRAPL, GRASP, GRB10, GRB14, GRB2, GRB7, GREB1, GREB1L, GREM1, GREM2, GRHL1, GRHL2, GRHL3, GRHPR, GRIA1, GRIA2, GRIA3, GRIA4, GRID1, GRID2, GRID2IP, GRIFIN, GRIK1, GRIK2, GRIK3, GRIK4, GRIK5, GRIN1, GRIN2A, GRIN2B, GRIN2C, GRIN2D, GRIN3A, GRIN3B, GRINA, GRIP1, GRIP2, GRIPAP1, GRK1, GRK2, GRK3, GRK4, GRK5, GRK6, GRK7, GRM1, GRM2, GRM3, GRM4, GRM5, GRM6, GRM7, GRM8, GRN, GRP, GRPEL1, GRPEL2, GRPR, GRSF1, GRTP1, GRWD1, GRXCR1, GRXCR2, GSAP, GSC, GSC2, GSDMA, GSDMB, GSDMC, GSDMD, GSE1, GSG1, GSG1L, GSG1L2, GSK3A, GSK3B, GSKIP, GSN, GSPT1, GSPT2, GSR, GSS, GSTA1, GSTA2, GSTA3, GSTA4, GSTA5, GSTCD, GSTK1, GSTM1, GSTM2, GSTM3, GSTM4, GSTM5, GSTO1, GSTG2, GSTP1, GSTT1, GSTT2, GSTT2B, GSTTP1, GSTZ1, GSX1, GSX2, GTDC1, GTF2A1, GTF2A1L, GTF2A2, GTF2B, GTF2E1, GTF2E2, GTF2F1, GTF2F2, GTF2H1, GTF2H2, GTF2H2C, GTF2H2C2, GTF2H3, GTF2H4, GTF2H5, GTF2I, GTF2IRD1, GTF2IRD2, GTF2IRD2B, GTF3A, GTF3C1, GTF3C2, GTF3C3, GTF3C4, GTF3C5, GTF3C6, GTPBP1, GTPBP10, GTPBP2, GTPBP3, GTPBP4, GTPBP6, GTPBP8, GTSE1, GTSF1, GTSF1L, GU182339.1, GU182339.3, GU182343.1, GU182343.2, GU182345.1, GU182345.2, GU182347.1, GU182351.2, GU182352.2, GU182353.1, GU182355.1, GU182355.2, GU182355.3, GU182357.1, GU182357.3, GU182359.1, GU182359.2, GUCA1A, GUCA1B, GUCA1C, GUCA2A, GUCA2B, GUCD1, GUCY1A2, GUCY1A3, GUCY1B3, GUCY2C, GUCY2D, GUCY2F, GUF1, GUK1, GULP1, GUSB, GVQW2, GXYLT1, GXYLT2, GYG1, GYG2, GYPA, GYPB, GYPC, GYPE, GYS1, GYS2, GZF1, GZMA, GZMB, GZMH, GZMK, GZMM, HIF0, H1FNT, H1FOO, H1FX, H2AFB1, H2AFB2, H2AFB3, H2AFJ, H2AFV, H2AFX, H2AFY, H2AFY2, H2AFZ, H2BFM, H2BFS, H2BFWT, H3F3A, H3F3B, H3F3C, H6PD, HAAO, HABP2, HABP4, HACD1, HACD2, HACD3, HACD4, HACE1, HACL1, HADH, HADHA, HADHB, HAGH, HAGHL, HAL, HAMP, HAND1, HAND2, HAO1, HAG2, HAP1, HAPLN1, HAPLN2, HAPLN3, HAPLN4, HARBI1, HARS, HARS2, HAS1, HAS2, HAS3, HASPIN, HAT1, HAUS1, HAUS2, HAUS3, HAUS4, HAUS5, HAUS6, HAUS7, HAUS8, HAVCR1, HAVCR2, HAX1, HBA1, HBA2, HBB, HBD, HBE1, HBEGF, HBG1, HBG2, HBM, HBP1, HBQ1, HBS1L, HBZ, HCAR1, HCAR2, HCAR3, HCCS, HCFC1, HCFC1R1, HCFC2, HCK, HCLS1, HCN1, HCN2, HCN3, HCN4, HCRT, HCRTR1, HCRTR2, HCST, HDAC1, HDAC10, HDAC11, HDAC2, HDAC3, HDAC4, HDAC5, HDAC6, HDAC7, HDAC8, HDAC9, HDC, HDDC2, HDDC3, HDGF, HDGFL1, HDGFL2, HDGFL3, HDHD2, HDHD3, HDHD5, HDLBP, HDX, HEATR1, HEATR3, HEATR4, HEATR5A, HEATR5B, HEATR6, HEATR9, HEBP1, HEBP2, HECA, HECTD1, HECTD2, HECTD3, HECTD4, HECW1, HECW2, HEG1, HELB, HELLS, HELQ, HELT, HELZ, HELZ2, HEMGN, HEMK1, HENMT1, HEPACAM, HEPACAM2, HEPH, HEPHL1, HEPN1, HERC1, HERC2, HERC3, HERC4, HERC5, HERC6, HERPUD1, HERPUD2, HES1, HES2, HES3, HES4, HES5, HES6, HES7, HESX1, HEXA, HEXB, HEXDC, HEXIM1, HEXIM2, HEY1, HEY2, HEYL, HFE, HFE2, HFM1, HGD, HGF, HGFAC, HGH1, HGNC: 18790, HGNC: 24955, HGS, HGSNAT, HHAT, HHATL, HHEX, HHIP, HHIPL1, HHIPL2, HHLA1, HHLA2, HHLA3, HIBADH, HIBCH, HIC1, HIC2, HID1, HIF1A, HIF1AN, HIF3A, HIGD1A, HIGD1B, HIGD1C, HIGD2A, HIGD2B, HIKESHI, HILPDA, HINFP, HINT1, HINT2, HINT3, HIP1, HIP1R, HIPK1, HIPK2, HIPK3, HIPK4, HIRA, HIRIP3, HIST1H1A, HIST1H1B, HIST1H1C, HIST1H1D, HIST1H1E, HIST1H1T, HIST1H2AA, HIST1H2AB, HIST1H2AC, HIST1H2AD, HIST1H2AE, HIST1H2AG, HIST1H2AH, HIST1H2AI, HIST1H2AJ, HIST1H2AK, HIST1H2AL, HIST1H2AM, HIST1H2BA, HIST1H2BB, HIST1H2BC, HIST1H2BD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, HIST1H3A, HIST1H3B, HIST1H3C, HIST1H3D, HIST1H3E, HIST1H3F, HIST1H3G, HIST1H3H, HIST1H3I, HIST1H3J, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, HIST2H2AA3, HIST2H2AA4, HIST2H2AB, HIST2H2AC, HIST2H2BE, HIST2H2BF, HIST2H3A, HIST2H3C, HIST2H3D, HIST2H3PS2, HIST2H4A, HIST2H4B, HIST3H2A, HIST3H2BB, HIST3H3, HIST4H4, HIVEP1, HIVEP2, HIVEP3, HJURP, HK1, HK2, HK3, HKDC1, HKR1, HLA-A, HLA-B, HLA-C, HLA-DMA, HLA-DMB, HLA-DOA, HLA-DOB, HLA-DPA1, HLA-DPB1, HLA-DQA1, HLA-DQA2, HLA-DQB1, HLA-DQB2, HLA-DRA, HLA-DRB1, HLA-DRB3, HLA-DRB4, HLA-DRB5, HLA-E, HLA-F, HLA-G, HLCS, HLF, HLTF, HLX, HM13, HM190170.1, HMBOXI, HMBS, HMCES, HMCN1, HMCN2, HMG20A, HMG20B, HMGA1, HMGA2, HMGB1, HMGB2, HMGB3, HMGB4, HMGCL, HMGCLL1, HMGCR, HMGCS1, HMGCS2, HMGN1, HMGN2, HMGN3, HMGN4, HMGN5, HMGXB3, HMGXB4, HMHB1, HMMR, HMOX1, HMOX2, HMSD, HMX1, HMX2, HMX3, HNF1A, HNF1B, HNF4A, HNF4G, HNMT, HNRNPA0, HNRNPA1, HNRNPA1L2, HNRNPA2B1, HNRNPA3, HNRNPAB, HNRNPC, HNRNPCL1, HNRNPCL2, HNRNPCL3, HNRNPCL4, HNRNPD, HNRNPDL, HNRNPF, HNRNPH1, HNRNPH2, HNRNPH3, HNRNPK, HNRNPL, HNRNPLL, HNRNPM, HNRNPR, HNRNPU, HNRNPUL1, HNRNPUL2, HNRNPUL2-BSCL2, HOGA1, HOMER1, HOMER2, HOMER3, HOMEZ, HOOK1, HOOK2, HOOK3, HOPX, HORMAD1, HORMAD2, HOXA1, HOXA10, HOXA11, HOXA13, HOXA2, HOXA3, HOXA4, HOXA5, HOXA6, HOXA7, HOXA9, HOXB1, HOXB13, HOXB2, HOXB3, HOXB4, HOXB5, HOXB6, HOXB7, HOXB8, HOXB9, HOXC10, HOXC11, HOXC12, HOXC13, HOXC4, HOXC5, HOXC6, HOXC8, HOXC9, HOXD1, HOXD10, HOXD11, HOXD12, HOXD13, HOXD3, HOXD4, HOXD8, HOXD9, HP, HP1BP3, HPCA, HPCAL1, HPCAL4, HPD, HPDL, HPF1, HPGD, HPGDS, HPN, HPR, HPRT1, HPS1, HPS3, HPS4, HPS5, HPS6, HPSE, HPSE2, HPX, HR, HRAS, HRASLS, HRASLS2, HRASLS5, HRC, HRCT1, HRG, HRH1, HRH2, HRH3, HRH4, HRK, HRNR, HS1BP3, HS2ST1, HS3ST1, HS3ST2, HS3ST3A1, HS3ST3B1, HS3ST4, HS3ST5, HS3ST6, HS6ST1, HS6ST2, HS6ST3, HSBP1, HSBP1L1, HSCB, HSD11B1, HSD11B1L, HSD11B2, HSD17B1, HSD17B10, HSD17B11, HSD17B12, HSD17B13, HSD17B14, HSD17B2, HSD17B3, HSD17B4, HSD17B6, HSD17B7, HSD17B8, HSD3B1, HSD3B2, HSD3B7, HSDL1, HSDL2, HSF1, HSF2, HSF2BP, HSF4, HSF5, HSFX1, HSFX2, HSFX3, HSFX4, HSFY1, HSFY2, HSH2D, HSP90AA1, HSP90AB1, HSP90B1, HSPA12A, HSPA12B, HSPA13, HSPA14, HSPA1A, HSPA1B, HSPA1L, HSPA2, HSPA4, HSPA4L, HSPA5, HSPA6, HSPA8, HSPA9, HSPB1, HSPB11, HSPB2, HSPB2-C11orf52, HSPB3, HSPB6, HSPB7, HSPB8, HSPB9, HSPBAP1, HSPBP1, HSPD1, HSPE1, HSPE1-MOB4, HSPG2, HSPH1, HTATIP2, HTATSF1, HTD2, HTN1, HTN3, HTR1A, HTR1B, HTR1D, HTR1E, HTR1F, HTR2A, HTR2B, HTR2C, HTR3A, HTR3B, HTR3C, HTR3D, HTR3E, HTR4, HTR5A, HTR6, HTR7, HTRA1, HTRA2, HTRA3, HTRA4, HTT, HUNK, HUS1, HUS1B, HUWE1, HVCN1, HYAL1, HYAL2, HYAL3, HYAL4, HYDIN, HYI, HYKK, HYLS1, HYOU1, HYPK, HYPM, IAH1, IAPP, IARS, IARS2, IBA57, IBSP, IBTK, ICA1, ICA1L, ICAM1, ICAM2, ICAM3, ICAM4, ICAM5, ICE1, ICE2, ICK, ICMT, ICOS, ICOSLG, ID1, ID2, ID3, ID4, IDE, IDH1, IDH2, IDH3A, IDH3B, IDH3G, IDI1, IDI2, IDNK, IDO1, IDO2, IDS, IDUA, IER2, IER3, IER3IP1, IER5, IER5L, IFFO1, IFFO2, IFI16, IFI27, IFI27L1, IFI27L2, IFI30, IFI35, IFI44, IFI44L, IFI6, IFIH1, IFIT1, IFIT1B, IFIT2, IFIT3, IFIT5, IFITM1, IFITM10, IFITM2, IFITM3, IFITM5, IFNA1, IFNA10, IFNA13, IFNA14, IFNA16, IFNA17, IFNA2, IFNA21, IFNA4, IFNA5, IFNA6, IFNA7, IFNA8, IFNAR1, IFNAR2, IFNB1, IFNE, IFNG, IFNGR1, IFNGR2, IFNK, IFNL1, IFNL2, IFNL3, IFNL4, IFNLR1, IFNW1, IFRD1, IFRD2, IFT122, IFT140, IFT172, IFT20, IFT22, IFT27, IFT43, IFT46, IFT52, IFT57, IFT74, IFT80, IFT81, IFT88, IGBP1, IGDCC3, IGDCC4, IGF1, IGF1R, IGF2, IGF2BP1, IGF2BP2, IGF2BP3, IGF2R, IGFALS, IGFBP1, IGFBP2, IGFBP3, IGFBP4, IGFBP5, IGFBP6, IGFBP7, IGFBPL1, IGFL1, IGFL2, IGFL3, IGFL4, IGFLR1, IGFN1, IGHA1, IGHA2, IGHD, IGHD1-1, IGHD1-14, IGHD1-20, IGHD1-26, IGHD1-7, IGHD1OR15-1A, IGHD1OR15-1B, IGHD2-15, IGHD2-2, IGHD2-21, IGHD2-8, IGHD2OR15-2A, IGHD2OR15-2B, IGHD3-10, IGHD3-16, IGHD3-22, IGHD3-3, IGHD3-9, IGHD30R15-3A, IGHD30R15-3B, IGHD4-11, IGHD4-17, IGHD4-23, IGHD4-4, IGHD40R15-4A, IGHD40R15-4B, IGHD5-12, IGHD5-18, IGHD5-24, IGHD5-5, IGHD50R15-5A, IGHD50R15-5B, IGHD6-13, IGHD6-19, IGHD6-25, IGHD6-6, IGHD7-27, IGHE, IGHG1, IGHG2, IGHG3, IGHG4, IGHJ1, IGHJ2, IGHJ3, IGHJ4, IGHJ5, IGHJ6, IGHM, IGHMBP2, IGHV1-18, IGHV1-2, IGHV1-24, IGHV1-3, IGHV1-45, IGHV1-46, IGHV1-58, IGHV1-69, IGHV1OR15-1, IGHV1OR15-9, IGHV1OR21-1, IGHV2-26, IGHV2-5, IGHV2-70, IGHV2OR16-5, IGHV3-11, IGHV3-13, IGHV3-15, IGHV3-16, IGHV3-20, IGHV3-21, IGHV3-23, IGHV3-30, IGHV3-33, IGHV3-35, IGHV3-38, IGHV3-43, IGHV3-48, IGHV3-49, IGHV3-53, IGHV3-64, IGHV3-66, IGHV3-7, IGHV3-72, IGHV3-73, IGHV3-74, IGHV30R15-7, IGHV30R16-10, IGHV30R16-12, IGHV30R16-13, IGHV30R16-8, IGHV30R16-9, IGHV4-28, IGHV4-31, IGHV4-34, IGHV4-39, IGHV4-4, IGHV4-59, IGHV4-61, IGHV4OR15-8, IGHV5-51, IGHV6-1, IGHV7-81, IGIP, IGKC, IGKJ1, IGKJ2, IGKJ3, IGKJ4, IGKJ5, IGKV1-12, IGKV1-16, IGKV1-17, IGKV1-27, IGKV1-33, IGKV1-37, IGKV1-39, IGKV1-5, IGKV1-6, IGKV1-8, IGKV1-9, IGKV1D-12, IGKV1D-13, IGKV1D-16, IGKV1D-17, IGKV1D-33, IGKV1D-37, IGKV1D-39, IGKV1D-42, IGKV1D-43, IGKV1D-8, IGKV1OR2-108, IGKV2-24, IGKV2-28, IGKV2-30, IGKV2-40, IGKV2D-24, IGKV2D-26, IGKV2D-28, IGKV2D-29, IGKV2D-30, IGKV2D-40, IGKV3-11, IGKV3-15, IGKV3-20, IGKV3-7, IGKV3D-11, IGKV3D-15, IGKV3D-20, IGKV3D-7, IGKV3OR2-268, IGKV4-1, IGKV5-2, IGKV6-21, IGKV6D-21, IGKV6D-41, IGLC1, IGLC2, IGLC3, IGLC7, IGLJ1, IGLJ2, IGLJ3, IGLJ4, IGLJ5, IGLJ6, IGLJ7, IGLL1, IGLL5, IGLON5, IGLV10-54, IGLV11-55, IGLV1-36, IGLV1-40, IGLV1-44, IGLV1-47, IGLV1-50, IGLV1-51, IGLV2-11, IGLV2-14, IGLV2-18, IGLV2-23, IGLV2-33, IGLV2-8, IGLV3-1, IGLV3-10, IGLV3-12, IGLV3-16, IGLV3-19, IGLV3-21, IGLV3-22, IGLV3-25, IGLV3-27, IGLV3-32, IGLV3-9, IGLV4-3, IGLV4-60, IGLV4-69, IGLV5-37, IGLV5-45, IGLV5-48, IGLV5-52, IGLV6-57, IGLV7-43, IGLV7-46, IGLV8-61, IGLV9-49, IGSF1, IGSF10, IGSF11, IGSF21, IGSF22, IGSF23, IGSF3, IGSF5, IGSF6, IGSF8, IGSF9, IGSF9B, IHH, IK, IKBIP, IKBKB, IKBKE, IKBKG, IKZF1, IKZF2, IKZF3, IKZF4, IKZF5, IL10, IL10RA, IL10RB, IL11, IL11RA, IL12A, IL12B, IL12RB1, IL12RB2, IL13, IL13RA1, IL13RA2, IL15, IL15RA, IL16, IL17A, IL17B, IL17C, IL17D, IL17F, IL17RA, IL17RB, IL17RC, IL17RD, IL17RE, IL17REL, IL18, IL18BP, IL18R1, IL18RAP, IL19, IL1A, IL1B, IL1F10, IL1R1, IL1R2, IL1RAP, IL1RAPL1, IL1RAPL2, IL1RL1, IL1RL2, IL1RN, IL2, IL20, IL20RA, IL20RB, IL21, IL21R, IL22, IL22RA1, IL22RA2, IL23A, IL23R, IL24, IL25, IL26, IL27, IL27RA, IL2RA, IL2RB, IL2RG, IL3, IL31, IL31RA, IL32, IL33, IL34, IL36A, IL36B, IL36G, IL36RN, IL37, IL3RA, IL4, IL4I1, IL4R, IL5, IL5RA, IL6, IL6R, IL6ST, IL7, TL7R, IL9, IL9R, ILDR1, ILDR2, ILF2, ILF3, ILK, ILKAP, ILVBL, IMMP1L, IMMP2L, IMMT, IMP3, IMP4, IMPA1, IPA2, IMPACT, IMPAD1, IMPDH1, IMPDH2, IMPG1, IMPG2, INA, INAFM1, INAFM2, INAVA, INCA1, INCENP, INF2, ING1, ING2, ING3, ING4, ING5, INHA, INHBA, INHBB, INHBC, INHBE, INIP, INMT, INMT-MINDY4, INO80, INO80B, INO80B-WBP1, INO80C, INO80D, INO80E, INPP1, INPP4A, INPP4B, INPP5A, INPP5B, INPP5D, INPP5E, INPP5F, INPP5J, INPP5K, INPPL1, INS, INSC, INSIG1, INSIG2, INS-IGF2, INSL3, INSL4, INSL5, INSL6, INSM1, INSM2, INSR, INSRR, INTS1, INTS10, INTS11, INTS12, INTS13, INTS14, INTS2, INTS3, INTS4, INTS5, INTS6, INTS6L, INTS7, INTS8, INTS9, INTU, INVS, IP6K1, IP6K2, IP6K3, IPCEF1, IPMK, IPO11, IPO13, IPO4, IPO5, IPO7, IPO8, IP09, IPP, IPPK, IQANK1, IQCA1, IQCA1L, IQCB1, IQCC, IQCD, IQCE, IQCF1, IQCF2, IQCF3, IQCF5, IQCF6, IQCG, IQCH, IQCJ, IQCJ-SCHIP1, IQCK, IQCM, IQGAP1, IQGAP2, IQGAP3, IQSEC1, IQSEC2, IQSEC3, IQUB, IRAK1, IRAK1BP1, IRAK2, IRAK3, IRAK4, IREB2, IRF1, IRF2, IRF2BP1, IRF2BP2, IRF2BPL, IRF3, IRF4, IRF5, IRF6, IRF7, IRF8, IRF9, IRGC, IRGM, IRGQ, IRS1, IRS2, IRS4, IRX1, IRX2, IRX3, IRX4, IRX5, IRX6, ISCA1, ISCA2, ISCU, ISG15, ISG20, ISG20L2, ISL1, ISL2, ISLR, ISLR2, ISM1, ISM2, ISOC1, ISOC2, ISPD, IST1, ISX, ISY1, ISY1-RAB43, ISYNA1, ITCH, ITFG1, ITFG2, ITGA1, ITGA10, ITGA11, ITGA2, ITGA2B, ITGA3, ITGA4, ITGA5, ITGA6, ITGA7, ITGA8, ITGA9, ITGAD, ITGAE, ITGAL, ITGAM, ITGAV, ITGAX, ITGB1, ITGB1BP1, ITGB1BP2, ITGB2, ITGB3, ITGB3BP, ITGB4, ITGB5, ITGB6, ITGB7, ITGB8, ITGBL1, ITIH1, ITIH2, ITIH3, ITIH4, ITIH5, ITIH6, ITK, ITLN1, ITLN2, ITM2A, ITM2B, ITM2C, ITPA, ITPK1, ITPKA, ITPKB, ITPKC, ITPR1, ITPR2, ITPR3, ITPRIP, ITPRIPL1, ITPRIPL2, ITSN1, ITSN2, IVD, IVL, IVNS1ABP, IWS1, IYD, IZUMO1, IZUMOIR, IZUMO2, IZUMO3, IZUMO4, JADE1, JADE2, JADE3, JAG1, JAG2, JAGN1, JAK1, JAK2, JAK3, JAKMIP1, JAKMIP2, JAKMIP3, JAM2, JAM3, JAML, JARID2, JAZF1, JCAD, JCHAIN, JDP2, JKAMP, JMJD1C, JMJD4, JMJD6, JMJD7, JMJD7-PLA2G4B, JMJD8, JMY, JOSD1, JOSD2, JPH1, JPH2, JPH3, JPH4, JPT1, JPT2, JRK, JRKL, JSRP1, JTB, JUN, JUNB, JUND, JUP, KAAG1, KALRN, KANK1, KANK2, KANK3, KANK4, KANSL1, KANSL1L, KANSL2, KANSL3, KANTR, KARS, KAT14, KAT2A, KAT2B, KAT5, KAT6A, KAT6B, KAT7, KAT8, KATNA1, KATNAL1, KATNAL2, KATNB1, KATNBL1, KAZALD1, KAZN, KBTBD11, KBTBD11-OT1, KBTBD12, KBTBD13, KBTBD2, KBTBD3, KBTBD4, KBTBD6, KBTBD7, KBTBD8, KCMF1, KCNA1, KCNA10, KCNA2, KCNA3, KCNA4, KCNA5, KCNA7, KCNAB1, KCNAB2, KCNAB3, KCNB1, KCNB2, KCNC1, KCNC2, KCNC3, KCNC4, KCND1, KCND2, KCND3, KCNE1, KCNE1B, KCNE2, KCNE3, KCNE4, KCNE5, KCNF1, KCNG1, KCNG2, KCNG3, KCNG4, KCNH1, KCNH2, KCNH3, KCNH4, KCNH5, KCNH6, KCNH7, KCNH8, KCNIP1, KCNIP2, KCNIP3, KCNIP4, KCNJ1, KCNJ10, KCNJ11, KCNJ12, KCNJ13, KCNJ14, KCNJ15, KCNJ16, KCNJ18, KCNJ2, KCNJ3, KCNJ4, KCNJ5, KCNJ6, KCNJ8, KCNJ9, KCNK1, KCNK10, KCNK12, KCNK13, KCNK15, KCNK16, KCNK17, KCNK18, KCNK2, KCNK3, KCNK4, KCNK5, KCNK6, KCNK7, KCNK9, KCNMA1, KCNMB1, KCNMB2, KCNMB3, KCNMB4, KCNN1, KCNN2, KCNN3, KCNN4, KCNQ1, KCNQ2, KCNQ3, KCNQ4, KCNQ5, KCNRG, KCNS1, KCNS2, KCNS3, KCNT1, KCNT2, KCNU1, KCNV1, KCNV2, KCP, KCTD1, KCTD10, KCTD11, KCTD12, KCTD13, KCTD14, KCTD15, KCTD16, KCTD17, KCTD18, KCTD19, KCTD2, KCTD20, KCTD21, KCTD3, KCTD4, KCTD5, KCTD6, KCTD7, KCTD8, KCTD9, KDELC1, KDELC2, KDELR1, KDELR2, KDELR3, KDF1, KDM1A, KDM1B, KDM2A, KDM2B, KDM3A, KDM3B, KDM4A, KDM4B, KDM4C, KDM4D, KDM4E, KDM4F, KDM5A, KDM5B, KDM5C, KDM5D, KDM6A, KDM6B, KDM7A, KDM8, KDR, KDSR, KEAP1, KEL, KERA, KF459570.1, KHDC1, KHDC1L, KHDC3L, KHDRBS1, KHDRBS2, KHDRBS3, KHK, KHNYN, KHSRP, KIAA0040, KIAA0100, KIAA0141, KIAA0232, KIAA0319, KIAA0319L, KIAA0355, KIAA0368, KIAA0391, KIAA0408, KIAA0513, KIAA0556, KIAA0586, KIAA0753, KIAA0825, KIAA0895, KIAA0895L, KIAA0907, KIAA0930, KIAA1024, KIAA1024L, KIAA1107, KIAA1109, KIAA1143, KIAA1147, KIAA1161, KIAA1191, KIAA1210, KIAA1211, KIAA1211L, KIAA1217, KIAA1257, KIAA1324, KIAA1324L, KIAA1328, KIAA1456, KIAA1468, KIAA1522, KIAA1524, KIAA1549, KIAA1549L, KIAA1551, KIAA1586, KIAA1614, KIAA1644, KIAA1671, KIAA1683, KIAA1755, KIAA1841, KIAA1958, KIAA2012, KIAA2013, KIAA2026, KIDINS220, KIF11, KIF12, KIF13A, KIF13B, KIF14, KIF15, KIF16B, KIF17, KIF18A, KIF18B, KIF19, KIF1A, KIF1B, KIF1BP, KIF1C, KIF20A, KIF20B, KIF21A, KIF21B, KIF22, KIF23, KIF24, KIF25, KIF26A, KIF26B, KIF27, KIF2A, KIF2B, KIF2C, KIF3A, KIF3B, KIF3C, KIF4A, KIF4B, KIF5A, KIF5B, KIF5C, KIF6, KIF7, KIF9, KIFAP3, KIFC1, KIFC2, KIFC3, KIN, KIR2DL1, KIR2DL2, KIR2DL3, KIR2DL4, KIR2DL5A, KIR2DL5B, KIR2DP1, KIR2DS1, KIR2DS2, KIR2DS3, KIR2DS4, KIR2DS5, KIR3DL1, KIR3DL2, KIR3DL3, KIR3DP1, KIR3DS1, KIR3DX1, KIRREL1, KIRREL2, KIRREL3, KISS1, KISS1R, KIT, KITLG, KIZ, KL, KLB, KLC1, KLC2, KLC3, KLC4, KLF1, KLF10, KLF11, KLF12, KLF13, KLF14, KLF15, KLF16, KLF17, KLF18, KLF2, KLF3, KLF4, KLF5, KLF6, KLF7, KLF8, KLF9, KLHDC1, KLHDC10, KLHDC2, KLHDC3, KLHDC4, KLHDC7A, KLHDC7B, KLHDC8A, KLHDC8B, KLHDC9, KLHL1, KLHL10, KLHL11, KLHL12, KLHL13, KLHL14, KLHL15, KLHL17, KLHL18, KLHL2, KLHL20, KLHL21, KLHL22, KLHL23, KLHL24, KLHL25, KLHL26, KLHL28, KLHL29, KLHL3, KLHL30, KLHL31, KLHL32, KLHL33, KLHL34, KLHL35, KLHL36, KLHL38, KLHL4, KLHL40, KLHL41, KLHL42, KLHL5, KLHL6, KLHL7, KLHL8, KLHL9, KLK1, KLK10, KLK11, KLK12, KLK13, KLK14, KLK15, KLK2, KLK3, KLK4, KLK5, KLK6, KLK7, KLK8, KLK9, KLKB1, KLLN, KLRB1, KLRC1, KLRC2, KLRC3, KLRC4, KLRC4-KLRK1, KLRD1, KLRF1, KLRF2, KLRG1, KLRG2, KLRK1, KMO, KMT2A, KMT2B, KMT2C, KMT2D, KMT2E, KMT5A, KMT5B, KMT5C, KNCN, KNDC1, KNG1, KNL1, KNOP1, KNSTRN, KNTC1, KP420437.1, KP420437.2, KP420437.3, KP420439.1, KP420440.1, KP420440.2, KP420440.3, KP420440.4, KP420440.5, KP420440.6, KP420440.7, KP420440.8, KP420440.9, KP420441.1, KP420441.2, KP420441.3, KP420441.4, KP420441.5, KP420442.2, KP420442.3, KP420443.1, KP420444.1, KP420444.2, KP420444.3, KP420444.4, KP420444.5, KP420444.6, KP420444.7, KP420446.1, KP420446.2, KPNA1, KPNA2, KPNA3, KPNA4, KPNA5, KPNA6, KPNA7, KPNB1, KPRP, KPTN, KRAS, KRBA1, KRBA2, KRBOX1, KRBOX4, KRCC1, KREMEN1, KREMEN2, KRIl, KRIT1, KRR1, KRT1, KRT10, KRT12, KRT13, KRT14, KRT15, KRT16, KRT17, KRT18, KRT19, KRT2, KRT20, KRT222, KRT23, KRT24, KRT25, KRT26, KRT27, KRT28, KRT3, KRT31, KRT32, KRT33A, KRT33B, KRT34, KRT35, KRT36, KRT37, KRT38, KRT39, KRT4, KRT40, KRT5, KRT6A, KRT6B, KRT6C, KRT7, KRT71, KRT72, KRT73, KRT74, KRT75, KRT76, KRT77, KRT78, KRT79, KRT8, KRT80, KRT81, KRT82, KRT83, KRT84, KRT85, KRT86, KRT9, KRTAP10-1, KRTAP10-10, KRTAP10-11, KRTAP10-12, KRTAP10-2, KRTAP10-3, KRTAP10-4, KRTAP10-5, KRTAP10-6, KRTAP10-7, KRTAP10-8, KRTAP10-9, KRTAP1-1, KRTAP11-1, KRTAP12-1, KRTAP12-2, KRTAP12-3, KRTAP12-4, KRTAP1-3, KRTAP13-1, KRTAP13-2, KRTAP13-3, KRTAP13-4, KRTAP1-4, KRTAP1-5, KRTAP15-1, KRTAP16-1, KRTAP17-1, KRTAP19-1, KRTAP19-2, KRTAP19-3, KRTAP19-4, KRTAP19-5, KRTAP19-6, KRTAP19-7, KRTAP19-8, KRTAP20-1, KRTAP20-2, KRTAP20-3, KRTAP20-4, KRTAP2-1, KRTAP21-1, KRTAP21-2, KRTAP21-3, KRTAP2-2, KRTAP22-1, KRTAP22-2, KRTAP2-3, KRTAP23-1, KRTAP2-4, KRTAP24-1, KRTAP25-1, KRTAP26-1, KRTAP27-1, KRTAP29-1, KRTAP3-1, KRTAP3-2, KRTAP3-3, KRTAP4-1, KRTAP4-11, KRTAP4-12, KRTAP4-16, KRTAP4-2, KRTAP4-3, KRTAP4-4, KRTAP4-5, KRTAP4-6, KRTAP4-7, KRTAP4-8, KRTAP4-9, KRTAP5-1, KRTAP5-10, KRTAP5-11, KRTAP5-2, KRTAP5-3, KRTAP5-4, KRTAP5-5, KRTAP5-6, KRTAP5-7, KRTAP5-8, KRTAP5-9, KRTAP6-1, KRTAP6-2, KRTAP6-3, KRTAP7-1, KRTAP8-1, KRTAP9-1, KRTAP9-2, KRTAP9-3, KRTAP9-4, KRTAP9-6, KRTAP9-7, KRTAP9-8, KRTAP9-9, KRTCAP2, KRTCAP3, KRTDAP, KSR1, KSR2, KTI12, KTN1, KU645196.1, KU645196.2, KU645196.3, KU645196.4, KU645196.5, KU645196.6, KU645196.7, KU645196.8, KU645196.9, KU645197.1, KU645197.2, KU645197.3, KU645197.4, KU645197.5, KU645198.1, KXD1, KY, KYAT1, KYAT3, KYNU, L1CAM, L1TD1, L2HGDH, L34079.1, L3HYPDH, L3MBTL1, L3MBTL2, L3MBTL3, L3MBTL4, LACC1, LACRT, LACTB, LACTB2, LACTBL1, LAD1, LAG3, LAGE3, LAIR1, LAIR2, LALBA, LAMA1, LAMA2, LAMA3, LAMA4, LAMA5, LAMB1, LAMB2, LAMB3, LAMB4, LAMC1, LAMC2, LAMC3, LAMP1, LAMP2, LAMP3, LAMP5, LAMTOR1, LAMTOR2, LAMTOR3, LAMTOR4, LAMTOR5, LANCL1, LANCL2, LANCL3, LAP3, LAPTM4A, LAPTM4B, LAPTM5, LARGE1, LARGE2, LARP1, LARP1B, LARP4, LARP4B, LARP6, LARP7, LARS, LARS2, LAS1L, LASP1, LAT, LAT2, LATS1, LATS2, LAX1, LAYN, LBH, LBHD1, LBP, LBR, LBX1, LBX2, LCA5, LCA5L, LCAT, LCE1A, LCE1B, LCE1C, LCE1D, LCE1E, LCE1F, LCE2A, LCE2B, LCE2C, LCE2D, LCE3A, LCE3B, LCE3C, LCE3D, LCE3E, LCE4A, LCE5A, LCE6A, LCK, LCLAT1, LCMT1, LCMT2, LCN1, LCN10, LCN12, LCN15, LCN2, LCN6, LCN8, LCN9, LCNL1, LCOR, LCORL, LCP1, LCP2, LCT, LCTL, LDAH, LDB1, LDB2, LDB3, LDHA, LDHAL6A, LDHAL6B, LDHB, LDHC, LDHD, LDLR, LDLRAD1, LDLRAD2, LDLRAD3, LDLRAD4, LDLRAP1, LDOC1, LEAP2, LECT2, LEF1, LEFTY1, LEFTY2, LEKR1, LELP1, LEMD1, LEMD2, LEMD3, LENEP, LENG1, LENG8, LENG9, LEO1, LEP, LEPR, LEPROT, LEPROTL1, LETM1, LETM2, LETMD1, LEUTX, LEXM, LFNG, LGALS1, LGALS12, LGALS13, LGALS14, LGALS16, LGALS2, LGALS3, LGALS3BP, LGALS4, LGALS7, LGALS7B, LGALS8, LGALS9, LGALS9B, LGALS9C, LGALSL, LGI1, LGI2, LGI3, LGI4, LGMN, LGR4, LGR5, LGR6, LGSN, LHB, LHCGR, LHFPL1, LHFPL2, LHFPL3, LHFPL4, LHFPL5, LHFPL6, LHPP, LHX1, LHX2, LHX3, LHX4, LHX5, LHX6, LHX8, LHX9, LIAS, LIF, LIFR, LIGI, LIG3, LIG4, LILRA1, LILRA2, LILRA3, LILRA4, LILRA5, LILRA6, LILRB1, LILRB2, LILRB3, LILRB4, LILRB5, LIM2, LIMA1, LIMCH1, LIMDI1, LIMD2, LIME1, LIMK1, LIMK2, LIMS1, LIMS2, LIMS3, LIMS4, LIN28A, LIN28B, LIN37, LIN52, LIN54, LIN7A, LIN7B, LIN7C, LIN9, LINC00094, LINC00116, LINC00282, LINC00672, LINC00675, LINC00694, LINC00854, LINC00890, LINC00959, LINC01125, LINC01556, LINC02210-CRHR1, LINGO1, LINGO2, LINGO3, LINGO4, LINS1, LIPA, LIPC, LIPE, LIPF, LIPG, LIPH, LIPI, LIPJ, LIPK, LIPM, LIPN, LIPT1, LIPT2, LITAF, LIX1, LIX1L, LKAAEAR1, LLGL1, LLGL2, LLPH, LMAN1, LMAN1L, LMAN2, LMAN2L, LMBR1, LMBR1L, LMBRD1, LMBRD2, LMCD1, LMF1, LMF2, LMLN, LMNA, LMNB1, LMNB2, LMNTD1, LMNTD2, LMO1, LMO2, LMO3, LMO4, LMO7, LMO7DN, LMOD1, LMOD2, LMOD3, LMTK2, LMTK3, LMX1A, LMX1B, LNP1, LNPEP, LNPK, LNX1, LNX2, LO000005.1, LONP1, LONP2, LONRF1, LONRF2, LONRF3, LOR, LOX, LOXHD1, LOXL1, LOXL2, LOXL3, LOXL4, LPA, LPAR1, LPAR2, LPAR3, LPAR4, LPAR5, LPAR6, LPCAT1, LPCAT2, LPCAT3, LPCAT4, LPGAT1, LPIN1, LPIN2, LPIN3, LPL, LPO, LPP, LPXN, LRAT, LRBA, LRCH1, LRCH2, LRCH3, LRCH4, LRCOL1, LRFN1, LRFN2, LRFN3, LRFN4, LRFN5, LRG1, LRGUK, LRIF1, LRIG1, LRIG2, LRIG3, LRIT1, LRIT2, LRIT3, LRMDA, LRMP, LRP1, LRP10, LRP11, LRP12, LRP1B, LRP2, LRP2BP, LRP3, LRP4, LRP5, LRP5L, LRP6, LRP8, LRPAP1, LRPPRC, LRR1, LRRC1, LRRC10, LRRC10B, LRRC14, LRRC14B, LRRC15, LRRC17, LRRC18, LRRC19, LRRC2, LRRC20, LRRC23, LRRC24, LRRC25, LRRC26, LRRC27, LRRC28, LRRC29, LRRC3, LRRC30, LRRC31, LRRC32, LRRC34, LRRC36, LRRC37A, LRRC37A2, LRRC37A3, LRRC37B, LRRC38, LRRC39, LRRC3B, LRRC3C, LRRC4, LRRC40, LRRC41, LRRC42, LRRC43, LRRC45, LRRC46, LRRC47, LRRC49, LRRC4B, LRRC4C, LRRC52, LRRC53, LRRC55, LRRC56, LRRC57, LRRC58, LRRC59, LRRC6, LRRC61, LRRC63, LRRC66, LRRC69, LRRC7, LRRC70, LRRC71, LRRC72, LRRC73, LRRC74A, LRRC74B, LRRC75A, LRRC75B, LRRC8A, LRRC8B, LRRC8C, LRRC8D, LRRC8E, LRRC9, LRRCC1, LRRD1, LRRFIP1, LRRFIP2, LRRIQ1, LRRIQ3, LRRIQ4, LRRK1, LRRK2, LRRN1, LRRN2, LRRN3, LRRN4, LRRN4CL, LRRTM1, LRRTM2, LRRTM3, LRRTM4, LRSAM1, LRTM1, LRTM2, LRTOMT, LRWD1, LSAMP, LSG1, LSM1, LSM10, LSM11, LSM12, LSM14A, LSM14B, LSM2, LSM3, LSM4, LSM5, LSM6, LSM7, LSM8, LSMEM1, LSMEM2, LSP1, LSR, LSS, LST1, LTA, LTA4H, LTB, LTB4R, LTB4R2, LTBP1, LTBP2, LTBP3, LTBP4, LTBR, LTC4S, LTF, LTK, LTN1, LTV1, LUC7L, LUC7L2, LUC7L3, LUM, LURAP1, LURAP1L, LUZP1, LUZP2, LUZP4, LUZP6, LVRN, LXN, LY6D, LY6E, LY6G5B, LY6G5C, LY6G6C, LY6G6D, LY6G6E, LY6G6F, LY6H, LY6K, LY6L, LY75, LY75-CD302, LY86, LY9, LY96, LYAR, LYG1, LYG2, LYL1, LYN, LYNX1, LYPD1, LYPD2, LYPD3, LYPD4, LYPD5, LYPD6, LYPD6B, LYPD8, LYPLA1, LYPLA2, LYPLAL1, LYRM1, LYRM2, LYRM4, LYRM7, LYRM9, LYSMD1, LYSMD2, LYSMD3, LYSMD4, LYST, LYVE1, LYZ, LYZL1, LYZL2, LYZL4, LYZL6, LZIC, LZTFL1, LZTR1, LZTS1, LZTS2, LZTS3, M1AP, M6PR, MAATS1, MAB21L1, MAB21L2, MAB21L3, MACC1, MACF1, MACROD1, MACROD2, MAD1L1, MAD2L1, MAD2L1BP, MAD2L2, MADCAM1, MADD, MAEA, MAEL, MAF, MAF1, MAFA, MAFB, MAFF, MAFG, MAFK, MAG, MAGEA1, MAGEA10, MAGEA11, MAGEA12, MAGEA2, MAGEA2B, MAGEA3, MAGEA4, MAGEA6, MAGEA8, MAGEA9, MAGEA9B, MAGEB1, MAGEB10, MAGEB16, MAGEB17, MAGEB18, MAGEB2, MAGEB3, MAGEB4, MAGEB5, MAGEB6, MAGEB6P1, MAGEC1, MAGEC2, MAGEC3, MAGED1, MAGED2, MAGED4, MAGED4B, MAGEE1, MAGEE2, MAGEF1, MAGEH1, MAGEL2, MAGI1, MAGI2, MAGI3, MAGIX, MAGOH, MAGOHB, MAGT1, MAIP1, MAJIN, MAK, MAK16, MAL, MAL2, MALL, MALRD1, MALSU1, MALT1, MAMDC2, MAMDC4, MAML1, MAML2, MAML3, MAMLD1, MAMSTR, MAN1A1, MAN1A2, MAN1B1, MAN1C1, MAN2A1, MAN2A2, MAN2B1, MAN2B2, MAN2C1, MANBA, MANBAL, MANEA, MANEAL, MANF, MANSC1, MANSC4, MAOA, MAOB, MAP10, MAP1A, MAP1B, MAP1LC3A, MAP1LC3B, MAP1LC3B2, MAP1LC3C, MAP1S, MAP2, MAP2K1, MAP2K2, MAP2K3, MAP2K4, MAP2K5, MAP2K6, MAP2K7, MAP3K1, MAP3K10, MAP3K11, MAP3K12, MAP3K13, MAP3K14, MAP3K15, MAP3K19, MAP3K2, MAP3K20, MAP3K21, MAP3K3, MAP3K4, MAP3K5, MAP3K6, MAP3K7, MAP3K7CL, MAP3K8, MAP3K9, MAP4, MAP4K1, MAP4K2, MAP4K3, MAP4K4, MAP4K5, MAP6, MAP6D1, MAP7, MAP7D1, MAP7D2, MAP7D3, MAP9, MAPK1, MAPK10, MAPK11, MAPK12, MAPK13, MAPK14, MAPK15, MAPK1IP1L, MAPK3, MAPK4, MAPK6, MAPK7, MAPK8, MAPK8IP1, MAPK8IP2, MAPK8IP3, MAPK9, MAPKAP1, MAPKAPK2, MAPKAPK3, MAPKAPK5, MAPKBP1, MAPRE1, MAPRE2, MAPRE3, MAPT, MARC1, MARC2, MARCH1, MARCH10, MARCH11, MARCH2, MARCH3, MARCH4, MARCH5, MARCH6, MARCH7, MARCH8, MARCH9, MARCKS, MARCKSL1, MARCO, MARF1, MARK1, MARK2, MARK3, MARK4, MARS, MARS2, MARVELD1, MARVELD2, MARVELD3, MAS1, MAS1L, MASP1, MASP2, MAST1, MAST2, MAST3, MAST4, MASTL, MAT1A, MAT2A, MAT2B, MATK, MATN1, MATN2, MATN3, MATN4, MATR3, MAU2, MAVS, MAX, MAZ, MB, MB21D1, MB21D2, MBD1, MBD2, MBD3, MBD3L1, MBD3L2, MBD3L2B, MBD3L3, MBD3L4, MBD3L5, MBD4, MBD5, MBD6, MBIP, MBL2, MBLAC1, MBLAC2, MBNL1, MBNL2, MBNL3, MBOAT1, MBOAT2, MBOAT4, MBOAT7, MBP, MBTD1, MBTPS1, MBTPS2, MC1R, MC2R, MC3R, MC4R, MC5R, MCAM, MCAT, MCC, MCCC1, MCCC2, MCCD1, MCEE, MCEMP1, MCF2, MCF2L, MCF2L2, MCFD2, MCHR1, MCHR2, MCIDAS, MCL1, MCM10, MCM2, MCM3, MCM3AP, MCM4, MCM5, MCM6, MCM7, MCM8, MCM9, MCMBP, MCMDC2, MCOLN1, MCOLN2, MCOLN3, MCPH1, MCRIP1, MCRIP2, MCRS1, MCTP1, MCTP2, MCTS1, MCU, MCUB, MCUR1, MDC1, MDFI, MDFIC, MDFIC2, MDGA1, MDGA2, MDH1, MDH1B, MDH2, MDK, MDM1, MDM2, MDM4, MDN1, MDP1, MDS2, ME1, ME2, ME3, MEA1, MEAF6, MECOM, MECP2, MECR, MED1, MED10, MED11, MED12, MED12L, MED13, MED13L, MED14, MED14OS, MED15, MED16, MED17, MED18, MED19, MED20, MED21, MED22, MED23, MED24, MED25, MED26, MED27, MED28, MED29, MED30, MED31, MED4, MED6, MED7, MED8, MED9, MEDAG, MEF2A, MEF2B, MEF2C, MEF2D, MEFV, MEGF10, MEGF11, MEGF6, MEGF8, MEGF9, MEI1, MEI4, MEIG1, MEIKIN, MEIOB, MEIOC, MEIS1, MEIS2, MEIS3, MELK, MELTF, MEMO1, MEN1, MEOX1, MEOX2, MEP1A, MEP1B, MEPCE, MEPE, MERTK, MESD, MESP1, MESP2, MEST, MET, METAP1, METAP1D, METAP2, METRN, METRNL, METTL1, METTL11B, METTL12, METTL13, METTL14, METTL15, METTL16, METTL17, METTL18, METTL21A, METTL21C, METTL22, METTL23, METTL24, METTL25, METTL26, METTL27, METTL2A, METTL2B, METTL3, METTL4, METTL5, METTL6, METTL7A, METTL7B, METTL8, METTL9, MEX3A, MEX3B, MEX3C, MEX3D, MFAP1, MFAP2, MFAP3, MFAP3L, MFAP4, MFAP5, MFF, MFGE8, MFHAS1, MFN1, MFN2, MFNG, MFRP, MFSD1, MFSD10, MFSD11, MFSD12, MFSD13A, MFSD14A, MFSD14B, MFSD14C, MFSD2A, MFSD2B, MFSD3, MFSD4A, MFSD4B, MFSD5, MFSD6, MFSD6L, MFSD7, MFSD8, MFSD9, MGA, MGAM, MGAM2, MGARP, MGAT1, MGAT2, MGAT3, MGAT4A, MGAT4B, MGAT4C, MGAT4D, MGAT5, MGAT5B, MGEA5, MGLL, MGME1, MGMT, MGP, MGRN1, MGST1, MGST2, MGST3, MIA, MIA3, MIA-RAB4B, MIB1, MIB2, MICA, MICAL1, MICAL2, MICAL3, MICALCL, MICALL1, MICALL2, MICB, MICU1, MICU2, MICU3, MID1, MID1IP1, MID2, MIDN, MIEF1, MIEF2, MIEN1, MIER1, MIER2, MIER3, MIF, MIF4GD, MIGA1, MIGA2, MIIP, MILR1, MINDY1, MINDY2, MINDY3, MINDY4, MINDY4B, MINK1, MINOS1, MINOS1-NBL1, MINPP1, MIOS, MIOX, MIP, MIPEP, MIPOL1, MIS12, MIS18A, MIS18BP1, MISP, MISP3, MITD1, MITF, MIXL1, MKI67, MKKS, MKL1, MKL2, MKLN1, MKNK1, MKNK2, MKRN1, MKRN2, MKRN2OS, MKRN3, MKS1, MKX, MLANA, MLC1, MLEC, MLF1, MLF2, MLH1, MLH3, MLIP, MLKL, MLLT1, MLLT10, MLLT11, MLLT3, MLLT6, MLN, MLNR, MLPH, MLST8, MLX, MLXIP, MLXIPL, MLYCD, MMAA, MMAB, MMACHC, MMADHC, MMD, MMD2, MME, MMEL1, MMGT1, MMP1, MMP10, MMP11, MMP12, MMP13, MMP14, MMP15, MMP16, MMP17, MMP19, MMP2, MMP20, MMP21, MMP23B, MMP24, MMP24-AS1, MMP25, MMP26, MMP27, MMP28, MMP3, MMP7, MMP8, MMP9, MMRN1, MMRN2, MMS19, MMS22L, MN1, MNAT1, MND1, MNDA, MNS1, MNT, MNX1, MOAP1, MOB1A, MOB1B, MOB2, MOB3A, MOB3B, MOB3C, MOB4, MOBP, MOCOS, MOCS1, MOCS2, MOCS3, MOG, MOGAT1, MOGAT2, MOGAT3, MOGS, MOK, MON1A, MON1B, MON2, MORC1, MORC2, MORC3, MORC4, MORF4L1, MORF4L2, MORN1, MORN2, MORN3, MORN4, MORN5, MOS, MOSPD1, MOSPD2, MOSPD3, MOV10, MOV10L1, MOXD1, MPC1, MPC1L, MPC2, MPDU1, MPDZ, MPEG1, MPG, MPHOSPH10, MPHOSPH6, MPHOSPH8, MPHOSPH9, MPI, MPIG6B, MPL, MPLKIP, MPND, MPO, MPP1, MPP2, MPP3, MPP4, MPP5, MPP6, MPP7, MPPE1, MPPED1, MPPED2, MPRIP, MPST, MPV17, MPV17L, MPV17L2, MPZ, MPZL1, MPZL2, MPZL3, MR1, MRAP, MRAP2, MRAS, MRC1, MRC2, MRE11, MREG, MRFAP1, MRFAPIL1, MRGBP, MRGPRD, MRGPRE, MRGPRF, MRGPRG, MRGPRX1, MRGPRX2, MRGPRX3, MRGPRX4, MRI1, MRLN, MRM1, MRM2, MRM3, MRNIP, MRO, MROH1, MROH2A, MROH2B, MROH5, MROH6, MROH7, MROH7-TTC4, MROH8, MROH9, MRPL1, MRPL10, MRPL11, MRPL12, MRPL13, MRPL14, MRPL15, MRPL16, MRPL17, MRPL18, MRPL19, MRPL2, MRPL20, MRPL21, MRPL22, MRPL23, MRPL24, MRPL27, MRPL28, MRPL3, MRPL30, MRPL32, MRPL33, MRPL34, MRPL35, MRPL36, MRPL37, MRPL38, MRPL39, MRPL4, MRPL40, MRPL41, MRPL42, MRPL43, MRPL44, MRPL45, MRPL46, MRPL47, MRPL48, MRPL49, MRPL50, MRPL51, MRPL52, MRPL53, MRPL54, MRPL55, MRPL57, MRPL58, MRPL9, MRPS10, MRPS11, MRPS12, MRPS14, MRPS15, MRPS16, MRPS17, MRPS18A, MRPS18B, MRPS18C, MRPS2, MRPS21, MRPS22, MRPS23, MRPS24, MRPS25, MRPS26, MRPS27, MRPS28, MRPS30, MRPS31, MRPS33, MRPS34, MRPS35, MRPS36, MRPS5, MRPS6, MRPS7, MRPS9, MRRF, MRS2, MRTO4, MRVI1, MS4A1, MS4A10, MS4A12, MS4A13, MS4A14, MS4A15, MS4A2, MS4A3, MS4A4A, MS4A4E, MS4A5, MS4A6A, MS4A6E, MS4A7, MS4A8, MSANTD1, MSANTD2, MSANTD3, MSANTD3-TMEFF1, MSANTD4, MSC, MSGN1, MSH2, MSH3, MSH4, MSH5, MSH5-SAPCD1, MSH6, MSI1, MSI2, MSL1, MSL2, MSL3, MSLN, MSLNL, MSMB, MSMO1, MSMP, MSN, MSR1, MSRA, MSRB1, MSRB2, MSRB3, MSS51, MST1, MST1R, MSTN, MSTO1, MSX1, MSX2, MT1A, MT1B, MT1E, MT1F, MT1G, MT1H, MT1IL1, MT1M, MT1X, MT2A, MT3, MT4, MTA1, MTA2, MTA3, MTAP, MT-ATP6, MT-ATP8, MTBP, MTCH1, MTCH2, MTCL1, MT-CO1, MT-CO2, MT-CO3, MTCP1, MT-CYB, MTDH, MTERF1, MTERF2, MTERF3, MTERF4, MTF1, MTF2, MTFMT, MTFP1, MTFR1, MTFR1L, MTFR2, MTG1, MTG2, MTHFD1, MTHFD1L, MTHFD2, MTHFD2L, MTHFR, MTHFS, MTHFSD, MTIF2, MTIF3, MTM1, MTMR1, MTMR10, MTMR11, MTMR12, MTMR14, MTMR2, MTMR3, MTMR4, MTMR6, MTMR7, MTMR8, MTMR9, MT-ND1, MT-ND2, MT-ND3, MT-ND4, MT-ND4L, MT-ND5, MT-ND6, MTNR1A, MTNR1B, MTO1, MTOR, MTPAP, MTPN, MTR, MTRF1, MTRF1L, MTRNR2L1, MTRNR2L10, MTRNR2L11, MTRNR2L12, MTRNR2L13, MTRNR2L3, MTRNR2L4, MTRNR2L5, MTRNR2L6, MTRNR2L7, MTRNR2L8, MTRR, MTSS1, MTSS1L, MTTP, MTURN, MTUS1, MTUS2, MTX1, MTX2, MTX3, MUC1, MUC12, MUC13, MUC15, MUC16, MUC17, MUC2, MUC20, MUC21, MUC22, MUC3A, MUC4, MUC5AC, MUC5B, MUC6, MUC7, MUCL1, MUL1, MUM1, MUM1L1, MUS81, MUSK, MUSTN1, MUT, MUTYH, MVB12A, MVB12B, MVD, MVK, MVP, MX1, MX2, MXD1, MXD3, MXD4, MXI1, MXRA5, MXRA7, MXRA8, MYADM, MYADML2, MYB, MYBBP1A, MYBL1, MYBL2, MYBPC1, MYBPC2, MYBPC3, MYBPH, MYBPHL, MYC, MYCBP, MYCBP2, MYCBPAP, MYCL, MYCN, MYCT1, MYD88, MYDGF, MYEF2, MYEOV, MYF5, MYF6, MYH1, MYH10, MYH11, MYH13, MYH14, MYH15, MYH2, MYH3, MYH4, MYH6, MYH7, MYH7B, MYH8, MYH9, MYL1, MYL10, MYL12A, MYL12B, MYL2, MYL3, MYL4, MYL5, MYL6, MYL6B, MYL7, MYL9, MYLIP, MYLK, MYLK2, MYLK3, MYLK4, MYLPF, MYMK, MYMX, MYNN, MYO10, MYO15A, MYO15B, MYO16, MYO18A, MYO18B, MYO19, MYO1A, MYO1B, MYO1C, MYO1D, MYO1E, MYO1F, MYO1G, MYO1H, MYO3A, MYO3B, MYO5A, MYO5B, MYO5C, MYO6, MYO7A, MYO7B, MYO9A, MYO9B, MYOC, MYOCD, MYOCOS, MYOD1, MYOF, MYOG, MYOM1, MYOM2, MYOM3, MYOT, MYOZ1, MYOZ2, MYOZ3, MYPN, MYPOP, MYRF, MYRFL, MYRIP, MYSM1, MYT1, MYT1L, MYZAP, MZB1, MZF1, MZT1, MZT2A, MZT2B, N4BP1, N4BP2, N4BP2L1, N4BP2L2, N4BP3, N6AMT1, NAA10, NAA11, NAA15, NAA16, NAA20, NAA25, NAA30, NAA35, NAA38, NAA40, NAA50, NAA60, NAAA, NAALAD2, NAALADL1, NAALADL2, NAB1, NAB2, NABP1, NABP2, NACA, NACA2, NACAD, NACC1, NACC2, NADK, NADK2, NADSYN1, NAE1, NAF1, NAGA, NAGK, NAGLU, NAGPA, NAGS, NAIF1, NAIP, NALCN, NAMPT, NANOG, NANOGNB, NANOGP8, NANOS1, NANOS2, NANOS3, NANP, NANS, NAP1L1, NAP1L2, NAP1L3, NAP1L4, NAP1L5, NAPA, NAPB, NAPEPLD, NAPG, NAPRT, NAPSA, NARF, NARFL, NARS, NARS2, NASP, NAT1, NAT10, NAT14, NAT16, NAT2, NAT6, NAT8, NAT8B, NAT8L, NAT9, NATD1, NAV1, NAV2, NAV3, NAXD, NAXE, NBAS, NBDY, NBEA, NBEAL1, NBEAL2, NBL1, NBN, NBPF1, NBPF10, NBPF11, NBPF12, NBPF14, NBPF15, NBPF19, NBPF20, NBPF26, NBPF3, NBPF4, NBPF6, NBPF9, NBR1, NCALD, NCAM1, NCAM2, NCAN, NCAPD2, NCAPD3, NCAPG, NCAPG2, NCAPH, NCAPH2, NCBP1, NCBP2, NCBP2-AS2, NCBP2L, NCBP3, NCCRP1, NCDN, NCEH1, NCF1, NCF2, NCF4, NCK1, NCK2, NCKAP1, NCKAP1L, NCKAP5, NCKAP5L, NCKIPSD, NCL, NCLN, NCMAP, NCOA1, NCOA2, NCOA3, NCOA4, NCOA5, NCOA6, NCOA7, NCOR1, NCOR2, NCR1, NCR2, NCR3, NCR3LG1, NCS1, NCSTN, NDC1, NDC80, NDE1, NDEL1, NDFIP1, NDFIP2, NDN, NDNF, NDOR1, NDP, NDRG1, NDRG2, NDRG3, NDRG4, NDST1, NDST2, NDST3, NDST4, NDUFA1, NDUFA10, NDUFA11, NDUFA12, NDUFA13, NDUFA2, NDUFA3, NDUFA4, NDUFA4L2, NDUFA5, NDUFA6, NDUFA7, NDUFA8, NDUFA9, NDUFAB1, NDUFAF1, NDUFAF2, NDUFAF3, NDUFAF4, NDUFAF5, NDUFAF6, NDUFAF7, NDUFAF8, NDUFB1, NDUFB10, NDUFB11, NDUFB2, NDUFB3, NDUFB4, NDUFB5, NDUFB6, NDUFB7, NDUFB8, NDUFB9, NDUFC1, NDUFC2, NDUFC2-KCTD14, NDUFS1, NDUFS2, NDUFS3, NDUFS4, NDUFS5, NDUFS6, NDUFS7, NDUFS8, NDUFV1, NDUFV2, NDUFV3, NEB, NEBL, NECAB1, NECAB2, NECAB3, NECAP1, NECAP2, NECTIN1, NECTIN2, NECTIN3, NECTIN4, NEDD1, NEDD4, NEDD4L, NEDD8, NEDD8-MDP1, NEDD9, NEFH, NEFL, NEFM, NEGR1, NEIL1, NEIL2, NEIL3, NEK1, NEK10, NEK11, NEK2, NEK3, NEK4, NEK5, NEK6, NEK7, NEK8, NEK9, NELFA, NELFB, NELFCD, NELFE, NELL1, NELL2, NEMF, NEMP1, NEMP2, NENF, NEO1, NEPRO, NES, NET1, NETO1, NETO2, NEU1, NEU2, NEU3, NEU4, NEURL1, NEURL1B, NEURL2, NEURL3, NEURL4, NEUROD1, NEUROD2, NEUROD4, NEUROD6, NEUROG1, NEUROG2, NEUROG3, NEXMIF, NEXN, NF1, NF2, NFAM1, NFASC, NFAT5, NFATC1, NFATC2, NFATC2IP, NFATC3, NFATC4, NFE2, NFE2L1, NFE2L2, NFE2L3, NFE4, NFIA, NFIB, NFIC, NFIL3, NFIX, NFKB1, NFKB2, NFKBIA, NFKBIB, NFKBID, NFKBIE, NFKBIL1, NFKBIZ, NFRKB, NFS1, NFU1, NFX1, NFXL1, NFYA, NFYB, NFYC, NGB, NGDN, NGEF, NGF, NGFR, NGLY1, NGRN, NHEJ1, NHLH1, NHLH2, NHLRC1, NHLRC2, NHLRC3, NHLRC4, NHP2, NHS, NHSL1, NHSL2, NICN1, NID1, NID2, NIF3L1, NIFK, NIM1K, NIN, NINJ1, NINJ2, NINL, NIP7, NIPA1, NIPA2, NIPAL1, NIPAL2, NIPAL3, NIPAL4, NIPBL, NIPSNAP1, NIPSNAP2, NIPSNAP3A, NIPSNAP3B, NISCH, NIT1, NIT2, NKAIN1, NKAIN2, NKAIN3, NKAIN4, NKAP, NKAPL, NKD1, NKD2, NKG7, NKIRAS1, NKIRAS2, NKPD1, NKRF, NKTR, NKX1-1, NKX1-2, NKX2-1, NKX2-2, NKX2-3, NKX2-4, NKX2-5, NKX2-6, NKX2-8, NKX3-1, NKX3-2, NKX6-1, NKX6-2, NKX6-3, NLE1, NLGN1, NLGN2, NLGN3, NLGN4X, NLGN4Y, NLK, NLN, NLRC3, NLRC4, NLRC5, NLRP1, NLRP10, NLRP11, NLRP12, NLRP13, NLRP14, NLRP2, NLRP2B, NLRP3, NLRP4, NLRP5, NLRP6, NLRP7, NLRP8, NLRP9, NLRX1, NMB, NMBR, NMD3, NME1, NME1-NME2, NME2, NME3, NME4, NME5, NME6, NME7, NME8, NME9, NMI, NMNAT1, NMNAT2, NMNAT3, NMRAL1, NMRK1, NMRK2, NMS, NMT1, NMT2, NMU, NMUR1, NMUR2, NNAT, NNMT, NNT, NOA1, NOB1, NOBOX, NOC2L, NOC3L, NOC4L, NOCT, NOD1, NOD2, NODAL, NOG, NOL10, NOL11, NOL12, NOL3, NOL4, NOL4L, NOL6, NOL7, NOL8, NOL9, NOLC1, NOM1, NOMO1, NOMO2, NOMO3, NONO, NOP10, NOP14, NOP16, NOP2, NOP53, NOP56, NOP58, NOP9, NOS1, NOS1AP, NOS2, NOS3, NOSIP, NOSTRIN, NOTCH1, NOTCH2, NOTCH2NL, NOTCH3, NOTCH4, NOTO, NOTUM, NOV, NOVA1, NOVA2, NOX1, NOX3, NOX4, NOX5, NOXA1, NOXO1, NOXRED1, NPAP1, NPAS1, NPAS2, NPAS3, NPAS4, NPAT, NPB, NPBWR1, NPBWR2, NPC1, NPC1L1, NPC2, NPDC1, NPEPL1, NPEPPS, NPFF, NPFFR1, NPFFR2, NPHP1, NPHP3, NPHP3-ACAD11, NPHP4, NPHS1, NPHS2, NPIPA1, NPIPA2, NPIPA3, NPIPA5, NPIPA7, NPIPA8, NPIPB11, NPIPB12, NPIPB13, NPIPB15, NPIPB2, NPIPB3, NPIPB4, NPIPB5, NPIPB6, NPIPB7, NPIPB8, NPIPB9, NPL, NPLOC4, NPM1, NPM2, NPM3, NPNT, NPPA, NPPB, NPPC, NPR1, NPR2, NPR3, NPRL2, NPRL3, NPS, NPSR1, NPTN, NPTX1, NPTX2, NPTXR, NPVF, NPW, NPY, NPY1R, NPY2R, NPY4R, NPY4R2, NPY5R, NQO1, NQO2, NR0B1, NR0B2, NR1D1, NR1D2, NR1H2, NR1H3, NR1H4, NR1I2, NR1I3, NR2C1, NR2C2, NR2C2AP, NR2E1, NR2E3, NR2F1, NR2F2, NR2F6, NR3C1, NR3C2, NR4A1, NR4A2, NR4A3, NR5A1, NR5A2, NR6A1, NRAP, NRARP, NRAS, NRBF2, NRBP1, NRBP2, NRCAM, NRDC, NRDE2, NREP, NRF1, NRG1, NRG2, NRG3, NRG4, NRGN, NRIP1, NRIP2, NRIP3, NRK, NRL, NRM, NRN1, NRN1L, NRP1, NRP2, NRROS, NRSN1, NRSN2, NRTN, NRXN1, NRXN2, NRXN3, NSA2, NSD1, NSD2, NSD3, NSDHL, NSF, NSFL1C, NSL1, NSMAF, NSMCE1, NSMCE2, NSMCE3, NSMCE4A, NSMF, NSRP1, NSUN2, NSUN3, NSUN4, NSUN5, NSUN6, NSUN7, NT5C, NT5C1A, NT5C1B, NT5C1B-RDH14, NT5C2, NT5C3A, NT5C3B, NT5DC1, NT5DC2, NT5DC3, NT5DC4, NT5E, NT5M, NTAN1, NTF3, NTF4, NTHL1, NTM, NTMT1, NTN1, NTN3, NTN4, NTN5, NTNG1, NTNG2, NTPCR, NTRK1, NTRK2, NTRK3, NTS, NTSR1, NTSR2, NUAK1, NUAK2, NUB1, NUBP1, NUBP2, NUBPL, NUCB1, NUCB2, NUCKS1, NUDC, NUDCD1, NUDCD2, NUDCD3, NUDT1, NUDT10, NUDT11, NUDT12, NUDT13, NUDT14, NUDT15, NUDT16, NUDT16L1, NUDT17, NUDT18, NUDT19, NUDT2, NUDT21, NUDT22, NUDT3, NUDT4, NUDT4P1, NUDT5, NUDT6, NUDT7, NUDT8, NUDT9, NUF2, NUFIP1, NUFIP2, NUGGC, NUMA1, NUMB, NUMBL, NUP107, NUP133, NUP153, NUP155, NUP160, NUP188, NUP205, NUP210, NUP210L, NUP214, NUP35, NUP37, NUP43, NUP50, NUP54, NUP58, NUP62, NUP62CL, NUP85, NUP88, NUP93, NUP98, NUPL2, NUPR1, NUPR2, NUS1, NUSAP1, NUTF2, NUTM1, NUTM2A, NUTM2B, NUTM2D, NUTM2E, NUTM2F, NUTM2G, NVL, NWD1, NWD2, NXF1, NXF2, NXF2B, NXF3, NXF5, NXN, NXNL1, NXNL2, NXPE1, NXPE2, NXPE3, NXPE4, NXPH1, NXPH2, NXPH3, NXPH4, NXT1, NXT2, NYAP1, NYAP2, NYNRIN, NYX, OAF, OARD1, OAS1, OAS2, OAS3, OASL, OAT, OAZ1, OAZ2, OAZ3, OBP2A, OBP2B, OBSCN, OBSCN-AS1, OBSL1, OC90, OCA2, OCEL1, OCIAD1, OCIAD2, OCLM, OCLN, OCM, OCM2, OCRL, OCSTAMP, ODAM, ODC1, ODF1, ODF2, ODF2L, ODF3, ODF3B, ODF3L1, ODF3L2, ODF4, OFCC1, OFD1, OGDH, OGDHL, OGFOD1, OGFOD2, OGFOD3, OGFR, OGFRL1, OGG1, OGN, OGT, OIP5, OIT3, OLA1, OLAH, OLFM1, OLFM2, OLFM3, OLFM4, OLFML1, OLFML2A, OLFML2B, OLFML3, OLIG1, OLIG2, OLIG3, OLR1, OMA1, OMD, OMG, OMP, ONECUT1, ONECUT2, ONECUT3, OOEP, OOSP2, OPA1, OPA3, OPALIN, OPCML, OPHN1, OPLAH, OPN1LW, OPN1MW, OPN1MW2, OPN1MW3, OPN1SW, OPN3, OPN4, OPN5, OPRD1, OPRK1, OPRL1, OPRM1, OPRPN, OPTC, OPTN, OR10A2, OR10A3, OR10A4, OR10A5, OR10A6, OR10A7, OR10AC1, OR10AD1, OR10AG1, OR10C1, OR10D3, OR10G2, OR10G3, OR10G4, OR10G6, OR10G7, OR10G8, OR10G9, OR10H1, OR10H2, OR10H3, OR10H4, OR10H5, OR10J1, OR10J3, OR10J4, OR10J5, OR10K1, OR10K2, OR10P1, OR10Q1, OR10R2, OR10S1, OR10T2, OR10V1, OR10W1, OR10X1, OR10Z1, OR11A1, OR11G2, OR11H1, OR11H12, OR11H2, OR11H4, OR11H6, OR11H7, OR11L1, OR12D1, OR12D2, OR12D3, OR13A1, OR13C2, OR13C3, OR13C4, OR13C5, OR13C7, OR13C8, OR13C9, OR13D1, OR13F1, OR13G1, OR13H1, OR13J1, OR14A16, OR14A2, OR14C36, OR14I1, OR14J1, OR14K1, OR1A1, OR1A2, OR1B1, OR1C1, OR1D2, OR1D5, OR1E1, OR1E2, OR1F1, OR1G1, OR1I1, OR1J1, OR1J2, OR1J4, OR1K1, OR1L1, OR1L3, OR1L4, OR1L6, OR1L8, OR1M1, OR1N1, OR1N2, OR1P1, OR1Q1, OR1S1, OR1S2, OR2A1, OR2A12, OR2A14, OR2A2, OR2A25, OR2A4, OR2A42, OR2A5, OR2A7, OR2AE1, OR2AG1, OR2AG2, OR2AJ1, OR2AK2, OR2AP1, OR2AT4, OR2B11, OR2B2, OR2B3, OR2B6, OR2C1, OR2C3, OR2D2, OR2D3, OR2F1, OR2F2, OR2G2, OR2G3, OR2G6, OR2H1, OR2H2, OR2J1, OR2J2, OR2J3, OR2K2, OR2L13, OR2L2, OR2L3, OR2L5, OR2L8, OR2M2, OR2M3, OR2M4, OR2M5, OR2M7, OR2S2, OR2T1, OR2T10, OR2T11, OR2T12, OR2T2, OR2T27, OR2T29, OR2T3, OR2T33, OR2T34, OR2T35, OR2T4, OR2T5, OR2T6, OR2T7, OR2T8, OR2V1, OR2V2, OR2W1, OR2W3, OR2Y1, OR2Z1, OR3A1, OR3A2, OR3A3, OR4A15, OR4A16, OR4A47, OR4A5, OR4A8, OR4B1, OR4C11, OR4C12, OR4C13, OR4C15, OR4C16, OR4C3, OR4C45, OR4C46, OR4C5, OR4C6, OR4D1, OR4D10, OR4D11, OR4D2, OR4D5, OR4D6, OR4D9, OR4E1, OR4E2, OR4F15, OR4F16, OR4F17, OR4F21, OR4F29, OR4F3, OR4F4, OR4F5, OR4F6, OR4K1, OR4K13, OR4K14, OR4K15, OR4K17, OR4K2, OR4K3, OR4K5, OR4L1, OR4M1, OR4M2, OR4N2, OR4N4, OR4N5, OR4P4, OR4Q2, OR4Q3, OR4S1, OR4S2, OR4X1, OR4X2, OR51A2, OR51A4, OR51A7, OR51B2, OR51B4, OR51B5, OR51B6, OR51D1, OR51E1, OR51E2, OR51F1, OR51F2, OR51G1, OR51G2, OR51H1, OR51I1, OR51I2, OR51J1, OR51L1, OR51M1, OR51Q1, OR51S1, OR51T1, OR51V1, OR52A1, OR52A5, OR52B2, OR52B4, OR52B6, OR52D1, OR52E2, OR52E4, OR52E5, OR52E6, OR52E8, OR52H1, OR52I1, OR52I2, OR52J3, OR52K1, OR52K2, OR52L1, OR52M1, OR52N1, OR52N2, OR52N4, OR52N5, OR52R1, OR52W1, OR52Z1, OR56A1, OR56A3, OR56A4, OR56A5, OR56B1, OR56B4, OR5A1, OR5A2, OR5AC1, OR5AC2, OR5AK2, OR5AN1, OR5AP2, OR5AR1, OR5AS1, OR5AU1, OR5B12, OR5B17, OR5B2, OR5B21, OR5B3, OR5C1, OR5D13, OR5D14, OR5D16, OR5D18, OR5F1, OR5G3, OR5H1, OR5H14, OR5H15, OR5H2, OR5H6, OR5H8, OR5I1, OR5J2, OR5K1, OR5K2, OR5K3, OR5K4, OR5L1, OR5L2, OR5M1, OR5M10, OR5M11, OR5M3, OR5M8, OR5M9, OR5P2, OR5P3, OR5R1, OR5T1, OR5T2, OR5T3, OR5V1, OR5W2, OR6A2, OR6B1, OR6B2, OR6B3, OR6C1, OR6C2, OR6C3, OR6C4, OR6C6, OR6C65, OR6C68, OR6C70, OR6C74, OR6C75, OR6C76, OR6F1, OR6J1, OR6K2, OR6K3, OR6K6, OR6M1, OR6N1, OR6N2, OR6P1, OR6Q1, OR6S1, OR6T1, OR6V1, OR6X1, OR6Y1, OR7A10, OR7A17, OR7A5, OR7C1, OR7C2, OR7D2, OR7D4, OR7E24, OR7G1, OR7G2, OR7G3, OR8A1, OR8B12, OR8B2, OR8B3, OR8B4, OR8B8, OR8D1, OR8D2, OR8D4, OR8G1, OR8G5, OR8H1, OR8H2, OR8H3, OR8I2, OR8J1, OR8J2, OR8J3, OR8K1, OR8K3, OR8K5, OR8S1, OR8U1, OR8U8, OR9A2, OR9A4, OR9G1, OR9G4, OR9G9, OR9H1P, OR9I1, OR9K2, OR9Q1, OR9Q2, ORAI1, ORAI2, ORAI3, ORAOV1, ORC1, ORC2, ORC3, ORC4, ORC5, ORC6, ORM1, ORM2, ORMDL1, ORMDL2, ORMDL3, OS9, OSBP, OSBP2, OSBPL10, OSBPL11, OSBPL1A, OSBPL2, OSBPL3, OSBPL5, OSBPL6, OSBPL7, OSBPL8, OSBPL9, OSCAR, OSCP1, OSER1, OSGEP, OSGEPL1, OSGIN1, OSGIN2, OSM, OSMR, OSR1, OSR2, OST4, OSTC, OSTF1, OSTM1, OSTN, OTC, OTOA, OTOF, OTOG, OTOGL, OTOL1, OTOP1, OTOP2, OTOP3, OTOR, OTOS, OTP, OTUB1, OTUB2, OTUD1, OTUD3, OTUD4, OTUD5, OTUD6A, OTUD6B, OTUD7A, OTUD7B, OTULIN, OTX1, OTX2, OVCA2, OVCH1, OVCH2, OVGP1, OVOL1, OVOL2, OVOL3, OXA1L, OXCT1, OXCT2, OXER1, OXGR1, OXLD1, OXNAD1, OXR1, OXSM, OXSR1, OXT, OXTR, P2RX1, P2RX2, P2RX3, P2RX4, P2RX5, P2RX5-TAX1BP3, P2RX6, P2RX7, P2RY1, P2RY10, P2RY11, P2RY12, P2RY13, P2RY14, P2RY2, P2RY4, P2RY6, P2RY8, P3H1, P3H2, P3H3, P3H4, P4HA1, P4HA2, P4HA3, P4HB, P4HTM, PA2G4, PAAF1, PABPC1, PABPC1L, PABPC1L2A, PABPC1L2B, PABPC3, PABPC4, PABPC4L, PABPC5, PABPN1, PABPN1L, PACRG, PACRGL, PACS1, PACS2, PACSIN1, PACSIN2, PACSIN3, PADI1, PADI2, PADI3, PADI4, PADI6, PAEP, PAF1, PAFAH1B1, PAFAH1B2, PAFAH1B3, PAFAH2, PAG1, PAGE1, PAGE2, PAGE2B, PAGE3, PAGE4, PAGE5, PAGR1, PAH, PAICS, PAIP1, PAIP2, PAIP2B, PAK1, PAK1IP1, PAK2, PAK3, PAK4, PAK5, PAK6, PALB2, PALD1, PALLD, PALM, PALM2, PALM2-AKAP2, PALM3, PALMD, PAM, PAM16, PAMR1, PAN2, PAN3, PANK1, PANK2, PANK3, PANK4, PANO1, PANX1, PANX2, PANX3, PAOX, PAPD4, PAPD5, PAPD7, PAPLN, PAPOLA, PAPOLB, PAPOLG, PAPPA, PAPPA2, PAPSS1, PAPSS2, PAQR3, PAQR4, PAQR5, PAQR6, PAQR7, PAQR8, PAQR9, PARD3, PARD3B, PARD6A, PARD6B, PARD6G, PARG, PARK7, PARL, PARM1, PARN, PARP1, PARP10, PARP11, PARP12, PARP14, PARP15, PARP16, PARP2, PARP3, PARP4, PARP6, PARP8, PARP9, PARPBP, PARS2, PARVA, PARVB, PARVG, PASD1, PASK, PATE1, PATE2, PATE3, PATE4, PATJ, PATL1, PATL2, PATZ1, PAWR, PAX1, PAX2, PAX3, PAX4, PAX5, PAX6, PAX7, PAX8, PAX9, PAXBP1, PAXIP1, PAXX, PBDC1, PBK, PBLD, PBOV1, PBRM1, PBX1, PBX2, PBX3, PBX4, PBXIP1, PC, PCBD1, PCBD2, PCBP1, PCBP2, PCBP3, PCBP4, PCCA, PCCB, PCDH1, PCDH10, PCDH11X, PCDH11Y, PCDH12, PCDH15, PCDH17, PCDH18, PCDH19, PCDH20, PCDH7, PCDH8, PCDH9, PCDHA1, PCDHA10, PCDHA11, PCDHA12, PCDHA13, PCDHA2, PCDHA3, PCDHA4, PCDHA5, PCDHA6, PCDHA7, PCDHA8, PCDHA9, PCDHAC1, PCDHAC2, PCDHB1, PCDHB10, PCDHB11, PCDHB12, PCDHB13, PCDHB14, PCDHB15, PCDHB16, PCDHB2, PCDHB3, PCDHB4, PCDHB5, PCDHB6, PCDHB7, PCDHB8, PCDHB9, PCDHGA1, PCDHGA10, PCDHGA11, PCDHGA12, PCDHGA2, PCDHGA3, PCDHGA4, PCDHGA5, PCDHGA6, PCDHGA7, PCDHGA8, PCDHGA9, PCDHGB1, PCDHGB2, PCDHGB3, PCDHGB4, PCDHGB5, PCDHGB6, PCDHGB7, PCDHGC3, PCDHGC4, PCDHGC5, PCED1A, PCED1B, PCF11, PCGF1, PCGF2, PCGF3, PCGF5, PCGF6, PCID2, PCIF1, PCK1, PCK2, PCLAF, PCLO, PCM1, PCMT1, PCMTD1, PCMTD2, PCNA, PCNP, PCNT, PCNX1, PCNX2, PCNX3, PCNX4, PCOLCE, PCOLCE2, PCOTH, PCP2, PCP4, PCP4L1, PCSK1, PCSK1N, PCSK2, PCSK4, PCSK5, PCSK6, PCSK7, PCSK9, PCTP, PCYOX1, PCYOX1L, PCYT1A, PCYT1B, PCYT2, PDAP1, PDC, PDCD1, PDCD10, PDCD11, PDCD1LG2, PDCD2, PDCD2L, PDCD4, PDCD5, PDCD6, PDCD6IP, PDCD7, PDCL, PDCL2, PDCL3, PDE10A, PDE11A, PDE12, PDE1A, PDE1B, PDE1C, PDE2A, PDE3A, PDE3B, PDE4A, PDE4B, PDE4C, PDE4D, PDE4DIP, PDE5A, PDE6A, PDE6B, PDE6C, PDE6D, PDE6G, PDE6H, PDE7A, PDE7B, PDE8A, PDE8B, PDE9A, PDF, PDGFA, PDGFB, PDGFC, PDGFD, PDGFRA, PDGFRB, PDGFRL, PDHA1, PDHA2, PDHB, PDHX, PDIA2, PDIA3, PDIA4, PDIA5, PDIA6, PDIK1L, PDILT, PDK1, PDK2, PDK3, PDK4, PDLIM1, PDLIM2, PDLIM3, PDLIM4, PDLIM5, PDLIM7, PDP1, PDP2, PDPK1, PDPN, PDPR, PDRG1, PDS5A, PDS5B, PDSS1, PDSS2, PDX1, PDXDC1, PDXK, PDXP, PDYN, PDZD11, PDZD2, PDZD3, PDZD4, PDZD7, PDZD8, PDZD9, PDZK1, PDZK1IP1, PDZRN3, PDZRN4, PEA15, PEAK1, PEAR1, PEBP1, PEBP4, PECAM1, PECR, PEF1, PEG10, PEG3, PELI1, PELI2, PELI3, PELO, PELP1, PEMT, PENK, PEPD, PER1, PER2, PER3, PERM1, PERP, PES1, PET100, PET117, PEX1, PEX10, PEX11A, PEX11B, PEX11G, PEX12, PEX13, PEX14, PEX16, PEX19, PEX2, PEX26, PEX3, PEX5, PEX5L, PEX6, PEX7, PF4, PF4V1, PFAS, PFDN1, PFDN2, PFDN4, PFDN5, PFDN6, PFKFB1, PFKFB2, PFKFB3, PFKFB4, PFKL, PFKM, PFKP, PFN1, PFN2, PFN3, PFN4, PGA3, PGA4, PGA5, PGAM1, PGAM2, PGAM4, PGAM5, PGAP1, PGAP2, PGAP3, PGBD1, PGBD2, PGBD4, PGBD5, PGC, PGD, PGF, PGGHG, PGGT1B, PGK1, PGK2, PGLS, PGLYRP1, PGLYRP2, PGLYRP3, PGLYRP4, PGM1, PGM2, PGM2L1, PGM3, PGM5, PGP, PGPEP1, PGPEP1L, PGR, PGRMC1, PGRMC2, PGS1, PHACTR1, PHACTR2, PHACTR3, PHACTR4, PHAX, PHB, PHB2, PHC1, PHC2, PHC3, PHEX, PHF1, PHF10, PHF11, PHF12, PHF13, PHF14, PHF19, PHF2, PHF20, PHF20L1, PHF21A, PHF21B, PHF23, PHF24, PHF3, PHF5A, PHF6, PHF7, PHF8, PHGDH, PHGR1, PHIP, PHKA1, PHKA2, PHKB, PHKG1, PHKG2, PHLDA1, PHLDA2, PHLDA3, PHLDB1, PHLDB2, PHLDB3, PHLPP1, PHLPP2, PHOSPHO1, PHOSPHO2, PHOX2A, PHOX2B, PHPT1, PHRF1, PHTF1, PHTF2, PHYH, PHYHD1, PHYHIP, PHYHIPL, PHYKPL, PI15, PI16, PI3, PI4K2A, PI4K2B, PI4KA, PI4 KB, PIANP, PIAS1, PIAS2, PIAS3, PIAS4, PIBF1, PICALM, PICK1, PID1, PIDD1, PIEZO1, PIEZO2, PIF1, PIFO, PIGA, PIGB, PIGBOS1, PIGC, PIGF, PIGG, PIGH, PIGK, PIGL, PIGM, PIGN, PIGO, PIGP, PIGQ, PIGR, PIGS, PIGT, PIGU, PIGV, PIGW, PIGX, PIGY, PIGZ, PIH1D1, PIH1D2, PIH1D3, PIK3AP1, PIK3C2A, PIK3C2B, PIK3C2G, PIK3C3, PIK3CA, PIK3CB, PIK3CD, PIK3CG, PIK3IP1, PIK3R1, PIK3R2, PIK3R3, PIK3R4, PIK3R5, PIK3R6, PIKFYVE, PILRA, PILRB, PITM1, PIM2, PIM3, PIMREG, PIN1, PIN4, PINK1, PINLYP, PINX1, PIP, PIP4K2A, PIP4K2B, PIP4K2C, PIP5K1A, PIP5K1B, PIP5K1C, PIP5KL1, PIPOX, PIR, PIRT, PISD, PITHD1, PITPNA, PITPNB, PITPNC1, PITPNM1, PITPNM2, PITPNM3, PITRM1, PITX1, PITX2, PITX3, PIWIL1, PIWIL2, PIWIL3, PIWIL4, PJA1, PJA2, PKD1, PKD1L1, PKD1L2, PKD1L3, PKD2, PKD2L1, PKD2L2, PKDCC, PKDREJ, PKHD1, PKHD1L1, PKIA, PKIB, PKIG, PKLR, PKM, PKMYT1, PKN1, PKN2, PKN3, PKNOX1, PKNOX2, PKP1, PKP2, PKP3, PKP4, PLA1A, PLA2G10, PLA2G12A, PLA2G12B, PLA2G15, PLA2G16, PLA2G1B, PLA2G2A, PLA2G2C, PLA2G2D, PLA2G2E, PLA2G2F, PLA2G3, PLA2G4A, PLA2G4B, PLA2G4C, PLA2G4D, PLA2G4E, PLA2G4F, PLA2G5, PLA2G6, PLA2G7, PLA2R1, PLAA, PLAC1, PLAC4, PLAC8, PLAC8L1, PLAC9, PLAG1, PLAGL1, PLAGL2, PLAT, PLAU, PLAUR, PLB1, PLBD1, PLBD2, PLCB1, PLCB2, PLCB3, PLCB4, PLCD1, PLCD3, PLCD4, PLCE1, PLCG1, PLCG2, PLCH1, PLCH2, PLCL1, PLCL2, PLCXD1, PLCXD2, PLCXD3, PLCZ1, PLD1, PLD2, PLD3, PLD4, PLD5, PLD6, PLEC, PLEK, PLEK2, PLEKHA1, PLEKHA2, PLEKHA3, PLEKHA4, PLEKHA5, PLEKHA6, PLEKHA7, PLEKHA8, PLEKHB1, PLEKHB2, PLEKHD1, PLEKHF1, PLEKHF2, PLEKHG1, PLEKHG2, PLEKHG3, PLEKHG4, PLEKHG4B, PLEKHG5, PLEKHG6, PLEKHG7, PLEKHH1, PLEKHH2, PLEKHH3, PLEKHJ1, PLEKHM1, PLEKHM2, PLEKHM3, PLEKHN1, PLEKHO1, PLEKHO2, PLEKHS1, PLET1, PLG, PLGLB1, PLGLB2, PLGRKT, PLIN1, PLIN2, PLIN3, PLIN4, PLIN5, PLK1, PLK2, PLK3, PLK4, PLK5, PLLP, PLN, PLOD1, PLOD2, PLOD3, PLP1, PLP2, PLPBP, PLPP1, PLPP2, PLPP3, PLPP4, PLPP5, PLPP6, PLPP7, PLPPR1, PLPPR2, PLPPR3, PLPPR4, PLPPR5, PLRG1, PLS1, PLS3, PLSCR1, PLSCR2, PLSCR3, PLSCR4, PLSCR5, PLTP, PLVAP, PLXDC1, PLXDC2, PLXNA1, PLXNA2, PLXNA3, PLXNA4, PLXNB1, PLXNB2, PLXNB3, PLXNC1, PLXND1, PM20D1, PM20D2, PMAIP1, PMCH, PMEL, PMEPA1, PMF1, PMF1-BGLAP, PMFBP1, PML, PMM1, PMM2, PMP2, PMP22, PMPCA, PMPCB, PMS1, PMS2, PMVK, PNCK, PNISR, PNKD, PNKP, PNLDC1, PNLIP, PNLIPRP1, PNLIPRP2, PNLIPRP3, PNMA1, PNMA2, PNMA3, PNMA5, PNMA6A, PNMA6E, PNMA6F, PNMA8A, PNMA8B, PNMA8C, PNMT, PNN, PNO1, PNOC, PNP, PNPLA1, PNPLA2, PNPLA3, PNPLA4, PNPLA5, PNPLA6, PNPLA7, PNPLA8, PNPO, PNPT1, PNRC1, PNRC2, POC1A, POC1B, POC1B-GALNT4, POC5, PODN, PODNL1, PODXL, PODXL2, POF1B, POFUT1, POFUT2, POGK, POGLUT1, POGZ, POLA1, POLA2, POLB, POLD1, POLD2, POLD3, POLD4, POLDIP2, POLDIP3, POLE, POLE2, POLE3, POLE4, POLG, POLG2, POLH, POLI, POLK, POLL, POLM, POLN, POLQ, POLR1A, POLR1B, POLR1C, POLR1D, POLR1E, POLR2A, POLR2B, POLR2C, POLR2D, POLR2E, POLR2F, POLR2G, POLR2H, POLR2I, POLR2J, POLR2J2, POLR2J3, POLR2K, POLR2L, POLR2M, POLR3A, POLR3B, POLR3C, POLR3D, POLR3E, POLR3F, POLR3G, POLR3GL, POLR3H, POLR3K, POLRMT, POM121, POM121C, POM121L12, POM121L2, POMC, POMGNT1, POMGNT2, POMK, POMP, POMT1, POMT2, POMZP3, PON1, PON2, PON3, POP1, POP4, POP5, POP7, POPDC2, POPDC3, POR, PORCN, POSTN, POT1, POTEA, POTEB, POTEB2, POTEB3, POTEC, POTED, POTEE, POTEF, POTEG, POTEH, POTEI, POTEJ, POTEM, POU1F1, POU2AF1, POU2F1, POU2F2, POU2F3, POU3F1, POU3F2, POU3F3, POU3F4, POU4F1, POU4F2, POU4F3, POU5F1, POU5F1B, POU5F2, POU6F1, POU6F2, PP2D1, PPA1, PPA2, PPAN, PPAN-P2RY11, PPARA, PPARD, PPARG, PPARGC1A, PPARGC1B, PPAT, PPBP, PPCDC, PPCS, PPDPF, PPEF1, PPEF2, PPFIA1, PPFIA2, PPFIA3, PPFIA4, PPFIBP1, PPFIBP2, PPHLN1, PPIA, PPIAL4A, PPIAL4C, PPIAL4D, PPIAL4E, PPIAL4F, PPIAL4G, PPIB, PPIC, PPID, PPIE, PPIF, PPIG, PPIH, PPIL1, PPIL2, PPIL3, PPIL4, PPIL6, PPIP5K1, PPIP5K2, PPL, PPM1A, PPM1B, PPM1D, PPM1E, PPM1F, PPM1G, PPM1H, PPM1J, PPM1K, PPM1L, PPM1M, PPM1N, PPME1, PPOX, PPP1CA, PPP1CB, PPP1CC, PPP1R10, PPP1R11, PPP1R12A, PPP1R12B, PPP1R12C, PPP1R13B, PPP1R13L, PPP1R14A, PPP1R14B, PPP1R14C, PPP1R14D, PPP1R15A, PPP1R15B, PPP1R16A, PPP1R16B, PPP1R17, PPP1R18, PPP1R1A, PPP1R1B, PPP1R1C, PPP1R2, PPP1R21, PPP1R26, PPP1R27, PPP1R2P3, PPP1R2P9, PPP1R32, PPP1R35, PPP1R36, PPP1R37, PPP1R3A, PPP1R3B, PPP1R3C, PPP1R3D, PPP1R3E, PPP1R3F, PPP1R3G, PPP1R42, PPP1R7, PPP1R8, PPP1R9A, PPP1R9B, PPP2CA, PPP2CB, PPP2R1A, PPP2R1B, PPP2R2A, PPP2R2B, PPP2R2C, PPP2R2D, PPP2R3A, PPP2R3B, PPP2R3C, PPP2R5A, PPP2R5B, PPP2R5C, PPP2R5D, PPP2R5E, PPP3CA, PPP3CB, PPP3CC, PPP3R1, PPP3R2, PPP4C, PPP4R1, PPP4R2, PPP4R3A, PPP4R3B, PPP4R3CP, PPP4R4, PPP5C, PPP5D1, PPP6C, PPP6R1, PPP6R2, PPP6R3, PPRC1, PPT1, PPT2, PPT2-EGFL8, PPTC7, PPWD1, PPY, PQBP1, PQLC1, PQLC2, PQLC2L, PQLC3, PRAC1, PRAC2, PRADC1, PRAF2, PRAG1, PRAM1, PRAME, PRAMEF1, PRAMEF10, PRAMEF11, PRAMEF12, PRAMEF13, PRAMEF14, PRAMEF15, PRAMEF17, PRAMEF18, PRAMEF19, PRAMEF2, PRAMEF20, PRAMEF25, PRAMEF26, PRAMEF27, PRAMEF33, PRAMEF4, PRAMEF5, PRAMEF6, PRAMEF7, PRAMEF8, PRAMEF9, PRAP1, PRB1, PRB2, PRB3, PRB4, PRC1, PRCC, PRCD, PRCP, PRDM1, PRDM10, PRDM11, PRDM12, PRDM13, PRDM14, PRDM15, PRDM16, PRDM2, PRDM4, PRDM5, PRDM6, PRDM7, PRDM8, PRDM9, PRDX1, PRDX2, PRDX3, PRDX4, PRDX5, PRDX6, PREB, PRELID1, PRELID2, PRELID3A, PRELID3B, PRELP, PREP, PREPL, PREX1, PREX2, PRF1, PRG2, PRG3, PRG4, PRH1, PRH2, PRICKLE1, PRICKLE2, PRICKLE3, PRICKLE4, PRIM1, PRIM2, PRIMA1, PRIMPOL, PRKAA1, PRKAA2, PRKAB1, PRKAB2, PRKACA, PRKACB, PRKACG, PRKAG1, PRKAG2, PRKAG3, PRKAR1A, PRKAR1B, PRKAR2A, PRKAR2B, PRKCA, PRKCB, PRKCD, PRKCE, PRKCG, PRKCH, PRKCI, PRKCQ, PRKCSH, PRKCZ, PRKD1, PRKD2, PRKD3, PRKDC, PRKG1, PRKG2, PRKN, PRKRA, PRKRIP1, PRKX, PRL, PRLH, PRLHR, PRLR, PRM1, PRM2, PRM3, PRMT1, PRMT2, PRMT3, PRMT5, PRMT6, PRMT7, PRMT8, PRMT9, PRND, PRNP, PRNT, PROB1, PROC, PROCA1, PROCR, PRODH, PRODH2, PROK1, PROK2, PROKR1, PROKR2, PROM1, PROM2, PROP1, PRORY, PROS1, PROSER1, PROSER2, PROSER3, PROX1, PROX2, PROZ, PRPF18, PRPF19, PRPF3, PRPF31, PRPF38A, PRPF38B, PRPF39, PRPF4, PRPF40A, PRPF40B, PRPF4B, PRPF6, PRPF8, PRPH, PRPH2, PRPS1, PRPS1L1, PRPS2, PRPSAP1, PRPSAP2, PRR11, PRR12, PRR13, PRR14, PRR14L, PRR15, PRR15L, PRR16, PRR18, PRR19, PRR20A, PRR20B, PRR20C, PRR20D, PRR20E, PRR21, PRR22, PRR23A, PRR23B, PRR23C, PRR23D1, PRR23D2, PRR25, PRR26, PRR27, PRR29, PRR3, PRR30, PRR32, PRR34, PRR35, PRR36, PRR4, PRR5, PRR5-ARHGAP8, PRR5L, PRR7, PRR9, PRRC1, PRRC2A, PRRC2B, PRRC2C, PRRG1, PRRG2, PRRG3, PRRG4, PRRT1, PRRT2, PRRT3, PRRT4, PRRX1, PRRX2, PRSS1, PRSS12, PRSS16, PRSS2, PRSS21, PRSS22, PRSS23, PRSS27, PRSS3, PRSS33, PRSS35, PRSS36, PRSS37, PRSS38, PRSS41, PRSS42, PRSS45, PRSS46, PRSS48, PRSS50, PRSS51, PRSS53, PRSS54, PRSS55, PRSS56, PRSS57, PRSS58, PRSS8, PRTFDC1, PRTG, PRTN3, PRUNE1, PRUNE2, PRX, PRY, PRY2, PSAP, PSAPL1, PSAT1, PSCA, PSD, PSD2, PSD3, PSD4, PSEN1, PSEN2, PSENEN, PSG1, PSG11, PSG2, PSG3, PSG4, PSG5, PSG6, PSG7, PSG8, PSG9, PSIP1, PSKH1, PSKH2, PSMA1, PSMA2, PSMA3, PSMA4, PSMA5, PSMA6, PSMA7, PSMA8, PSMB1, PSMB10, PSMB11, PSMB2, PSMB3, PSMB4, PSMB5, PSMB6, PSMB7, PSMB8, PSMB9, PSMC1, PSMC2, PSMC3, PSMC3IP, PSMC4, PSMC5, PSMC6, PSMD1, PSMD10, PSMD11, PSMD12, PSMD13, PSMD14, PSMD2, PSMD3, PSMD4, PSMD5, PSMD6, PSMD7, PSMD8, PSMD9, PSME1, PSME2, PSME3, PSME4, PSMF1, PSMG1, PSMG2, PSMG3, PSMG4, PSORS1C1, PSORS1C2, PSPC1, PSPH, PSPN, PSRC1, PSTK, PSTPIP1, PSTPIP2, PTAFR, PTAR1, PTBP1, PTBP2, PTBP3, PTCD1, PTCD2, PTCD3, PTCH1, PTCH2, PTCHD1, PTCHD3, PTCHD4, PTCRA, PTDSS1, PTDSS2, PTEN, PTER, PTF1A, PTGDR, PTGDR2, PTGDS, PTGER1, PTGER2, PTGER3, PTGER4, PTGES, PTGES2, PTGES3, PTGES3L, PTGES3L-AARSD1, PTGFR, PTGFRN, PTGIR, PTGIS, PTGR1, PTGR2, PTGS1, PTGS2, PTH, PTH1R, PTH2, PTH2R, PTHLH, PTK2, PTK2B, PTK6, PTK7, PTMA, PTMS, PTN, PTOV1, PTP4A1, PTP4A2, PTP4A3, PTPA, PTPDC1, PTPMT1, PTPN1, PTPN11, PTPN12, PTPN13, PTPN14, PTPN18, PTPN2, PTPN20, PTPN21, PTPN22, PTPN23, PTPN3, PTPN4, PTPN5, PTPN6, PTPN7, PTPN9, PTPRA, PTPRB, PTPRC, PTPRCAP, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRZ1, PTRH1, PTRH2, PTRHD1, PTS, PTTG1, PTTG1IP, PTTG2, PTX3, PTX4, PUDP, PUF60, PUM1, PUM2, PUM3, PURA, PURB, PURG, PUS1, PUS10, PUS3, PUS7, PUS7L, PUSL1, PVALB, PVR, PVRIG, PWP1, PWP2, PWWP2A, PWWP2B, PXDC1, PXDN, PXDNL, PXK, PXMP2, PXMP4, PXN, PXT1, PXYLP1, PYCARD, PYCR1, PYCR2, PYCR3, PYDC1, PYDC2, PYGB, PYGL, PYGM, PYGO1, PYGO2, PYHIN1, PYM1, PYROXD1, PYROXD2, PYURF, PYY, PZP, QARS, QDPR, QKI, QPCT, QPCTL, QPRT, QRFP, QRFPR, QRICH1, QRICH2, QRSL1, QSER1, QSOX1, QSOX2, QTRT1, QTRT2, R3HCC1, R3HCC1L, R3HDM1, R3HDM2, R3HDM4, R3HDML, RAB10, RAB11A, RAB11B, RAB11FIP1, RAB11FIP2, RAB11FIP3, RAB11FIP4, RAB11FIP5, RAB12, RAB13, RAB14, RAB15, RAB17, RAB18, RAB19, RAB1A, RAB1B, RAB20, RAB21, RAB22A, RAB23, RAB24, RAB25, RAB26, RAB27A, RAB27B, RAB28, RAB29, RAB2A, RAB2B, RAB30, RAB31, RAB32, RAB33A, RAB33B, RAB34, RAB35, RAB36, RAB37, RAB38, RAB39A, RAB39B, RAB3A, RAB3B, RAB3C, RAB3D, RAB3GAP1, RAB3GAP2, RAB3IL1, RAB3IP, RAB40A, RAB40AL, RAB40B, RAB40C, RAB41, RAB42, RAB43, RAB44, RAB4A, RAB4B, RAB4B-EGLN2, RAB5A, RAB5B, RAB5C, RAB6A, RAB6B, RAB6C, RAB7A, RAB7B, RAB8A, RAB8B, RAB9A, RAB9B, RABAC1, RABEP1, RABEP2, RABEPK, RABGAP1, RABGAP1L, RABGEF1, RABGGTA, RABGGTB, RABIF, RABL2A, RABL2B, RABL3, RABL6, RAC1, RAC2, RAC3, RACGAP1, RACK1, RAD1, RAD17, RAD18, RAD21, RAD21L1, RAD23A, RAD23B, RAD50, RAD51, RAD51AP1, RAD51AP2, RAD51B, RAD51C, RAD51D, RAD52, RAD54B, RAD54L, RAD54L2, RAD9A, RAD9B, RADTL, RAE1, RAET1E, RAET1G, RAET1L, RAF1, RAG1, RAG2, RAI1, RAI14, RAI2, RALA, RALB, RALBP1, RALGAPA1, RALGAPA2, RALGAPB, RALGDS, RALGPS1, RALGPS2, RALY, RALYL, RAMP1, RAMP2, RAMP3, RAN, RANBP1, RANBP10, RANBP17, RANBP2, RANBP3, RANBP3L, RANBP6, RANBP9, RANGAP1, RANGRF, RAP1A, RAP1B, RAP1GAP, RAP1GAP2, RAP1GDS1, RAP2A, RAP2B, RAP2C, RAPGEF1, RAPGEF2, RAPGEF3, RAPGEF4, RAPGEF5, RAPGEF6, RAPGEFL1, RAPH1, RAPSN, RARA, RARB, RARG, RARRES1, RARRES2, RARRES3, RARS, RARS2, RASA1, RASA2, RASA3, RASA4, RASA4B, RASAL1, RASAL2, RASAL3, RASD1, RASD2, RASEF, RASGEF1A, RASGEF1B, RASGEF1C, RASGRF1, RASGRF2, RASGRP1, RASGRP2, RASGRP3, RASGRP4, RASIP1, RASL10A, RASL10B, RASLI1A, RASL11B, RASL12, RASSF1, RASSF10, RASSF2, RASSF3, RASSF4, RASSF5, RASSF6, RASSF7, RASSF8, RASSF9, RAVER1, RAVER2, RAX, RAX2, RB1, RB1CC1, RBAK, RBAK-RBAKDN, RBBP4, RBBP5, RBBP6, RBBP7, RBBP8, RBBP8NL, RBBP9, RBCK1, RBFA, RBFOX1, RBFOX2, RBFOX3, RBKS, RBL1, RBL2, RBM10, RBM11, RBM12, RBM12B, RBM14, RBM14-RBM4, RBM15, RBM15B, RBM17, RBM18, RBM19, RBM20, RBM22, RBM23, RBM24, RBM25, RBM26, RBM27, RBM28, RBM3, RBM33, RBM34, RBM38, RBM39, RBM4, RBM41, RBM42, RBM43, RBM44, RBM45, RBM46, RBM47, RBM48, RBM4B, RBM5, RBM6, RBM7, RBM8A, RBMS1, RBMS2, RBMS3, RBMX, RBMX2, RBMXL1, RBMXL2, RBMXL3, RBMY1A1, RBMY1B, RBMY1D, RBMY1E, RBMY1F, RBMY1J, RBP1, RBP2, RBP3, RBP4, RBP5, RBP7, RBPJ, RBPJL, RBPMS, RBPMS2, RBSN, RBX1, RC3H1, RC3H2, RCAN1, RCAN2, RCAN3, RCBTB1, RCBTB2, RCC1, RCC1L, RCC2, RCCD1, RCE1, RCHY1, RCL1, RCN1, RCN2, RCN3, RCOR1, RCOR2, RCOR3, RCSD1, RCVRN, RD3, RD3L, RDH10, RDH11, RDH12, RDH13, RDH14, RDH16, RDH5, RDH8, RDM1, RDX, REC114, REC8, RECK, RECQL, RECQL4, RECQL5, REEP1, REEP2, REEP3, REEP4, REEP5, REEP6, REG1A, REG1B, REG3A, REG3G, REG4, REL, RELA, RELB, RELL1, RELL2, RELN, RELT, REM1, REM2, REN, RENBP, REP15, REPIN1, REPS1, REPS2, RER1, RERE, RERG, RERGL, RESP18, REST, RET, RETN, RETNLB, RETREG1, RETREG2, RETREG3, RETSAT, REV1, REV3L, REXO1, REXO2, REXO4, REXO5, RFC1, RFC2, RFC3, RFC4, RFC5, RFESD, RFFL, RFK, RFLNA, RFLNB, RFNG, RFPL1, RFPL2, RFPL3, RFPL3S, RFPL4A, RFPL4AL1, RFPL4B, RFT1, RFTN1, RFTN2, RFWD2, RFWD3, RFX1, RFX2, RFX3, RFX4, RFX5, RFX6, RFX7, RFX8, RFXANK, RFXAP, RGCC, RGL1, RGL2, RGL3, RGL4, RGMA, RGMB, RGN, RGP1, RGPD1, RGPD2, RGPD3, RGPD4, RGPD5, RGPD6, RGPD8, RGR, RGS1, RGS10, RGS11, RGS12, RGS13, RGS14, RGS16, RGS17, RGS18, RGS19, RGS2, RGS20, RGS21, RGS22, RGS3, RGS4, RGS5, RGS6, RGS7, RGS7BP, RGS8, RGS9, RGS9BP, RGSL1, RHAG, RHBDD1, RHBDD2, RHBDD3, RHBDF1, RHBDF2, RHBDL1, RHBDL2, RHBDL3, RHBG, RHCE, RHCG, RHD, RHEB, RHEBL1, RHNO1, RHO, RHOA, RHOB, RHOBTB1, RHOBTB2, RHOBTB3, RHOC, RHOD, RHOF, RHOG, RHOH, RHOJ, RHOQ, RHOT1, RHOT2, RHOU, RHOV, RHOXF1, RHOXF2, RHOXF2B, RHPN1, RHPN2, RIBC1, RIBC2, RIC1, RIC3, RIC8A, RIC8B, RICTOR, RIDA, RIF1, RIIAD1, RILP, RILPL1, RILPL2, RIMBP2, RIMBP3, RIMBP3B, RIMBP3C, RIMKLA, RIMKLB, RIMS1, RIMS2, RIMS3, RIMS4, RIN1, RIN2, RIN3, RING1, RINL, RINT1, RIOK1, RIOK2, RIOK3, RIOX1, RIOX2, RIPK1, RIPK2, RIPK3, RIPK4, RIPOR1, RIPOR2, RIPOR3, RIPPLY1, RIPPLY2, RIPPLY3, RIT1, RIT2, RITA1, RLBP1, RLF, RLIM, RLN1, RLN2, RLN3, RMDN1, RMDN2, RMDN3, RMI1, RMI2, RMND1, RMND5A, RMND5B, RNASE1, RNASE10, RNASE11, RNASE12, RNASE13, RNASE2, RNASE3, RNASE4, RNASE6, RNASE7, RNASE8, RNASE9, RNASEH1, RNASEH2A, RNASEH2B, RNASEH2C, RNASEK, RNASEK-C17orf49, RNASEL, RNASET2, RND1, RND2, RND3, RNF10, RNF103, RNF103-CHMP3, RNF11, RNF111, RNF112, RNF113A, RNF113B, RNF114, RNF115, RNF121, RNF122, RNF123, RNF125, RNF126, RNF128, RNF13, RNF130, RNF133, RNF135, RNF138, RNF139, RNF14, RNF141, RNF144A, RNF144B, RNF145, RNF146, RNF148, RNF149, RNF150, RNF151, RNF152, RNF157, RNF165, RNF166, RNF167, RNF168, RNF169, RNF17, RNF170, RNF175, RNF180, RNF181, RNF182, RNF183, RNF185, RNF186, RNF187, RNF19A, RNF19B, RNF2, RNF20, RNF207, RNF208, RNF212, RNF212B, RNF213, RNF214, RNF215, RNF216, RNF217, RNF219, RNF220, RNF222, RNF223, RNF224, RNF225, RNF24, RNF25, RNF26, RNF31, RNF32, RNF34, RNF38, RNF39, RNF4, RNF40, RNF41, RNF43, RNF44, RNF5, RNF6, RNF7, RNF8, RNFT1, RNFT2, RNGTT, RNH1, RNLS, RNMT, RNPC3, RNPEP, RNPEPL1, RNPS1, ROBO1, ROBO2, ROBO3, ROBO4, ROCK1, ROCK2, ROGDI, ROM1, ROMO1, ROPN1, ROPN1B, ROPN1L, ROR1, ROR2, RORA, RORB, RORC, ROS1, RP1, RP1L1, RP2, RP9, RPA1, RPA2, RPA3, RPA4, RPAIN, RPAP1, RPAP2, RPAP3, RPE, RPE65, RPEL1, RPF1, RPF2, RPGR, RPGRIP1, RPGRIP1L, RPH3A, RPH3AL, RPIA, RPL10, RPL10A, RPL10L, RPL11, RPL12, RPL13, RPL13A, RPL14, RPL15, RPL17, RPL17-C18orf32, RPL18, RPL18A, RPL19, RPL21, RPL22, RPL22L1, RPL23, RPL23A, RPL24, RPL26, RPL26L1, RPL27, RPL27A, RPL28, RPL29, RPL3, RPL30, RPL31, RPL32, RPL34, RPL35, RPL35A, RPL36, RPL36A, RPL36A-HNRNPH2, RPL36AL, RPL37, RPL37A, RPL38, RPL39, RPL39L, RPL3L, RPL4, RPL41, RPL5, RPL6, RPL7, RPL7A, RPL7L1, RPL8, RPL9, RPLP0, RPLP1, RPLP2, RPN1, RPN2, RPP14, RPP21, RPP25, RPP25L, RPP30, RPP38, RPP40, RPRD1A, RPRD1B, RPRD2, RPRM, RPRML, RPS10, RPS10-NUDT3, RPS11, RPS12, RPS13, RPS14, RPS15, RPS15A, RPS16, RPS17, RPS18, RPS19, RPS19BP1, RPS2, RPS20, RPS21, RPS23, RPS24, RPS25, RPS26, RPS27, RPS27A, RPS27L, RPS28, RPS29, RPS3, RPS3A, RPS4X, RPS4Y1, RPS4Y2, RPS5, RPS6, RPS6KA1, RPS6KA2, RPS6KA3, RPS6KA4, RPS6KA5, RPS6KA6, RPS6KB1, RPS6KB2, RPS6KC1, RPS6KL1, RPS7, RPS8, RPS9, RPSA, RPTN, RPTOR, RPUSD1, RPUSD2, RPUSD3, RPUSD4, RRAD, RRAGA, RRAGB, RRAGC, RRAGD, RRAS, RRAS2, RRBP1, RREB1, RRH, RRM1, RRM2, RRM2B, RRN3, RRNAD1, RRP1, RRP12, RRP15, RRP1B, RRP36, RRP7A, RRP8, RRP9, RRS1, RS1, RSAD1, RSAD2, RSBN1, RSBN1L, RSC1A1, RSF1, RSG1, RSL1D1, RSL24D1, RSPH1, RSPH10B, RSPH10B2, RSPH14, RSPH3, RSPH4A, RSPH6A, RSPH9, RSPO1, RSPO2, RSPO3, RSPO4, RSPRY1, RSRC1, RSRC2, RSRP1, RSU1, RTBDN, RTCA, RTCB, RTEL1, RTEL1-TNFRSF6B, RTF1, RTFDC1, RTKN, RTKN2, RTL1, RTL10, RTL3, RTL4, RTL5, RTL6, RTL8A, RTL8B, RTL8C, RTL9, RTN1, RTN2, RTN3, RTN4, RTN4IP1, RTN4R, RTN4RL1, RTN4RL2, RTP1, RTP2, RTP3, RTP4, RTP5, RTTN, RUBCN, RUBCNL, RUFY1, RUFY2, RUFY3, RUFY4, RUNDC1, RUNDC3A, RUNDC3B, RUNX1, RUNX1T1, RUNX2, RUNX3, RUSC1, RUSC2, RUVBL1, RUVBL2, RWDD1, RWDD2A, RWDD2B, RWDD3, RWDD4, RXFP1, RXFP2, RXFP3, RXFP4, RXRA, RXRB, RXRG, RYBP, RYK, RYR1, RYR2, RYR3, S100A1, S100A10, S100A11, S100A12, S100A13, S100A14, S100A16, S100A2, S100A3, S100A4, S100A5, S100A6, S100A7, S100A7A, S100A7L2, S100A8, S100A9, S100B, S100G, S100P, S100PBP, S100Z, S1PR1, S1PR2, S1PR3, S1PR4, S1PR5, SAA1, SAA2, SAA2-SAA4, SAA4, SAAL1, SAC3D1, SACM1L, SACS, SAE1, SAFB, SAFB2, SAG, SAGE1, SALL1, SALL2, SALL3, SALL4, SAMD1, SAMD10, SAMD11, SAMD12, SAMD13, SAMD14, SAMD15, SAMD3, SAMD4A, SAMD4B, SAMD5, SAMD7, SAMD8, SAMD9, SAMD9L, SAMIHD1, SAMM50, SAMSN1, SAP130, SAP18, SAP25, SAP30, SAP30BP, SAP30L, SAPCD1, SAPCD2, SAR1A, SAR1B, SARAF, SARDH, SARM1, SARNP, SARS, SARS2, SART1, SART3, SASH1, SASH3, SASS6, SAT1, SAT2, SATB1, SATB2, SATL1, SAV1, SAXO1, SAXO2, SAYSD1, SBDS, SBF1, SBF2, SBK1, SBK2, SBK3, SBNO1, SBNO2, SBSN, SBSPON, SC5D, SCAF1, SCAF11, SCAF4, SCAF8, SCAI, SCAMPI, SCAMP2, SCAMP3, SCAMP4, SCAMP5, SCAND1, SCAP, SCAPER, SCARA3, SCARA5, SCARB1, SCARB2, SCARF1, SCARF2, SCART1, SCCPDH, SCD, SCD5, SCEL, SCFD1, SCFD2, SCG2, SCG3, SCG5, SCGBlA1, SCGB1C1, SCGB1C2, SCGB1D1, SCGB1D2, SCGB1D4, SCGB2A1, SCGB2A2, SCGB2B2, SCGB3A1, SCGB3A2, SCGN, SCH1P1, SCIMP, SCIN, SCLT1, SCLY, SCMH1, SCML1, SCML2, SCML4, SCN10A, SCN11A, SCN1A, SCN1B, SCN2A, SCN2B, SCN3A, SCN3B, SCN4A, SCN4B, SCN5A, SCN7A, SCN8A, SCN9A, SCNM1, SCNN1A, SCNN1B, SCNN1D, SCNN1G, SCO1, SCO2, SCOC, SCP2, SCP2D1, SCPEP1, SCRG1, SCRIB, SCRN1, SCRN2, SCRN3, SCRT1, SCRT2, SCT, SCTR, SCUBE1, SCUBE2, SCUBE3, SCX, SCYL1, SCYL2, SCYL3, SDAD1, SDC1, SDC2, SDC3, SDC4, SDCBP, SDCBP2, SDCCAG3, SDCCAG8, SDE2, SDF2, SDF2L1, SDF4, SDHA, SDHAF1, SDHAF2, SDHAF3, SDHAF4, SDHB, SDHC, SDHD, SDK1, SDK2, SDR16C5, SDR39U1, SDR42E1, SDR42E2, SDR9C7, SDS, SDSL, SEBOX, SEC11A, SEC11C, SEC13, SEC14L1, SEC14L2, SEC14L3, SEC14L4, SEC14L5, SEC14L6, SEC16A, SEC16B, SEC22A, SEC22B, SEC22C, SEC23A, SEC23B, SEC23IP, SEC24A, SEC24B, SEC24C, SEC24D, SEC31A, SEC31B, SEC61A1, SEC61A2, SEC61B, SEC61G, SEC62, SEC63, SECISBP2, SECISBP2L, SECTM1, SEH1L, SEL1L, SEL1L2, SEL1L3, SELE, SELENBP1, SELENOF, SELENOH, SELENOI, SELENOK, SELENOM, SELENON, SELENOO, SELENOP, SELENOS, SELENOT, SELENOV, SELENOW, SELL, SELP, SELPLG, SEM1, SEMA3A, SEMA3B, SEMA3C, SEMA3D, SEMA3E, SEMA3F, SEMA3G, SEMA4A, SEMA4B, SEMA4C, SEMA4D, SEMA4F, SEMA4G, SEMA5A, SEMA5B, SEMA6A, SEMA6B, SEMA6C, SEMA6D, SEMA7A, SEMG1, SEMG2, SENP1, SENP2, SENP3, SENP3-EIF4A1, SENP5, SENP6, SENP7, SENP8, SEPHS1, SEPHS2, SEPSECS, SEPT1, SEPT10, SEPT11, SEPT12, SEPT14, SEPT2, SEPT3, SEPT4, SEPT5, SEPT6, SEPT7, SEPT8, SEPT9, SERAC1, SERBP1, SERF1A, SERF1B, SERF2, SERGEF, SERHL2, SERINC1, SERINC2, SERINC3, SERINC4, SERINC5, SERP1, SERP2, SERPINA1, SERPINA10, SERPINA11, SERPINA12, SERPINA2, SERPINA3, SERPINA4, SERPINA5, SERPINA6, SERPINA7, SERPINA9, SERPINB1, SERPINB10, SERPINB11, SERPINB12, SERPINB13, SERPINB2, SERPINB3, SERPINB4, SERPINB5, SERPINB6, SERPINB7, SERPINB8, SERPINB9, SERPINC1, SERPIND1, SERPINE1, SERPINE2, SERPINE3, SERPINF1, SERPINF2, SERPING1, SERPINH1, SERPINI1, SERPINI2, SERTAD1, SERTAD2, SERTAD3, SERTAD4, SERTM1, SESN1, SESN2, SESN3, SESTD1, SET, SETBP1, SETD1A, SETD1B, SETD2, SETD3, SETD4, SETD5, SETD6, SETD7, SETD9, SETDB1, SETDB2, SETMAR, SETSIP, SETX, SEZ6, SEZ6L, SEZ6L2, SF1, SF3A1, SF3A2, SF3A3, SF3B1, SF3B2, SF3B3, SF3B4, SF3B5, SF3B6, SFI1, SFMBT1, SFMBT2, SFN, SFPQ, SFR1, SFRP1, SFRP2, SFRP4, SFRP5, SFSWAP, SFT2D1, SFT2D2, SFT2D3, SFTA2, SFTA3, SFTPA1, SFTPA2, SFTPB, SFTPC, SFTPD, SFXN1, SFXN2, SFXN3, SFXN4, SFXN5, SGCA, SGCB, SGCD, SGCE, SGCG, SGCZ, SGF29, SGIP1, SGK1, SGK2, SGK3, SGK494, SGMS1, SGMS2, SGO1, SGO2, SGPL1, SGPP1, SGPP2, SGSH, SGSM1, SGSM2, SGSM3, SGTA, SGTB, SH2B1, SH2B2, SH2B3, SH2D1A, SH2D1B, SH2D2A, SH2D3A, SH2D3C, SH2D4A, SH2D4B, SH2D5, SH2D6, SH2D7, SH3BGR, SH3BGRL, SH3BGRL2, SH3BGRL3, SH3BP1, SH3BP2, SH3BP4, SH3BP5, SH3BP5L, SH3D19, SH3D21, SH3GL1, SH3GL2, SH3GL3, SH3GLB1, SH3GLB2, SH3KBP1, SH3PXD2A, SH3PXD2B, SH3RF1, SH3RF2, SH3RF3, SH3TC1, SH3TC2, SH3YL1, SHANK1, SHANK2, SHANK3, SHARPIN, SHB, SHBG, SHC1, SHC2, SHC3, SHC4, SHCBP1, SHCBP1L, SHD, SHE, SHF, SHH, SHISA2, SHISA3, SHISA4, SHISA5, SHISA6, SHISA7, SHISA8, SHISA9, SHKBP1, SHMT1, SHMT2, SHOC2, SHOX, SHOX2, SHPK, SHPRH, SHQ1, SHROOM1, SHROOM2, SHROOM3, SHROOM4, SHTN1, SI, SIAE, SIAH1, SIAH2, SIAH3, SIDT1, SIDT2, SIGIRR, SIGLEC1, SIGLEC10, SIGLEC11, SIGLEC12, SIGLEC14, SIGLEC15, SIGLEC5, SIGLEC6, SIGLEC7, SIGLEC8, SIGLEC9, SIGLECL1, SIGMAR1, SIK1, SIK2, SIK3, SIKE1, SIL1, SIM1, SIM2, SIMC1, SIN3A, SIN3B, SIPA1, SIPA1L1, SIPA1L2, SIPA1L3, SIRPA, SIRPB1, SIRPB2, SIRPD, SIRPG, SIRT1, SIRT2, SIRT3, SIRT4, SIRT5, SIRT6, SIRT7, SIT1, SIVA1, SIX1, SIX2, SIX3, SIX4, SIX5, SIX6, SKA1, SKA2, SKA3, SKAP1, SKAP2, SKI, SKIDA1, SKIL, SKIV2L, SKIV2L2, SKOR1, SKOR2, SKP1, SKP2, SLA, SLA2, SLAIN1, SLAIN2, SLAMF1, SLAMF6, SLAMF7, SLAMF8, SLAMF9, SLBP, SLC10A1, SLC10A2, SLC10A3, SLC10A4, SLC10A5, SLC10A6, SLC10A7, SLC11A1, SLC11A2, SLC12A1, SLC12A2, SLC12A3, SLC12A4, SLC12A5, SLC12A6, SLC12A7, SLC12A8, SLC12A9, SLC13A1, SLC13A2, SLC13A3, SLC13A4, SLC13A5, SLC14A1, SLC14A2, SLC15A1, SLC15A2, SLC15A3, SLC15A4, SLC15A5, SLC16A1, SLC16A10, SLC16A11, SLC16A12, SLC16A13, SLC16A14, SLC16A2, SLC16A3, SLC16A4, SLC16A5, SLC16A6, SLC16A7, SLC16A8, SLC16A9, SLC17A1, SLC17A2, SLC17A3, SLC17A4, SLC17A5, SLC17A6, SLC17A7, SLC17A8, SLC17A9, SLC18A1, SLC18A2, SLC18A3, SLC18B1, SLC19A1, SLC19A2, SLC19A3, SLC1A1, SLC1A2, SLC1A3, SLC1A4, SLC1A5, SLC1A6, SLC1A7, SLC20A1, SLC20A2, SLC22A1, SLC22A10, SLC22A11, SLC22A12, SLC22A13, SLC22A14, SLC22A15, SLC22A16, SLC22A17, SLC22A18, SLC22A18AS, SLC22A2, SLC22A23, SLC22A24, SLC22A25, SLC22A3, SLC22A31, SLC22A4, SLC22A5, SLC22A6, SLC22A7, SLC22A8, SLC22A9, SLC23A1, SLC23A2, SLC23A3, SLC24A1, SLC24A2, SLC24A3, SLC24A4, SLC24A5, SLC25A1, SLC25A10, SLC25A11, SLC25A12, SLC25A13, SLC25A14, SLC25A15, SLC25A16, SLC25A17, SLC25A18, SLC25A19, SLC25A2, SLC25A20, SLC25A21, SLC25A22, SLC25A23, SLC25A24, SLC25A25, SLC25A26, SLC25A27, SLC25A28, SLC25A29, SLC25A3, SLC25A30, SLC25A31, SLC25A32, SLC25A33, SLC25A34, SLC25A35, SLC25A36, SLC25A37, SLC25A38, SLC25A39, SLC25A4, SLC25A40, SLC25A41, SLC25A42, SLC25A43, SLC25A44, SLC25A45, SLC25A46, SLC25A47, SLC25A48, SLC25A5, SLC25A51, SLC25A52, SLC25A53, SLC25A6, SLC26A1, SLC26A10, SLC26A11, SLC26A2, SLC26A3, SLC26A4, SLC26A5, SLC26A6, SLC26A7, SLC26A8, SLC26A9, SLC27A1, SLC27A2, SLC27A3, SLC27A4, SLC27A5, SLC27A6, SLC28A1, SLC28A2, SLC28A3, SLC29A1, SLC29A2, SLC29A3, SLC29A4, SLC2A1, SLC2A10, SLC2A11, SLC2A12, SLC2A13, SLC2A14, SLC2A2, SLC2A3, SLC2A4, SLC2A4RG, SLC2A5, SLC2A6, SLC2A7, SLC2A8, SLC2A9, SLC30A1, SLC30A10, SLC30A2, SLC30A3, SLC30A4, SLC30A5, SLC30A6, SLC30A7, SLC30A8, SLC30A9, SLC31A1, SLC31A2, SLC32A1, SLC33A1, SLC34A1, SLC34A2, SLC34A3, SLC35A1, SLC35A2, SLC35A3, SLC35A4, SLC35A5, SLC35B1, SLC35B2, SLC35B3, SLC35B4, SLC35C1, SLC35C2, SLC35D1, SLC35D2, SLC35D3, SLC35E1, SLC35E2, SLC35E2B, SLC35E3, SLC35E4, SLC35F1, SLC35F2, SLC35F3, SLC35F4, SLC35F5, SLC35F6, SLC35G1, SLC35G2, SLC35G3, SLC35G4, SLC35G5, SLC35G6, SLC36A1, SLC36A2, SLC36A3, SLC36A4, SLC37A1, SLC37A2, SLC37A3, SLC37A4, SLC38A1, SLC38A10, SLC38A11, SLC38A2, SLC38A3, SLC38A4, SLC38A5, SLC38A6, SLC38A7, SLC38A8, SLC38A9, SLC39A1, SLC39A10, SLC39A11, SLC39A12, SLC39A13, SLC39A14, SLC39A2, SLC39A3, SLC39A4, SLC39A5, SLC39A6, SLC39A7, SLC39A8, SLC39A9, SLC3A1, SLC3A2, SLC40A1, SLC41A1, SLC41A2, SLC41A3, SLC43A1, SLC43A2, SLC43A3, SLC44A1, SLC44A2, SLC44A3, SLC44A4, SLC44A5, SLC45A1, SLC45A2, SLC45A3, SLC45A4, SLC46A1, SLC46A2, SLC46A3, SLC47A1, SLC47A2, SLC48A1, SLC4A1, SLC4A10, SLC4A11, SLC4A1AP, SLC4A2, SLC4A3, SLC4A4, SLC4A5, SLC4A7, SLC4A8, SLC4A9, SLC50A1, SLC51A, SLC51B, SLC52A1, SLC52A2, SLC52A3, SLC5A1, SLC5A10, SLC5A11, SLC5A12, SLC5A2, SLC5A3, SLC5A4, SLC5A5, SLC5A6, SLC5A7, SLC5A8, SLC5A9, SLC6A1, SLC6A11, SLC6A12, SLC6A13, SLC6A14, SLC6A15, SLC6A16, SLC6A17, SLC6A18, SLC6A19, SLC6A2, SLC6A20, SLC6A3, SLC6A4, SLC6A5, SLC6A6, SLC6A7, SLC6A8, SLC6A9, SLC7A1, SLC7A10, SLC7A11, SLC7A13, SLC7A14, SLC7A2, SLC7A3, SLC7A4, SLC7A5, SLC7A6, SLC7A6OS, SLC7A7, SLC7A8, SLC7A9, SLC8A1, SLC8A2, SLC8A3, SLC8B1, SLC9A1, SLC9A2, SLC9A3, SLC9A3R1, SLC9A3R2, SLC9A4, SLC9A5, SLC9A6, SLC9A7, SLC9A8, SLC9A9, SLC9B1, SLC9B2, SLC9C1, SLC9C2, SLCO1A2, SLCO1B1, SLCO1B3, SLCO1B7, SLCO1C1, SLCO2A1, SLCO2B1, SLCO3A1, SLCO4A1, SLCO4C1, SLCO5A1, SLCO6A1, SLF1, SLF2, SLFN11, SLFN12, SLFN12L, SLFN13, SLFN14, SLFN5, SLFNL1, SLIRP, SLIT1, SLIT2, SLIT3, SLITRK1, SLITRK2, SLITRK3, SLITRK4, SLITRK5, SLITRK6, SLK, SLMAP, SLN, SLPI, SLTM, SLU7, SLURP1, SLURP2, SLX1A, SLX1B, SLX4, SLX4IP, SMAD1, SMAD2, SMAD3, SMAD4, SMAD5, SMAD6, SMAD7, SMAD9, SMAGP, SMAP1, SMAP2, SMARCA1, SMARCA2, SMARCA4, SMARCA5, SMARCAD1, SMARCAL1, SMARCB1, SMARCC1, SMARCC2, SMARCD1, SMARCD2, SMARCD3, SMARCE1, SMC1A, SMC1B, SMC2, SMC3, SMC4, SMC5, SMC6, SMCHD1, SMCO1, SMCO2, SMCO3, SMCO4, SMCP, SMCR8, SMDT1, SMG1, SMG5, SMG6, SMG7, SMG8, SMG9, SMIM1, SMIM10, SMIM10L1, SMIM10L2A, SMIM10L2B, SMIM11A, SMIM11B, SMIM12, SMIM13, SMIM14, SMIM15, SMIM17, SMIM18, SMIM19, SMIM2, SMIM20, SMIM21, SMIM22, SMIM23, SMIM24, SMIM26, SMIM27, SMIM28, SMIM29, SMIM3, SMIM30, SMIM31, SMIM4, SMIM5, SMIM6, SMIM7, SMIM8, SMIM9, SMKR1, SMLR1, SMN1, SMN2, SMNDC1, SMO, SMOC1, SMOC2, SMOX, SMPD1, SMPD2, SMPD3, SMPD4, SMPDL3A, SMPDL3B, SMPX, SMR3A, SMR3B, SMS, SMTN, SMTNL1, SMTNL2, SMU1, SMUG1, SMURF1, SMURF2, SMYD1, SMYD2, SMYD3, SMYD4, SMYD5, SNAIl, SNAI2, SNAI3, SNAP23, SNAP25, SNAP29, SNAP47, SNAP91, SNAPC1, SNAPC2, SNAPC3, SNAPC4, SNAPC5, SNAPIN, SNCA, SNCAIP, SNCB, SNCG, SND1, SNED1, SNF8, SNHG28, SNIP1, SNN, SNPH, SNRK, SNRNP200, SNRNP25, SNRNP27, SNRNP35, SNRNP40, SNRNP48, SNRNP70, SNRPA, SNRPA1, SNRPB, SNRPB2, SNRPC, SNRPD1, SNRPD2, SNRPD3, SNRPE, SNRPF, SNRPG, SNRPN, SNTA1, SNTB1, SNTB2, SNTG1, SNTG2, SNTN, SNU13, SNUPN, SNURF, SNW1, SNX1, SNX10, SNX11, SNX12, SNX13, SNX14, SNX15, SNX16, SNX17, SNX18, SNX19, SNX2, SNX20, SNX21, SNX22, SNX24, SNX25, SNX27, SNX29, SNX3, SNX30, SNX31, SNX32, SNX33, SNX4, SNX5, SNX6, SNX7, SNX8, SNX9, SOAT1, SOAT2, SOBP, SOCS1, SOCS2, SOCS3, SOCS4, SOCS5, SOCS6, SOCS7, SOD1, SOD2, SOD3, SOGA1, SOGA3, SOHLH1, SOHLH2, SON, SORBS1, SORBS2, SORBS3, SORCS1, SORCS2, SORCS3, SORD, SORL1, SORT1, SOS1, SOS2, SOST, SOSTDC1, SOWAHA, SOWAHB, SOWAHC, SOWAHD, SOX1, SOX10, SOX11, SOX12, SOX13, SOX14, SOX15, SOX17, SOX18, SOX2, SOX21, SOX3, SOX30, SOX4, SOX5, SOX6, SOX7, SOX8, SOX9, SP1, SP100, SP110, SP140, SP140L, SP2, SP3, SP4, SP5, SP6, SP7, SP8, SP9, SPA17, SPAAR, SPACA1, SPACA3, SPACA4, SPACA5, SPACA5B, SPACA6, SPACA7, SPACA9, SPAG1, SPAG11A, SPAG11B, SPAG16, SPAG17, SPAG4, SPAG5, SPAG6, SPAG7, SPAG8, SPAG9, SPAM1, SPANXA1, SPANXA2, SPANXB1, SPANXC, SPANXD, SPANXN1, SPANXN2, SPANXN3, SPANXN4, SPANXN5, SPARC, SPARCL1, SPART, SPAST, SPATA1, SPATA12, SPATA13, SPATA16, SPATA17, SPATA18, SPATA19, SPATA2, SPATA20, SPATA21, SPATA22, SPATA24, SPATA25, SPATA2L, SPATA3, SPATA31A1, SPATA31A3, SPATA31A5, SPATA31A6, SPATA31A7, SPATA31D1, SPATA31D3, SPATA31D4, SPATA31E1, SPATA32, SPATA33, SPATA4, SPATA45, SPATA46, SPATA5, SPATA5L1, SPATA6, SPATA6L, SPATA7, SPATA8, SPATA9, SPATC1, SPATC1L, SPATS1, SPATS2, SPATS2L, SPC24, SPC25, SPCS1, SPCS2, SPCS3, SPDEF, SPDL1, SPDYA, SPDYC, SPDYE1, SPDYE16, SPDYE2, SPDYE2B, SPDYE3, SPDYE4, SPDYE5, SPDYE6, SPECC1, SPECC1L, SPECC1L-ADORA2A, SPEF1, SPEF2, SPEG, SPEM1, SPEN, SPERT, SPESP1, SPG11, SPG21, SPG7, SPHAR, SPHK1, SPHK2, SPHKAP, SPI1, SPIB, SPIC, SPICE1, SPIDR, SPIN1, SPIN2A, SPIN2B, SPIN3, SPIN4, SPINK1, SPINK13, SPINK14, SPINK2, SPINK4, SPINK5, SPINK6, SPINK7, SPINK8, SPINK9, SPINT1, SPINT2, SPINT3, SPINT4, SPIRE1, SPIRE2, SPN, SPNS1, SPNS2, SPNS3, SPO11, SPOCD1, SPOCK1, SPOCK2, SPOCK3, SPON1, SPON2, SPOP, SPOPL, SPOUT1, SPP1, SPP2, SPPL2A, SPPL2B, SPPL2C, SPPL3, SPR, SPRED1, SPRED2, SPRED3, SPRN, SPRR1A, SPRR1B, SPRR2A, SPRR2B, SPRR2D, SPRR2E, SPRR2F, SPRR2G, SPRR3, SPRR4, SPRR5, SPRTN, SPRY1, SPRY2, SPRY3, SPRY4, SPRYD3, SPRYD4, SPRYD7, SPSB1, SPSB2, SPSB3, SPSB4, SPTA1, SPTAN1, SPTB, SPTBN1, SPTBN2, SPTBN4, SPTBN5, SPTLC1, SPTLC2, SPTLC3, SPTSSA, SPTSSB, SPTY2D1, SPTY2D1-AS1, SPX, SPZ1, SQLE, SQOR, SQSTM1, SRA1, SRBD1, SRC, SRCAP, SRCIN1, SRD5A1, SRD5A2, SRD5A3, SREBF1, SREBF2, SREK1, SREK1IP1, SRF, SRFBP1, SRGAP1, SRGAP2, SRGAP2B, SRGAP2C, SRGAP3, SRGN, SRI, SRL, SRM, SRMS, SRP14, SRP19, SRP54, SRP68, SRP72, SRP9, SRPK1, SRPK2, SRPK3, SRPRA, SRPRB, SRPX, SRPX2, SRR, SRRD, SRRM1, SRRM2, SRRM3, SRRM4, SRRM5, SRRT, SRSF1, SRSF10, SRSF11, SRSF12, SRSF2, SRSF3, SRSF4, SRSF5, SRSF6, SRSF7, SRSF8, SRSF9, SRXN1, SRY, SS18, SS18L1, SS18L2, SSB, SSBP1, SSBP2, SSBP3, SSBP4, SSC4D, SSC5D, SSFA2, SSH1, SSH2, SSH3, SSMEM1, SSNA1, SSPN, SSPO, SSR1, SSR2, SSR3, SSR4, SSRP1, SSSCA1, SST, SSTR1, SSTR2, SSTR3, SSTR4, SSTR5, SSU72, SSU72P8, SSUH2, SSX1, SSX2, SSX2B, SSX2IP, SSX3, SSX4, SSX4B, SSX5, SSX7, ST13, ST14, ST18, ST20, ST20-MTHFS, ST3GAL1, ST3GAL2, ST3GAL3, ST3GAL4, ST3GAL5, ST3GAL6, ST5, ST6GAL1, ST6GAL2, ST6GALNAC1, ST6GALNAC2, ST6GALNAC3, ST6GALNAC4, ST6GALNAC5, ST6GALNAC6, ST7, ST7L, ST8SIA1, ST8SIA2, ST8SIA3, ST8SIA4, ST8SIA5, ST8SIA6, STAB1, STAB2, STAC, STAC2, STAC3, STAG1, STAG2, STAG3, STAM, STAM2, STAMBP, STAMBPL1, STAP1, STAP2, STAR, STARD10, STARD13, STARD3, STARD3NL, STARD4, STARD5, STARD6, STARD7, STARD8, STARD9, STAT1, STAT2, STAT3, STAT4, STAT5A, STAT5B, STAT6, STATH, STAU1, STAU2, STBD1, STC1, STC2, STEAP1, STEAP1B, STEAP2, STEAP3, STEAP4, STH, STIL, STIM1, STIM2, STIP1, STK10, STK11, STK11IP, STK16, STK17A, STK17B, STK19, STK24, STK25, STK26, STK3, STK31, STK32A, STK32B, STK32C, STK33, STK35, STK36, STK38, STK38L, STK39, STK4, STK40, STKLD1, STMN1, STMN2, STMN3, STMN4, STMND1, STN1, STOM, STOML1, STOML2, STOML3, STON1, STON1-GTF2A1L, STON2, STOX1, STOX2, STPG1, STPG2, STPG3, STPG4, STRA6, STRA8, STRADA, STRADB, STRAP, STRBP, STRC, STRIP1, STRIP2, STRN, STRN3, STRN4, STS, STT3A, STT3B, STUB1, STUM, STX10, STX11, STX12, STX16, STX16-NPEPL1, STX17, STX18, STX19, STX1A, STX1B, STX2, STX3, STX4, STX5, STX6, STX7, STX8, STXBP1, STXBP2, STXBP3, STXBP4, STXBP5, STXBP5L, STXBP6, STYK1, STYX, STYXL1, SUB1, SUCLA2, SUCLG1, SUCLG2, SUCNR1, SUCO, SUDS3, SUFU, SUGCT, SUGP1, SUGP2, SUGT1, SULF1, SULF2, SULT1A1, SULT1A2, SULT1A3, SULT1A4, SULT1B1, SULT1C2, SULT1C3, SULT1C4, SULT1E1, SULT2A1, SULT2B1, SULT4A1, SULT6B1, SUMF1, SUMF2, SUMO1, SUMO2, SUMO3, SUMO4, SUN1, SUN2, SUN3, SUN5, SUOX, SUPT16H, SUPT20H, SUPT3H, SUPT4H1, SUPT5H, SUPT6H, SUPT7L, SUPV3L1, SURF1, SURF2, SURF4, SURF6, SUSD1, SUSD2, SUSD3, SUSD4, SUSD5, SUSD6, SUV39H1, SUV39H2, SUZ12, SV2A, SV2B, SV2C, SVBP, SVEP1, SVIL, SVIP, SVOP, SVOPL, SWAP70, SWI5, SWSAP1, SWT1, SYAP1, SYBU, SYCE1, SYCE1L, SYCE2, SYCE3, SYCN, SYCP1, SYCP2, SYCP2L, SYCP3, SYDE1, SYDE2, SYF2, SYK, SYMPK, SYN1, SYN2, SYN3, SYNC, SYNCRIP, SYNDIG1, SYNDIG1L, SYNE1, SYNE2, SYNE3, SYNE4, SYNGAP1, SYNGR1, SYNGR2, SYNGR3, SYNGR4, SYNJ1, SYNJ2, SYNJ2BP, SYNJ2BP-COX16, SYNM, SYNPO, SYNPO2, SYNPO2L, SYNPR, SYNRG, SYP, SYPL1, SYPL2, SYS1, SYS1-DBNDD2, SYT1, SYT10, SYT11, SYT12, SYT13, SYT14, SYT15, SYT16, SYT17, SYT2, SYT3, SYT4, SYT5, SYT6, SYT7, SYT8, SYT9, SYTL1, SYTL2, SYTL3, SYTL4, SYTL5, SYVN1, SZRD1, SZT2, T, TAAR1, TAAR2, TAAR5, TAAR6, TAAR8, TAAR9, TAB1, TAB2, TAB3, TAC1, TAC3, TAC4, TACC1, TACC2, TACC3, TACO1, TACR1, TACR2, TACR3, TACSTD2, TADA1, TADA2A, TADA2B, TADA3, TAF1, TAF10, TAF11, TAF12, TAF13, TAF15, TAF1A, TAF1B, TAF1C, TAF1D, TAF1L, TAF2, TAF3, TAF4, TAF4B, TAF5, TAF5L, TAF6, TAF6L, TAF7, TAF7L, TAF8, TAF9, TAF9B, TAGAP, TAGLN, TAGLN2, TAGLN3, TAL1, TAL2, TALDO1, TAMM41, TANC1, TANC2, TANGO2, TANGO6, TANK, TAOK1, TAOK2, TAOK3, TAP1, TAP2, TAPBP, TAPBPL, TAPT1, TARBP1, TARBP2, TARDBP, TARM1, TARS, TARS2, TARSL2, TAS1R1, TAS1R2, TAS1R3, TAS2R1, TAS2R10, TAS2R13, TAS2R14, TAS2R16, TAS2R19, TAS2R20, TAS2R3, TAS2R30, TAS2R31, TAS2R38, TAS2R39, TAS2R4, TAS2R40, TAS2R41, TAS2R42, TAS2R43, TAS2R46, TAS2R5, TAS2R50, TAS2R60, TAS2R7, TAS2R8, TAS2R9, TASP1, TAT, TATDN1, TATDN2, TATDN3, TAX1BP1, TAX1BP3, TAZ, TBATA, TBC1D1, TBC1D10A, TBC1D10B, TBC1D10C, TBC1D12, TBC1D13, TBC1D14, TBC1D15, TBC1D16, TBC1D17, TBC1D19, TBC1D2, TBC1D20, TBC1D21, TBC1D22A, TBC1D22B, TBC1D23, TBC1D24, TBC1D25, TBC1D26, TBC1D28, TBC1D29, TBC1D2B, TBC1D3, TBC1D30, TBC1D31, TBC1D32, TBC1D3B, TBC1D3C, TBC1D3D, TBC1D3E, TBC1D3F, TBC1D3G, TBC1D3H, TBC1D3I, TBC1D3K, TBC1D3L, TBC1D4, TBC1D5, TBC1D7, TBC1D8, TBC1D8B, TBC1D9, TBC1D9B, TBCA, TBCB, TBCC, TBCCD1, TBCD, TBCE, TBCEL, TBCK, TBK1, TBKBP1, TBL1X, TBL1XR1, TBL1Y, TBL2, TBL3, TBP, TBPL1, TBPL2, TBR1, TBRG1, TBRG4, TBX1, TBX10, TBX15, TBX18, TBX19, TBX2, TBX20, TBX21, TBX22, TBX3, TBX4, TBX5, TBX6, TBXA2R, TBXAS1, TC2N, TCAF1, TCAF2, TCAIM, TCAP, TCEA1, TCEA2, TCEA3, TCEAL1, TCEAL2, TCEAL3, TCEAL4, TCEAL5, TCEAL6, TCEAL7, TCEAL8, TCEAL9, TCEANC, TCEANC2, TCERG1, TCERG1L, TCF12, TCF15, TCF19, TCF20, TCF21, TCF23, TCF24, TCF25, TCF3, TCF4, TCF7, TCF7L1, TCF7L2, TCFL5, TCHH, TCHHL1, TCHP, TCIRG1, TCL1A, TCL1B, TCN1, TCN2, TCOF1, TCP1, TCP10, TCP10L, TCP10L2, TCP11, TCP11L1, TCP11L2, TCP11X2, TCTA, TCTE1, TCTE3, TCTEX1D1, TCTEX1D2, TCTEX1D4, TCTN1, TCTN2, TCTN3, TDG, TDGF1, TDO2, TDP1, TDP2, TDRD1, TDRD10, TDRD12, TDRD15, TDRD3, TDRD5, TDRD6, TDRD7, TDRD9, TDRKH, TDRP, TEAD1, TEAD2, TEAD3, TEAD4, TEC, TECPR1, TECPR2, TECR, TECRL, TECTA, TECTB, TEDDM1, TEF, TEFM, TEK, TEKT1, TEKT2, TEKT3, TEKT4, TEKT5, TELO2, TEN1, TEN1-CDK3, TENM1, TENM2, TENM3, TENM4, TEP1, TEPP, TEPSIN, TERB1, TERB2, TERF1, TERF2, TERF2IP, TERT, TES, TESC, TESK1, TESK2, TESMIN, TESPA1, TET1, TET2, TET3, TEX10, TEX101, TEX11, TEX12, TEX13A, TEX13B, TEX13C, TEX13D, TEX14, TEX15, TEX19, TEX2, TEX22, TEX26, TEX261, TEX264, TEX28, TEX29, TEX30, TEX33, TEX35, TEX36, TEX37, TEX38, TEX43, TEX44, TEX45, TEX46, TEX47, TEX48, TEX49, TEX50, TEX51, TEX9, TF, TFAM, TFAP2A, TFAP2B, TFAP2C, TFAP2D, TFAP2E, TFAP4, TFB1M, TFB2M, TFCP2, TFCP2L1, TFDP1, TFDP2, TFDP3, TFE3, TFEB, TFEC, TFF1, TFF2, TFF3, TFG, TFIP11, TFPI, TFPI2, TFPT, TFR2, TFRC, TG, TGDS, TGFA, TGFB1, TGFB1I1, TGFB2, TGFB3, TGFBI, TGFBR1, TGFBR2, TGFBR3, TGFBR3L, TGFBRAP1, TGIF1, TGIF2, TGIF2-C20orf24, TGIF2LX, TGIF2LY, TGM1, TGM2, TGM3, TGM4, TGM5, TGM6, TGM7, TGOLN2, TGS1, TH, THADA, THAP1, THAP10, THAP11, THAP12, THAP2, THAP3, THAP4, THAP5, THAP6, THAP7, THAP8, THAP9, THBD, THBS1, THBS2, THBS3, THBS4, THEG, THEGL, THEM4, THEM5, THEM6, THEMIS, THEMIS2, THG1L, THNSL1, THNSL2, THOC1, THOC2, THOC3, THOC5, THOC6, THOC7, THOP1, THPO, THRA, THRAP3, THRB, THRSP, THSD1, THSD4, THSD7A, THSD7B, THTPA, THUMPD1, THUMPD2, THUMPD3, THY1, THYN1, TIA1, TIAF1, TIAL1, TIAM1, TIAM2, TICAM1, TICAM2, TICRR, TIE1, TIFA, TIFAB, TIGAR, TIGD1, TIGD2, TIGD3, TIGD4, TIGD5, TIGD6, TIGD7, TIGIT, TIMD4, TIMELESS, TIMM10, TIMM10B, TIMM13, TIMM17A, TIMM17B, TIMM21, TIMM22, TIMM23, TIMM23B, TIMM29, TIMM44, TIMM50, TIMM8A, TIMM8B, TIMM9, TIMMDC1, TIMP1, TIMP2, TIMP3, TIMP4, TINAG, TINAGL1, TINCR, TINF2, TIPARP, TIPIN, TIPRL, TIRAP, TISP43, TJAP1, TJP1, TJP2, TJP3, TK1, TK2, TKFC, TKT, TKTL1, TKTL2, TLCD1, TLCD2, TLDC1, TLDC2, TLE1, TLE2, TLE3, TLE4, TLE6, TLK1, TLK2, TLL1, TLL2, TLN1, TLN2, TLNRD1, TLR1, TLR10, TLR2, TLR3, TLR4, TLR5, TLR6, TLR7, TLR8, TLR9, TLX1, TLX2, TLX3, TM2D1, TM2D2, TM2D3, TM4SF1, TM4SF18, TM4SF19, TM4SF19-TCTEX1D2, TM4SF20, TM4SF4, TM4SF5, TM6SF1, TM6SF2, TM7SF2, TM7SF3, TM9SF1, TM9SF2, TM9SF3, TM9SF4, TMA16, TMA7, TMBIM1, TMBIM4, TMBIM6, TMC1, TMC2, TMC3, TMC4, TMC5, TMC6, TMC7, TMC8, TMCC1, TMCC2, TMCC3, TMCO1, TMCO2, TMCO3, TMCO4, TMCO5A, TMCO6, TMED1, TMED10, TMED2, TMED3, TMED4, TMED5, TMED6, TMED7, TMED7-TICAM2, TMED8, TMED9, TMEFF1, TMEFF2, TMEM100, TMEM101, TMEM102, TMEM104, TMEM105, TMEM106A, TMEM106B, TMEM106C, TMEM107, TMEM108, TMEM109, TMEM11, TMEM110, TMEM110-MUSTN1, TMEM114, TMEM115, TMEM116, TMEM117, TMEM119, TMEM120A, TMEM120B, TMEM121, TMEM121B, TMEM123, TMEM125, TMEM126A, TMEM126B, TMEM127, TMEM128, TMEM129, TMEM130, TMEM131, TMEM131L, TMEM132A, TMEM132B, TMEM132C, TMEM132D, TMEM132E, TMEM133, TMEM134, TMEM135, TMEM136, TMEM138, TMEM139, TMEM140, TMEM141, TMEM143, TMEM144, TMEM145, TMEM147, TMEM14A, TMEM14B, TMEM14C, TMEM150A, TMEM150B, TMEM150C, TMEM151A, TMEM151B, TMEM154, TMEM155, TMEM156, TMEM158, TMEM159, TMEM160, TMEM161A, TMEM161B, TMEM163, TMEM164, TMEM165, TMEM167A, TMEM167B, TMEM168, TMEM169, TMEM17, TMEM170A, TMEM170B, TMEM171, TMEM173, TMEM174, TMEM175, TMEM176A, TMEM176B, TMEM177, TMEM178A, TMEM178B, TMEM179, TMEM179B, TMEM18, TMEM181, TMEM182, TMEM183A, TMEM184A, TMEM184B, TMEM184C, TMEM185A, TMEM185B, TMEM186, TMEM187, TMEM189, TMEM189-UBE2V1, TMEM19, TMEM190, TMEM191B, TMEM191C, TMEM192, TMEM196, TMEM198, TMEM199, TMEM2, TMEM200A, TMEM200B, TMEM200C, TMEM201, TMEM202, TMEM203, TMEM204, TMEM205, TMEM206, TMEM207, TMEM208, TMEM209, TMEM210, TMEM211, TMEM212, TMEM213, TMEM214, TMEM215, TMEM216, TMEM217, TMEM218, TMEM219, TMEM220, TMEM221, TMEM222, TMEM223, TMEM225, TMEM225B, TMEM229A, TMEM229B, TMEM230, TMEM231, TMEM232, TMEM233, TMEM234, TMEM235, TMEM236, TMEM237, TMEM238, TMEM239, TMEM240, TMEM241, TMEM242, TMEM243, TMEM244, TMEM245, TMEM246, TMEM247, TMEM248, TMEM249, TMEM25, TMEM250, TMEM251, TMEM252, TMEM253, TMEM254, TMEM255A, TMEM255B, TMEM256, TMEM256-PLSCR3, TMEM257, TMEM258, TMEM259, TMEM26, TMEM260, TMEM262, TMEM263, TMEM265, TMEM266, TMEM267, TMEM268, TMEM269, TMEM27, TMEM270, TMEM30A, TMEM30B, TMEM31, TMEM33, TMEM35A, TMEM35B, TMEM37, TMEM38A, TMEM38B, TMEM39A, TMEM39B, TMEM40, TMEM41A, TMEM41B, TMEM42, TMEM43, TMEM44, TMEM45A, TMEM45B, TMEM47, TMEM5, TMEM50A, TMEM50B, TMEM51, TMEM52, TMEM52B, TMEM53, TMEM54, TMEM55A, TMEM55B, TMEM56, TMEM56-RWDD3, TMEM57, TMEM59, TMEM59L, TMEM60, TMEM61, TMEM62, TMEM63A, TMEM63B, TMEM63C, TMEM64, TMEM65, TMEM67, TMEM68, TMEM69, TMEM70, TMEM71, TMEM72, TMEM74, TMEM74B, TMEM78, TMEM79, TMEM80, TMEM81, TMEM82, TMEM86A, TMEM86B, TMEM87A, TMEM87B, TMEM88, TMEM88B, TMEM89, TMEM8A, TMEM8B, TMEM9, TMEM91, TMEM92, TMEM94, TMEM95, TMEM97, TMEM98, TMEM99, TMEM9B, TMF1, TMIE, TMIGD1, TMIGD2, TMIGD3, TMLHE, TMOD1, TMOD2, TMOD3, TMOD4, TMPO, TMPPE, TMPRSS11A, TMPRSS11B, TMPRSS11D, TMPRSS11E, TMPRSS11F, TMPRSS12, TMPRSS13, TMPRSS15, TMPRSS2, TMPRSS3, TMPRSS4, TMPRSS4-AS1, TMPRSS5, TMPRSS6, TMPRSS7, TMPRSS9, TMSB10, TMSB15A, TMSB15B, TMSB4X, TMSB4Y, TMTC1, TMTC2, TMTC3, TMTC4, TMUB1, TMUB2, TMX1, TMX2, TMX2-CTNND1, TMX3, TMX4, TNC, TNF, TNFAIP1, TNFAIP2, TNFAIP3, TNFAIP6, TNFAIP8, TNFAIP8L1, TNFAIP8L2, TNFAIP8L3, TNFRSF10A, TNFRSF10B, TNFRSF10C, TNFRSF10D, TNFRSF11A, TNFRSF11B, TNFRSF12A, TNFRSF13B, TNFRSF13C, TNFRSF14, TNFRSF17, TNFRSF18, TNFRSF19, TNFRSF1A, TNFRSF1B, TNFRSF21, TNFRSF25, TNFRSF4, TNFRSF6B, TNFRSF8, TNFRSF9, TNFSF10, TNFSF11, TNFSF12, TNFSF12-TNFSF13, TNFSF13, TNFSF13B, TNFSF14, TNFSF15, TNFSF18, TNFSF4, TNFSF8, TNFSF9, TNIK, TNIP1, TNIP2, TNIP3, TNK1, TNK2, TNKS, TNKS1BP1, TNKS2, TNMD, TNN, TNNC1, TNNC2, TNNI1, TNNI2, TNNI3, TNNI3K, TNNT1, TNNT2, TNNT3, TNP1, TNP2, TNPO1, TNPO2, TNPO3, TNR, TNRC18, TNRC6A, TNRC6B, TNRC6C, TNS1, TNS2, TNS3, TNS4, TNXB, TOB1, TOB2, TOE1, TOGARAM1, TOGARAM2, TOLLIP, TOM1, TOM1L1, TOM1L2, TOMM20, TOMM20L, TOMM22, TOMM34, TOMM40, TOMM40L, TOMM5, TOMM6, TOMM7, TOMM70, TONSL, TOP1, TOP1MT, TOP2A, TOP2B, TOP3A, TOP3B, TOPAZ1, TOPBP1, TOPORS, TOR1A, TOR1AIP1, TOR1AIP2, TOR1B, TOR2A, TOR3A, TOR4A, TOX, TOX2, TOX3, TOX4, TP53, TP53AIP1, TP53BP1, TP53BP2, TP53I11, TP53I13, TP53I3, TP53INP1, TP53INP2, TP53RK, TP53TG3, TP53TG3B, TP53TG3C, TP53TG3D, TP53TG3E, TP53TG3F, TP53TG5, TP63, TP73, TPBG, TPBGL, TPCN1, TPCN2, TPD52, TPD52L1, TPD52L2, TPD52L3, TPGS1, TPGS2, TPH1, TPH2, TPI1, TPK1, TPM1, TPM2, TPM3, TPM4, TPMT, TPO, TPP1, TPP2, TPPP, TPPP2, TPPP3, TPR, TPRA1, TPRG1, TPRG1L, TPRKB, TPRN, TPRX1, TPSAB1, TPSB2, TPSD1, TPSG1, TPST1, TPST2, TPT1, TPTE, TPTE2, TPX2, TRA2A, TRA2B, TRABD, TRABD2A, TRABD2B, TRAC, TRADD, TRAF1, TRAF2, TRAF3, TRAF3IP1, TRAF3IP2, TRAF3IP3, TRAF4, TRAF5, TRAF6, TRAF7, TRAFD1, TRAIP, TRAJ1, TRAJ10, TRAJ11, TRAJ12, TRAJ13, TRAJ14, TRAJ16, TRAJ17, TRAJ18, TRAJ19, TRAJ2, TRAJ20, TRAJ21, TRAJ22, TRAJ23, TRAJ24, TRAJ25, TRAJ26, TRAJ27, TRAJ28, TRAJ29, TRAJ3, TRAJ30, TRAJ31, TRAJ32, TRAJ33, TRAJ34, TRAJ35, TRAJ36, TRAJ37, TRAJ38, TRAJ39, TRAJ4, TRAJ40, TRAJ41, TRAJ42, TRAJ43, TRAJ44, TRAJ45, TRAJ46, TRAJ47, TRAJ48, TRAJ49, TRAJ5, TRAJ50, TRAJ52, TRAJ53, TRAJ54, TRAJ56, TRAJ57, TRAJ58, TRAJ59, TRAJ6, TRAJ61, TRAJ7, TRAJ9, TRAK1, TRAK2, TRAM1, TRAM1L1, TRAM2, TRANK1, TRAP1, TRAPPC1, TRAPPC10, TRAPPC11, TRAPPC12, TRAPPC13, TRAPPC2, TRAPPC2L, TRAPPC3, TRAPPC3L, TRAPPC4, TRAPPC5, TRAPPC6A, TRAPPC6B, TRAPPC8, TRAPPC9, TRAT1, TRAV10, TRAV1-1, TRAV1-2, TRAV12-1, TRAV12-2, TRAV12-3, TRAV13-1, TRAV13-2, TRAV14DV4, TRAV16, TRAV17, TRAV18, TRAV19, TRAV2, TRAV20, TRAV21, TRAV22, TRAV23DV6, TRAV24, TRAV25, TRAV26-1, TRAV26-2, TRAV27, TRAV29DV5, TRAV3, TRAV30, TRAV34, TRAV36DV7, TRAV38-1, TRAV38-2DV8, TRAV39, TRAV4, TRAV40, TRAV41, TRAV5, TRAV6, TRAV7, TRAV8-1, TRAV8-2, TRAV8-3, TRAV8-4, TRAV8-6, TRAV8-7, TRAV9-1, TRAV9-2, TRBC2, TRBJ2-1, TRBJ2-2, TRBJ2-2P, TRBJ2-3, TRBJ2-4, TRBJ2-5, TRBJ2-6, TRBJ2-7, TRBV10-1, TRBV10-2, TRBV10-3, TRBV11-1, TRBV19, TRBV2, TRBV20-1, TRBV200R9-2, TRBV21OR9-2, TRBV23-1, TRBV230R9-2, TRBV24-1, TRBV25-1, TRBV27, TRBV28, TRBV29-1, TRBV30, TRBV3-1, TRBV4-1, TRBV4-2, TRBV5-1, TRBV5-3, TRBV5-4, TRBV5-5, TRBV5-6, TRBV5-7, TRBV6-1, TRBV6-4, TRBV6-5, TRBV6-6, TRBV6-7, TRBV6-8, TRBV7-1, TRBV7-3, TRBV7-4, TRBV7-6, TRBV7-7, TRBV7-9, TRBV9, TRDC, TRDD1, TRDD2, TRDD3, TRDJ1, TRDJ2, TRDJ3, TRDJ4, TRDMT1, TRDN, TRDV1, TRDV2, TRDV3, TREH, TREM1, TREM2, TREML1, TREML2, TREML4, TRERF1, TREX1, TREX2, TRGC1, TRGC2, TRGJ1, TRGJ2, TRGJP, TRGJP1, TRGJP2, TRGV1, TRGV10, TRGV11, TRGV2, TRGV3, TRGV4, TRGV5, TRGV8, TRGV9, TRH, TRHDE, TRHR, TRIAP1, TRIB1, TRIB2, TRIB3, TRIL, TRIM10, TRIM11, TRIM13, TRIM14, TRIM15, TRIM16, TRIM16L, TRIM17, TRIM2, TRIM21, TRIM22, TRIM23, TRIM24, TRIM25, TRIM26, TRIM27, TRIM28, TRIM29, TRIM3, TRIM31, TRIM32, TRIM33, TRIM34, TRIM35, TRIM36, TRIM37, TRIM38, TRIM39, TRIM39-RPP21, TRIM4, TRIM40, TRIM41, TRIM42, TRIM43, TRIM43B, TRIM44, TRIM45, TRIM46, TRIM47, TRIM48, TRIM49, TRIM49B, TRIM49C, TRIM49D1, TRIM49D2, TRIM5, TRIM50, TRIM51, TRIM52, TRIM54, TRIM55, TRIM56, TRIM58, TRIM59, TRTM6, TRIM60, TRIM61, TRIM62, TRIM63, TRIM64, TRIM64B, TRIM64C, TRIM65, TRIM66, TRIM67, TRIM68, TRIM69, TRIM6-TRIM34, TRIM7, TRIM71, TRIM72, TRIM73, TRIM74, TRIM75P, TRIM77, TRIM8, TRIM9, TRIML1, TRIML2, TRIO, TRIOBP, TRIP10, TRIP11, TRIP12, TRIP13, TRIP4, TRIP6, TRIQK, TRIR, TRIT1, TRMO, TRMT1, TRMT10A, TRMT10B, TRMT10C, TRMT11, TRMT112, TRMT12, TRMT13, TRMT1L, TRMT2A, TRMT2B, TRMT44, TRMT5, TRMT6, TRMT61A, TRMT61B, TRMU, TRNAU1AP, TRNP1, TRNT1, TRO, TROAP, TROVE2, TRPA1, TRPC1, TRPC3, TRPC4, TRPC4AP, TRPC5, TRPC50S, TRPC6, TRPC7, TRPM1, TRPM2, TRPM3, TRPM4, TRPM5, TRPM6, TRPM7, TRPM8, TRPS1, TRPT1, TRPV1, TRPV2, TRPV3, TRPV4, TRPV5, TRPV6, TRRAP, TRUB1, TRUB2, TSACC, TSC1, TSC2, TSC22D1, TSC22D2, TSC22D3, TSC22D4, TSEN15, TSEN2, TSEN34, TSEN54, TSFM, TSG101, TSGA10, TSGA10IP, TSGA13, TSHB, TSHR, TSHZ1, TSHZ2, TSHZ3, TSKS, TSKU, TSLP, TSN, TSNARE1, TSNAX, TSNAX-DISC1, TSNAXIP1, TSPAN1, TSPAN10, TSPAN11, TSPAN12, TSPAN13, TSPAN14, TSPAN15, TSPAN16, TSPAN17, TSPAN18, TSPAN19, TSPAN2, TSPAN3, TSPAN31, TSPAN32, TSPAN33, TSPAN4, TSPAN5, TSPAN6, TSPAN7, TSPAN8, TSPAN9, TSPEAR, TSPO, TSPO2, TSPOAP1, TSPY1, TSPY10, TSPY2, TSPY3, TSPY4, TSPY8, TSPYL1, TSPYL2, TSPYL4, TSPYL5, TSPYL6, TSR1, TSR2, TSR3, TSSC4, TSSK1B, TSSK2, TSSK3, TSSK4, TSSK6, TST, TSTA3, TSTD1, TSTD2, TSTD3, TTBK1, TTBK2, TTC1, TTC12, TTC13, TTC14, TTC16, TTC17, TTC19, TTC21A, TTC21B, TTC22, TTC23, TTC23L, TTC24, TTC25, TTC26, TTC27, TTC28, TTC29, TTC3, TTC30A, TTC30B, TTC31, TTC32, TTC33, TTC34, TTC36, TTC37, TTC38, TTC39A, TTC39B, TTC39C, TTC4, TTC5, TTC6, TTC7A, TTC7B, TTC8, TTC9, TTC9B, TTC9C, TTF1, TTF2, TTI1, TTI2, TTK, TTL, TTLL1, TTLL10, TTLL11, TTLL12, TTLL13P, TTLL2, TTLL3, TTLL4, TTLL5, TTLL6, TTLL7, TTLL8, TTLL9, TTN, TTPA, TTPAL, TTR, TTYH1, TTYH2, TTYH3, TUB, TUBA1A, TUBA1B, TUBA1C, TUBA3C, TUBA3D, TUBA3E, TUBA4A, TUBA4B, TUBA8, TUBAL3, TUBB, TUBB1, TUBB2A, TUBB2B, TUBB3, TUBB4A, TUBB4B, TUBB6, TUBB8, TUBD1, TUBE1, TUBG1, TUBG2, TUBGCP2, TUBGCP3, TUBGCP4, TUBGCP5, TUBGCP6, TUFM, TUFT1, TULP1, TULP2, TULP3, TULP4, TUNAR, TUSC1, TUSC2, TUSC3, TUSC5, TUT1, TVP23A, TVP23B, TVP23C, TVP23C-CDRT4, TWF1, TWF2, TWIST1, TWIST2, TWISTNB, TWNK, TWSG1, TXK, TXLNA, TXLNB, TXLNG, TXN, TXN2, TXNDC11, TXNDC12, TXNDC15, TXNDC16, TXNDC17, TXNDC2, TXNDC5, TXNDC8, TXNDC9, TXNIP, TXNL1, TXNL4A, TXNL4B, TXNRD1, TXNRD2, TXNRD3, TXNRD3NB, TYK2, TYMP, TYMS, TYR, TYRO3, TYROBP, TYRP1, TYSND1, TYW1, TYW1B, TYW3, TYW5, U2AF1, U2AF1L4, U2AF1L5, U2AF2, U2SURP, UACA, UAP1, UAP1L1, UBA1, UBA2, UBA3, UBA5, UBA52, UBA6, UBA7, UBAC1, UBAC2, UBALD1, UBALD2, UBAP1, UBAP1L, UBAP2, UBAP2L, UBASH3A, UBASH3B, UBB, UBC, UBD, UBE2A, UBE2B, UBE2C, UBE2D1, UBE2D2, UBE2D3, UBE2D4, UBE2E1, UBE2E2, UBE2E3, UBE2F, UBE2F-SCLY, UBE2G1, UBE2G2, UBE2H, UBE2I, UBE2J1, UBE2J2, UBE2K, UBE2L3, UBE2L5P, UBE2L6, UBE2M, UBE2N, UBE2NL, UBE20, UBE2Q1, UBE2Q2, UBE2Q2L, UBE2QL1, UBE2R2, UBE2S, UBE2T, UBE2U, UBE2V1, UBE2V2, UBE2W, UBE2Z, UBE3A, UBE3B, UBE3C, UBE3D, UBE4A, UBE4B, UBFD1, UBIAD1, UBL3, UBL4A, UBL4B, UBL5, UBL7, UBLCP1, UBN1, UBN2, UBOX5, UBP1, UBQLN1, UBQLN2, UBQLN3, UBQLN4, UBQLNL, UBR1, UBR2, UBR3, UBR4, UBR5, UBR7, UBTD1, UBTD2, UBTF, UBTFL1, UBXN1, UBXN10, UBXN11, UBXN2A, UBXN2B, UBXN4, UBXN6, UBXN7, UBXN8, UCHL1, UCHL3, UCHL5, UCK1, UCK2, UCKL1, UCMA, UCN, UCN2, UCN3, UCP1, UCP2, UCP3, UEVLD, UFC1, UFD1, UFL1, UFM1, UFSP1, UFSP2, UGCG, UGDH, UGGT1, UGGT2, UGP2, UGT1A1, UGT1A10, UGT1A3, UGT1A4, UGT1A5, UGT1A6, UGT1A7, UGT1A8, UGT1A9, UGT2A1, UGT2A2, UGT2A3, UGT2B10, UGT2B11, UGT2B15, UGT2B17, UGT2B28, UGT2B4, UGT2B7, UGT3A1, UGT3A2, UGT8, UHMK1, UHRF1, UHRF1BP1, UHRF1BP1L, UHRF2, UIMC1, ULBP1, ULBP2, ULBP3, ULK1, ULK2, ULK3, ULK4, UMAD1, UMOD, UMODL1, UMPS, UNC119, UNC119B, UNC13A, UNC13B, UNC13C, UNC13D, UNC45A, UNC45B, UNC50, UNC5A, UNC5B, UNC5C, UNC5CL, UNC5D, UNC79, UNC80, UNC93A, UNC93B1, UNCX, UNG, UNK, UNKL, UPB1, UPF1, UPF2, UPF3A, UPF3B, UPK1A, UPK1B, UPK2, UPK3A, UPK3B, UPK3BL1, UPP1, UPP2, UPRT, UQCC1, UQCC2, UQCC3, UQCR10, UQCR11, UQCRB, UQCRC1, UQCRC2, UQCRF S1, UQCRH, UQCRHL, UQCRQ, URAD, URB1, URB2, URGCP, URGCP-MRPS24, URI1, URM1, UROC1, UROD, UROS, USB1, USE1, USF1, USF2, USF3, USH1C, USH1G, USH2A, USHBP1, USMG5, USO1, USP1, USP10, USP11, USP12, USP13, USP14, USP15, USP16, USP17L1, USP17L10, USP17L11, USP17L12, USP17L13, USP17L15, USP17L17, USP17L18, USP17L19, USP17L2, USP17L20, USP17L21, USP17L22, USP17L23, USP17L24, USP17L25, USP17L26, USP17L27, USP17L28, USP17L29, USP17L3, USP17L30, USP17L4, USP17L5, USP17L7, USP17L8, USP18, USP19, USP2, USP20, USP21, USP22, USP24, USP25, USP26, USP27X, USP28, USP29, USP3, USP30, USP31, USP32, USP33, USP34, USP35, USP36, USP37, USP38, USP39, USP4, USP40, USP41, USP42, USP43, USP44, USP45, USP46, USP47, USP48, USP49, USP5, USP50, USP51, USP53, USP54, USP6, USP6NL, USP7, USP8, USP9X, USP9Y, USPL1, UST, UTF1, UTP11, UTP14A, UTP14C, UTP15, UTP18, UTP20, UTP23, UTP3, UTP4, UTP6, UTRN, UTS2, UTS2B, UTS2R, UTY, UVRAG, UVSSA, UXS1, UXT, VAC14, VAMP1, VAMP2, VAMP3, VAMP4, VAMP5, VAMP7, VAMP8, VANGL1, VANGL2, VAPA, VAPB, VARS, VARS2, VASH1, VASH2, VASN, VASP, VAT 1, VAT 1L, VAV1, VAV2, VAV3, VAX1, VAX2, VBP1, VCAM1, VCAN, VCL, VCP, VCPIP1, VCPKMT, VCX, VCX2, VCX3A, VCX3B, VCY, VCY1B, VDAC1, VDAC2, VDAC3, VDR, VEGFA, VEGFB, VEGFC, VEGFD, VENTX, VEPH1, VEZF1, VEZT, VGF, VGLL1, VGLL2, VGLL3, VGLL4, VHL, VHLL, VIL1, VILL, VIM, VIP, VIPAS39, VIPR1, VIPR2, VIRMA, VIT, VKORC1, VKORC1L1, VLDLR, VMA21, VMAC, VMO1, VMP1, VN1R1, VN1R2, VN1R4, VN1R5, VNN1, VNN2, VNN3, VOPP1, VPREB1, VPREB3, VPS11, VPS13A, VPS13B, VPS13C, VPS13D, VPS16, VPS18, VPS25, VPS26A, VPS26B, VPS28, VPS29, VPS33A, VPS33B, VPS35, VPS36, VPS37A, VPS37B, VPS37C, VPS37D, VPS39, VPS41, VPS45, VPS4A, VPS4B, VPS50, VPS51, VPS52, VPS53, VPS54, VPS72, VPS8, VPS9D1, VRK1, VRK2, VRK3, VRTN, VSIG1, VSIG10, VSIG10L, VSIG10L2, VSIG2, VSIG4, VSIG8, VSIR, VSNL1, VSTM1, VSTM2A, VSTM2B, VSTM2L, VSTM4, VSTM5, VSX1, VSX2, VTA1, VTCN1, VTI1A, VTI1B, VTN, VWA1, VWA2, VWA3A, VWA3B, VWA5A, VWA5B1, VWA5B2, VWA7, VWA8, VWC2, VWC2L, VWCE, VWDE, VWF, WAC, WAPL, WARS, WARS2, WAS, WASF1, WASF2, WASF3, WASHC1, WASHC2A, WASHC2C, WASHC3, WASHC4, WASHC5, WASL, WBP1, WBP11, WBP1L, WBP2, WBP2NL, WBP4, WDCP, WDFY1, WDFY2, WDFY3, WDFY4, WDHD1, WDPCP, WDR1, WDR11, WDR12, WDR13, WDR17, WDR18, WDR19, WDR20, WDR24, WDR25, WDR26, WDR27, WDR3, WDR31, WDR33, WDR34, WDR35, WDR36, WDR37, WDR38, WDR4, WDR41, WDR43, WDR44, WDR45, WDR45B, WDR46, WDR47, WDR48, WDR49, WDR5, WDR53, WDR54, WDR55, WDR59, WDR5B, WDR6, WDR60, WDR61, WDR62, WDR63, WDR64, WDR66, WDR7, WDR70, WDR72, WDR73, WDR74, WDR75, WDR76, WDR77, WDR78, WDR81, WDR82, WDR83, WDR83OS, WDR86, WDR87, WDR88, WDR89, WDR90, WDR91, WDR92, WDR93, WDR97, WDSUB1, WDTC1, WDYHV1, WEE1, WEE2, WFDC1, WFDC10A, WFDC10B, WFDC11, WFDC12, WFDC13, WFDC2, WFDC3, WFDC5, WFDC6, WFDC8, WFDC9, WFIKKN1, WFIKKN2, WFS1, WHAMM, WHRN, WIF1, WIPF1, WIPF2, WIPF3, WIPI1, WIPI2, WISP1, WISP2, WISP3, WIZ, WLS, WNK1, WNK2, WNK3, WNK4, WNT1, WNT10A, WNT10B, WNT11, WNT16, WNT2, WNT2B, WNT3, WNT3A, WNT4, WNT5A, WNT5B, WNT6, WNT7A, WNT7B, WNT8A, WNT8B, WNT9A, WNT9B, WRAP53, WRAP73, WRB, WRN, WRNIP1, WSB1, WSB2, WSCD1, WSCD2, WT1, WTAP, WTH3DI, WTIP, WWC1, WWC2, WWC3, WWOX, WWP1, WWP2, WWTR1, XAB2, XAF1, XAGE1A, XAGE1B, XAGE2, XAGE3, XAGE5, XBP1, XCL1, XCL2, XCR1, XDH, XG, XIAP, XIRP1, XIRP2, XK, XKR3, XKR4, XKR5, XKR6, XKR7, XKR8, XKR9, XKRX, XPA, XPC, XPNPEP1, XPNPEP2, XPNPEP3, XPO1, XPO4, XPO5, XPO6, XPO7, XPOT, XPR1, XRCC1, XRCC2, XRCC3, XRCC4, XRCC5, XRCC6, XRN1, XRN2, XRRA1, XXYLT1, XYLB, XYLT1, XYLT2, YAE1D1, YAF2, YAP1, YARS, YARS2, YBEY, YBX1, YBX2, YBX3, YDJC, YEATS2, YEATS4, YES1, YIF1A, YIF1B, YIPF1, YIPF2, YIPF3, YIPF4, YIPF5, YIPF6, YIPF7, YJEFN3, YKT6, YLPM1, YME1L1, YOD1, YPEL1, YPEL2, YPEL3, YPEL4, YPEL5, YRDC, YTHDC1, YTHDC2, YTHDF1, YTHDF2, YTHDF3, YWHAB, YWHAE, YWHAG, YWHAH, YWHAQ, YWHAZ, YY1, YY1AP1, YY2, Z82206.1, Z83844.1, Z84492.1, Z98749.3, Z98752.3, ZACN, ZADH2, ZAN, ZAP70, ZAR1, ZAR1L, ZBBX, ZBED1, ZBED2, ZBED3, ZBED4, ZBED5, ZBED6, ZBED6CL, ZBED8, ZBED9, ZBP1, ZBTB1, ZBTB10, ZBTB11, ZBTB12, ZBTB14, ZBTB16, ZBTB17, ZBTB18, ZBTB2, ZBTB20, ZBTB21, ZBTB22, ZBTB24, ZBTB25, ZBTB26, ZBTB3, ZBTB32, ZBTB33, ZBTB34, ZBTB37, ZBTB38, ZBTB39, ZBTB4, ZBTB40, ZBTB41, ZBTB42, ZBTB43, ZBTB44, ZBTB45, ZBTB46, ZBTB47, ZBTB48, ZBTB49, ZBTB5, ZBTB6, ZBTB7A, ZBTB7B, ZBTB7C, ZBTB8A, ZBTB8B, ZBTB8OS, ZBTB9, ZC2HC1A, ZC2HC1B, ZC2HC1C, ZC3H10, ZC3H11A, ZC3H11B, ZC3H12A, ZC3H12B, ZC3H12C, ZC3H12D, ZC3H13, ZC3H14, ZC3H15, ZC3H18, ZC3H3, ZC3H4, ZC3H6, ZC3H7A, ZC3H7B, ZC3H8, ZC3HAV1, ZC3HAV1L, ZC3HC1, ZC4H2, ZCCHC10, ZCCHC11, ZCCHC12, ZCCHC13, ZCCHC14, ZCCHC17, ZCCHC18, ZCCHC2, ZCCHC24, ZCCHC3, ZCCHC4, ZCCHC6, ZCCHC7, ZCCHC8, ZCCHC9, ZCRB1, ZCWPW1, ZCWPW2, ZDBF2, ZDHHC1, ZDHHC11, ZDHHC11B, ZDHHC12, ZDHHC13, ZDHHC14, ZDHHC15, ZDHHC16, ZDHHC17, ZDHHC18, ZDHHC19, ZDHHC2, ZDHHC20, ZDHHC21, ZDHHC22, ZDHHC23, ZDHHC24, ZDHHC3, ZDHHC4, ZDHHC5, ZDHHC6, ZDHHC7, ZDHHC8, ZDHHC9, ZEB1, ZEB2, ZER1, ZFAND1, ZFAND2A, ZFAND2B, ZFAND3, ZFAND4, ZFAND5, ZFAND6, ZFAT, ZFC3H1, ZFHX2, ZFHX3, ZFHX4, ZFP1, ZFP14, ZFP2, ZFP28, ZFP3, ZFP30, ZFP36, ZFP36L1, ZFP36L2, ZFP37, ZFP41, ZFP42, ZFP57, ZFP62, ZFP64, ZFP69, ZFP69B, ZFP82, ZFP90, ZFP91, ZFP91-CNTF, ZFP92, ZFPL1, ZFPM1, ZFPM2, ZFR, ZFR2, ZFX, ZFY, ZFYVE1, ZFYVE16, ZFYVE19, ZFYVE21, ZFYVE26, ZFYVE27, ZFYVE28, ZFYVE9, ZG16, ZG16B, ZGLP1, ZGPAT, ZGRF1, ZHX1, ZHX1-C8orf76, ZHX2, ZHX3, ZIC1, ZIC2, ZIC3, ZIC4, ZIC5, ZIK1, ZIM2, ZIM3, ZKSCAN1, ZKSCAN2, ZKSCAN3, ZKSCAN4, ZKSCAN5, ZKSCAN7, ZKSCAN8, ZMAT1, ZMAT2, ZMAT3, ZMAT4, ZMAT5, ZMIZ1, ZMIZ2, ZMPSTE24, ZMYM1, ZMYM2, ZMYM3, ZMYM4, ZMYM5, ZMYM6, ZMYND10, ZMYND11, ZMYND12, ZMYND15, ZMYND19, ZMYND8, ZNF10, ZNF100, ZNF101, ZNF106, ZNF107, ZNF112, ZNF114, ZNF117, ZNF12, ZNF121, ZNF124, ZNF131, ZNF132, ZNF133, ZNF134, ZNF135, ZNF136, ZNF138, ZNF14, ZNF140, ZNF141, ZNF142, ZNF143, ZNF146, ZNF148, ZNF154, ZNF155, ZNF157, ZNF16, ZNF160, ZNF165, ZNF169, ZNF17, ZNF174, ZNF175, ZNF177, ZNF18, ZNF180, ZNF181, ZNF182, ZNF184, ZNF185, ZNF189, ZNF19, ZNF195, ZNF197, ZNF2, ZNF20, ZNF200, ZNF202, ZNF205, ZNF207, ZNF208, ZNF211, ZNF212, ZNF213, ZNF214, ZNF215, ZNF217, ZNF219, ZNF22, ZNF221, ZNF222, ZNF223, ZNF224, ZNF225, ZNF226, ZNF227, ZNF229, ZNF23, ZNF230, ZNF232, ZNF233, ZNF234, ZNF235, ZNF236, ZNF239, ZNF24, ZNF248, ZNF25, ZNF250, ZNF251, ZNF253, ZNF254, ZNF256, ZNF257, ZNF26, ZNF260, ZNF263, ZNF264, ZNF266, ZNF267, ZNF268, ZNF273, ZNF274, ZNF275, ZNF276, ZNF277, ZNF28, ZNF280A, ZNF280B, ZNF280C, ZNF280D, ZNF281, ZNF282, ZNF283, ZNF284, ZNF285, ZNF286A, ZNF286B, ZNF287, ZNF292, ZNF296, ZNF3, ZNF30, ZNF300, ZNF302, ZNF304, ZNF311, ZNF316, ZNF317, ZNF318, ZNF319, ZNF32, ZNF320, ZNF322, ZNF324, ZNF324B, ZNF326, ZNF329, ZNF330, ZNF331, ZNF333, ZNF334, ZNF335, ZNF337, ZNF33A, ZNF33B, ZNF34, ZNF341, ZNF343, ZNF345, ZNF346, ZNF347, ZNF35, ZNF350, ZNF354A, ZNF354B, ZNF354C, ZNF358, ZNF362, ZNF365, ZNF366, ZNF367, ZNF37A, ZNF382, ZNF383, ZNF384, ZNF385A, ZNF385B, ZNF385C, ZNF385D, ZNF391, ZNF394, ZNF395, ZNF396, ZNF397, ZNF398, ZNF404, ZNF407, ZNF408, ZNF41, ZNF410, ZNF414, ZNF415, ZNF416, ZNF417, ZNF418, ZNF419, ZNF420, ZNF423, ZNF425, ZNF426, ZNF428, ZNF429, ZNF43, ZNF430, ZNF431, ZNF432, ZNF433, ZNF436, ZNF438, ZNF439, ZNF44, ZNF440, ZNF441, ZNF442, ZNF443, ZNF444, ZNF445, ZNF446, ZNF449, ZNF45, ZNF451, ZNF454, ZNF460, ZNF461, ZNF462, ZNF467, ZNF468, ZNF469, ZNF470, ZNF471, ZNF473, ZNF474, ZNF479, ZNF48, ZNF480, ZNF483, ZNF484, ZNF485, ZNF486, ZNF487, ZNF488, ZNF490, ZNF491, ZNF492, ZNF493, ZNF496, ZNF497, ZNF500, ZNF501, ZNF502, ZNF503, ZNF506, ZNF507, ZNF510, ZNF511, ZNF512, ZNF512B, ZNF513, ZNF514, ZNF516, ZNF517, ZNF518A, ZNF518B, ZNF519, ZNF521, ZNF524, ZNF525, ZNF526, ZNF527, ZNF528, ZNF529, ZNF530, ZNF532, ZNF534, ZNF536, ZNF540, ZNF541, ZNF543, ZNF544, ZNF546, ZNF547, ZNF548, ZNF549, ZNF550, ZNF551, ZNF552, ZNF554, ZNF555, ZNF556, ZNF557, ZNF558, ZNF559, ZNF559-ZNF177, ZNF560, ZNF561, ZNF562, ZNF563, ZNF564, ZNF565, ZNF566, ZNF567, ZNF568, ZNF569, ZNF57, ZNF570, ZNF571, ZNF572, ZNF573, ZNF574, ZNF575, ZNF576, ZNF577, ZNF578, ZNF579, ZNF580, ZNF581, ZNF582, ZNF583, ZNF584, ZNF585A, ZNF585B, ZNF586, ZNF587, ZNF587B, ZNF589, ZNF592, ZNF593, ZNF594, ZNF595, ZNF596, ZNF597, ZNF598, ZNF599, ZNF600, ZNF605, ZNF606, ZNF607, ZNF608, ZNF609, ZNF610, ZNF611, ZNF613, ZNF614, ZNF615, ZNF616, ZNF618, ZNF619, ZNF620, ZNF621, ZNF622, ZNF623, ZNF624, ZNF625, ZNF625-ZNF20, ZNF626, ZNF627, ZNF628, ZNF629, ZNF630, ZNF638, ZNF639, ZNF641, ZNF644, ZNF645, ZNF646, ZNF648, ZNF649, ZNF652, ZNF653, ZNF654, ZNF655, ZNF658, ZNF66, ZNF660, ZNF662, ZNF664, ZNF665, ZNF667, ZNF668, ZNF669, ZNF670, ZNF670-ZNF695, ZNF671, ZNF672, ZNF674, ZNF675, ZNF676, ZNF677, ZNF678, ZNF679, ZNF680, ZNF681, ZNF682, ZNF683, ZNF684, ZNF687, ZNF688, ZNF689, ZNF69, ZNF691, ZNF692, ZNF695, ZNF696, ZNF697, ZNF699, ZNF7, ZNF70, ZNF700, ZNF701, ZNF703, ZNF704, ZNF705A, ZNF705B, ZNF705D, ZNF705E, ZNF705G, ZNF706, ZNF707, ZNF708, ZNF709, ZNF71, ZNF710, ZNF711, ZNF713, ZNF714, ZNF716, ZNF717, ZNF718, ZNF720, ZNF721, ZNF724, ZNF726, ZNF727, ZNF728, ZNF729, ZNF730, ZNF732, ZNF735, ZNF736, ZNF737, ZNF738, ZNF74, ZNF740, ZNF746, ZNF747, ZNF749, ZNF750, ZNF75A, ZNF75D, ZNF76, ZNF761, ZNF763, ZNF764, ZNF765, ZNF766, ZNF768, ZNF77, ZNF770, ZNF771, ZNF772, ZNF773, ZNF774, ZNF775, ZNF776, ZNF777, ZNF778, ZNF780A, ZNF780B, ZNF781, ZNF782, ZNF783, ZNF784, ZNF785, ZNF786, ZNF787, ZNF788, ZNF789, ZNF79, ZNF790, ZNF791, ZNF792, ZNF793, ZNF799, ZNF8, ZNF80, ZNF800, ZNF804A, ZNF804B, ZNF805, ZNF808, ZNF81, ZNF813, ZNF814, ZNF816, ZNF816-ZNF321P, ZNF821, ZNF823, ZNF827, ZNF829, ZNF83, ZNF830, ZNF831, ZNF835, ZNF836, ZNF837, ZNF839, ZNF84, ZNF841, ZNF843, ZNF844, ZNF845, ZNF846, ZNF85, ZNF850, ZNF852, ZNF853, ZNF860, ZNF862, ZNF865, ZNF878, ZNF879, ZNF880, ZNF883, ZNF888, ZNF891, ZNF90, ZNF91, ZNF92, ZNF93, ZNF98, ZNF99, ZNFX1, ZNHIT1, ZNHIT2, ZNHIT3, ZNHIT6, ZNRD1, ZNRF1, ZNRF2, ZNRF3, ZNRF4, ZP1, ZP2, ZP3, ZP4, ZPBP, ZPBP2, ZPLD1, ZPR1, ZRANB1, ZRANB2, ZRANB3, ZRSR1, ZRSR2, ZSCAN1, ZSCAN10, ZSCAN12, ZSCAN16, ZSCAN18, ZSCAN2, ZSCAN20, ZSCAN21, ZSCAN22, ZSCAN23, ZSCAN25, ZSCAN26, ZSCAN29, ZSCAN30, ZSCAN31, ZSCAN32, ZSCAN4, ZSCAN5A, ZSCAN5B, ZSCAN5C, ZSCAN9, ZSWIM1, ZSWIM2, ZSWIM3, ZSWIM4, ZSWIM5, ZSWIM6, ZSWIM7, ZSWIM8, ZUFSP, ZW10, ZWILCH, ZWINT, ZXDA, ZXDB, ZXDC, ZYG11A, ZYG11B, ZYX, ZZEF1, and ZZZ3.

Furthermore, the invention provides the use of a compound according to the definitions herein, or a pharmaceutically acceptable salt, or a hydrate or solvate thereof for the preparation of a medicament for the treatment of an autoimmune disorder, an inflammatory disorder, or a proliferative disorder, or a disorder commonly occurring in connection with transplantation.

Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

In one embodiment, the present invention provides a composition comprising a provided compound and one or more additional therapeutic agents. The therapeutic agent may be administered together with a provided compound, or may be administered prior to or following administration of a provided compound. Suitable therapeutic agents are described in further detail below. In certain embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-Bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron®) in combination with lenalidomide (Revlimid®), or any combination(s) thereof.

In another embodiment, the present invention provides a method of treating rheumatoid arthritis comprising administering to a patient in need thereof a provided compound and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating systemic lupus erythematosus comprising administering to a patient in need thereof a provided compound and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating Crohn's disease, ulcerative colitis, or inflammatory bowel disease comprising administering to a patient in need thereof a provided compound and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-Bid®, Uniphyl®, Theo-24®) and aminophylline, and IgE antibodies such as omalizumab (Xolair®).

In some embodiments, the present invention provides a method of treating COPD comprising administering to a patient in need thereof a provided compound and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-Bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In another embodiment, the present invention provides a method of treating a hematological malignancy comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a solid tumor comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a PI3K inhibitor, a SYK inhibitor, and combinations thereof.

In another embodiment, the present invention provides a method of treating a hematological malignancy comprising administering to a patient in need thereof a provided compound and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In another embodiment, the present invention provides a method of treating multiple myeloma comprising administering to a patient in need thereof a provided compound and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating or lessening the severity of a disease comprising administering to a patient in need thereof a provided compound and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments the present invention provides a method of treating or lessening the severity of a disease comprising administering to a patient in need thereof a provided compound and a Bcl-2 inhibitor, wherein the disease is an inflammatory disorder, an autoimmune disorder, a proliferative disorder, an endocrine disorder, a neurological disorder, or a disorder associated with transplantation. In some embodiments, the disorder is a proliferative disorder, lupus, or lupus nephritis. In some embodiments, the proliferative disorder is chronic lymphocytic leukemia, diffuse large B-cell lymphoma, Hodgkin's disease, small-cell lung cancer, non-small-cell lung cancer, myelodysplastic syndrome, lymphoma, a hematological neoplasm, or solid tumor.

The compounds and compositions, according to the method of the present invention, may be administered using any amount and any route of administration effective for treating or lessening the severity of an autoimmune disorder, an inflammatory disorder, a proliferative disorder, an endocrine disorder, a neurological disorder, or a disorder associated with transplantation. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of modulating CRBN activity in a biological sample comprising the step of contacting said biological sample with a compound of this invention, or a composition comprising said compound.

According to another embodiment, the invention relates to a method of binding CRBN, or a mutant thereof, activity in a biological sample comprising the step of contacting said biological sample with a compound of this invention, or a composition comprising said compound.

The term “biological sample”, as used herein, includes, without limitation, cell cultures or extracts thereof; biopsied material obtained from a mammal or extracts thereof, and blood, saliva, urine, feces, semen, tears, or other body fluids or extracts thereof.

Binding CRBN (or a mutant thereof) activity in a biological sample is useful for a variety of purposes that are known to one of skill in the art. Examples of such purposes include, but are not limited to, biological specimen storage and biological assays.

Another embodiment of the present invention relates to a method of modulating CRBN activity in a patient comprising the step of administering to said patient a compound of the present invention, or a composition comprising said compound.

According to another embodiment, the invention relates to a method of modulating the activity of CRBN, or a mutant thereof, in a patient comprising the step of administering to said patient a compound of the present invention, or a composition comprising said compound. According to certain embodiments, the invention relates to a method of reversibly or irreversibly modulating one or more of CRBN, or a mutant thereof, activity in a patient comprising the step of administering to said patient a compound of the present invention, or a composition comprising said compound. In other embodiments, the present invention provides a method for treating a disorder mediated by CRBN, or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other therapeutic compounds. In some embodiments, the other therapeutic compounds are antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal©); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin. The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™ Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™ Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™) cetuximab (Erbitux™), Iressa, Tarceva, OSI-774, Cl-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof, see WO2008118802), navitoclax (and analogs thereof, see U.S. Pat. No. 7,390,799), NH-1 (Shenayng Pharmaceutical University), obatoclax (and analogs thereof, see WO2004106328), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

Further examples of BTK inhibitory compounds, and conditions treatable by such compounds in combination with compounds of this invention can be found in WO2008039218, U.S. Pat. No. 7,514,444, WO2011090760, and U.S. Pat. No. 8,338,439, the entirety of each of which is herein incorporated by reference.

Further examples of SYK inhibitory compounds, and conditions treatable by such compounds in combination with compounds of this invention can be found in WO2003063794, U.S. Pat. No. 7,557,210, WO2005007623, U.S. Pat. No. 7,173,015, WO2006078846, and U.S. Pat. No. 7,449,458, the entirety of each of which is herein incorporated by reference.

Further examples of PI3K inhibitory compounds, and conditions treatable by such compounds in combination with compounds of this invention can be found in WO2004019973, U.S. Pat. No. 7,713,943, WO2004089925, U.S. Pat. No. 6,949,537, WO2007016176, U.S. Pat. Nos. 7,402,325, 8,138,347, WO2002088112, U.S. Pat. No. 7,071,189, WO2007084786, U.S. Pat. No. 8,217,035, WO2007129161, U.S. Pat. No. 7,781,433, WO2006122806, U.S. Pat. No. 7,667,039, WO2005113554, U.S. Pat. No. 7,932,260, WO2007044729, and U.S. Pat. No. 7,989,622, the entirety of each of which is herein incorporated by reference.

Further examples of JAK inhibitory compounds, and conditions treatable by such compounds in combination with compounds of this invention can be found in WO2009114512, U.S. Pat. No. 8,185,616, WO2008109943, U.S. Pat. No. 8,486,941, WO2007053452, U.S. Pat. No. 7,528,143, WO200142246, U.S. Pat. No. 6,627,754, WO2007070514, and U.S. Pat. No. 7,598,257, the entirety of each of which is herein incorporated by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ-tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™) and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1β-O-D-arabinofuransylcytosine (ara-c) and bisulfan; ALK inhibitors, which are compounds which target, decrease or inhibit anaplastic lymphoma kinase, and Bcl-2 inhibitors.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan®), PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412. In some embodiments, the present invention provides a method of treating AML associated with an ITD and/or D835Y mutation, comprising administering a compound of the present invention together with a one or more FLT3 inhibitors. In some embodiments, the FLT3 inhibitors are selected from quizartinib (AC220), a staurosporine derivative (e.g. midostaurin or lestaurtinib), sorafenib, tandutinib, LY-2401401, LS-104, EB-10, famitinib, NOV-110302, NMS-P948, AST-487, G-749, SB-1317, S-209, SC-110219, AKN-028, fedratinib, tozasertib, and sunitinib. In some embodiments, the FLT3 inhibitors are selected from quizartinib, midostaurin, lestaurtinib, sorafenib, and sunitinib.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1, pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCR5, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

The compounds of this invention, or pharmaceutical compositions thereof, may also be incorporated into compositions for coating an implantable medical device, such as prostheses, artificial valves, vascular grafts, stents and catheters. Implantable devices coated with a compound of this invention are another embodiment of the present invention.

Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTOR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO 2011/070024, US 2011/0165156, WO 2011/0107553, US 2012/0329997, WO 2011/131407, US 2013/0005949, WO 2013/087699, US 2014/0336363, WO 2013/119716, WO 2013/132044, US 2014/0079706) or FPA-008 (WO 2011/140249, US 2011/0274683; WO 2013/169264; WO 2014/036357, US 2014/0079699).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgGI, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO 2010/077634, US 2010/0203056), durvalumab (MEDI4736), BMS-936559 (WO 2007/005874, US 2009/0055944), and MSB0010718C (WO 2013/079174, US 2014/0341917).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO 2010/019570, US 2010/0150892, WO 2014/008218, US 2014/0093511), or IMP-731 or IMP-321 (WO 2008/132601, US 2010/0233183, WO 2009/044273, US 2011/0008331).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO 2006/105021, US 2007/0098719, WO 2009/009116, US 2009/0136494), or MK-4166 (WO 2011/028683, US 2012/0189639).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS: F001287 (Bristol-Myers Squibb); Phy906/KD108 (Phytoceutica); an enzyme that breaks down kynurenine (Kynase, Kyn Therapeutics); and NLG-919 (WO 2009/073620, US 2011/0053941, WO 2009/132238, US 2011/0136796, WO 2011/056652, US 2012/0277217, WO 2012/142237, US 2014/0066625).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO 2006/029879, U.S. Pat. No. 7,501,496).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO 2011/109400, US 2013/0149236).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682, the entirety of each of which is herein incorporated by reference, which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15: sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams ET. AL., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams ET. AL. In some embodiments, an immuno-oncology agent is a small molecule targeting an immuno-oncology target selected from those listed in Table 2 of Jerry L. Adams ET. AL. In some embodiments, an immuno-oncology agent is a small molecule agent selected from those listed in Table 2 of Jerry L. Adams ET. AL.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018 28:319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-activated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof. In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an aspect, the checkpoint inhibitor is an immunostimulatory agent, a T cell growth factor, an interleukin, an antibody, a vaccine or a combination thereof. In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgGI anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgGI Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgGI, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMN/C-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

TABLE 2
Analytical instruments

| | |
|---|---|
| LCMS | Shimadzu UFLC MS: LCMS-2020 |
| | Agilent Technologies 1200 series MS: Agilent Technologies 6110 |
| | Agilent Technologies 1200 series MS: LC/MSD VL |
| NMR | BRUKER AVANCE III/400; Frequency (MHz) 400.13; Nucleus: 1H; |
| | Number of Transients: 8 |
| Prep-HPLC | Gilson GX-281 systems: instruments GX-A, GX-B, GX-C, GX-D, |
| | GX-E, GX-F, GX-G and GX-H |
| GCMS | SHIMADZU GCMS-QP20I0 Ultra |
| Analytical | Agilent Technologies 1290 Infinity |
| cSFC | |
| Prep-cSFC | Waters SFC Prep 80 |

For acidic LCMS data: LCMS was recorded on an Agilent 1200 Series LC/MSD or Shimadzu LCMS2020 equipped with electro-spray ionization and quadruple MS detector [ES+ve to give MH⁺] and equipped with Chromolith Flash RP-18e 25*2.0 mm, eluting with 0.0375 vol % TFA in water (solvent A) and 0.01875 vol % TFA in acetonitrile (solvent B). Other LCMS was recorded on an Agilent 1290 Infinity RRLC attached with Agilent 6120 Mass detector. The column used was BEH C18 50*2.1 mm, 1.7 micron. Column flow was 0.55 ml/min and mobile phase were used (A) 2 mM Ammonium Acetate in 0.1% Formic Acid in Water and (B) 0.1% Formic Acid in Acetonitrile.

For basic LCMS data: LCMS was recorded on an Agilent 1200 Series LC/MSD or Shimadzu LCMS 2020 equipped with electro-spray ionization and quadruple MS detector [ES+ve to give MH⁺] and equipped with Xbridge C18, 2.1×50 mm columns packed with 5 mm C18-coated silica or Kinetex EVO C18 2.1×30 mm columns packed with 5 mm C18-coated silica, eluting with 0.05 vol % NH₃.H₂O in water (solvent A) and acetonitrile (solvent B).

HPLC Analytical Method: HPLC was carried out on X Bridge C18 150*4.6 mm, 5 micron. Column flow was 1.0 ml/min and mobile phase were used (A) 0.1% Ammonia in water and (B) 0.1% Ammonia in Acetonitrile.

Prep HPLC Analytical Method: The compound was purified on Shimadzu LC-20AP and UV detector. The column used was X-BRIDGE C18 (250*19)mm, 5. Column flow was 16.0 ml/min. Mobile phase were used (A) 0.1% Formic Acid in Water and (B) Acetonitrile Basic method used (A) 5 mM ammonium bicarbonate and 0.1% NH3 in Water and (B) Acetonitrile or (A) 0.1% Ammonium Hydroxide in Water and (B) Acetonitrile. The UV spectra were recorded at 202 nm & 254 nm.

NMR Method: The 1H NMR spectra were recorded on a Bruker Ultra Shield Advance 400 MHz/5 mm Probe (BBFO). The chemical shifts are reported in part-per-million.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### Example 1. 8-(2,3-Dihydroindole-1-carbonyl)-2,4-dihydroisoquinoline-1,3-dione (I-1)

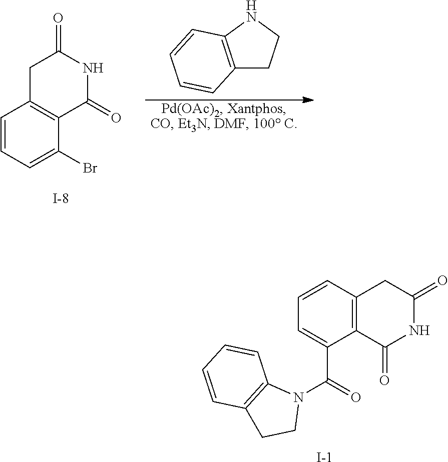

To a mixture of 8-bromo-2,4-dihydroisoquinoline-1,3-dione (240 mg, 1.00 mmol), indoline (239 mg, 2.01 mmol), Et₃N (304 mg, 3.00 mmol) and XantPhos (174 mg, 0.30 mmol) in DMF (10.0 mL) was added Pd(OAc)₂ (33.7 mg, 0.15 mmol) at room temperature under CO atmosphere. The above mixture was stirred at 100° C. under CO atmosphere for 16 h in a sealed tube. After being cooled down to room temperature. The resulting mixture was acidified to pH 5 with HCl (2.0 M). The resulting mixture was extracted with DCM (2×50.0 mL). The combined organic layers were washed with brine (2×30.0 mL), dried over anhydrous Na₂SO₄ and filtered. The filtrate was concentrated under reduced pressure. The residue was purified by Prep-HPLC with the following conditions (Column: XBridge Shield RP18 OBD Column, 30×150 mm, 5 um; Mobile Phase A: water (plus 0.05% TFA); Mobile Phase B: ACN; Flow rate: 60 mL/min; Gradient: 15% B to 45% B in 8 min; Detector: UV 220/254 nm). Desired fractions were collected at 6.15 min and lyophilized to afford the titled compound (50 mg) as a light yellow solid. ¹H NMR (400 MHz, DMSO-d₆) δ 11.34 (s, 1H), 8.16 (q, J=7.0 Hz, 1H), 7.96 (q, J=7.3 Hz, 1H), 7.74 (t, J=7.8 Hz, 1H), 7.51-7.36 (m, 1H), 7.32-7.17 (m, 2H), 7.06 (dt, J=10.4, 7.4 Hz, 1H), 4.28-3.94 (m, 2H), 3.72-3.48 (m, 2H), 3.18-2.95 (m, 2H). LC/MS (ESI, m/z): [(M+1)]⁺=307.10.

### Example 2. 2-(1,3-Dioxo-2,4-dihydroisoquinolin-8-yl)benzamide (I-2) and 2-(1,3-dioxo-2,4-dihydroisoquinolin-8-1)benzonitrile (I-52)

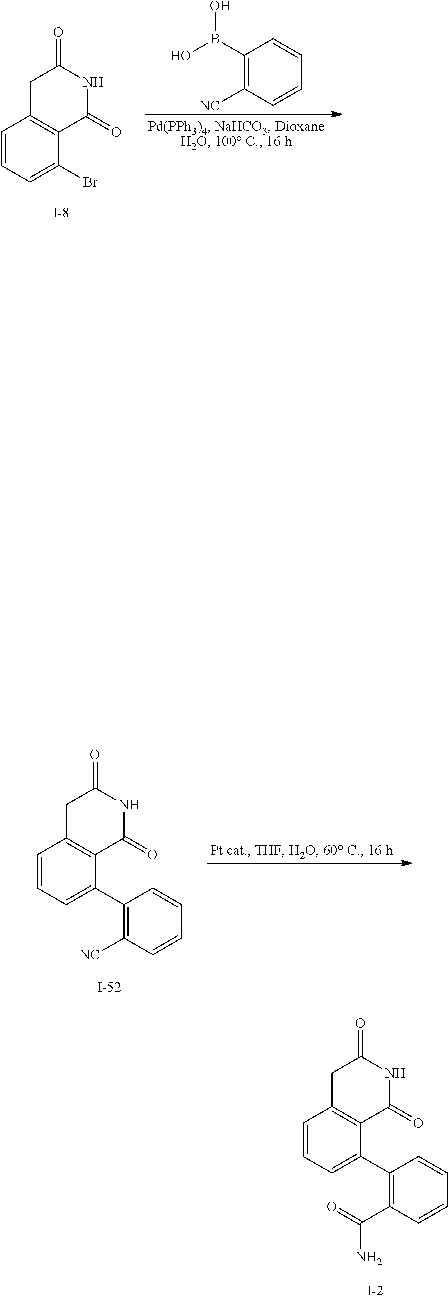

### Step 1: 2-(1,3-dioxo-2,4-dihydroisoquinolin-8-1)benzonitrile

To a solution of 8-bromo-2,4-dihydroisoquinoline-1,3-dione (300 mg, 1.25 mmol) in 1,4-dioxane (8.00 mL) were added H₂O (8.00 mL), 2-cyanophenylboronic acid (276 mg, 1.88 mmol), NaHCO₃ (1.05 g, 12.5 mmol) and Pd(PPh₃)₄ (145 mg, 0.13 mmol) at room temperature. The resulting mixture was purged with nitrogen 3 times and stirred for 16 h at 100° C. After cooling down to room temperature, the resulting mixture was concentrated under reduced pressure. The residue was purified by Prep-TLC (DCM:MeOH=10:1) to afford the titled compound (92.0 mg, 28%) as a light brown solid. ¹H NMR (400 MHz, DMSO-d₆) δ 11.19 (s, 1H), 7.85 (dd, J=7.7, 1.3 Hz, 1H), 7.77-7.68 (m, 2H), 7.57-7.51 (m, 2H), 7.39 (dd, J=7.9, 1.2 Hz, 1H), 7.25 (dd, J=7.6, 1.2 Hz, 1H), 4.36-4.00 (m, 2H). LC/MS (ESI, m/z): [(M+1)]⁺=263.10.

### Step 2: 2-(1,3-dioxo-2,4-dihydroisoquinolin-8-yl)benzamide

To a solution of 2-(1,3-dioxo-2,4-dihydroisoquinolin-8-yl)benzonitrile (50.0 mg, 0.19 mmol) in THF (2.00 mL) were added H₂O (2.00 mL) and Hydrido(dimethylphosphinous acid-kP)[hydrogen bis(dimethylphosphinito-kP)]platinum(II) (CAS: 173416-05-2) (8.14 mg, 0.019 mmol) at room temperature. The resulting mixture was stirred for 16 h at 60° C. After cooling down to room temperature, the resulting mixture was concentrated under reduced pressure and the residue was purified by Prep-HPLC with the following conditions: (Column: Xselect CSH OBD Column 30×150 mm 5 um; Mobile Phase A: water (plus 0.05% TFA); Mobile Phase B: ACN; Flow rate: 60 mL/min; Gradient: 3% B to 18% B in 7 min; Detector: UV 220/254 nm). Desired fractions were collected at 6.50 min and lyophilized to afford the titled compound (1.6 mg, 3%) as a white solid. ¹H NMR (400 MHz, DMSO-d₆) δ 10.98 (s, 1H), 7.62-7.49 (m, 2H), 7.47-7.31 (m, 3H), 7.26-7.16 (m, 1H), 7.12-6.96 (m, 3H), 4.08 (s, 2H). LC/MS (ESI, m/z): [(M−1)]⁻=279.10.

### Example 3. 8-(2,3-Dihydro-1,4-benzoxazine-4-carbonyl)-2,4-dihydroisoquinoline-1,3-dione (I-3)

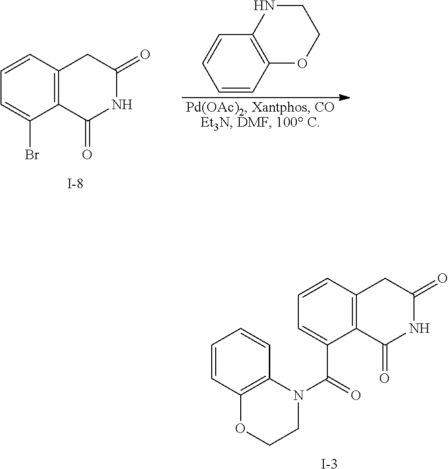

To a solution of 8-bromo-2,4-dihydroisoquinoline-1,3-dione (241 mg, 1.01 mmol) in DMF (10.0 mL) were added 3,4-dihydro-2H-1,4-benzoxazine (272 mg, 2.01 mmol), Et₃N (304 mg, 3.00 mmol), XantPhos (174 mg, 0.30 mmol) and Pd(OAc)₂ (33.7 mg, 0.15 mmol) at room temperature under CO atmosphere. The resulting mixture was stirred at 100° C. under CO atmosphere for 16 h. After cooling down to room temperature, the resulting mixture was acidified to pH 5 with HCl (2.0 M). The resulting mixture was extracted with DCM (2×50.0 mL). The combined organic layers were washed with brine (2×30.0 mL), dried over anhydrous Na₂SO₄ and filtered. The filtrate was concentrated under reduced pressure and the residue was purified by Prep-HPLC with the following conditions: (Column: XBridge Prep OBD C¹⁸ Column, 30×150 mm 5 um; Mobile Phase A: water (plus 0.1% FA); Mobile Phase B: ACN; Flow rate: 60 mL/min; Gradient: 20% B to 40% B in 8 min; Detector: UV 254/220 nm). Desired fractions were collected at 7.00 min and lyophilized to afford the titled compound (90 mg, 28%) as a pink solid. ¹H NMR (400 MHz, DMSO-d₆) δ 11.40 (s, 1H), 8.28 (dd, J=8.3, 1.6 Hz, 1H), 7.78-7.66 (m, 1H), 7.53-7.34 (m, 2H), 7.14-7.04 (m, 1H), 6.99-6.90 (m, 2H), 4.52-4.38 (m, 1H), 4.24 (ddd, J=10.8, 4.8, 3.0 Hz, 1H), 4.14 (dt, J=6.4, 3.2 Hz, 1H), 4.01 (d, J=7.3 Hz, 1H), 3.49-3.38 (m, 2H). LC/MS (ESI, m/z): [(M+1)]⁺=323.05.

### Example 4. 7-(2-Methylphenyl)-2,4-dihydroisoquinoline-1,3-dione (I-4)

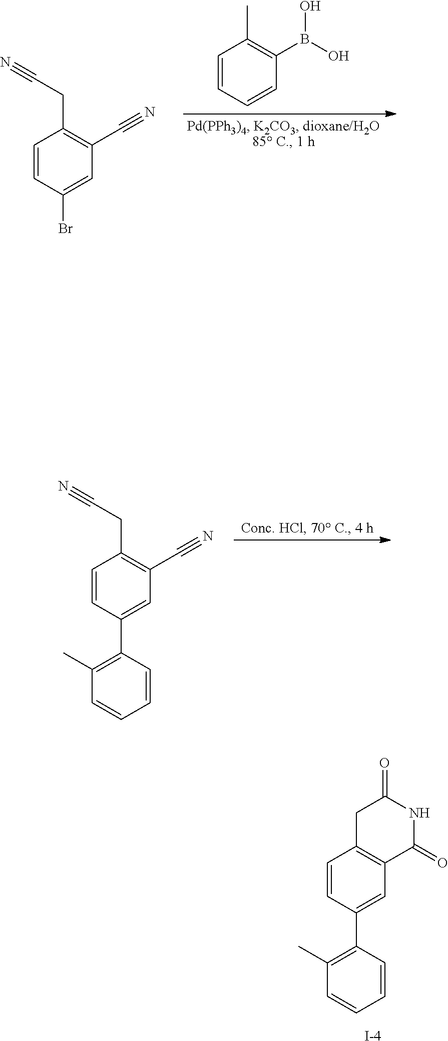

### Step 1: 4-(cyanomethyl)-2′-methyl-[1,1′-biphenyl]-3-carbonitrile

To a solution of 5-bromo-2-(cyanomethyl)benzonitrile (200 mg, 0.91 mmol) in dioxane (3.00 mL) were added H₂O (1.00 mL), 2-methylphenylboronic acid (185 mg, 1.36 mmol), K₂CO₃ (375 mg, 2.71 mmol) and Pd(PPh₃)₄ (105 mg, 0.09 mmol) at room temperature under nitrogen atmosphere. The resulting mixture was stirred for 1 h at 85° C. under nitrogen atmosphere. Upon completion, the resulting mixture was cooled down to room temperature and purified by reversed phase flash chromatography with the following conditions: (Column: WelFlash™ C18-I, 20-40 um, 120 g; Eluent A: water (plus 10 mmol/L FA); Eluent B: ACN; Gradient: 40%-60% B in 15 min; Flow rate: 50 mL/min; Detector: UV 220/254 nm; Desired fractions were collected at 55% B and concentrated under reduced pressure to afford the titled compound (200 mg, 95%) as a white solid. ¹H NMR (400 MHz, DMSO-d₆) δ 7.94 (d, J=1.8 Hz, 1H), 7.80-7.69 (m, 2H), 7.37-7.22 (m, 4H), 4.35 (s, 2H), 2.24 (s, 3H). LC/MS (ESI, m/z): [(M−1)]⁻=231.10.

### Step 2: 7-(2-methylphenyl)-2,4-dihydroisoquinoline-1,3-dione

A solution of 4-(cyanomethyl)-2′-methyl-[1,1′-biphenyl]-3-carbonitrile (100 mg, 0.43 mmol) in conc. HCl (3.00 mL) was stirred for 4 h at 70° C. After cooling down to room temperature, the precipitated solids were collected by filtration and washed with water (3×5.00 mL). The crude product was purified by Prep-HPLC with following conditions: (Column: Xbridge Prep OBD C¹⁸, 30×150 mm 5 um; Mobile Phase A: water (plus 0.1% FA); Mobile Phase B: ACN; Flow rate: 60 mL/min; Gradient: 30% B to 55% B in 8 min; Detector: UV 254/220 nm). Desired fractions were collected at 7.43 min and concentrated under reduced pressure to afford the titled compound (60.0 mg, 55%) as a white solid. ¹H NMR (400 MHz, DMSO-d₆) δ 11.36 (s, 1H), 7.92 (d, J=2.0 Hz, 1H), 7.66 (dd, J=7.9, 2.0 Hz, 1H), 7.47 (d, J=7.9 Hz, 1H), 7.41-7.26 (m, 3H), 7.26-7.20 (m, 1H), 4.10 (s, 2H), 2.24 (s, 3H). LC/MS (ESI, m/z): [(M−1)]⁻=250.10.

### Example 5. 2,4-Dihydroisoquinoline-1,3-dione (I-5)

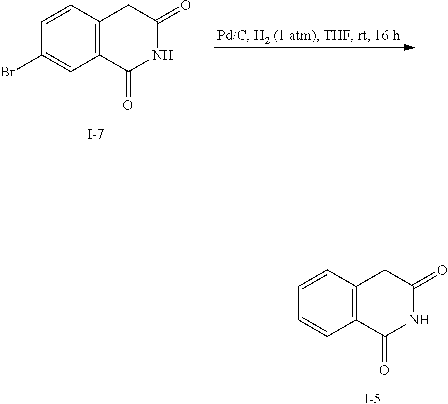

To a solution of 7-bromo-2,4-dihydroisoquinoline-1,3-dione (100 mg, 0.42 mmol) in THF (10.0 mL) was added 10% palladium on activated carbon (20.0 mg) at room temperature under nitrogen atmosphere. The mixture was purged with hydrogen for 3 times and stirred for 16 h at room temperature. The resulting mixture was filtered. The filter cake was washed with DCM (2×10.0 mL). The filtrate was concentrated under reduced pressure and the residue was purified by Prep-HPLC with following conditions: (Column: Xselect CSH OBD, 30×150 mm, 5 um; Mobile Phase A: water (plus 0.1% FA); Mobile Phase B: ACN; Flow rate: 60 mL/min; Gradient: 5% B to 27% B in 7 min; Detector: UV 220/254 nm). Desired fractions were collected at 6.20 min and lyophilized to afford the titled compound (40.0 mg, 60%) as a white solid. ¹H NMR (400 MHz, DMSO-d₆) δ 11.30 (s, 1H), 8.02 (dd, J=7.7, 1.4 Hz, 1H), 7.66 (td, J=7.5, 1.4 Hz, 1H), 7.51-7.42 (m, 1H), 7.39 (d, J=7.7 Hz, 1H), 4.04 (s, 2H). LC/MS (ESI, m/z): [(M+1)]⁺=162.25

### Example 6. 7-(4-Methyl-1,3-oxazol-5-yl)-2,4-dihydroisoquinoline-1,3-dione (I-6)

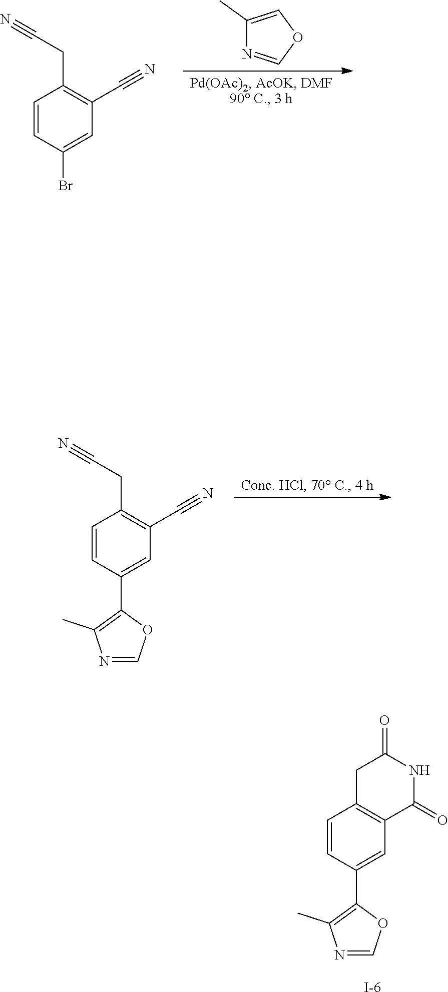

### Step 1: 2-(cyanomethyl)-5-(4-methyl-1,3-oxazol-5-yl)benzonitrile

To a solution of 5-bromo-2-(cyanomethyl)benzonitrile (200 mg, 0.91 mmol) in DMF (4.00 mL) were added 4-methyloxazol (113 mg, 1.36 mmol), KOAc (267 mg, 2.71 mmol) and Pd(OAc)₂ (20.3 mg, 0.09 mmol) at room temperature under nitrogen atmosphere. The resulting mixture was stirred for 3 h at 90° C. After cooling down to room temperature, the resulting mixture was purified by reversed phase flash chromatography with the following conditions: (Column: WelFlash™ C18-I, 20-40 um, 120 g; Eluent A: water (plus 10 mmol/L FA); Eluent B: ACN; Gradient: 30%-50% B in 15 min; Flow rate: 50 mL/min; Detector: UV 220/254 nm; Desired fractions were collected at 40% B and concentrated under reduced pressure to afford the titled compound (100 mg, 50%) as a white solid. ¹H NMR (400 MHz, DMSO-d₆) δ 8.45 (s, 1H), 8.11 (d, J=2.0 Hz, 1H), 8.01 (dd, J=8.2, 2.0 Hz, 1H), 7.78 (d, J=8.3 Hz, 1H), 4.35 (s, 2H), 2.42 (s, 3H). LC/MS (ESI, m/z): [(M+1)]⁺=224.25

### Step 2: 7-(4-methyl-1,3-oxazol-5-yl)-2,4-dihydroisoquinoline-1,3-dione

A solution of 2-(cyanomethyl)-5-(4-methyl-1,3-oxazol-5-yl)benzonitrile (50.0 mg, 0.22 mmol) in conc. HCl (3.00 mL) was stirred for 4 h at 70° C. The precipitated solids were collected by filtration and washed with water (3×5.00 mL). The crude product was purified by Prep-HPLC with following conditions: (Column: Xselect CSH OBD, 30×150 mm, 5 um; Mobile Phase A: water (plus 0.1% FA); Mobile Phase B: ACN; Flow rate: 60 mL/min; Gradient: 10% B to 35% B in 7 min; Detector: UV 220/254 nm). Desired fractions were collected at 6.10 min and lyophilized to afford the titled compound (40.0 mg, 74%) as a white solid. ¹H NMR (400 MHz, DMSO-d₆) δ 11.43 (s, 1H), 8.40 (s, 1H), 8.20 (d, J=2.0 Hz, 1H), 7.90 (dd, J=8.1, 2.0 Hz, 1H), 7.53 (d, J=8.2 Hz, 1H), 4.08 (s, 2H), 2.41 (s, 3H). LC/MS (ESI, m/z): [(M−1)]⁻=241.10

### Example 7. 7-Bromo-2,4-dihydroisoquinoline-1,3-dione (I-7)

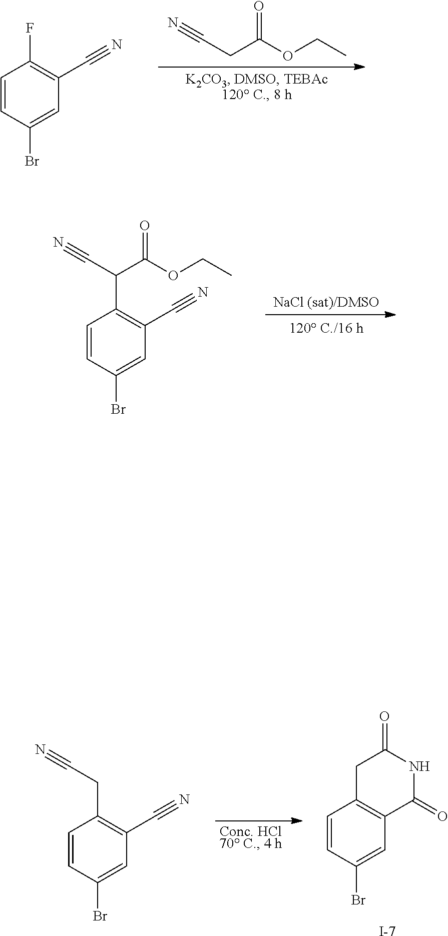

### Step 1: ethyl 2-(4-bromo-2-cyanophenyl)-2-cyanoacetate

To a solution of 5-bromo-2-fluorobenzonitrile (5.00 g, 25.0 mmol) in DMSO (25.0 mL) were added ethyl cyanoacetate (2.83 g, 25.0 mmol), TEBAc (0.11 g, 0.50 mmol), K₂CO₃ (10.4 g, 75.0 mmol) at room temperature under nitrogen atmosphere. The resulting mixture was stirred for 8 h at 120° C. Upon completion, the resulting mixture was cooled down to room temperature and poured into HCl (6 N, 100 mL). The resulting mixture was extracted with EtOAc (3×100 mL). The combined organic layers were washed with brine (100 mL) and dried over anhydrous Na₂SO₄. After filtration, the filtrate was concentrated under reduced pressure to give the titled compound (7.20 g, 98%) as a dark yellow semi-solid. ¹H NMR (400 MHz, DMSO-d₆) δ 8.35 (d, J=2.2 Hz, 1H), 8.06 (dd, J=8.4, 2.2 Hz, 1H), 7.63 (d, J=8.4 Hz, 1H), 6.09 (s, 1H), 4.26 (q, J=7.1 Hz, 2H), 1.22 (t, J=7.1 Hz, 3H). LC/MS (ESI, m/z): [(M+18)]⁺=310.05, 312.05.

### Step 2: 5-bromo-2-(cyanomethyl)benzonitrile

To a solution of ethyl 2-(4-bromo-2-cyanophenyl)-2-cyanoacetate (6.50 g, 22.2 mmol) in DMSO (25.0 mL) was added brine (10.0 mL) at room temperature. The resulting solution was stirred for 16 h at 120° C. The resulting solution was cooled down to room temperature and concentrated under reduced pressure. The residue was purified by reversed phase flash chromatography with the following conditions: (Column: WelFlash™ C18-I, 20-40 um, 330 g; Eluent A: water (plus 10 mmol/L FA); Eluent B: ACN; Gradient: 40%-60% B in 20 min; Flow rate: 80 mL/min; Detector: UV 220/254 nm); Desired fractions were collected at 54% B and concentrated under reduced pressure to afford the titled compound (4.00 g, 82%) as a light yellow solid. ¹H NMR (400 MHz, DMSO-d₆) δ 8.25 (d, J=2.2 Hz, 1H), 8.00 (dd, J=8.4, 2.2 Hz, 1H), 7.60 (d, J=8.4 Hz, 1H), 4.28 (s, 2H). LC/MS (ESI, m/z): [(M+18)]⁺=238.05, 240.05.

### Step 3: 7-bromo-2,4-dihydroisoquinoline-1,3-dione

A suspension of 5-bromo-2-(cyanomethyl)benzonitrile (500 mg, 2.62 mmol) in conc. HCl (10.0 mL) was stirred for 4 h at 70° C. After cooling down to room temperature, the precipitated solids were collected by filtration and washed with water (3×10.0 mL). The crude product was purified by Prep-HPLC with following conditions: (Column: Xselect CSH OBD, 30×150 mm, 5 um, Mobile Phase A: water (plus 0.1% FA); Mobile Phase B: ACN; Flow rate: 60 mL/min; Gradient: 15% B to 40% B in 7 min; Detector: UV 220/254 nm; Desired fractions were collected at 6.82 min and concentrated under reduced pressure to afford the titled compound (500 mg, 92%) as a white solid. ¹H NMR (400 MHz, DMSO-d₆) δ 11.45 (s, 1H), 8.09 (d, J=2.2 Hz, 1H), 7.85 (dd, J=8.3, 2.3 Hz, 1H), 7.38 (d, J=8.3 Hz, 1H), 4.01 (s, 2H). LC/MS (ESI, m/z): [(M+1)]⁺=339.95, 341.95.

### Example 8. 8-Bromo-2,4-dihydroisoquinoline-1,3-dione (I-8)

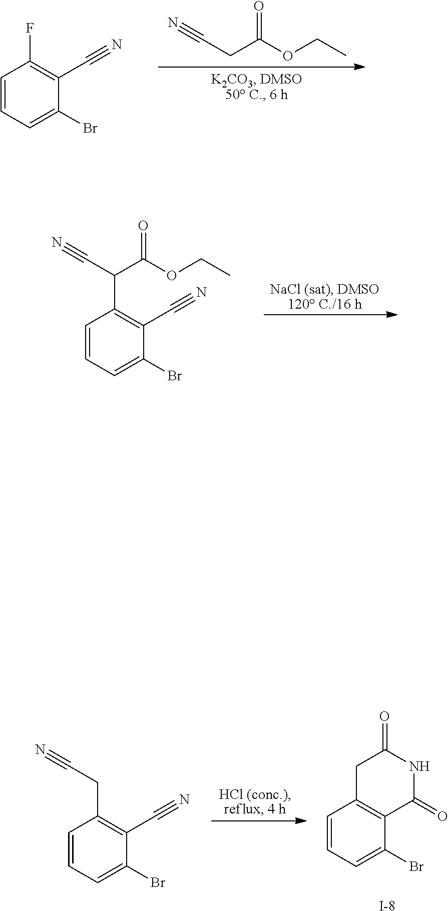

To a solution of 2-bromo-6-fluorobenzonitrile (2.20 g, 11.0 mmol) in DMSO (15.0 mL) were added ethyl cyanoacetate (1.24 g, 11.0 mmol), K₂CO₃ (2.28 g, 16.5 mmol) at room temperature under nitrogen atmosphere. The resulting mixture was stirred for 6 h at 50° C. The resulting solution was cooled to room temperature and purified by reversed phase flash chromatography with the following conditions: (Column: WelFlash™ C18-I, 20-40 um, 330 g; Eluent A: water (plus 10 mmol/L NH₄HCO₃); Eluent B: ACN; Gradient: 25%-45% B in 20 min; Flow rate: 80 mL/min; Detector: UV 220/254 nm); Desired fractions were collected at 33% B and concentrated under reduced pressure to afford the titled compound (1.00 g, 30%) as a yellow solid. ¹H NMR (400 MHz, CDCl₃) δ. 7.79 (dd, J=8.1, 1.1 Hz, 1H), 7.72 (dd, J=7.9, 1.1 Hz, 1H), 7.59 (t, J=8.0 Hz, 1H), 5.20 (s, 1H), 4.35 (qd, J=7.2, 5.0 Hz, 2H), 1.36 (t, J=7.1 Hz, 3H). LC/MS (ESI, m/z): [(M+18)]⁺=310.05, 312.05.

### Step 2: 2-bromo-6-(cyanomethyl)benzonitrile

To a solution of ethyl 2-(3-bromo-2-cyanophenyl)-2-cyanoacetate (10.5 g, 35.8 mmol) in DMSO (11.0 mL) was added brine (5.50 mL). The resulting solution was stirred at 120° C. for 16 h. The resulting solution was cooled down to room temperature and purified by reversed phase flash chromatography with the following conditions: (Column: WelFlash™ C18-I, 20-40 um, 80 g; Eluent A: water (plus 10 mmol/L AcOH); Eluent B: ACN; Gradient: 30%-50% B in 20 min; Flow rate: 70 mL/min; Detector: UV 220/254 nm); Desired fractions were collected at 43% B and concentrated under reduced pressure to afford the titled compound (2.30 g, 29%) as a white solid. ¹H NMR (400 MHz, DMSO-d₆) δ 7.90 (dd, J=7.6, 1.6 Hz, 1H), 7.75-7.64 (m, 2H), 4.35 (s, 2H).

### Step 3: 8-bromo-2,4-dihydroisoquinoline-1,3-dione

A mixture of 2-bromo-6-(cyanomethyl)benzonitrile (1.00 g, 4.53 mmol) in conc. HCl (25.0 mL) was stirred at 75° C. for 4 h under N₂ atmosphere. The resulting solution was cooled down to room temperature and concentrated under reduced pressure. The residue was purified by Prep-HPLC with the following conditions: (Column: XBridge Prep OBD C¹⁸, 30×150 mm 5 um; Mobile Phase A: water (plus 0.1% FA), Mobile Phase B: ACN; Flow rate: 60 mL/min; Gradient: 15% B to 40% B in 8 min; Detector: UV 254/220 nm; Desired fractions were collected at 7.02 min and concentrated under reduced pressure to afford the titled compound (1.02 g, 94%) as a white solid. ¹H NMR (400 MHz, DMSO-d₆) δ 11.29 (s, 1H), 7.72 (dd, J=7.9, 1.2 Hz, 1H), 7.49 (t, J=7.8 Hz, 1H), 7.40 (dd, J=7.6, 1.2 Hz, 1H), 4.08 (s, 2H). LC/MS (ESI, m/z): [(M−1)]⁻=238.00, 240.00.

### Example 9. In Vitro Assays

### Time-Resolved Fluorescence Resonance Energy Transfer (TR-FRET) Assay.

Equal volumes of His-tagged CRBN-DDB1 complex (56 nM) was mixed with Eu-cryptate labeled Anti-6HIS-monoclonal antibody (50× dilution from the commercial stock solution, Vender: Cisbio, Cat. #61HI2KLA) in a final buffer containing 20 mM HEPES pH 7.0, 150 mM NaCl, 0.005% Tween-20. The solution was then mixed with Cy5-labeled thalidomide (final 8 nM) and various concentrations of compounds (a serial 3-fold dilution with the top concentration 200 uM). The mixture were incubated at room temperature for 1 hour. FRET signals were measured on an EnVision plate reader (Perkin Elmer) by exciting at 340 nm and recording emission at both 615 nm as no FRET control and 665 nm as the FRET signals with a 60 microsecond delay. FRET efficiency was calculated as the ratio of fluorescent signals at 665 nM/615 nM. Quantitative loss of FRET efficiency as a function of compound concentrations was fitted by a four-parameter Logistic Function using GraphPad Prism 7.0 and the IC₅₀ values were reported for each compound.

### Fluorescence Polarization (FP) Assay

Materials: PerkinElmer EnVision 2104 Multilabel Reader, PerkinElmer FITC FP dual emission Label 2100-8060, Corning 384-well microplate #4514, V-CMR-TM-493 (FAM-BB-100), and CRBN-DDB1-180605.

Methods: Dilution buffer (50 mM HEPES-Na pH 7.5, 100 mM NaCl, 2 mM DTT, and 0.005% Tween-20) and compound DMSO dilutions were prepared. 4× compound working solutions were made with 8% DMSO (4×) and 13.8 μL/well of dilution buffer was placed in a new 96-well plate and 1.2 μL/well of above compound DMSO dilutions were added to the wells. 40 nM (4×) TM-493 in dilution buffer was prepared from a 10 μM DMSO stock and 15 μL per well of the TM-493 working solution was transferred into the compound working solution. 160 nM (2×) CRBN-DDB1 working solution was prepared with Dilution Buffer and 10 μL/well were added into a 384-well plate. 10 μL per well of the TM-493/compound mixture were transferred into the 384-well plate. The “positive control” was sample without protein and compound in 2% DMSO. After incubation at room temperature for 60 min, data was collected and read on data on an EnVision with the following setting: Excitation Light (%): 100; Measurement Height: 9.4; G-Factor: 1; Detector Gain 1: 200; Detector Gain 2: 200; Flash Number: 100. Data was processed using GraphPad Prism 7.0

Table 3 shows the results for selected compounds in the time-resolved fluorescence resonance energy transfer (TR-FRET) and a fluorescence polarization (FP) assay. The compound numbers correspond to the compound numbers in Table 1. Compounds having an activity designated as “A” provided an IC₅₀ of <10 μM; compounds having an activity designated as “B” provided an IC₅₀ of 10-30 μM; compounds having an activity designated as “C” provided an IC₅₀ of 30-100 μM; and compounds having an activity designated as “D” provided an IC₅₀ of >100 μM. For reference, the known CRBN binders provided the following IC₅₀ values in the TR-FRET assay: thalidomide (IC₅₀=2.9 μM), lenalidomide (IC₅₀=1.17 μM) and pomalidomide (IC₅₀=1.28 μM).

TABLE 3
TR-FRET and FP Assay Results

| | CRBN | |
|---|---|---|
| | binding | CRBN |
| | Human | binding |
| | HTRF: | Human FP: |
| | Average IC₅₀ | average IC₅₀ |
| I-# | (μM) | (μM) |
| I-1 | D | B |
| I-2 | D | C |
| I-3 | D | C |
| I-4 | C | D |
| I-5 | D | C |
| I-6 | D | D |
| I-7 | C | D |
| I-8 | B | D |
| I-12 | A | B |
| I-13 | D | — |
| I-17 | D | C |
| I-18 | D | C |
| I-19 | A | D |
| I-20 | C | D |
| I-21 | C | B |
| I-22 | D | B |
| I-23 | B | C |
| I-24 | — | B |
| I-25 | — | D |
| I-26 | A | A |
| I-27 | C | C |
| I-28 | D | — |
| I-29 | D | D |
| I-30 | D | — |
| I-31 | C | D |
| I-32 | D | — |
| I-33 | D | D |
| I-35 | D | — |
| I-42 | B | C |
| I-49 | D | D |
| I-50 | D | D |
| I-52 | C | C |
| I-53 | D | D |
| I-54 | D | D |
| I-55 | D | — |
| I-56 | D | — |
| I-58 | D | D |
| I-59 | D | — |
| I-60 | D | — |
| I-62 | A | C |
| I-68 | B | B |
| I-71 | A | A |
| I-72 | B | D |
| I-73 | B | D |
| I-74 | C | C |
| I-75 | B | C |
| I-77 | B | B |
| I-78 | B | D |
| I-79 | C | D |
| I-86 | D | — |
| I-87 | D | — |
| I-88 | D | — |
| I-89 | D | — |
| I-90 | D | — |
| I-91 | D | — |
| I-92 | C | — |
| I-93 | D | — |
| I-94 | D | — |
| I-95 | C | — |
| I-96 | D | — |
| I-97 | D | — |
| I-98 | D | — |
| I-99 | D | — |
| I-100 | D | — |
| I-101 | D | — |
| I-102 | D | — |
| I-103 | D | — |
| I-104 | D | — |
| I-105 | D | — |
| I-106 | D | — |
| I-107 | D | — |
| I-108 | C | — |
| I-109 | D | — |
| I-110 | D | — |
| I-111 | D | — |
| I-112 | D | — |
| I-113 | D | — |
| I-114 | C | — |
| I-115 | D | — |
| I-116 | D | — |
| I-117 | D | — |
| I-118 | D | — |
| I-119 | D | — |
| I-120 | D | — |
| I-121 | D | — |
| I-122 | D | — |
| I-123 | C | — |
| I-124 | C | D |
| I-125 | D | — |
| I-126 | D | — |
| I-127 | D | — |
| I-128 | D | — |
| I-129 | D | — |
| I-130 | C | — |
| I-131 | D | — |
| I-132 | C | D |
| I-133 | B | D |
| I-134 | D | — |
| I-135 | D | — |
| I-136 | D | — |
| I-137 | D | — |
| I-138 | D | — |
| I-139 | D | — |
| I-140 | D | — |
| I-141 | D | — |
| I-142 | D | — |
| I-143 | D | — |
| I-144 | D | — |
| I-145 | D | — |
| I-146 | D | — |
| I-147 | D | — |
| I-148 | D | — |
| I-149 | D | — |
| I-150 | D | — |
| I-151 | C | D |
| I-153 | D | — |
| I-154 | D | — |
| I-155 | D | — |
| I-156 | D | — |
| I-157 | D | — |
| I-158 | D | — |
| I-159 | D | D |
| I-160 | D | — |
| I-161 | D | — |
| I-162 | D | — |
| I-163 | D | — |
| I-164 | D | — |
| I-165 | D | — |
| I-166 | D | — |
| I-167 | D | — |
| I-168 | D | — |
| I-169 | D | — |
| I-170 | D | — |
| I-171 | D | — |
| I-172 | D | — |
| I-173 | D | — |
| I-174 | D | — |
| I-175 | D | D |
| I-176 | D | D |
| I-177 | D | — |
| I-178 | D | — |
| I-179 | D | — |
| I-180 | D | — |
| I-181 | D | — |
| I-182 | D | — |
| I-183 | D | — |
| I-184 | D | — |
| I-185 | D | D |
| I-186 | C | D |
| I-189 | D | — |
| I-190 | D | — |
| I-191 | D | — |
| I-192 | D | — |
| I-193 | B | C |
| I-194 | B | C |
| I-195 | D | — |
| I-196 | D | D |
| I-197 | D | D |
| I-198 | A | B |
| I-199 | A | B |
| I-200 | A | A |
| I-202 | A | A |
| I-203 | A | A |
| I-204 | A | A |
| I-205 | — | A |
| I-206 | — | A |
| I-207 | — | A |
| I-208 | — | B |
| I-209 | D | — |
| I-210 | A | — |
| I-211 | A | — |
| I-212 | C | — |
| I-213 | B | B |
| I-214 | D | C |
| I-215 | A | B |
| I-216 | A | C |
| I-217 | D | C |
| I-218 | D | D |
| I-219 | A | B |
| I-220 | B | C |
| I-221 | A | D |
| I-222 | C | C |
| I-223 | C | C |
| I-224 | A | D |
| I-225 | D | D |
| I-226 | D | D |
| I-227 | A | D |
| I-228 | D | D |
| I-229 | D | D |
| I-230 | D | D |
| I-231 | D | D |
| I-232 | D | D |
| I-233 | D | D |
| I-234 | A | A |
| I-235 | A | B |
| I-236 | D | D |
| I-237 | A | B |
| I-238 | C | C |
| I-239 | A | A |
| I-240 | A | B |
| I-241 | A | C |
| I-242 | A | — |
| I-243 | A | C |
| I-244 | B | C |
| I-245 | A | B |
| I-246 | A | A |
| I-247 | A | B |
| I-248 | D | C |
| I-249 | B | B |
| I-250 | A | A |
| I-251 | A | A |
| I-252 | B | C |
| I-253 | B | A |
| I-254 | B | C |
| I-255 | B | B |
| I-256 | — | — |
| I-257 | D | D |
| I-258 | D | C |
| I-259 | D | B |
| I-260 | D | D |
| I-261 | — | B |
| I-262 | — | D |
| I-263 | — | C |
| I-264 | A | B |
| I-265 | A | A |
| I-266 | B | A |
| I-267 | B | A |
| I-268 | C | B |
| I-269 | — | — |
| I-270 | — | — |
| I-271 | — | B |
| I-272 | — | — |
| I-273 | B | B |
| I-274 | C | C |
| I-275 | B | B |
| I-276 | C | C |
| I-277 | B | B |
| I-278 | B | B |
| I-279 | C | — |
| I-280 | C | — |
| I-281 | A | — |
| I-282 | D | — |
| I-283 | D | — |
| I-284 | D | — |
| I-285 | — | — |
| I-286 | D | — |
| I-287 | D | — |
| I-288 | D | — |
| I-289 | D | — |
| I-290 | D | — |
| I-291 | D | — |
| I-292 | B | D |
| I-293 | D | — |
| I-294 | D | — |
| I-295 | C | — |
| I-296 | D | — |
| I-297 | D | — |
| I-298 | D | — |
| I-299 | D | — |
| I-300 | D | — |
| I-301 | D | — |
| I-302 | D | — |
| I-303 | D | — |
| I-304 | D | — |
| I-305 | D | — |
| I-306 | D | — |
| I-307 | D | — |
| I-308 | D | — |
| I-309 | D | — |
| I-310 | D | — |
| I-311 | C | — |
| I-312 | D | — |
| I-313 | D | — |
| I-314 | D | — |
| I-315 | D | — |
| I-316 | C | — |
| I-317 | C | — |
| I-318 | D | — |
| I-319 | C | D |
| I-320 | D | — |
| I-321 | D | — |
| I-322 | D | — |
| I-323 | D | — |
| I-324 | D | — |
| I-325 | D | — |
| I-326 | D | — |
| I-327 | D | — |
| I-328 | D | — |
| I-329 | D | — |
| I-330 | C | — |
| I-331 | D | — |
| I-332 | D | — |
| I-333 | D | — |
| I-334 | D | — |
| I-335 | D | — |
| I-336 | D | — |
| I-337 | D | — |
| I-338 | D | — |
| I-339 | D | — |
| I-340 | D | — |
| I-341 | D | — |
| I-342 | D | — |
| I-343 | D | — |
| I-344 | D | — |
| I-345 | D | — |
| I-346 | D | — |
| I-347 | D | — |
| I-348 | D | — |
| I-349 | D | — |
| I-350 | D | — |
| I-351 | D | — |
| I-352 | D | — |
| I-353 | D | — |
| I-354 | D | — |
| I-355 | D | — |
| I-356 | D | — |
| I-357 | D | — |
| I-358 | D | — |
| I-359 | D | — |
| I-360 | D | — |
| I-361 | D | — |
| I-362 | D | — |
| I-363 | D | — |
| I-364 | D | — |
| I-365 | D | — |
| I-366 | D | — |
| I-367 | D | — |
| I-368 | D | — |
| I-369 | D | — |
| I-370 | D | — |
| I-370 | D | — |
| I-371 | D | — |
| I-372 | D | — |
| I-373 | D | — |
| I-374 | D | — |
| I-375 | C | — |
| I-376 | C | — |
| I-377 | C | — |
| I-378 | C | — |

### Example 10. Synthesis of 7-bromo-2,4-dihydro-2,6-naphthyridine-1,3-dione (I-13)

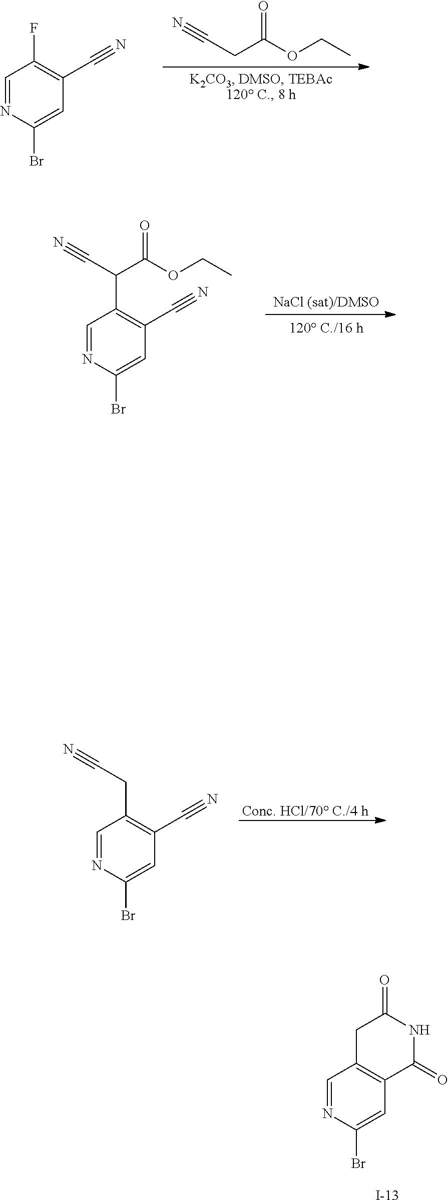

### Step 1: ethyl 2-(6-bromo-4-cyanopyridin-3-yl)-2-cyanoacetate

To a stirred mixture of 2-bromo-5-fluoropyridine-4-carbonitrile (1.05 g, 5.224 mmol), ethyl cyanoacetate (0.59 g, 5.224 mmol) and TEBAc (0.02 g, 0.104 mmol) in DMSO (5 mL) was added K₂CO₃ (2.17 g, 15.672 mmol) at room temperature under nitrogen atmosphere. The mixture was stirred for 8 h at 120° C. The reaction was monitored by LCMS. Upon completion, the mixture was allowed to cool down to room temperature. The mixture was poured into HCl (6 M, 100 mL) and the resulting mixture was extracted with EtOAc (3×100 mL). The combined organic layers were washed with brine (100 mL), dried over anhydrous Na₂SO₄. After filtration, the filtrate was concentrated under reduced pressure to give ethyl 2-(6-bromo-4-cyanopyridin-3-yl)-2-cyanoacetate (1.19 g, 77%) as a light brown liquid. ¹H NMR (400 MHz, DMSO-d₆) δ 8.47 (s, 1H), 7.76-7.62 (m, 1H), 6.22 (s, 1H), 4.03 (q, J=7.1 Hz, 2H), 1.36 (tdd, J=13.1, 10.6, 6.7 Hz, 3H). LC/MS (ESI, m/z): [(M−1)]⁻=292.00, 294.00.

### Step 2: 2-bromo-5-(cyanomethyl)pyridine-4-carbonitrile

To a stirred solution of ethyl 2-(6-bromo-4-cyanopyridin-3-yl)-2-cyanoacetate (1.19 g, 1 equiv) in DMSO (5 mL) was added brine (2 mL) at room temperature. The solution was stirred for 16 h at 120° C. The reaction was monitored by LCMS. The solution was concentrated under reduced pressure and the residue was purified by reverse phase flash chromatography with the following conditions: Column: WelFlash™ C18-I, 20-40 um, 330 g; Eluent A: water (plus 10 mmol/L FA); Eluent B: ACN; Gradient: 20%-60% B in 20 min; Flow rate: 80 mL/min; Detector: 220/254 nm; desired fractions were collected at 50% B and concentrated under reduced pressure to afford 2-bromo-5-(cyanomethyl)pyridine-4-carbonitrile (150 mg, 17%) as a brown solid. ¹H NMR (400 MHz, DMSO-d₆) δ 8.38 (s, 2H), 8.27 (s, 1H), 2.08 (s, 1H). LC/MS (ESI, m/z): [(M−1)]⁻=220.00, 222.00.

### Step 3: 7-bromo-2,4-dihydro-2,6-naphthyridine-1,3-dione

A mixture of 2-bromo-5-(cyanomethyl)pyridine-4-carbonitrile (150.00 mg) in conc. HCl (10 mL) was stirred for 4 h at 70° C. under air atmosphere. The reaction was monitored by LCMS. After being cooling down to room temperature. The precipitated solids were collected by filtration and washed with water (3×10 mL). It was purified by Pre-HPLC with following conditions: Column: X select CSH OBD Column 30×150 mm 5 um; Mobile Phase A: water (0.1% FA), Mobile Phase B: ACN; Flow rate: 60 mL/min; Gradient: 15 B to 40 B in 7 min; 220 nm; RT1: 6.82 min to afford 7-bromo-2,4-dihydro-2,6-naphthyridine-1,3-dione (36 mg) as alight yellow solid. ¹H NMR (400 MHz, DMSO-d₆) δ 11.69 (s, 1H), 8.54 (s, 1H), 7.99 (s, 1H), 4.01 (s, 2H). LC/MS (ESI, m/z): [(M−1)]⁻=239.00, 241.00.

### Example 11. Synthesis of 7-(2-aminophenyl)-2,4-dihydroisoquinoline-1,3-dione (I-17)

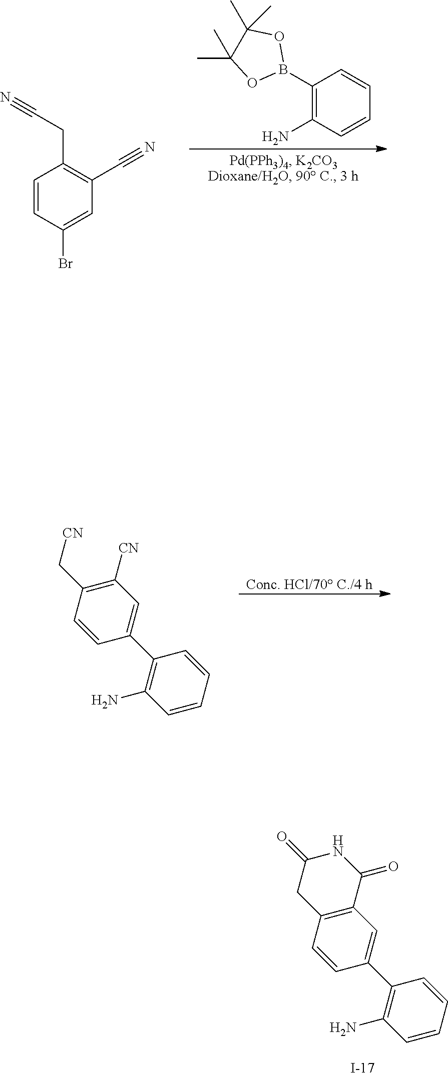

### Step 1: 2′-Amino-4-(cyanomethyl)-[1,1′-biphenyl]-3-carbonitrile

To a solution of 5-bromo-2-(cyanomethyl)benzonitrile (200.00 mg, 0.905 mmol, 1.00 equiv.) and 2-(4,4,5,5-tetramethyl-1,3,2-dioxaborolan-2-yl)aniline (396.44 mg, 1.809 mmol, 2.00 equiv) in H₂O (5.00 mL) and dioxane (5.00 mL) were added K₂CO₃ (375.12 mg, 2.714 mmol, 3.00 equiv.) and Pd(PPh₃)₄ (104.55 mg, 0.090 mmol, 0.10 equiv.). After stirring for 4 h at 90° C. under a nitrogen atmosphere, the resulting mixture was cooled down to room temperature and was concentrated under reduced pressure. The residue was purified by reverse phase flash chromatography with the following conditions: Column: Spherical C¹⁸, 20-40 um, 330 g; Mobile Phase A: water (plus 10 mM FA); Mobile Phase B: ACN; Flow rate: 80 mL/min; Gradient: 5%-5% B, 10 min, 55% B-75% B gradient in 20 min; Detector: 254 nm. The fractions containing the desired product were collected at 60% B and concentrated under reduced pressure to afford to afford 2-amino-4-(cyanomethyl)-[1,1-biphenyl]-3-carbonitrile (80 mg, 38%) as a white solid. ¹H NMR (400 MHz, DMSO-d₆) δ 7.92 (d, J=1.9 Hz, 1H), 7.80 (dd, J=8.0, 2.0 Hz, 1H), 7.70 (d, J=8.1 Hz, 1H), 7.09 (td, J=7.6, 1.6 Hz, 1H), 7.02 (dd, J=7.6, 1.6 Hz, 1H), 6.82-6.75 (m, 1H), 6.65 (td, J=7.4, 1.2 Hz, 1H), 4.99 (s, 2H), 4.31 (s, 2H). LC/MS (ESI, m/z): [(M+1)]⁻=234.15.

### Step 2: 7-(2-Aminophenyl)-2,4-dihydroisoquinoline-1,3-dione

A solution of 2-amino-4-(cyanomethyl)-[1,1-biphenyl]-3-carbonitrile (70.00 mg, 0.300 mmol, 1.00 equiv.) in con. HCl (10 mL) was stirred for overnight at 70° C. the resulting mixture was cooled down to room temperature. The precipitated solids were collected by filtration and washed with water (3×10 mL). Then it was purified by reverse phase flash chromatography with the following conditions: Column: Xselect CSH OBD Column 30×150 mm 5 um; Mobile Phase A: water (0.1% FA), Mobile Phase B: ACN; Flow rate: 60 mL/min; Gradient: 5% B to 20% B in 7 min; 220 nm; Rt: 6.18 min to afford 7-(2-aminophenyl)-2,4-dihydroisoquinoline-1,3-dione (10 mg, 13%) as a yellow solid. ¹H NMR (400 MHz, DMSO-d₆) δ 11.35 (s, 1H), 8.04 (d, J=2.0 Hz, 1H), 7.70 (dd, J=7.9, 2.0 Hz, 1H), 7.46 (d, J=7.9 Hz, 1H), 7.17-6.98 (m, 2H), 6.79 (dd, J=8.1, 1.2 Hz, 1H), 6.67 (td, J=7.4, 1.3 Hz, 1H), 4.84 (s, 2H), 4.08 (s, 2H). LC/MS (ESI, m/z): [(M−1)]⁻=251.05.

### Example 12. Synthesis of 7-(2-Methoxyphenyl)-2,4-dihydroisoquinoline-1,3-dione (I-18)

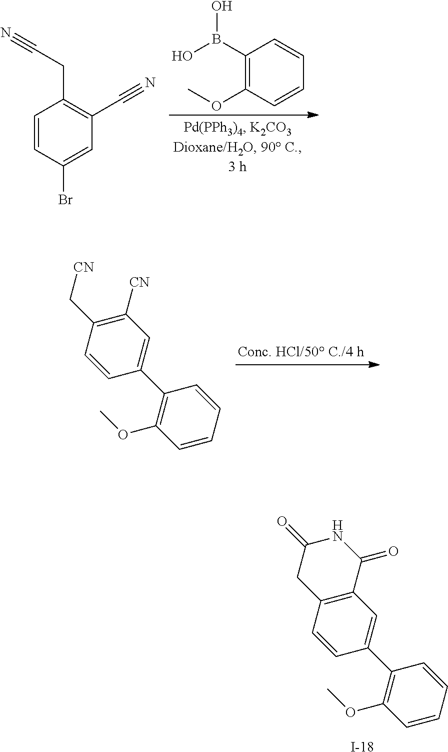

### Step 1: 4-(Cyanomethyl)-2′-methoxy-[1,1′-biphenyl]-3-carbonitrile

To a solution of 5-bromo-2-(cyanomethyl)benzonitrile (200.00 mg, 0.905 mmol, 1.00 equiv.) and 2-methoxyphenylboronic acid (274.97 mg, 1.809 mmol, 2.00 equiv.) in H₂O (5.00 mL) and dioxane (5.00 mL) were added K₂CO₃ (375.12 mg, 2.714 mmol, 3.00 equiv.) and Pd(PPh₃)₄ (104.55 mg, 0.090 mmol, 0.10 equiv.). After stirring for 4 h at 90° C. under a nitrogen atmosphere, the resulting mixture was cooled down to room temperature and was concentrated under reduced pressure. The residue was purified by reverse phase flash chromatography with the following conditions: Column: Spherical C¹⁸, 20-40 um, 330 g; Mobile Phase A: water (plus 10 mM FA); Mobile Phase B: ACN; Flow rate: 80 mL/min; Gradient: 5%-5% B, 10 min, 55% B-75% B gradient in 20 min; Detector: 254 nm. The fractions containing the desired product were collected at 60% B and concentrated under reduced pressure to afford to afford 4-(cyanomethyl)-2-methoxy-[1,1-biphenyl]-3-carbonitrile (80 mg, 36%) as a white solid. ¹H NMR (400 MHz, DMSO-d₆) δ 8.00 (d, J=2.0 Hz, 1H), 7.87 (dd, J=8.1, 2.0 Hz, 1H), 7.69 (d, J=8.1 Hz, 1H), 7.46-7.40 (m, 1H), 7.40-7.34 (m, 1H), 7.16 (dd, J=8.4, 1.1 Hz, 1H), 7.07 (td, J=7.4, 1.1 Hz, 1H), 4.31 (s, 2H), 3.80 (s, 3H). LC/MS (ESI, m/z): [(M−1)]⁻=247.00

### Step 2: 7-(2-Methoxyphenyl)-2,4-dihydroisoquinoline-1,3-dione

A solution of 4-(cyanomethyl)-2-methoxy-[1,1-biphenyl]-3-carbonitrile (100.00 mg, 0.403 mmol, 1.00 equiv.) in con. HCl (10.00 mL) was stirred for overnight at 70° C. under nitrogen atmosphere. The resulting mixture was cooled down to room temperature. The precipitated solids were collected by filtration and washed with water (3×10 mL). it was purified by reverse phase flash chromatography with the following conditions: Column: Xselect CSH OBD Column 30×150 mm 5 um; Mobile Phase A: water (0.1% FA), Mobile Phase B: ACN; Flow rate: 60 mL/min; Gradient: 5% B to 20% B in 7 min; 220 nm; Rt: 6.18 min to afford 7-(2-methoxyphenyl)-2,4-dihydroisoquinoline-1,3-dione (40 mg, 37%) as a yellow solid. ¹H NMR (400 MHz, DMSO-d₆) δ 11.33 (s, 1H), 8.09 (d, J=2.0 Hz, 1H), 7.75 (dd, J=7.9, 2.0 Hz, 1H), 7.46-7.36 (m, 2H), 7.33 (dd, J=7.5, 1.8 Hz, 1H), 7.15 (dd, J=8.4, 1.1 Hz, 1H), 7.06 (td, J=7.5, 1.1 Hz, 1H), 4.07 (s, 2H), 3.78 (s, 3H). LC/MS (ESI, m z): [(M−1)]⁻=266.00.

### Example 13. Synthesis of 7-hydroxy-2,4-dihydroisoquinoline-1,3-dione (I-19)

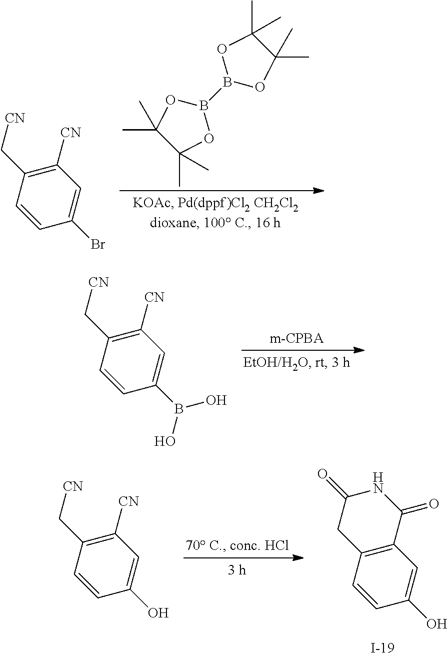

### Step 1: 3-cyano-4-(cyanomethyl)phenylboronic acid

To a stirred mixture of 5-bromo-2-(cyanomethyl)benzonitrile (2.00 g, 9.05 mmol), bis(pinacolato)diboron (3.45 g, 13.57 mmol) and AcOK (2.66 g, 27.14 mmol) in dioxane (40 mL) was added Pd(dppf)Cl₂ CH₂Cl₂ (738.85 mg, 0.91 mmol) at room temperature under nitrogen atmosphere. The mixture was allowed to react for 16 h at 100° C. The reaction was monitored by LCMS. The mixture was allowed to cool down to room temperature. The resulting mixture was filtered, the filter cake was washed with CH₂Cl₂ (2×10 mL). The filtrate was concentrated under reduced pressure. The residue was purified by reverse phase flash chromatography with the following conditions: Column: Wel Flash™ C18-I, 20-40 um, 330 g; Eluent A: water (plus 10 mmol/L FA); Eluent B: ACN; Gradient: 25%-55% B in 20 min; Flow rate: 80 mL/min; Detector: 220/254 nm; desired fractions were collected at 35% B and concentrated under reduced pressure to afford 3-cyano-4-(cyanomethyl)phenylboronic acid (1.2 g, 71%) as a white solid. ¹H NMR (400 MHz, DMSO-d₆) δ 8.46 (s, 2H), 8.20 (d, J=1.3 Hz, 1H), 8.11 (dd, J=7.7, 1.4 Hz, 1H), 7.64 (d, J=7.8 Hz, 1H), 4.31 (s, 2H). LC/MS (ESI, m/z): [(M+18)]⁺=198.60.

### Step 2: 2-(cyanomethyl)-5-hydroxybenzonitrile

To a stirred solution of 3-cyano-4-(cyanomethyl)phenylboronic acid (100.00 mg, 0.54 mmol) in EtOH (4.00 mL)/H₂O (2.00 mL) was added m-CPBA (111.34 mg, 0.65 mmol) at 0° C. under nitrogen atmosphere. The solution was allowed to react for 2 h at 0° C. The reaction was monitored by LCMS. The resulting solution was concentrated under reduced pressure and the residue was purified by reverse phase flash chromatography with the following conditions: Column: Wel Flash m C18-I, 20-40 um, 80 g; Eluent A: water (plus 10 mmol/L FA); Eluent B: ACN; Gradient: 20%-45% B in 15 min; Flow rate: 50 mL/min; Detector: 220/254 nm; desired fractions were collected at 35% B and concentrated under reduced pressure to afford 2-(cyanomethyl)-5-hydroxybenzonitrile (80 mg, 94%) as a white solid. ¹H NMR (400 MHz, DMSO-d₆) δ 10.40 (s, 1H), 7.45 (d, J=8.6 Hz, 1H), 7.21 (d, J=2.6 Hz, 1H), 7.14 (dd, J=8.5, 2.6 Hz, 1H), 4.11 (s, 2H). LC/MS (ESI, m/z): [(M+18)]⁺=176.15.

### Step 3: 7-hydroxy-2,4-dihydroisoquinoline-1,3-dione

A solution of 2-(cyanomethyl)-5-hydroxybenzonitrile (80 mg, 0.51 mmol) in conc. HCl (5 mL) was stirred for 2 h at 70° C. The reaction was monitored by LCMS. The mixture was allowed to cool down to room temperature. The precipitated solids were collected by filtration and washed with water (3×5 mL). This afford 7-hydroxy-2,4-dihydroisoquinoline-1,3-dione (70 mg, 78%) as a light yellow solid. ¹H NMR (400 MHz, DMSO-d₆) δ 11.22 (s, 1H), 9.82 (s, 1H), 7.38 (d, J=2.7 Hz, 1H), 7.20 (d, J=8.4 Hz, 1H), 7.06 (dd, J=8.3, 2.7 Hz, 1H), 3.90 (s, 2H). LC/MS (ESI, m/z): [(M−1)]⁻=175.95.

### Example 14. Synthesis of 1,3-dioxo-2,4-dihydroisoquinoline-7-carboxamide (I-20)

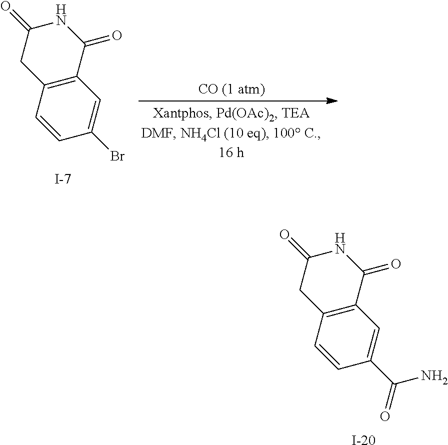

To a stirred mixture of 7-bromo-2,4-dihydroisoquinoline-1,3-dione (100 mg, 0.42 mmol), XantPhos (48.21 mg, 0.08 mmol), NH₄Cl (222.83 mg, 4.17 mmol) and TEA (505.83 mg, 5.00 mmol) in DMF (5 mL) was added Pd(AcO)₂ (9.35 mg, 0.04 mmol) at room temperature under nitrogen atmosphere. The mixture was allowed to react for 16 h at 100° C. The reaction was monitored by LCMS. The residue was cooled to room temperature and was purified by reverse phase flash chromatography with the following conditions: Column: Wel Flash™ C18-I, 20-40 um, 80 g; Eluent A: water (plus 10 mmol/L FA); Eluent B: ACN; Gradient: 15%-35% B in 15 min; Flow rate: 50 mL/min; Detector: 220/254 nm; desired fractions were collected at 26% B and concentrated under reduced pressure. Then further purified by Pre-HPLC with following conditions: Column: Sun fire Prep C¹⁸ OBD Column, 10 um, 19×250 mm; Mobile Phase A: 0.05% TFA, Mobile Phase B: ACN; Flow rate: 25 mL/min; Gradient: 4 B to 10 B in 12 min; 254 nm; RT: 10.88 min to give 1,3-dioxo-2,4-dihydroisoquinoline-7-carboxamide (10 mg, 11.76%) as a white solid. ¹H NMR (400 MHz, DMSO-d₆) δ 11.40 (s, 1H), 8.54 (d, J=1.9 Hz, 1H), 8.21 (s, 1H), 8.11 (dd, J=8.0, 2.0 Hz, 1H), 7.52-7.44 (m, 2H), 4.09 (s, 2H). LC/MS (ESI, m/z): [(M−1)]⁻=203.05.

### Example 15. Synthesis of 8-Amino-2,4-dihydroisoquinoline-1,3-dione (I-22)

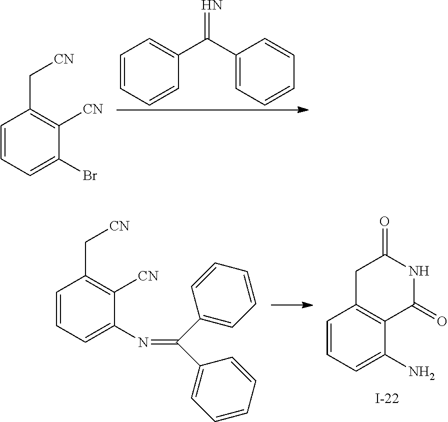

### Step 1: 8-[(Diphenylmethylidene)amino]-2,4-dihydroisoquinoline-1,3-dione

Into a 20 mL sealed tube were added 2-bromo-6-(cyanomethyl)benzonitrile (600 mg, 2.714 mmol, 1.00 equiv.), 2-bromo-6-(cyanomethyl)benzonitrile (590.31 mg, 3.257 mmol, 1.20 equiv.), Cs₂CO₃ (2653.05 mg, 8.143 mmol, 3.00 equiv.), BINAP (507.02 mg, 0.814 mmol, 0.30 equiv.) and Pd₂(dba)₃ (372.82 mg, 0.407 mmol, 0.15 equiv.) in DMF (17 mL) at room temperature under nitrogen atmosphere. The resulting mixture was stirred at 110° C. for 3 h. The mixture was allowed to cool down to room temperature. The residue was concentrated under reduced pressure. The residue was purified by silica gel column chromatography, eluted with petroleum ether:EtOAc (2:1) to afford 8-[(diphenylmethylidene)amino]-2,4-dihydroisoquinoline-1,3-dione (400 mg, 43%) as a yellow solid. ¹H NMR (400 MHz, DMSO-d₆) δ 7.76-7.69 (m, 2H), 7.54-7.46 (m, 4H), 7.40-7.34 (m, 4H), 7.16 (dd, J=7.7, 1.0 Hz, 2H), 6.89 (dd, J=8.2, 1.0 Hz, 1H), 4.18 (s, 2H). LC/MS (ESI, m/z): [(M+1)]⁺=322.20.

### Step 2: 8-Amino-2,4-dihydroisoquinoline-1,3-dione

A mixture of 8-[(diphenylmethylidene)amino]-2,4-dihydroisoquinoline-1,3-dione (400.00 mg, 1.175 mmol, 1.00 equiv.) in con. HCl (12 M, 8.00 mL) was stirred at 75° C. under air atmosphere for 3 h. The mixture was allowed to cool down to room temperature. The reaction mixture was basified to pH 6 with saturated Na₂CO₃ (aq.). The resulting solution was extracted with DCM (3×100 mL). The combined organic layers were washed with brine (2×100 mL), dried over anhydrous Na₂SO₄. After filtration, the filtrate was concentrated under reduced pressure. The residue was purified by reverse phase flash with the following conditions: (Column: Xselect CSH OBD Column 30×150 mm 5 um; Mobile Phase A: water (0.1% FA), Mobile Phase B: ACN; Flow rate: 60 mL/min; Gradient: 5 B to 25 B in 7 min; 220 nm; RT1:6.37 min) to afford 8-amino-2,4-dihydroisoquinoline-1,3-dione (30 mg, 14%) as a white solid. ¹H NMR (400 MHz, DMSO-d₆) δ 10.90 (s, 1H), 7.21 (t, J=7.8 Hz, 1H), 7.12 (s, 2H), 6.64 (d, J=8.3 Hz, 1H), 6.39 (d, J=7.2 Hz, 1H), 3.88 (s, 2H). LC/MS (ESI, m/z): [(M+1)]⁺=177.25.

### Example 16. Synthesis of 8-phenoxy-2,4-dihydroisoquinoline-1,3-dione (I-26)

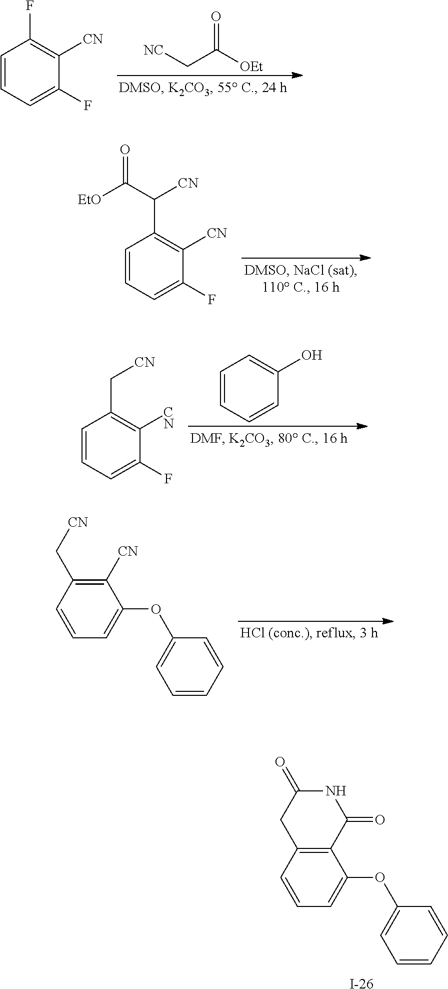

### Step 1. Ethyl 2-cyano-2-(2-cyano-3-fluorophenyl)acetate

To a stirred solution of 2,6-difluorobenzonitrile (11.14 g, 80.083 mmol, 1.00 equiv) and ethyl cyanoacetate (9.96 g, 88.092 mmol, 1.10 equiv) in DMSO (100 mL) was added K₂CO₃ (22.14 g, 160.167 mmol, 2.00 equiv) in portions at room temperature under nitrogen atmosphere. The resulting mixture was stirred for 16 h at 50° C. under nitrogen atmosphere. The resulting mixture was cooled to room temperature and was diluted with ice water (250 mL). The mixture was basified to pH 4 with HCl (aq.)(6M). The resulting mixture was extracted with EtOAc (4×250 mL). The combined organic layers were washed with water (2×100 mL), dried over anhydrous Na₂SO₄. After filtration, the filtrate was concentrated under reduced pressure. The residue was purified by silica gel column chromatography, eluted with Petroleum ether/EtOAc (2:1) to afford ethyl 2-cyano-2-(2-cyano-3-fluorophenyl)acetate (15.8 g, 84.96%) as a pink solid. ¹H NMR (400 MHz, DMSO-d₆) δ 7.91 (td, J=8.2, 6.0 Hz, 1H), 7.70-7.60 (m, 1H), 7.57 (dd, J=7.9, 0.8 Hz, 1H), 6.13 (s, 1H), 4.27 (qd, J=7.1, 1.3 Hz, 2H), 1.23 (t, J=7.1 Hz, 3H); LC/MS (ESI, m/z): [(M−1)]⁻=231.20.

### Step 2. 2-(cyanomethyl)-6-fluorobenzonitrile

To a stirred solution of ethyl 2-cyano-2-(2-cyano-3-fluorophenyl)acetate (5.00 g, 21.532 mmol, 1.00 equiv) in DMSO (10.00 mL) was added saturated NaCl (aq.) (10.00 mL) dropwise at room temperature under nitrogen atmosphere. The resulting mixture was stirred for 16 h at 120° C. under nitrogen atmosphere. The resulting mixture was cooled to room temperature and was diluted with EtOAc (200 mL). The resulting mixture was washed with water (3×150 mL). The organic layer was dried over anhydrous Na₂SO₄. After filtration, the filtrate was concentrated under reduced pressure. The residue was purified by silica gel column chromatography, eluted with Petroleum ether/EtOAc (4:1) to afford 2-(cyanomethyl)-6-fluorobenzonitrile (3.3 g, 96%) as a yellow solid. ¹H NMR (400 MHz, DMSO-d₆) δ 7.85 (tdd, J=7.8, 6.0, 1.4 Hz, 1H), 7.59-7.49 (m, 2H), 4.35 (s, 2H); LC/MS (ESI, m/z): [(M−1)]⁻=159.20.

### Step 3. 2-(cyanomethyl)-6-phenoxybenzonitrile

To a stirred solution of 2-(cyanomethyl)-6-fluorobenzonitrile (2.00 g, 12.488 mmol, 1.00 equiv) and phenol (1175.30 mg, 12.488 mmol, 1 equiv) in DMF (30.00 mL) was added K₂CO₃ (1725.94 mg, 12.488 mmol, 1.00 equiv) in portions at room temperature under nitrogen atmosphere. The resulting mixture was stirred for 16 h at 80° C. under nitrogen atmosphere. The resulting mixture was cooled to room temperature and was diluted with water (50 mL). The resulting mixture was extracted with EtOAc (4×50 mL). The combined organic layers were washed with brine (50 mL), dried over anhydrous Na₂SO₄. After filtration, the filtrate was concentrated under reduced pressure. The residue was purified by silica gel column chromatography, eluted with Petroleum ether/EtOAc (4:1) to afford 2-(cyanomethyl)-6-phenoxybenzonitrile (145 mg, 5%) as a yellow solid. ¹H NMR (400 MHz, Chloroform-d) δ 7.53 (dd, J=8.6, 7.7 Hz, 1H), 7.50-7.41 (m, 2H), 7.38-7.25 (m, 2H), 7.16-7.08 (m, 2H), 6.85 (dd, J=8.6, 0.8 Hz, 1H), 4.04 (s, 2H). LC/MS (ESI, m/z): [(M−1)]⁻=233.00.

### Step 4. 8-phenoxy-2,4-dihydroisoquinoline-1,3-dione

A solution of 2-(cyanomethyl)-6-phenoxybenzonitrile (120.00 mg, 0.512 mmol, 1.00 equiv) in conc. HCl (8 mL) was stirred for 2 h at 70° C. under air atmosphere. The resulting mixture was cooled to room temperature and was concentrated under reduced pressure. The crude product was purified by Prep-HPLC with the following conditions (Column: XBridge Prep OBD C¹⁸ Column, 30×150 mm 5 um; Mobile Phase A: water (0.1% FA), Mobile Phase B: ACN; Flow rate: 60 mL/min; Gradient: 25 B to 55 B in 7 min; 220 nm; RT: 6.12 min) to afford 8-phenoxy-2,4-dihydroisoquinoline-1,3-dione (44.6 mg, 34%) as an off-white solid. ¹H NMR (400 MHz, DMSO-d₆) δ 11.06 (s, 1H), 7.61 (t, J=7.9 Hz, 1H), 7.46-7.31 (m, 2H), 7.20 (dd, J=7.6, 1.1 Hz, 1H), 7.14-7.05 (m, 1H), 6.98-6.87 (m, 3H), 3.34 (s, 2H). LC/MS (ESI, m/z): [(M+1)]⁺=254.05.

### Example 17. Synthesis of 8-(Phenylamino)-2,4-dihydroisoquinoline-1,3-dione (I-27)

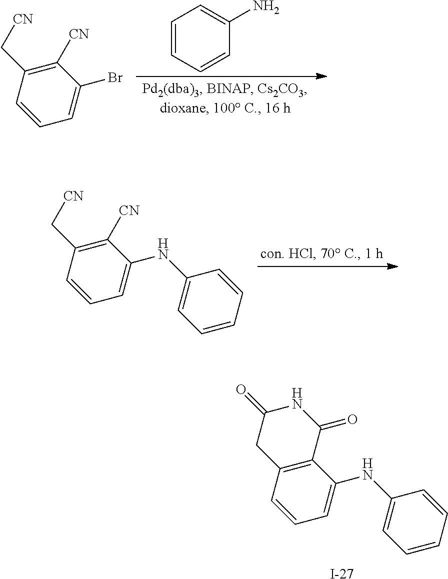

### Step 1: 2-(Cyanomethyl)-6-(phenylamino)benzonitrile

To a stirred mixture of 2-bromo-6-(cyanomethyl)benzonitrile (400.00 mg, 1.809 mmol, 1.00 equiv.), aniline (202.22 mg, 2.171 mmol, 1.20 equiv.), Cs₂CO₃ (1768.70 mg, 5.428 mmol, 3.00 equiv.), BINAP (338.02 mg, 0.543 mmol, 0.30 equiv.) in 1,4-dioxane (17.00 mL) was added Pd₂(dba)₃ (165.70 mg, 0.181 mmol, 0.10 equiv.) at room temperature under nitrogen atmosphere. The resulting mixture was stirred at 100° C. for 16 h. The mixture was allowed to cool down to room temperature and extracted with DCM (3×100 mL). The combined organic layers were washed with brine (2×100 mL), dried over anhydrous Na₂SO₄. After filtration, the filtrate was concentrated under reduced pressure. The residue was purified by silica gel column chromatography, eluted with petroleum ether:EtOAc (1:1) to afford 2-(cyanomethyl)-6-(phenylamino)benzonitrile (160 mg, 38%) as an orange solid. ¹H NMR (400 MHz, DMSO-d₆) δ 8.58 (s, 1H), 7.50 (dd, J=8.6, 7.5 Hz, 1H), 7.35-7.30 (m, 2H), 7.21-7.15 (m, 3H), 7.03 (tdd, J=7.4, 6.0, 1.0 Hz, 2H), 4.20 (s, 2H). LC/MS (ESI, m/z): [(M−1)]⁻=232.00.

### Step 2: 8-(Phenylamino)-2,4-dihydroisoquinoline-1,3-dione

A mixture of 2-(cyanomethyl)-6-(phenylamino)benzonitrile (160.00 mg, 0.686 mmol, 1.00 equiv.) in con. HCl (12 M, 5.00 mL) was stirred at 75° C. under air atmosphere for 3 h. The mixture was allowed to cool down to room temperature and was concentrated under reduced pressure. The residue was purified by reverse phase flash with the following conditions (Column: XBridge Prep OBD C¹⁸ Column, 30×150 mm 5 um; Mobile Phase A: water (0.05% FA), Mobile Phase B: ACN; Flow rate: 60 mL/min; Gradient: 33 B to 55 B in 8 min; 220 nm; RT1: 7.62 min) to afford 8-(phenylamino)-2,4-dihydroisoquinoline-1,3-dione (41 mg, 24%) as a brown yellow solid. ¹H NMR (400 MHz, DMSO-d₆) δ 11.22 (s, 1H), 10.34 (s, 1H), 7.44-7.32 (m, 3H), 7.28 (d, J=7.9 Hz, 2H), 7.13 (t, J=7.3 Hz, 1H), 7.09 (d, J=8.5 Hz, 1H), 6.66 (d, J=7.4 Hz, 1H), 4.00 (s, 2H). LC/MS (ESI, m/z): [(M−1)]⁻=250.95.

### Example 18. Synthesis of 5-bromo-2H-isoindole-1,3-dione (I-28)

5-Bromo-2H-isoindole-1,3-dione was purchased as a white solid. ¹H NMR (400 MHz, DMSO-d₆) δ 11.47 (s, 1H), 8.06-7.98 (m, 2H), 7.76 (dd, J=7.7, 0.8 Hz, 1H). LC/MS (ESI, m/z): [(M+1)]⁺=225.85.

### Example 19. Synthesis of 4-bromo-2H-isoindole-1,3-dione (I-29)

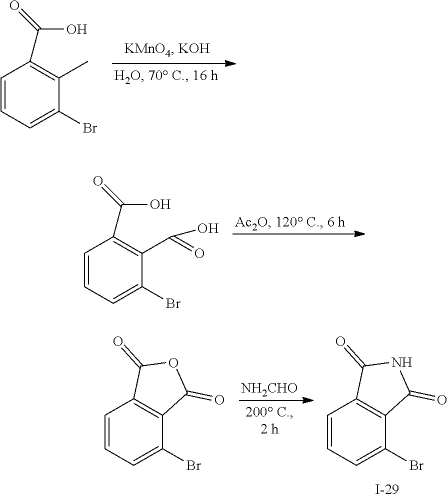

### Step 1: 3-bromobenzene-1,2-dicarboxylic acid

To a stirred solution of 3-bromo-2-methylbenzoic acid (50 g, 232.51 mmol, 1 equiv) and KOH (78.3 g, 1395.05 mmol, 6.0 equiv) in H₂O (1.5 L) was added KMnO₄ (73.5 g, 465.02 mmol, 2.0 equiv) in portions at 0° C. under nitrogen atmosphere. The resulting mixture was stirred for overnight at 70° C. under nitrogen atmosphere. The mixture was allowed to cool down to room temperature. The precipitated solids were collected by filtration and washed with water (2×30 mL). The aqueous phase was acidified to pH 2 with HCl (2 M). The combined organic layer was washed with ethyl acetate (2×500 mL), dried over anhydrous MgSO₄. After filtration, the filtrate was concentrated under reduced pressure to afford 3-bromobenzene-1,2-dicarboxylic acid (45 g, 79%) as a brown solid. ¹H NMR (400 MHz, MeOD-d₆) δ 8.04 (d, J=7.8 Hz, 1H), 7.87 (d, J=8.0 Hz, 1H), 7.45 (t, J=7.9 Hz, 1H).

### Step 2: 4-bromo-1,3-dihydro-2-benzofuran-1,3-dione

A solution of 3-bromobenzene-1,2-dicarboxylic acid (54 g, 220.38 mmol, 1 equiv) in Ac₂O (52.1 mL) was stirred for 6 h at 120° C. under nitrogen atmosphere. The mixture was allowed to cool down to room temperature. The resulting mixture was concentrated under reduced pressure. The residue was trituration with Et₂O (500 mL). The resulting mixture was filtered. The filter cake was washed with Et₂O (50 mL) and dried under reduced pressure to afford 4-bromo-1,3-dihydro-2-benzofuran-1,3-dione (45 g, 90%) as a brown solid. ¹H NMR (400 MHz, DMSO-d₆) δ 8.20 (d, J=8.0 Hz, 1H), 8.08 (d, J=7.5 Hz, 1H), 7.88 (t, J=7.7 Hz, 1H). LC/MS (ESI, m/z): [(M+1)]⁺=226.85, 228.85.

### Step 3: 4-bromo-2H-isoindole-1,3-dione

A mixture of 4-bromo-2-benzofuran-1,3-dione (500.00 mg, 2.203 mmol, 1.00 equiv) and formamide (793.63 mg, 17.620 mmol, 8.00 equiv) was stirred for 2 h at 200° C. under nitrogen atmosphere. The reaction was monitored by LCMS. The mixture was allowed to cool down to room temperature. The reaction was quenched with water/ice at 0° C. The precipitated solids were collected by filtration and washed with water (4×2 mL). The resulting solid was dried in an oven under reduced pressure. This resulted in 4-bromo-2H-isoindole-1,3-dione (443.8 mg, 89%) as a yellow solid. ¹H NMR (400 MHz, DMSO-d₆) δ 11.52 (s, 1H), 7.97 (dd, J=8.0, 0.9 Hz, 1H), 7.81 (dd, J=7.3, 0.8 Hz, 1H), 7.70 (t, J=7.7 Hz, 1H). LC/MS (ESI, m/z): [(M−1)]⁻=223.85, 225.85.

### Example 20. Synthesis of 5-(2-methylphenyl)-2H-isoindole-1,3-dione (I-30)

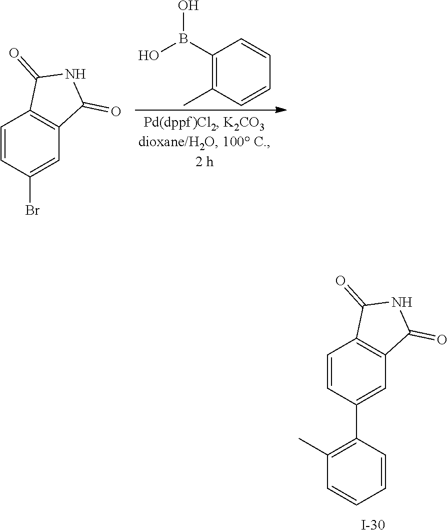

To a stirred mixture of 5-bromo-2H-isoindole-1,3-dione (100.00 mg, 0.442 mmol, 1.00 equiv) and 2-methylphenylboronic acid (66.17 mg, 0.487 mmol, 1.10 equiv) in dioxane (2.00 mL) were added K₂CO₃ (122.29 mg, 0.885 mmol, 2.00 equiv), H₂O (0.20 mL) and [1,1′-Bis(diphenylphosphino)ferrocene]dichloropalladium(II) (32.52 mg, 0.044 mmol, 0.10 equiv) at room temperature under nitrogen atmosphere. The resulting mixture was stirred for 1 h at 100° C. under nitrogen atmosphere. The reaction was monitored by LCMS. The mixture was allowed to cool down to room temperature. The resulting mixture was concentrated under reduced pressure. The residue was purified by reverse phase Flash chromatography with the following conditions: Column: WelFlash™ C18-I, 20-40 nm, 120 g; Eluent A: water (plus 10 mmol/L TFA); Eluent B: ACN; Gradient: 30%-50% B in 30 min; Flow rate: 60 mL/min; Detector: 220/254 nm; desired fractions were collected at 45% B and concentrated under reduced pressure to afford 5-(2-methylphenyl)-2H-isoindole-1,3-dione (46.6 mg, 44%) as a yellow solid. ¹H NMR (400 MHz, DMSO-d₆) δ 11.39 (s, 1H), 7.89 (d, J=7.7 Hz, 1H), 7.79 (d, J=7.8 Hz, 1H), 7.74 (s, 1H), 7.42-7.24 (m, 4H), 2.25 (s, 3H). LC/MS (ESI, m/z): [(M+1)]⁺=238.10.

### Example 21. Synthesis of 4-(2-methylphenyl)-2H-isoindole-1,3-dione (I-31)

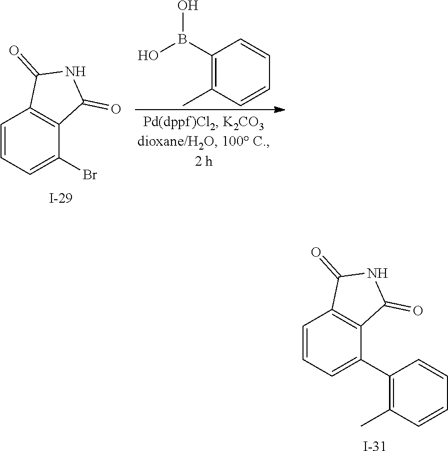

To a stirred mixture of 4-bromo-2H-isoindole-1,3-dione (100.00 mg, 0.442 mmol, 1.00 equiv) and 2-methylphenylboronic acid (66.17 mg, 0.487 mmol, 1.10 equiv) in dioxane (2 mL) were added K₂CO₃ (122.29 mg, 0.885 mmol, 2 equiv), H₂O (0.2 mL) and [1,1′-Bis(diphenylphosphino)ferrocene]dichloropalladium(II) (32.52 mg, 0.044 mmol, 0.10 equiv) in portions at room temperature under air atmosphere. The resulting mixture was stirred for 1 h at 100° C. under nitrogen atmosphere. The reaction was monitored by LCMS. The mixture was allowed to cool down to room temperature. The resulting mixture was concentrated under reduced pressure. The residue was purified by reverse phase Flash chromatography with the following conditions: Column: WelFlash™ C18-I, 20-40 nm, 120 g; Eluent A: water (plus 10 mmol/L TFA); Eluent B: ACN; Gradient: 30%-50% B in 30 min; Flow rate: 60 mL/min; Detector: 220/254 nm; desired fractions were collected at 45% B and concentrated under reduced pressure to afford 4-(2-methylphenyl)-2H-isoindole-1,3-dione (49.7 mg, 47%) as a yellow solid. ¹H NMR (400 MHz, DMSO-d₆) δ 11.27 (s, 1H), 7.91-7.80 (m, 2H), 7.58 (dd, J=6.3, 2.3 Hz, 1H), 7.37-7.28 (m, 2H), 7.25 (td, J=7.0, 2.0 Hz, 1H), 7.18 (dd, J=7.4, 1.4 Hz, 1H), 2.07 (s, 3H). LC/MS (ESI, m/z): [(M+1)]⁺=238.10.

### Example 22. Synthesis of 5-(4-methyl-1,3-oxazol-5-yl)-2H-isoindole-1,3-dione (I-32)

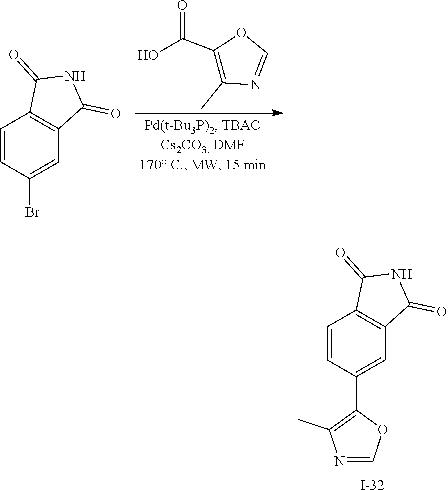

To a stirred mixture of 5-bromo-2H-isoindole-1,3-dione (50.00 mg, 0.221 mmol, 1.00 equiv), Cs₂CO₃ (108.11 mg, 0.332 mmol, 1.50 equiv) and 4-methyl-1,3-oxazole-5-carboxylic acid (56.23 mg, 0.442 mmol, 2.00 equiv) in DMF (4.00 mL) were added Pd(P(t-Bu)₃)₂ (5.65 mg, 0.011 mmol, 0.05 equiv) and tetrabutyl ammonium chloride (61.48 mg, 0.221 mmol, 1.00 equiv) in one portions at room temperature under nitrogen atmosphere. The final reaction mixture was irradiated with microwave radiation for 15 min at 170° C. The reaction was monitored by LCMS. The mixture was allowed to cool down to room temperature. The residue was purified by reverse phase Flash chromatography with the following conditions: Column: WelFlash™ C18-I, 20-40 nm, 120 g; Eluent A: water (plus 10 mmol/L TFA); Eluent B: ACN; Gradient: 25%-45% B in 25 min; Flow rate: 60 mL/min; Detector: 220/254 nm; desired fractions were collected at 38% B and concentrated under reduced pressure to afford 5-(4-methyl-1,3-oxazol-5-yl)-2H-isoindole-1,3-dione (25 mg, 50%) as an off-white solid. ¹H NMR (400 MHz, DMSO-d₆) δ 8.49 (s, 1H), 8.07-8.03 (m, 1H), 7.94 (d, J=7.6 Hz, 2H), 2.46 (s, 3H). LC/MS (ESI, m/z): [(M+1)]⁺=229.15.

### Example 23. Synthesis of 4-(4-methyl-1,3-oxazol-5-yl)-2H-isoindole-1,3-dione (I-33)

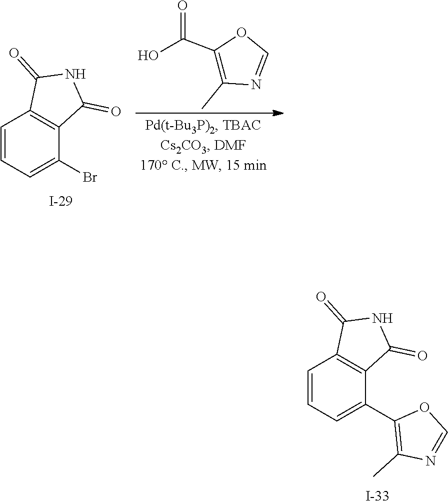

To a stirred mixture of 4-bromo-2H-isoindole-1,3-dione (50.00 mg, 0.221 mmol, 1.00 equiv), Cs₂CO₃ (108.11 mg, 0.332 mmol, 1.5 equiv) and 4-methyl-1,3-oxazole-5-carboxylic acid (56.23 mg, 0.442 mmol, 2 equiv) in DMF (4.00 mL) were added Pd(P(t-Bu)₃)₂ (5.65 mg, 0.011 mmol, 0.05 equiv) and tetrabutyl ammonium chloride (61.48 mg, 0.221 mmol, 1 equiv) in one portions at room temperature under nitrogen atmosphere. The final reaction mixture was irradiated with microwave radiation for 15 min at 170° C. The reaction was monitored by LCMS. The mixture was allowed to cool down to room temperature. The residue was purified by reverse phase Flash chromatography with the following conditions: Column: WelFlash™ C18-I, 20-40 m, 120 g; Eluent A: water (plus 10 mmol/L TFA); Eluent B: ACN; Gradient: 25%-45% B in 25 min; Flow rate: 60 mL/min; Detector: 220/254 nm; desired fractions were collected at 38% B and concentrated under reduced pressure to afford 4-(4-methyl-1,3-oxazol-5-yl)-2H-isoindole-1,3-dione (26 mg, 52%) as an off-white solid. ¹H NMR (400 MHz, DMSO-d₆) δ 11.43 (s, 1H), 8.46 (s, 1H), 7.95-7.84 (m, 3H), 2.13 (s, 3H). LC/MS (ESI, m/z): [(M+18)]⁺=229.05.

### Example 24. 2-(2-methylphenyl)-5H,7H-pyrazolo[4,3-c]pyridine-4,6-dione (I-35)

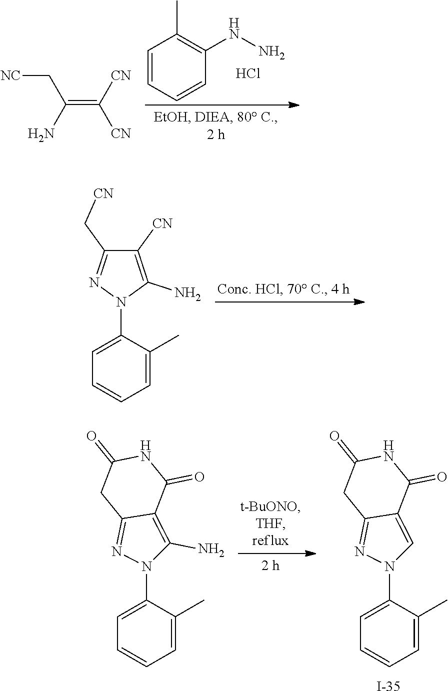

### Step 1: 5-amino-3-(cyanomethyl)-1-(2-methylphenyl)pyrazole-4-carbonitrile

To a stirred solution of (2-methylphenyl)hydrazine hydrochloride (1.20 g, 7.565 mmol, 1.00 equiv) in EtOH (10.00 mL) were added DIEA (1.96 g, 15.137 mmol, 2 equiv) and tricyanoaminopropene (1.00 g, 7.569 mmol, 1.00 equiv) at room temperature under nitrogen atmosphere. The resulting mixture was stirred for 2 h at 80° C. under nitrogen atmosphere. The resulting mixture was cooled down to room temperature and was concentrated under reduced pressure. The residue was purified by reverse phase flash chromatography with the following conditions: Column: WelFlash™ C18-I, 20-40 um, 330 g; Eluent A: water (plus 10 mmol/L FA); Eluent B: ACN; Gradient: 30%-50% B in 20 min; Flow rate: 80 mL/min; Detector: 220/254 nm; desired fractions were collected at 40% B and concentrated under reduced pressure to afford 5-amino-3-(cyanomethyl)-1-(2-methylphenyl)pyrazole-4-carbonitrile (700 mg, 39%) as a brown solid. ¹H NMR (400 MHz, DMSO-d₆) δ 7.48-7.39 (m, 2H), 7.39-7.26 (m, 2H), 6.67 (s, 2H), 4.07 (s, 2H), 2.06 (s, 3H); LC/MS (ESI, m/z): [(M+18)]⁺=238.10.

### Step 2: 3-amino-2-(2-methylphenyl)-5H,7H-pyrazolo[4,3-c]pyridine-4,6-dione

A stirred solution of 5-amino-3-(cyanomethyl)-1-(2-methylphenyl)pyrazole-4-carbonitrile (200.00 mg, 0.843 mmol, 1.00 equiv) in con. HCl (5.00 mL) was stirred for 2 h at 80° C. under nitrogen atmosphere. The resulting mixture was cooled down to room temperature and was diluted with water (20 mL). The mixture was neutralized to pH 7 with saturated aq. Na₂CO₃. The resulting mixture was extracted with EtOAc (3×50 mL). The combined organic layers were washed with brine (50 mL) and dried over anhydrous Na₂SO₄. After filtration, the filtrate was concentrated under reduced pressure. The residue was purified by reverse phase flash chromatography with the following conditions: Column: WelFlash™ C18-I, 20-40 um, 120 g; Eluent A: water (plus 10 mmol/L FA); Eluent B: ACN; Gradient: 20%-40% B in 20 min; Flow rate: 80 mL/min; Detector: 220/254 nm; desired fractions were collected at 28% B and concentrated under reduced pressure to afford 3-amino-2-(2-methylphenyl)-5H,7H-pyrazolo[4,3-c]pyridine-4,6-dione (211 mg, 98%) as a brown solid. ¹H NMR (400 MHz, DMSO-d₆) δ 10.56 (s, 1H), 7.48-7.41 (m, 2H), 7.41-7.32 (m, 1H), 7.32-7.25 (m, 1H), 6.24 (s, 2H), 3.76 (s, 2H), 2.10 (s, 3H). LC/MS (ESI, m/z): [(M+18)]⁺=257.10.

### Step 3: 2-(2-methylphenyl)-5H,7H-pyrazolo[4,3-c]pyridine-4,6-dione

To a stirred solution of 3-amino-2-(2-methylphenyl)-5H,7H-pyrazolo[4,3-c]pyridine-4,6-dione (100.00 mg, 0.390 mmol, 1.00 equiv) in THE (3.00 mL) was added t-BuNO₂ (80.48 mg, 0.780 mmol, 2.00 equiv) at room temperature under nitrogen atmosphere. The resulting mixture was stirred for 2 h at 70° C. under nitrogen atmosphere. The resulting mixture was concentrated under reduced pressure. The crude product was purified by Prep-HPLC with the following conditions (Column: SunFire Prep C¹⁸ OBD Column, 19×150 mm 5 um; Mobile Phase A: water (0.05% FA), Mobile Phase B: ACN; Flow rate: 25 mL/min; Gradient: 13 B to 49 B in 11 min; 220 nm; RT1: 9.1 min) to afford 2-(2-methylphenyl)-5H,7H-pyrazolo[4,3-c]pyridine-4,6-dione (17.6 mg) as a light yellow solid. ¹H NMR (400 MHz, DMSO-d₆) δ 11.01 (s, 1H), 8.76 (s, 1H), 7.47-7.33 (m, 4H), 3.97 (s, 2H), 2.23 (s, 3H). LC/MS (ESI, m/z): [(M+18)]⁺=242.05.

### Example 25. Synthesis of 2-(1,3-Dioxo-2,4-dihydroisoquinolin-8-yl)benzonitrile (I-52)

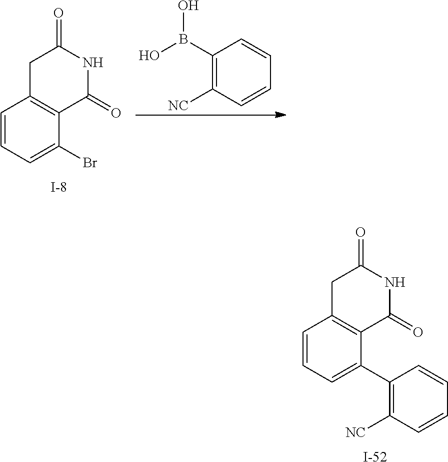

To a mixture of 8-bromo-2,4-dihydroisoquinoline-1,3-dione (478.00 mg, 1.991 mmol, 1.00 equiv.), 2-cyanophenylboronic acid (321.85 mg, 2.190 mmol, 1.10 equiv.) and NaHCO₃ (836.37 mg, 9.956 mmol, 5 equiv.) in dioxane (15.00 mL) and H₂O (3.00 mL) was added Pd(PPh₃)₄ (230.10 mg, 0.199 mmol, 0.1 equiv.) at room temperature under nitrogen atmosphere. The above mixture was stirred at 100° C. under nitrogen atmosphere for 16 h. The resulting mixture was allowed to cool down to the room temperature and was extracted with DCM (2×50 mL). The combined organic layers were washed with brine (2×30 mL), dried with anhydrous Na₂SO₄ and filtered. The filtrate was concentrated under reduced pressure. The residue was purified by reverse phase flash with the following conditions: (Column: YMC-Actus Triart C¹⁸, 30×250 mm, 5 um; Mobile Phase A: water (0.1% FA), Mobile Phase B: ACN; Flow rate: 50 mL/min; Gradient: 30 B to 53 B in 8 min; 220 nm; RT1: 6.82 min) to afford 2-(1,3-dioxo-2,4-dihydroisoquinolin-8-yl)benzonitrile (9.1 mg, 2%) as a white solid. ¹H NMR (400 MHz, DMSO-d₆) δ 11.19 (s, 1H), 7.85 (dd, J=7.7, 1.3 Hz, 1H), 7.80-7.66 (m, 2H), 7.59-7.44 (m, 2H), 7.39 (dd, J=7.9, 1.2 Hz, 1H), 7.25 (dd, J=7.6, 1.2 Hz, 1H), 4.17 (d, J=6.4 Hz, 2H). LC/MS (ESI, m/z): [(M+1)]⁺=263.10.

### Example 26. Synthesis of 6-Bromo-3H-1,3-benzoxazine-2,4-dione (I-53)

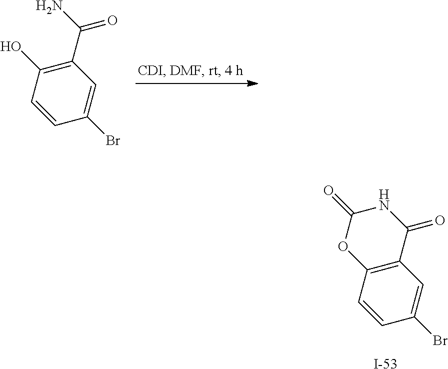

A mixture of 5-bromo-2-hydroxybenzamide (100.00 mg, 0.463 mmol, 1.00 equiv.) and CDI (112.59 mg, 0.694 mmol, 1.50 equiv.) in DMF (2.00 mL) was stirred at room temperature under air atmosphere for 4 h. The reaction mixture was purified by reverse phase flash with the following conditions: (Column: Xselect CSH OBD Column 30×150 mm 5 um; Mobile Phase A: water (0.1% FA), Mobile Phase B: ACN; Flow rate: 60 mL/min; Gradient: 20 B to 40 B in 7 min; 220 nm; RT1: 6.43 min) to afford 6-bromo-3H-1,3-benzoxazine-2,4-dione (72 mg, 64%) as a white solid. ¹H NMR (400 MHz, DMSO-d₆) δ 12.21 (s, 1H), 8.00 (d, J=2.5 Hz, 1H), 7.95 (dd, J=8.8, 2.6 Hz, 1H), 7.40 (d, J=8.7 Hz, 1H). LC/MS (ESI, m/z): [(M−1)]⁻=239.85.

### Example 27. Synthesis of 5-Bromo-3H-1,3-benzoxazine-2,4-dione (I-54)

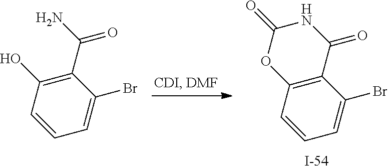

A mixture of 2-bromo-6-hydroxybenzamide (100.00 mg, 0.463 mmol, 1.00 equiv.) and CDI (112.59 mg, 0.694 mmol, 1.50 equiv.) in DMF (2.00 mL) was stirred at room temperature under air atmosphere for 4 h. The reaction mixture was purified by reverse phase flash with the following conditions: (Column: XBridge Prep OBD C¹⁸ Column, 30×150 mm 5 um; Mobile Phase A: water (0.1% FA), Mobile Phase B: ACN; Flow rate: 60 mL/min; Gradient: 17 B to 42 B in 8 min; 254/220 nm; RT1: 7.5 min) to afford 5-bromo-3H-1,3-benzoxazine-2,4-dione (64.8 mg, 58%) as a white solid. ¹H NMR (400 MHz, DMSO-d₆) δ 12.07 (s, 1H), 7.66 (dd, J=8.0, 1.8 Hz, 1H), 7.62 (t, J=7.8 Hz, 1H), 7.41 (dd, J=7.6, 1.8 Hz, 1H). LC/MS (ESI, m/z): [(M+1)]⁺=242.00.

### Example 28. Synthesis of 6-Bromo-3H-1,3-benzoxazine-2,4-dione (I-55)

6-Bromo-1,3-dihydroquinazoline-2,4-dione was purchased as a white solid. ¹H NMR (400 MHz, DMSO-d₆) δ 11.44 (s, 1H), 11.30 (s, 1H), 7.94 (dt, J=5.1, 2.4 Hz, 1H), 7.79 (ddt, J=9.0, 4.2, 2.4 Hz, 1H), 7.13 (d, J=8.7 Hz, 1H). LC/MS (ESI, m/z): [(M+1)]⁺=241.10.

### Example 29. Synthesis of 5-Bromo-1,3-dihydroquinazoline-2,4-dione (I-56)

5-Bromo-1,3-dihydroquinazoline-2,4-dione was purchased as a white solid. ¹H NMR (400 MHz, DMSO-d₆) δ 11.28 (d, J=15.3 Hz, 2H), 7.50-7.37 (m, 2H), 7.17 (dd, J=8.0, 1.4 Hz, 1H). LC/MS (ESI, m/z): [(M−1)]⁻=238.95.

### Example 30. Synthesis of 8′-Bromo-2′H-spiro[cyclopropane-1,4′-isoquinoline]-1′,3′-dione

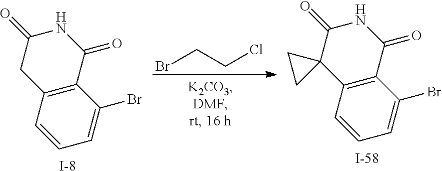

### 8′-Bromo-2′H-spiro[cyclopropane-1,4′-isoquinoline]-1′3′-dione

A mixture of 8-bromo-2,4-dihydroisoquinoline-1,3-dione (300.00 mg, 1.250 mmol, 1.00 equiv.), 1-bromo-2-chloroethane (1792.21 mg, 12.497 mmol, 10.00 equiv.) and K₂CO₃ (345.43 mg, 2.499 mmol, 2 equiv.) in DMF (15.00 mL) was stirred at room temperature for 16 h. The reaction mixture was purified by reverse phase flash with the following conditions: (Column: Xselect CSH OBD Column 30×150 mm 5 um; Mobile Phase A: water (0.1% FA), Mobile Phase B: ACN; Flow rate: 60 mL/min; Gradient: 20 B to 40 B in 7 min; 220 nm; RT 1: 6.43 min) to afford 8′-bromo-2′H-spiro[cyclopropane-1,4′-isoquinoline]-1′,3′-dione (19.8 mg, 6%) as a beige solid. ¹H NMR (400 MHz, DMSO-d₆) δ 11.52 (s, 1H), 7.70 (dd, J=7.9, 1.0 Hz, 1H), 7.47 (t, J=8.0 Hz, 1H), 7.08 (dd, J=8.1, 1.0 Hz, 1H), 1.92 (q, J=3.9 Hz, 2H), 1.71 (q, J=4.0 Hz, 2H). LC/MS (ESI, m/z): [(M+1)]⁺=266.00.

### Example 31. Synthesis of 5-bromo-1-methyl-3H-quinazoline-2,4-dione (I-59)

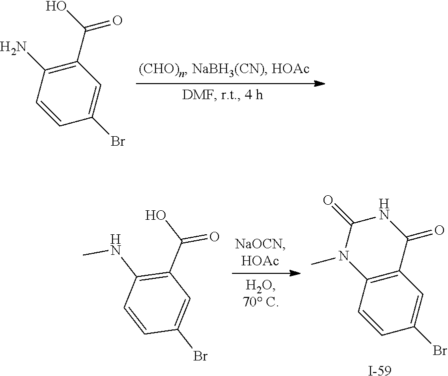

### Step 1: 5-bromo-2-(methylamino)benzoic acid

To a stirred mixture of 5-bromoanthranilic acid (2.00 g, 9.258 mmol, 1.00 equiv) and (HCHO)ₙ (0.83 g, 27.773 mmol, 3 equiv) in DMF (20.00 mL) were added HOAc (5.56 g, 92.578 mmol, 10 equiv) and NaBH₃CN (1.75 g, 27.773 mmol, 3 equiv) in portions at room temperature under air atmosphere. The resulting mixture was stirred for 6 h at room temperature under air atmosphere. The reaction was monitored by LCMS. The reaction was quenched by the addition of water (2 mL) at room temperature. The resulting mixture was concentrated under reduced pressure. The residue was purified by reverse phase Flash chromatography with the following conditions: Column: WelFlash™ C18-I, 20-40 um, 120 g; Eluent A: water (plus 10 mmol/L FA); Eluent B: ACN; Gradient: 25%-45% B in 25 min; Flow rate: 60 mL/min; Detector: 220/254 nm; desired fractions were collected at 42% B and concentrated under reduced pressure to afford 5-bromo-2-(methylamino)benzoic acid (284 mg, 13%) as a white solid. ¹H NMR (400 MHz, DMSO-d₆) δ 7.84 (d, J=2.6 Hz, 1H), 7.51 (dd, J=9.0, 2.6 Hz, 1H), 6.68 (d, J=9.0 Hz, 1H), 2.83 (s, 3H). LC/MS (ESI, m/z): [(M+18)]⁺=290.90, 292.90.

### Step 2: 2-(cyanomethyl)-5-hydroxybenzonitrile

To a stirred mixture of 5-bromo-2-(methylamino) benzoic acid (280.00 mg, 1.217 mmol, 1.00 equiv) in AcOH/H₂O (16.00 mL, 1/50) at room temperature under air atmosphere was added NaOCN (112.00 mg, 1.723 mmol, 1.42 equiv) at room temperature. The resulting mixture was stirred for additional 30 min at 40° C. To the above mixture was added NaOH (1.38 g, 0.035 mmol, 0.03 equiv) in portions over 10 min at 40° C. The resulting mixture was stirred for additional 1 h at 70° C. The reaction was monitored by LCMS. The resulting mixture was cooled down to room temperature and was diluted with water (50 mL). The mixture was acidified to pH 6 with aq. HCl (1 M). The resulting mixture was extracted with EtOAc (30 mL), dried over anhydrous Na₂SO₄. After filtration, the filtrate was concentrated under reduced pressure. The residue was purified by reverse phase Flash chromatography with the following conditions: Column: WelFlash™ C18-I, 20-40 um, 120 g; Eluent A: water (plus 10 mmol/L NH₄HCO₃); Eluent B: ACN; Gradient: 30%-50% B in 30 min; Flow rate: 60 mL/min; Detector: 220/254 nm; desired fractions were collected at 42% B and concentrated under reduced pressure to afford 6-bromo-1-methyl-3H-quinazoline-2,4-dione (50 mg, 16%) as a yellow solid. ¹H NMR (400 MHz, DMSO-d₆) δ 11.71 (s, 1H), 8.05 (d, J=2.5 Hz, 1H), 7.92 (dd, J=8.9, 2.5 Hz, 1H), 7.41 (d, J=9.0 Hz, 1H), 3.43 (s, 3H). LC/MS (ESI, m/z): [(M+18)]⁺=254.75, 256.75.

### Example 32. Synthesis of 5-bromo-1-methyl-3H-quinazoline-2,4-dione (I-60)

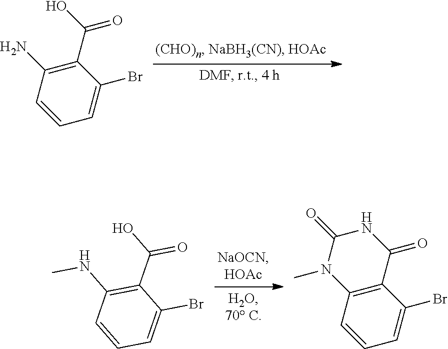

### Step 1: 2-bromo-6-(methylamino)benzoic acid

To a stirred mixture of 2-amino-6-bromobenzoic acid (2.00 g, 9.258 mmol, 1.00 equiv) and (HCHO)ₙ (0.83 g, 27.667 mmol, 2.99 equiv) in DMF (20.00 mL) were added HOAc (5.56 g, 92.586 mmol, 10.00 equiv) and NaBH₃CN (1.75 g, 0.028 mmol, 3 equiv) at room temperature under air atmosphere. The resulting mixture was stirred for 6 h at room temperature under air atmosphere. The reaction was monitored by LCMS. The reaction was quenched by the addition of water (2 mL) at room temperature. The resulting mixture was concentrated under reduced pressure. The residue was purified by reverse phase Flash chromatography with the following conditions: Column: WelFlash™ C18-I, 20-40 m, 120 g; Eluent A: water (plus 10 mmol/L FA); Eluent B: ACN; Gradient: 25%-45% B in 25 min; Flow rate: 60 mL/min; Detector: 220/254 nm; desired fractions were collected at 42% B and concentrated under reduced pressure to afford 2-bromo-6-(methylamino)benzoic acid (230 mg, 11%) as a yellow solid. ¹H NMR (400 MHz, DMSO-d₆) δ 7.13 (t, J=8.1 Hz, 1H), 6.81 (d, J=7.8 Hz, 1H), 6.60 (d, J=8.4 Hz, 1H), 2.73 (d, J=7.8 Hz, 3H). LC/MS (ESI, m/z): [(M+1)]⁺=229.95, 231.95.

### Step 2: 5-bromo-1-methyl-3H-quinazoline-2,4-dione

To a stirred mixture of 2-bromo-6-(methylamino)benzoic acid (200.00 mg, 0.869 mmol, 1.00 equiv) in AcOH/H₂O (11.50 mL, 1/50) was added NaOCN (100.00 mg, 1.538 mmol, 1.77 equiv) dropwise at room temperature. The resulting mixture was stirred for additional 30 min at 40° C. To the above mixture was added NaOH (1.00 g, 0.025 mmol, 0.03 equiv) in portions over 10 min at 40° C. The resulting mixture was stirred for additional 1 h at 70° C. The reaction was monitored by LCMS. The mixture was allowed to cool down to room temperature. The resulting mixture was diluted with water (50 mL). The mixture was acidified to pH 6 with aq. HCl (2 M). The resulting mixture was extracted with EtOAc (30 mL), dried over anhydrous Na₂SO₄. After filtration, the filtrate was concentrated under reduced pressure. The residue was purified by reverse phase Flash chromatography with the following conditions: Column: WelFlash™ C18-I, 20-40 um, 120 g; Eluent A: water (plus 10 mmol/L NH₄HCO₃); Eluent B: ACN; Gradient: 30%-50% B in 30 min; Flow rate: 60 mL/min; Detector: 220/254 nm; desired fractions were collected at 42% B and concentrated under reduced pressure to afford 5-bromo-1-methyl-3H-quinazoline-2,4-dione (25 mg, 11%) as a yellow solid. ¹H NMR (400 MHz, DMSO-d₆) δ 11.56 (s, 1H), 7.62-7.56 (m, 1H), 7.53 (dd, J=7.9, 1.2 Hz, 1H), 7.45 (dd, J=8.3, 1.3 Hz, 1H), 3.44 (s, 3H). LC/MS (ESI, m/z): [(M+1)]⁺=254.95, 256.95.

### Example 33. Synthesis of 1H-spiro[indole-2,3′-pyrrolidine]-2′,3,5′-trione (I-195)

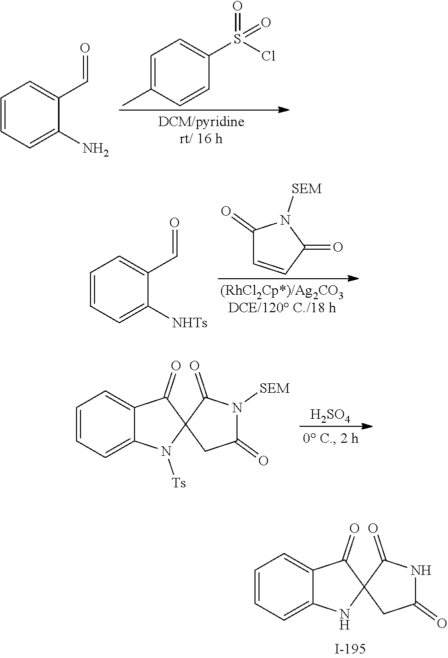

### Step 1: N-(2-formylphenyl)-4-methylbenzenesulfonamide

To a stirred solution of 2-aminobenzaldehyde (5.00 g, 41.28 mmol) and Pyridine (7.18 g, 90.81 mmol) in DCM (150 mL) was added p-toluenesulfonyl chloride (8.66 g, 45.40 mmol) at 0° C. under nitrogen atmosphere. The solution was stirred for 16 h at room temperature. The reaction was monitored by LCMS. The reaction was quenched by the addition of water/Ice (100 mL) at 0° C. The resulting mixture was extracted with DCM (2×80 mL). The combined organic layers were washed with brine (100 mL), dried over anhydrous Na₂SO₄. After filtration, the filtrate was concentrated under reduced pressure. The residue was purified by silica gel column chromatography, eluted with Petroleum ether/EtOAc (3:1) to afford N-(2-formylphenyl)-4-methylbenzenesulfonamide (3.7 g, 33%) as a white solid. ¹H NMR (400 MHz, DMSO-d₆) δ 10.56 (s, 1H), 10.01 (s, 1H), 7.82 (dd, J=7.7, 1.7 Hz, 1H), 7.65-7.54 (m, 3H), 7.39-7.29 (m, 3H), 7.19 (dd, J=8.2, 1.1 Hz, 1H), 2.35 (s, 3H). LC/MS (ESI, m/z): [(M−18)]⁻=274.35.

### Step 2: 1-(4-methylbenzenesulfonyl)-1′-[[2-(trimethylsilyl)ethoxy]methyl]spiro[indole-2,3′-pyrrolidine]-2′,3,5′-trione

To a stirred mixture of N-(2-formylphenyl)-4-methylbenzenesulfonamide (3.00 g, 10.90 mmol), Ag₂CO₃ (3.00 g, 10.90 mmol) and 1-[[2-(trimethylsilyl)ethoxy]methyl]pyrrole-2,5-dione (4.95 g, 21.79 mmol) in DCE (75 mL) was added (Cp*RhCl₂)₂ (168.37 mg, 0.27 mmol) at room temperature under nitrogen atmosphere. The mixture was allowed to react for 18 h at 120° C. The reaction was monitored by LCMS. The mixture was allowed to cool down to room temperature. The resulting mixture was filtered, the filter cake was washed with CH₂Cl₂ (2×10 mL). The filtrate was concentrated under reduced pressure. The residue was purified by silica gel column chromatography, eluted with Petroleum ether/EtOAc (3:1) to afford 1-(4-methylbenzenesulfonyl)-1-[[2-(trimethylsilyl)ethoxy]methyl]spiro[indole-2,3-pyrrolidine]-2,3,5-trione (1.7 g, 31%) as an off-white solid. ¹H NMR (400 MHz, DMSO-d₆) δ 8.06-7.99 (m, 2H), 7.85-7.76 (m, 2H), 7.54 (dd, J=8.2, 1.0 Hz, 1H), 7.49 (d, J=8.1 Hz, 2H), 7.31 (t, J=7.5 Hz, 1H), 5.03-4.92 (m, 2H), 3.63-3.53 (m, 2H), 3.48 (d, J=15.0 Hz, 2H), 2.41 (s, 3H), 0.88 (t, J=8.2 Hz, 2H), −0.02 (s, 9H). LC/MS (ESI, m/z): [(M−1)]⁻=499.00.

### Step 3: 1H-spiro[indole-2,3′-pyrrolidine]-2′,3,5′-trione

A solution of 1-(4-methylbenzenesulfonyl)-1′-[[2-(trimethylsilyl)ethoxy]methyl]spiro[indole-2,3′-pyrrolidine]-2′,3,5′-trione (1.00 g, 2.00 mmol) in conc. H₂SO₄ (5.00 mL) was stirred for 2 h at 0° C. under nitrogen atmosphere. The reaction was monitored by LCMS. The resulting solution was poured in ice/water (50 mL). The resulting mixture was extracted with EtOAc (3×30 mL). The combined organic layers were dried over anhydrous Na₂SO₄. After filtration, the filtrate was concentrated under reduced pressure to give 1H-spiro[indole-2,3′-pyrrolidine]-2′,3,5′-trione (300 mg, 69%) as a light yellow solid. ¹H NMR (400 MHz, DMSO-d₆) δ 11.74 (s, 1H), 7.62 (s, 1H), 7.55 (ddd, J=8.4, 6.9, 1.4 Hz, 1H), 7.47 (d, J=8.0 Hz, 1H), 7.06 (d, J=8.3 Hz, 1H), 6.82 (t, J=7.4 Hz, 1H), 2.98 (d, J=17.8 Hz, 1H), 2.87 (d, J=17.8 Hz, 1H). LC/MS (ESI, m/z): [(M−1)]⁻=214.90.

### Example 34. Synthesis of 8-Amino-4,4-difluoro-2H-isoquinoline-1,3-dione (I-71)

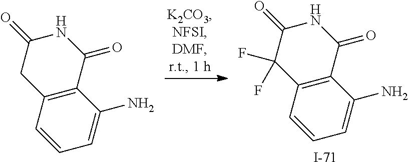

A mixture of 8-amino-2,4-dihydroisoquinoline-1,3-dione (160.00 mg, 0.908 mmol, 1.00 equiv) and K₂CO₃ (251.03 mg, 1.816 mmol, 2.00 equiv) in DMF (10.00 mL) was added NFSI (1.43 g, 4.535 mmol, 4.99 equiv) at room temperature under air atmosphere. The above mixture was stirred for 4 h at room temperature. The reaction mixture was quenched by the addition of H₂O (20 mL) and extracted with DCM (3×100 mL). The filtrate was concentrated under reduced pressure to give the residue, which was purified by reverse phase flash with the following conditions (Column: XBridge Shield RP18 OBD Column, 30×150 mm, 5 um; Mobile Phase A: water (0.05% TFA), Mobile Phase B: ACN; Flow rate: 60 mL/min; Gradient: 17 B to 38 B in 9 min; 220 nm; RT: 7.92 min) to afford 8-amino-4,4-difluoro-2H-isoquinoline-1,3-dione (61.3 mg, 32%) as a brown yellow solid. ¹H NMR (400 MHz, DMSO-d₆) δ 11.91 (s, 1H), 7.48 (dd, J=18.9, 10.7 Hz, 3H), 7.06 (d, J=8.6 Hz, 1H), 6.98 (d, J=7.2 Hz, 1H); LC/MS (ESI, m/z): [(M−1)]⁻=210.95.

### Example 35. Synthesis of 5-Fluoro-2,4-dihydroisoquinoline-1,3-dione (I-72)

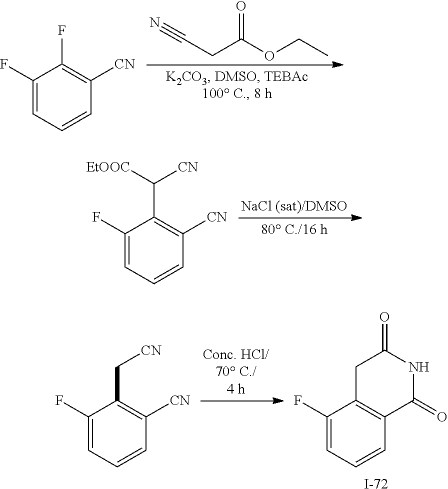

### Step 1: Ethyl 2-cyano-2-(2-cyano-6-fluorophenyl)acetate

A mixture of 2,3-difluorobenzonitrile (2.00 g, 14.378 mmol, 1.00 equiv), ethyl cyanoacetate (1.63 g, 14.378 mmol, 1.00 equiv), K₂CO₃ (5.96 g, 43.133 mmol, 3.00 equiv) and TEBAc (65.56 mg, 0.288 mmol, 0.02 equiv) in DMSO (10.00 mL) was stirred at 120° C. for 16 h. The mixture was allowed to cool down to room temperature. The resulting mixture was acidified to pH 6 with HCl (2 N aq.) and was extracted with EtOAc (3×100 mL). The combined organic layers were washed with brine (2×100 mL), dried over anhydrous Na₂SO₄. After filtration, the filtrate was concentrated under reduced pressure to give the residue, which was purified by reverse phase flash with the following conditions (Column: Spherical C¹⁸, 20-40 um, 330 g; Mobile Phase A: water (10 mM HCOOH), Mobile Phase B: ACN; Flow rate: 80 mL/min; Gradient (B %): 40%˜60%; 20 min; Detector: 254 nm; Rt: 6 min.) to afford ethyl 2-cyano-2-(2-cyano-6-fluorophenyl)acetate (1.8 g, 54%) as a yellow oil. ¹H NMR (400 MHz, DMSO-d₆) δ 8.07-7.56 (m, 3H), 6.16 (s, 1H), 4.27 (q, J=7.2 Hz, 2H), 1.21 (t, J=7.2 Hz, 3H); LC/MS (ESI, m/z): [(M−1)]⁻=230.95.

### Step 2: 2-(Cyanomethyl)-3-fluorobenzonitrile

To a stirred solution of ethyl 2-cyano-2-(2-cyano-6-fluorophenyl)acetate (1.80 g, 7.751 mmol, 1.00 equiv) in DMSO (10.00 mL) was added saturated NaCl (10.00 mL) at room temperature. The solution was stirred for 16 h at 110° C. The mixture was allowed to cool down to room temperature. The resulting mixture was concentrated under reduced pressure and the residue was purified by reverse phase flash chromatography with the following conditions: Column: WelFlash™ C18-I, 20-40 um, 330 g; Eluent A: water (plus 10 mmol/L FA); Eluent B: ACN; Gradient: 30%-50% B in 20 min; Flow rate: 80 mL/min; Detector: 220/254 nm; desired fractions were collected at 54% B and concentrated under reduced pressure to afford 2-(cyanomethyl)-3-fluorobenzonitrile (1.1 g, 89%) as a yellow solid. ¹H NMR (400 MHz, DMSO-d₆) δ 7.83 (dd, J=7.5, 1.3 Hz, 1H), 7.77-7.60 (m, 2H), 4.22 (d, J=1.2 Hz, 2H); LC/MS (ESI, m/z): [(M−1)]⁻=158.95.

### Step 3: 5-Fluoro-2,4-dihydroisoquinoline-1,3-dione

To a stirred solution of 2-(cyanomethyl)-3-fluorobenzonitrile (100.00 mg, 1 equiv) in conc. HCl (100.00 mL) was stirred for 2 h at 70° C. The mixture was allowed to cool down to room temperature. The resulting mixture was filtered. The filter cake was washed with water (3×100 mL) and dried to afford 5-fluoro-2,4-dihydroisoquinoline-1,3-dione (26.1 mg, 23%) as a yellow solid. ¹H NMR (400 MHz, DMSO-d₆) δ 11.45 (s, 1H), 7.88 (dd, J=7.3, 1.7 Hz, 1H), 7.60-7.47 (m, 2H), 3.94 (s, 2H); LC/MS (ESI, m/z): [(M+1)]⁺=180.15.

### Example 36. Synthesis of 6-Fluoro-2,4-dihydroisoquinoline-1,3-dione (I-73)

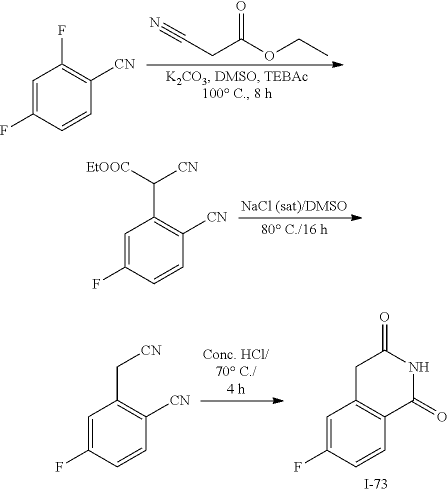

### Step 1: Ethyl 2-cyano-2-(2-cyano-5-fluorophenyl)acetate

A mixture of 2,4-difluorobenzonitrile (2.00 g, 14.378 mmol, 1.00 equiv), ethyl cyanoacetate (1.63 g, 14.378 mmol, 1.00 equiv), K₂CO₃ (5.96 g, 43.133 mmol, 3.00 equiv) and TEBAc (65.56 mg, 0.288 mmol, 0.02 equiv) in DMSO (10.00 mL) were stirred at 100° C. for 16 h. The mixture was allowed to cool down to room temperature. The resulting mixture was acidified to pH 6 with HCl (2 N aq.) and was extracted with EtOAc (3×100 mL). The combined organic layers were washed with brine (100 mL), dried over anhydrous Na₂SO₄. After filtration, the filtrate was concentrated under reduced pressure to give the residue, which was purified by reverse phase flash with the following conditions (Column: Spherical C¹⁸, 20˜40 um, 330 g; Mobile Phase A: water (10 mM HCOOH), Mobile Phase B: ACN; Flow rate: 80 mL/min; Gradient (B %): 40%˜60%; 20 min; Detector: 254 nm; Rt: 6 min.) to afford ethyl 2-cyano-2-(2-cyano-5-fluorophenyl)acetate (2.4 g, 72%) as an orange oil. ¹H NMR (400 MHz, DMSO-d₆) δ 8.14 (dd, J=8.4, 5.4 Hz, 1H), 7.73-7.40 (m, 2H), 6.10 (s, 1H), 4.47 (s, 1H), 4.27 (q, J=7.1 Hz, 1H), 1.21 (dt, J=14.7, 7.1 Hz, 3H); LC/MS (ESI, m z): [(M−1)]⁻=230.95.

### Step 2: 2-(Cyanomethyl)-4-fluorobenzonitrile

To a stirred solution of ethyl 2-cyano-2-(2-cyano-5-fluorophenyl)acetate (2.40 g, 10.335 mmol, 1.00 equiv) in DMSO (10.00 mL) was added saturated NaCl (aq.) (10.00 mL) at room temperature. The solution was stirred for 16 h at 80° C. The mixture was allowed to cool down to room temperature. The solution was concentrated under reduced pressure and the residue was purified by reverse phase flash with the following conditions: Column: WelFlash™ C18-I, 20-40 um, 330 g; Eluent A: water (plus 10 mmol/L FA); Eluent B: ACN; Gradient: 40%-60% B in 20 min; Flow rate: 80 mL/min; Detector: 220/254 nm; desired fractions were collected at 54% B and concentrated under reduced pressure to afford 2-(cyanomethyl)-4-fluorobenzonitrile (1.5 g, 91%) as a light yellow solid. ¹H NMR (400 MHz, DMSO-d₆) δ 7.99 (dd, J=8.0, 7.0 Hz, 1H), 7.51-7.40 (m, 2H), 4.23 (s, 2H); LC/MS (ESI, m/z): [(M−1)]⁻=158.95.

### Step 3: 6-Fluoro-2,4-dihydroisoquinoline-1,3-dione

To a stirred solution of 2-(cyanomethyl)-4-fluorobenzonitrile (100.00 mg, 0.624 mmol, 1.00 equiv) in conc. HCl (10.00 mL) was stirred for 2 h at 70° C. The mixture was allowed to cool down to room temperature and was filtered. The filter cake was washed with water (3×100 mL) and dried to afford 6-fluoro-2,4-dihydroisoquinoline-1,3-dione (21 mg, 19%) as a beige solid. ¹H NMR (400 MHz, DMSO-d₆) δ 11.34 (s, 1H), 8.07 (dd, J=8.7, 5.9 Hz, 1H), 7.35-7.24 (m, 2H), 4.05 (s, 2H); LC/MS (ESI, m/z): [(M−1)]⁻=177.95.

### Example 37. Synthesis of 7-Fluoro-2,4-dihydroisoquinoline-1,3-dione (I-74)

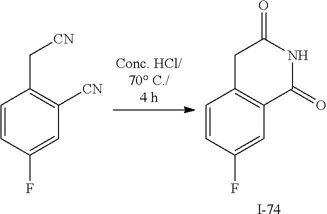

A mixture of 2-(cyanomethyl)-5-fluorobenzonitrile (100.00 mg, 0.624 mmol, 1.00 equiv) in HCl (12 M, 5.00 mL) was stirred at 75° C. for 2 h. The mixture was allowed to cool down to room temperature and was filtered. The filter cake was washed with H₂O (3×50 mL) and dried to afford 7-fluoro-2,4-dihydroisoquinoline-1,3-dione (74.6 mg, 67%) as an off-white solid. ¹H NMR (400 MHz, DMSO-d₆) δ 11.44 (s, 1H), 7.71 (dd, J=9.1, 2.8 Hz, 1H), 7.55 (td, J=8.6, 2.9 Hz, 1H), 7.46 (dd, J=8.6, 5.3 Hz, 1H), 4.02 (s, 2H); LC/MS (ESI, m/z): [(M−1)]⁻=178.00.

### Example 38. Synthesis of 8-Fluoro-2,4-dihydroisoquinoline-1,3-dione (I-75)

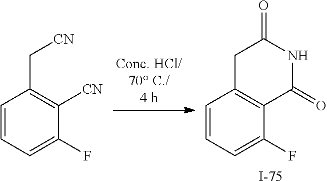

A mixture of 2-(cyanomethyl)-6-fluorobenzonitrile (100.00 mg, 0.624 mmol, 1.00 equiv) in HCl (12 M, 5.00 mL) was stirred at 75° C. for 2 h. The mixture was allowed to cool down to room temperature and was filtered. The filter cake was washed with H₂O (3×50 mL) and dried to afford 8-fluoro-2,4-dihydroisoquinoline-1,3-dione (66.6 mg, 60%) as a yellow brown solid. ¹H NMR (400 MHz, DMSO-d₆) δ 11.25 (s, 1H), 7.66 (td, J=8.0, 5.1 Hz, 1H), 7.29-7.12 (m, 2H), 4.05 (s, 2H); LC/MS (ESI, m/z): [(M−1)]⁻=177.95.

### Example 39. Synthesis of 1,3-dihydrospiro[indene-2,3′-pyrrolidine]-2′,5′-dione (I-186)

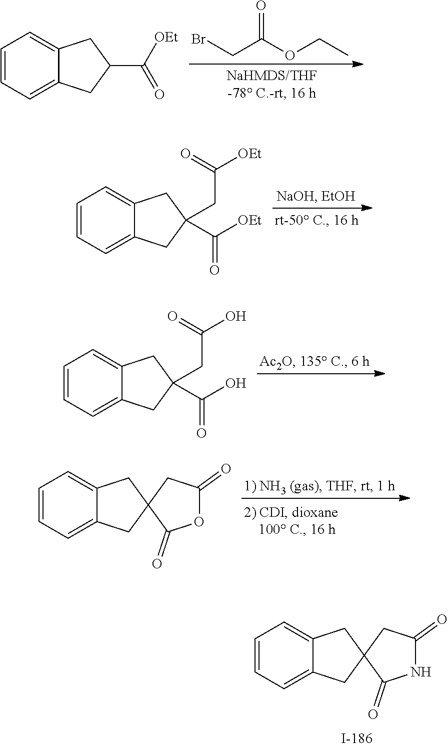

### Step 1: ethyl 2-(2-ethoxy-2-oxoethyl)-1,3-dihydroindene-2-carboxylate

To a stirred solution of ethyl 2,3-dihydro-1H-indene-2-carboxylate (800.00 mg, 4.21 mmol) in THF (10 mL) was added NaHMDS (2 M in THF, 6.31 mL, 12.62 mmol) dropwise at −78° C. under nitrogen atmosphere. The solution was stirred for 30 min at −78° C. Then ethyl bromoacetate (1755.68 mg, 10.51 mmol) was added to the reaction at −78° C. and the reaction was allowed to react for 2 h at room temperature. The reaction was monitored by LCMS. The resulting solution was concentrated under reduced pressure and the residue was purified by silica gel column chromatography, eluted with Petroleum ether/EtOAc (5:1) to afford ethyl 2-(2-ethoxy-2-oxoethyl)-1,3-dihydroindene-2-carboxylate (500 mg, 43%) as a colorless oil. ¹H NMR (400 MHz, DMSO-d₆) δ 7.20 (dt, J=7.0, 3.7 Hz, 2H), 7.17-7.11 (m, 2H), 4.14-3.99 (m, 4H), 3.39 (d, J=16.2 Hz, 2H), 2.98 (d, J=16.2 Hz, 2H), 2.78 (s, 2H), 1.25-1.10 (m, 6H). LC/MS (ESI, m/z): [(M+1)]⁺=277.05.

### Step 2: 2-(carboxymethyl)-1,3-dihydroindene-2-carboxylic acid

To a stirred solution of ethyl 2-(2-ethoxy-2-oxoethyl)-1,3-dihydroindene-2-carboxylate (500 mg, 1.81 mmol) in EtOH (10 mL)/H₂O (10 mL) was added NaOH (2 M, 1.80 mL, 3.60 mmol) at room temperature. The solution was stirred for 16 h at 50° C. The reaction was monitored by LCMS. The mixture was cooled to 0° C. and was acidified to pH 3 with 2 N HCl. The resulting solution was concentrated under reduced pressure to give 2-(carboxymethyl)-1,3-dihydroindene-2-carboxylic acid (600 mg, crude) as a white solid. ¹H NMR (400 MHz, DMSO-d₆) δ 12.25 (s, 2H), 7.19 (dd, J=5.4, 3.4 Hz, 2H), 7.17-7.09 (m, 2H), 3.39 (s, 2H), 2.94 (d, J=16.3 Hz, 2H), 2.68 (s, 2H). LC/MS (ESI, m/z): [(M−1)]⁻=219.05.

### Step 3: 1,3-dihydrospiro[indene-2,3′-oxolane]-2′,5′-dione

A solution of 2-(carboxymethyl)-1,3-dihydroindene-2-carboxylic acid (400.00 mg, 1.82 mmol) in Ac₂O (5.00 mL) was stirred for 6 h at 135° C. under nitrogen atmosphere. The reaction was monitored by LCMS. The mixture was allowed to cool down to room temperature. The resulting solution was concentrated under reduced pressure to give 1,3-dihydrospiro[indene-2,3′-oxolane]-2′,5′-dione (350 mg, 95%) as a white solid. ¹H NMR (400 MHz, DMSO-d₆) δ 7.27-7.16 (m, 4H), 3.42 (d, J=16.2 Hz, 2H), 3.25 (d, J=16.2 Hz, 2H), 3.13 (s, 2H).

### Step 4: 1,3-dihydrospiro[indene-2,3′-pyrrolidine]-2′,5′-dione

A mixture of 1,3-dihydrospiro[indene-2,3′-oxolane]-2′,5′-dione (100 mg, 0.50 mmol) in THE (5.00 mL) was stirred for 1 h at room temperature under NH3 (gas) atmosphere. The reaction was monitored by LCMS. The resulting mixture was concentrated under reduced. Then the residue was dissolved in dioxane (5 mL) and CDI (110.94 mg, 0.684 mmol, 1.5 equiv) was added to the solution at room temperature under nitrogen atmosphere. The reaction was stirred for 16 h at 100° C. The reaction was monitored by LCMS. The solution was cooled to room temperature and was concentrated under reduced pressure. The residue was purified by pre-HPLC with the following conditions: Column: X Bridge Prep OBD C¹⁸ Column, 30×150 mm 5 um; Mobile Phase A: water (10 mmoL/L NH₄HCO₃), Mobile Phase B: ACN; Flow rate: 60 mL/min; Gradient: 20 B to 40 B in 8 min; 254/220 nm; RT: 6.75 min to give 1,3-dihydrospiro[indene-2,3′-pyrrolidine]-2′,5′-dione (20 mg, 22%) as a white solid. ¹H NMR (400 MHz, DMSO-d₆) δ 11.25 (s, 1H), 7.25-7.13 (m, 4H), 3.29 (s, 2H), 3.05 (d, J=15.9 Hz, 2H), 2.72 (s, 2H). LC/MS (ESI, m/z): [(M−1)]⁻=200.15.

### Example 40. Synthesis of 8-(3-Fluorophenoxy)-2,4-dihydroisoquinoline-1,3-dione (I-198)

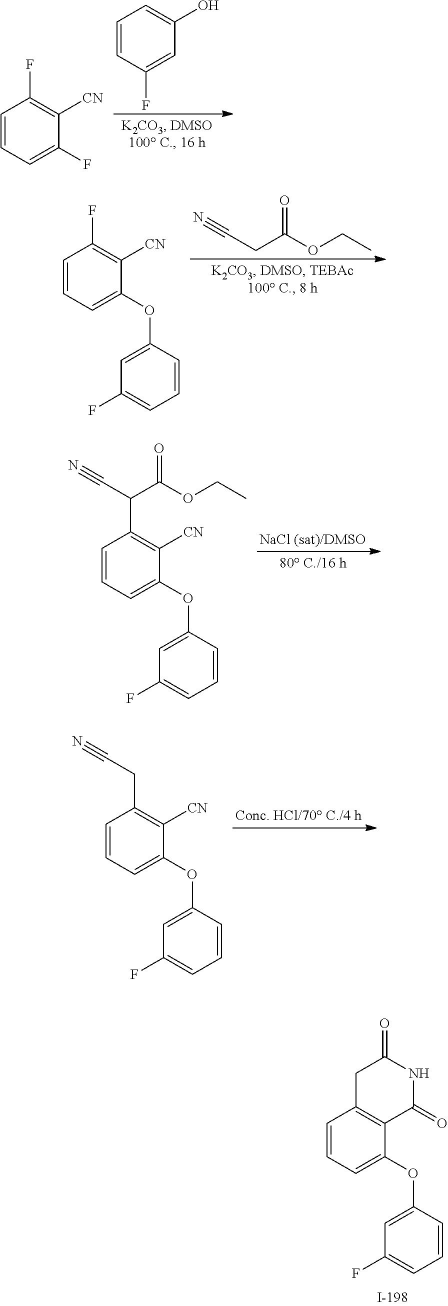

### Step 1: 2-Fluoro-6-(3-fluorophenoxy)benzonitrile

A mixture of 2,6-difluorobenzonitrile (2.00 g, 14.378 mmol, 1.00 equiv), m-fluorophenol (1.77 g, 15.815 mmol, 1.10 equiv) and K₂CO₃ (3.97 g, 28.755 mmol, 2 equiv) in DMSO (17.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 66% B and concentrated under reduced pressure to afford 2-fluoro-6-(3-fluorophenoxy)benzonitrile (2.6 g, 78%) as an off-white solid. ¹H NMR (400 MHz, DMSO-d₆) δ 7.74 (td, J=8.6, 6.8 Hz, 1H), 7.54 (td, J=8.3, 6.8 Hz, 1H), 7.30 (td, J=8.8, 0.8 Hz, 1H), 7.25-7.13 (m, 2H), 7.08 (ddd, J=8.4, 2.6, 0.8 Hz, 1H), 6.87 (dt, J=8.5, 0.8 Hz, 1H); LC/MS (ESI, m/z): [(M−1)]⁻=229.95.

### Step 2: Ethyl 2-cyano-2-[2-cyano-3-(3-fluorophenoxy)phenyl]acetate

A mixture of 2-fluoro-6-(3-fluorophenoxy)benzonitrile (2.58 g, 11.159 mmol, 1.00 equiv), ethyl cyanoacetate (1.26 g, 11.159 mmol, 1.00 equiv) and K₂CO₃ (4.63 g, 33.477 mmol, 3.00 equiv) in DMSO (20.00 mL) was stirred at 80° C. for 8 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 69% B and concentrated under reduced pressure to afford ethyl 2-cyano-2-[2-cyano-3-(3-fluorophenoxy)phenyl]acetate (3.2 g, 88%) as an off-white solid. ¹H NMR (400 MHz, DMSO-d₆) δ 7.83-7.73 (m, 1H), 7.54 (td, J=8.2, 6.7 Hz, 1H), 7.45 (dd, J=7.8, 0.9 Hz, 1H), 7.22-7.11 (m, 3H), 7.08-7.00 (m, 1H), 6.11 (s, 1H), 4.39-4.18 (m, 2H), 1.24 (t, J=7.1 Hz, 3H); LC/MS (ESI, m z): [(M−1)]⁻=322.90.

### Step 3: 2-(Cyanomethyl)-6-(3-fluorophenoxy)benzonitrile

To a stirred solution of ethyl 2-cyano-2-[2-cyano-3-(3-fluorophenoxy)phenyl]acetate (500.00 mg, 1.542 mmol, 1.00 equiv) in DMSO (5.00 mL) was added saturated NaCl (aq.) (5.00 mL) at room temperature. The solution was stirred for 8 h at 80° C. The mixture was allowed to cool down to room temperature and was concentrated under reduced pressure. The residue was purified by reverse phase flash chromatography with the following conditions: Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 66% B and concentrated under reduced pressure to afford 2-(cyanomethyl)-6-(3-fluorophenoxy)benzonitrile (260 mg, 66.86%) as an off-white solid. ¹H NMR (400 MHz, DMSO-d₆) δ 7.73 (dd, J=8.5, 7.8 Hz, 1H), 7.57-7.48 (m, 1H), 7.43 (dd, J=7.7, 1.0 Hz, 1H), 7.17-7.11 (m, 2H), 7.09-6.99 (m, 2H), 4.34 (s, 2H); LC/MS (ESI, m/z): [(M−1)]⁻=250.95.

### Step 4: 8-(3-Fluorophenoxy)-2,4-dihydroisoquinoline-1,3-dione

A solution of 2-(cyanomethyl)-6-(3-fluorophenoxy)benzonitrile (150.00 mg, 0.595 mmol, 1.00 equiv) in conc. HCl (5.00 mL) was stirred for 2 h at 70° C. The mixture was allowed to cool down to room temperature and was filtered. The filter cake was washed with water (3×50 mL) and dried to afford 8-(3-fluorophenoxy)-2,4-dihydroisoquinoline-1,3-dione (22.5 mg, 14%) as an orange solid. ¹H NMR (400 MHz, DMSO-d₆) δ 11.08 (s, 1H), 7.67 (t, J=7.9 Hz, 1H), 7.35 (q, J=7.9 Hz, 1H), 7.28 (d, J=7.7 Hz, 1H), 7.07 (d, J=8.1 Hz, 1H), 6.89 (td, J=8.5, 2.5 Hz, 1H), 6.73 (dt, J=10.6, 2.4 Hz, 1H), 6.68 (dd, J=8.3, 2.4 Hz, 1H), 4.08 (s, 2H); LC/MS (ESI, m/z): [(M−1)]⁻=269.90.

### Example 41. Synthesis of 8-(3-Methylphenoxy)-2,4-dihydroisoquinoline-1,3-dione (I-199)

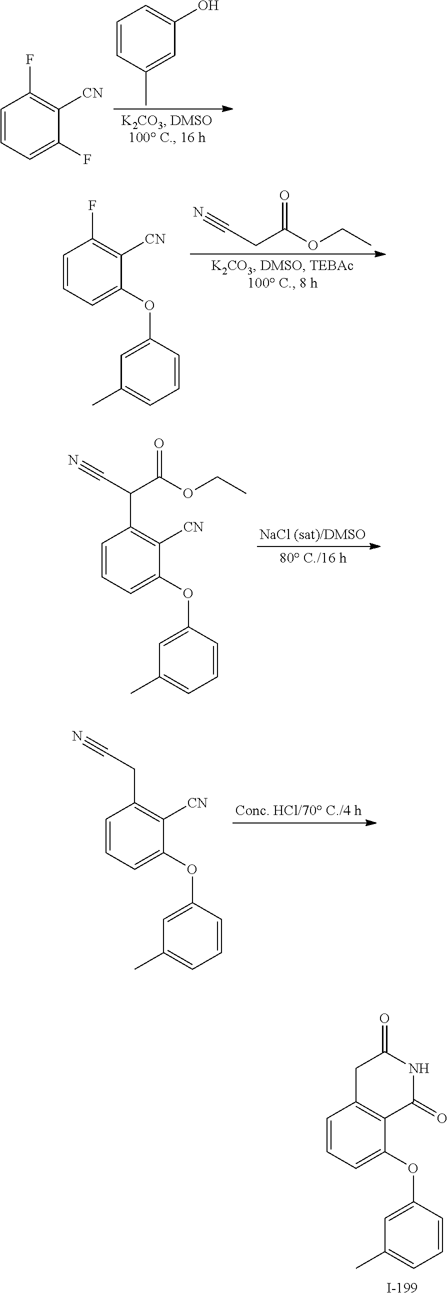

### Step 1: 2-Fluoro-6-(3-methylphenoxy)benzonitrile

A mixture of 2,6-difluorobenzonitrile (2.00 g, 14.378 mmol, 1.00 equiv), m-cresol (1.71 g, 15.815 mmol, 1.10 equiv) and K₂CO₃ (3.97 g, 28.755 mmol, 2.00 equiv) in DMSO (17.00 mL) was stirred at 100° C. for 16 h. The mixture was allowed to cool down to room temperature. The resulting mixture was purified by reverse phase flash with the following conditions Column: WelFlash™ C18-I, 20-40 um, 330 g; Eluent A: water (plus 10 mmol/L FA); Eluent B: ACN; Gradient: 55%-75% B in 20 min; Flow rate: 80 mL/min; Detector: 220/254 nm; desired fractions were collected at 70% B and concentrated under reduced pressure to afford 2-fluoro-6-(3-methylphenoxy)benzonitrile (2.1 g, 64%) as an off-white solid. ¹H NMR (400 MHz, DMSO-d₆) δ 7.70 (td, J=8.6, 6.9 Hz, 1H), 7.38 (t, J=7.8 Hz, 1H), 7.29-7.18 (m, 1H), 7.14 (ddt, J=7.6, 1.8, 0.9 Hz, 1H), 7.08-6.97 (m, 2H), 6.74 (dd, J=8.6, 0.8 Hz, 1H), 2.34 (s, 3H); LC/MS (ESI, m/z): [(M+1)]⁺=228.05.

### Step 2: Ethyl 2-cyano-2-[2-cyano-3-(3-methylphenoxy)phenyl]acetate

A mixture of 2-fluoro-6-(3-methylphenoxy)benzonitrile (2.12 g, 9.329 mmol, 1.00 equiv), ethyl cyanoacetate (1.06 g, 9.329 mmol, 1.00 equiv) and K₂CO₃ (3.87 g, 27.988 mmol, 3.00 equiv) in DMSO (18.00 mL) was stirred at 80° C. for 8 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 69% B and concentrated under reduced pressure to afford ethyl 2-cyano-2-[2-cyano-3-(3-methylphenoxy)phenyl]acetate (2.5 g, 84%) as an off-white solid. ¹H NMR (400 MHz, DMSO-d₆) δ 7.83-7.73 (m, 1H), 7.54 (td, J=8.2, 6.7 Hz, 1H), 7.45 (dd, J=7.8, 0.9 Hz, 1H), 7.22-7.11 (m, 3H), 7.08-7.00 (m, 1H), 6.11 (s, 1H), 4.39-4.18 (m, 2H), 1.24 (t, J=7.1 Hz, 3H); LC/MS (ESI, m z): [(M−1)]⁻=322.90.

### Step 3: 2-(Cyanomethyl)-6-(3-methylphenoxy)benzonitrile

To a stirred solution of ethyl 2-cyano-2-[2-cyano-3-(3-methylphenoxy)phenyl]acetate (500.00 mg, 1.561 mmol, 1.00 equiv) in DMSO (5.00 mL) was added saturated NaCl (aq.) (5.00 mL) at room temperature. The mixture was stirred for 8 h at 80° C. The mixture was allowed to cool down to room temperature and was concentrated under reduced pressure. The residue was purified by reverse phase flash chromatography with the following conditions: Column: WelFlash™ C18-I, 20-40 um, 330 g; Eluent A: water (plus 10 mmol/L FA); Eluent B: ACN; Gradient: 55%-75% B in 20 min; Flow rate: 80 mL/min; Detector: 220/254 nm; desired fractions were collected at 66% B and concentrated under reduced pressure to afford 2-(cyanomethyl)-6-(3-methylphenoxy)benzonitrile (370 mg, 95.48%) as an off-white solid. ¹H NMR (400 MHz, DMSO-d₆) δ 7.73 (dd, J=8.5, 7.8 Hz, 1H), 7.57-7.48 (m, 1H), 7.43 (dd, J=7.7, 1.0 Hz, 1H), 7.17-7.11 (m, 2H), 7.09-6.99 (m, 2H), 4.34 (s, 2H); LC/MS (ESI, m/z): [(M−1)]⁻=250.95.

### Step 4: 8-(3-Methylphenoxy)-2,4-dihydroisoquinoline-1,3-dione

To a stirred solution of 2-(cyanomethyl)-6-(3-methylphenoxy)benzonitrile (150.00 mg, 0.604 mmol, 1.00 equiv) in conc. HCl (5.00 mL) was stirred for 2 h at 70° C. The mixture was allowed to cool down to room temperature and was filtered. The filter cake was washed with water (3×100 mL) and dried to afford 8-(3-methylphenoxy)-2,4-dihydroisoquinoline-1,3-dione (65 mg, 40%) as an orange solid. ¹H NMR (400 MHz, DMSO-d₆) δ 11.08 (s, 1H), 7.67 (t, J=7.9 Hz, 1H), 7.35 (q, J=7.9 Hz, 1H), 7.28 (d, J=7.7 Hz, 1H), 7.07 (d, J=8.1 Hz, 1H), 6.89 (td, J=8.5, 2.5 Hz, 1H), 6.73 (dt, J=10.6, 2.4 Hz, 1H), 6.68 (dd, J=8.3, 2.4 Hz, 1H), 4.08 (s, 2H); LC/MS (ESI, m/z): [(M−1)]⁻=269.90.

### Example 42. Synthesis of 8-(2-Fluorophenoxy)-2,4-dihydroisoquinoline-1,3-dione (I-200)

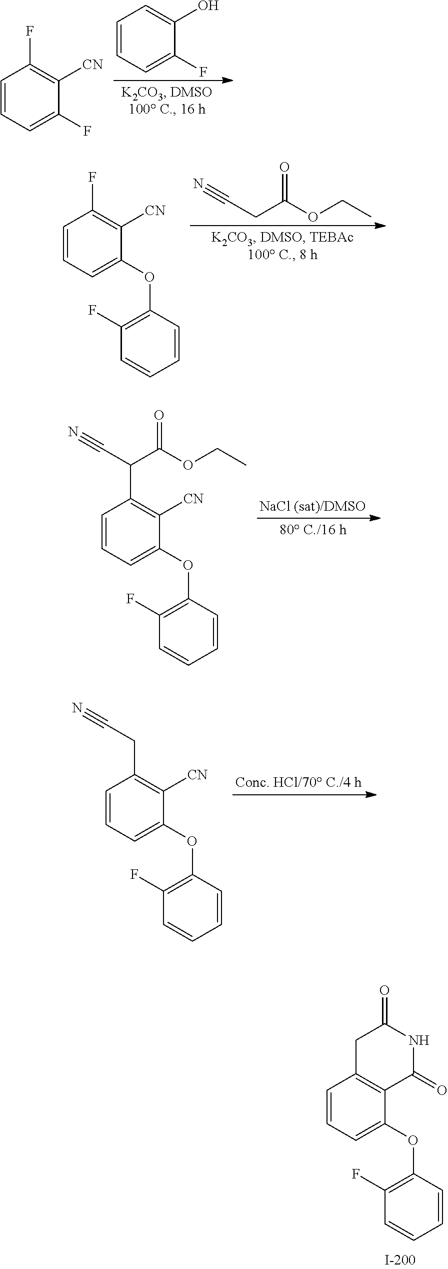

### Step 1: 2-fluoro-6-(3-methylphenoxy)benzonitrile2-fluoro-6-(2-fluorophenoxy)benzonitrile

A mixture of 2,6-difluorobenzonitrile (2.00 g, 14.378 mmol, 1.00 equiv), o-fluorophenol (1.77 g, 15.815 mmol, 1.10 equiv) and K₂CO₃ (3.97 g, 28.725 mmol, 2.00 equiv) in DMSO (17.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 55%-75% B in 20 min; Flow rate: 80 mL/min; Detector: 220/254 nm; desired fractions were collected at 70% B and concentrated under reduced pressure to afford 2-fluoro-6-(2-fluorophenoxy)benzonitrile (1.7 g, 51%) as an off-white solid. ¹HNMR (400 MHz, DMSO-d₆) δ 7.71 (td, J=8.6, 6.7 Hz, 1H), 7.55-7.23 (m, 5H), 6.73 (d, J=8.6 Hz, 1H); LC/MS (ESI, m/z): [(M−1)]⁻=229.95.

### Step 2: Ethyl 2-cyano-2-[2-cyano-3-(2-fluorophenoxy)phenyl]acetate

A mixture of 2-fluoro-6-(2-fluorophenoxy)benzonitrile (1.74 g, 7.526 mmol, 1.00 equiv), ethyl cyanoacetate (0.85 g, 7.526 mmol, 1.00 equiv) and K₂CO₃ (2.08 g, 15.052 mmol, 2.00 equiv) in DMSO (15.00 mL) was stirred at 80° C. for 8 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 69% B and concentrated under reduced pressure to afford ethyl 2-cyano-2-[2-cyano-3-(2-fluorophenoxy)phenyl]acetate (1.7 g, 70%) as a off-white solid. ¹H NMR (400 MHz, DMSO-d₆) δ 7.74 (dd, J=8.6, 7.7 Hz, 1H), 7.54-7.33 (m, 5H), 6.98 (d, J=8.7 Hz, 1H), 6.12 (s, 1H), 4.29 (q, J=7.1 Hz, 2H), 1.25 (t, J=7.1 Hz, 3H); LC/MS (ESI, m/z): [(M−1)]⁻=322.90.

### Step 3: 2-(Cyanomethyl)-6-(2-fluorophenoxy)benzonitrile

o a stirred solution of ethyl 2-cyano-2-[2-cyano-3-(2-fluorophenoxy)phenyl]acetate (500.00 mg, 1.542 mmol, 1.00 equiv) in DMSO (5.00 mL) was added saturated NaCl (aq.) (5.00 mL) at room temperature. The solution was stirred for 8 h at 80° C. The mixture was allowed to cool down to room temperature and was concentrated under reduced pressure. The residue was purified by reverse phase flash chromatography with the following conditions: Column: WelFlash™ C18-I, 20-40 um, 330 g; Eluent A: water (plus 10 mmol/L FA); Eluent B: ACN; Gradient: 55%-75% B in 20 min; Flow rate: 80 mL/min; Detector: 220/254 nm; desired fractions were collected at 66% B and concentrated under reduced pressure to afford 2-(cyanomethyl)-6-(2-fluorophenoxy)benzonitrile (380 mg, 98%) as an off-white solid. ¹H NMR (400 MHz, DMSO-d₆) δ 7.69 (dd, J=8.6, 7.7 Hz, 1H), 7.49 (ddd, J=11.1, 8.0, 1.6 Hz, 1H), 7.44-7.31 (m, 4H), 6.87 (d, J=8.6 Hz, 1H), 4.35 (s, 2H); LC/MS (ESI, m/z): [(M−1)]⁻=250.95.

### Step 4: 8-(2-Fluorophenoxy)-2,4-dihydroisoquinoline-1,3-dione

To a stirred solution of 2-(cyanomethyl)-6-(2-fluorophenoxy)benzonitrile (150.00 mg, 0.595 mmol, 1.00 equiv) in conc. HCl (5.00 mL) was stirred for 2 h at 70° C. The mixture was allowed to cool down to room temperature and was filtered. The filter cake was washed with water (3×100 mL) and dried to afford 8-(2-fluorophenoxy)-2,4-dihydroisoquinoline-1,3-dione (48.8 mg, 30%) as a light yellow solid. ¹H NMR (400 MHz, DMSO-d₆) δ 11.08 (s, 1H), 7.60 (t, J=8.0 Hz, 1H), 7.44-7.31 (m, 1H), 7.23-7.10 (m, 3H), 6.97-6.88 (m, 1H), 6.88 (d, J=8.2 Hz, 1H), 4.07 (s, 2H); LC/MS (ESI, m/z): [(M−1)]⁻=269.90.

### Example 43. Synthesis of 6-Methyl-8-phenoxy-2,4-dihydroisoquinoline-1,3-dione (I-203)

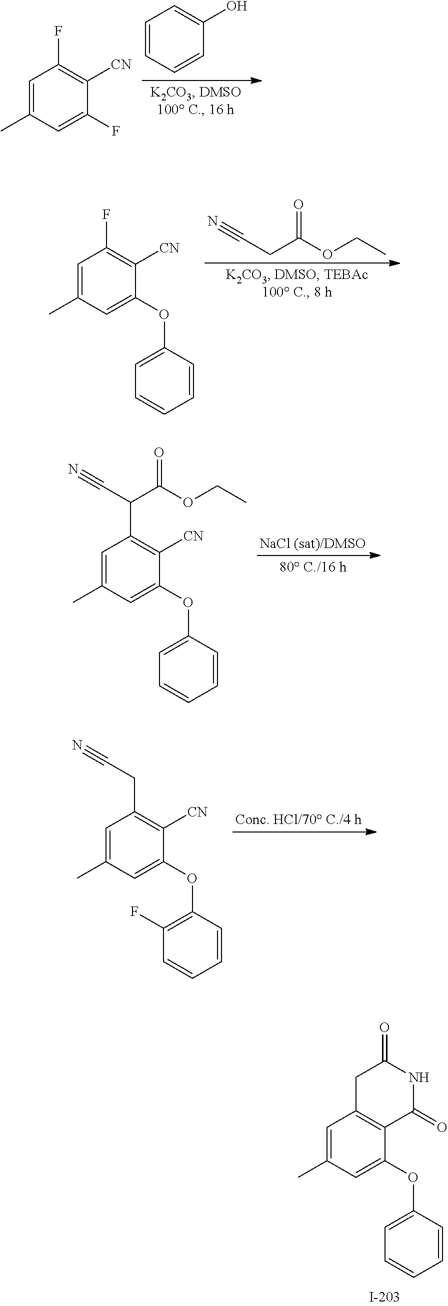

### Step 1: 2-Fluoro-4-methyl-6-phenoxybenzonitrile

A mixture of 2,6-difluoro-4-methylbenzonitrile (1.00 g, 6.530 mmol, 1.00 equiv), phenol (676.05 mg, 7.183 mmol, 1.10 equiv) and K₂CO₃ (1.81 g, 13.061 mmol, 2.00 equiv) in DMSO (15.00 mL) was stirred at 100° C. for 16 h. The mixture was allowed to cool down to room temperature. The resulting mixture was purified by reverse phase flash with the following conditions Column: WelFlash™ C18-I, 20-40 um, 330 g; Eluent A: water (plus 10 mmol/L FA); Eluent B: ACN; Gradient: 0%-80% B in 20 min; Flow rate: 80 mL/min; Detector: 220/254 nm; desired fractions were collected at 70% B and concentrated under reduced pressure to afford 2-fluoro-4-methyl-6-phenoxybenzonitrile (970 mg, 65%) as an off-white solid. ¹H NMR (400 MHz, DMSO-d₆) δ 7.58-7.45 (m, 2H), 7.37-7.27 (m, 1H), 7.24-7.18 (m, 2H), 7.15-7.09 (m, 1H), 6.58 (d, J=1.2 Hz, 1H), 2.31 (s, 3H); LC/MS (ESI, m/z): [(M−1)]⁻=225.95.

### Step 2: Ethyl 2-cyano-2-(2-cyano-5-methyl-3-phenoxyphenyl)acetate

To a stirred mixture of 2-fluoro-4-methyl-6-phenoxybenzonitrile (970.00 mg, 4.269 mmol, 1.00 equiv), ethyl cyanoacetate (482.85 mg, 4.269 mmol, 1 equiv) in DMSO (10.00 mL) was added K₂CO₃ (1.77 g, 12.806 mmol, 3 equiv) at room temperature. The mixture was stirred for 8 h at 100° C. The mixture was allowed to cool down to room temperature. The reaction was monitored by LCMS. The mixture was poured into HCl (6 M, 100 mL) and the resulting mixture was extracted with EtOAc (3×100 mL). The combined organic layers were washed with brine (1×100 mL), dried over anhydrous Na₂SO₄. After filtration, the filtrate was concentrated under reduced pressure to give ethyl 2-cyano-2-(2-cyano-5-methyl-3-phenoxyphenyl)acetate (880 mg, 64.35%) as a dark yellow semi-solid. ¹H NMR (400 MHz, DMSO-d₆) δ 7.51 (tdd, J=7.4, 3.7, 1.6 Hz, 2H), 7.35-7.28 (m, 1H), 7.25-7.15 (m, 3H), 6.85 (t, J=1.1 Hz, 1H), 6.03 (s, 1H), 4.33-4.21 (m, 2H), 2.35 (d, J=3.6 Hz, 3H), 1.25 (t, J=7.1 Hz, 3H); LC/MS (ESI, m/z): [(M−1)]⁻=318.90.

### Step 3: 2-(Cyanomethyl)-4-methyl-6-phenoxybenzonitrile

To a stirred solution of ethyl 2-cyano-2-(2-cyano-5-methyl-3-phenoxyphenyl)acetate (300.00 mg, 1 equiv) in DMSO (3.00 mL) was added saturated NaCl (aq.) (3.00 mL) at room temperature. The solution was stirred for 8 h at 80° C. The mixture was allowed to cool down to room temperature and was concentrated under reduced pressure and the residue was purified by reverse phase flash chromatography with the following conditions: Column: WelFlash™ C18-I, 20-40 ?m, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 66% B and concentrated under reduced pressure to afford 2-(cyanomethyl)-4-methyl-6-phenoxybenzonitrile (232 mg, 99%) as an off-white solid. ¹H NMR (400 MHz, DMSO-d₆) δ 7.57-7.42 (m, 2H), 7.32-7.26 (m, 1H), 7.22-7.14 (m, 3H), 6.76 (s, 1H), 4.27 (s, 2H), 2.34 (d, J=10.3 Hz, 3H); LC/MS (ESI, m/z): [(M−1)]⁻=247.00.

### Step 4: 6-Methyl-8-phenoxy-2,4-dihydroisoquinoline-1,3-dione

To a stirred solution of 2-(cyanomethyl)-4-methyl-6-phenoxybenzonitrile (150.00 mg, 0.604 mmol, 1.00 equiv) in conc. HCl (5.00 mL) was stirred for 2 h at 70° C. The mixture was allowed to cool down to room temperature and was filtered. The filter cake was washed with water (3×100 mL) and dried to afford 6-methyl-8-phenoxy-2,4-dihydroisoquinoline-1,3-dione (72.5 mg, 45%) as an orange solid. ¹H NMR (400 MHz, DMSO-d₆) δ 10.97 (s, 1H), 7.36 (dt, J=13.5, 7.7 Hz, 2H), 7.10 (dt, J=14.8, 7.4 Hz, 1H), 7.03 (s, 1H), 6.92 (dd, J=23.7, 8.0 Hz, 2H), 6.78 (s, 1H), 4.02 (s, 2H), 2.31 (s, 3H). 1.21 (d, J=7.0 Hz, 6H); LC/MS (ESI, m/z): [(M−1)]⁻=265.95.

### Example 44. Synthesis of 8-(2-Bromophenoxy)-2,4-dihydroisoquinoline-1,3-dione (I-204)

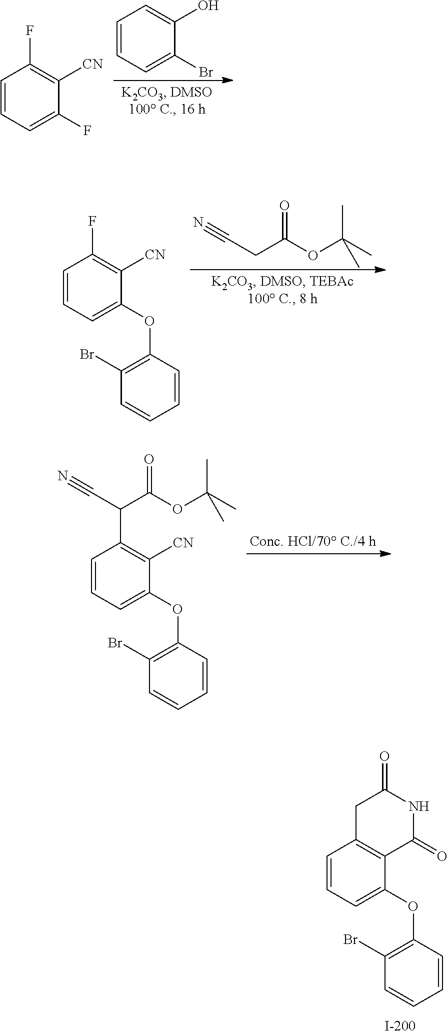

### Step 1: 2-(2-Bromophenoxy)-6-fluorobenzonitrile

A mixture of 2,6-difluorobenzonitrile (3.00 g, 21.566 mmol, 1.00 equiv), 2-bromophenol (4.10 g, 23.698 mmol, 1.10 equiv) and K₂CO₃ (5.96 g, 43.133 mmol, 2 equiv) in DMSO (20.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 69% B and concentrated under reduced pressure to afford 2-(2-bromophenoxy)-6-fluorobenzonitrile (4.45 g, 71%) as an off-white solid. ¹H NMR (400 MHz, DMSO-d₆) δ 7.83 (dd, J=8.0, 1.6 Hz, 1H), 7.70 (td, J=8.6, 6.8 Hz, 1H), 7.54 (ddd, J=8.0, 7.4, 1.6 Hz, 1H), 7.43 (dd, J=8.1, 1.6 Hz, 1H), 7.33 (td, J=7.7, 1.6 Hz, 1H), 7.31-7.22 (m, 1H), 6.58 (dd, J=8.6, 0.8 Hz, 1H); LC/MS (ESI, m/z): [(M−1)]⁻=210.95.

### Step 2: Tert-butyl 2-[3-(2-bromophenoxy)-2-cyanophenyl]-2-cyanoacetate

A mixture of 2-(2-bromophenoxy)-6-fluorobenzonitrile (2.00 g, 6.847 mmol, 1.00 equiv), tert-butyl 2-cyanoacetate (1.06 g, 7.509 mmol, 1.10 equiv) and K₂CO₃ (2.84 g, 20.540 mmol, 3.00 equiv) in DMSO (15.00 mL) was stirred at 110° C. for 8 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 66% B and concentrated under reduced pressure to afford tert-butyl 2-[3-(2-bromophenoxy)-2-cyanophenyl]-2-cyanoacetate (1.26 g, 44.53%) as an off-white solid. ¹H NMR (400 MHz, DMSO-d₆) δ 7.84 (dd, J=8.0, 1.5 Hz, 1H), 7.72 (dd, J=8.7, 7.7 Hz, 1H), 7.54 (ddd, J=8.1, 7.4, 1.6 Hz, 1H), 7.43-7.28 (m, 3H), 6.83 (dd, J=8.6, 0.9 Hz, 1H), 6.03 (s, 1H), 1.46 (s, 9H); LC/MS (ESI, m/z): [(M−1)]⁻=412.85.

### Step 3: 8-(2-Bromophenoxy)-2,4-dihydroisoquinoline-1,3-dione

To a stirred solution of tert-butyl 2-[3-(2-bromophenoxy)-2-cyanophenyl]-2-cyanoacetate (500.00 mg, 1.210 mmol, 1.00 equiv) in conc. HCl (10.00 mL) was stirred for 2 h at 70° C. The mixture was allowed to cool down to room temperature and was filtered. The filter cake was washed with water (3×100 mL) and dried to afford 8-(2-bromophenoxy)-2,4-dihydroisoquinoline-1,3-dione (260 mg, 65%) as an orange solid. ¹H NMR (400 MHz, DMSO-d₆) δ 11.08 (s, 1H), 7.72 (dd, J=7.9, 1.6 Hz, 1H), 7.61 (t, J=7.9 Hz, 1H), 7.33 (td, J=7.8, 1.6 Hz, 1H), 7.21 (dd, J=7.7, 1.1 Hz, 1H), 7.08 (td, J=7.7, 1.5 Hz, 1H), 6.82 (dt, J=8.2, 1.9 Hz, 2H), 4.07 (s, 2H); LC/MS (ESI, m/z): [(M−1)]⁺=331.80.

### Example 45. Synthesis of 8-(4-Bromophenoxy)-2,4-dihydroisoquinoline-1,3-dione (I-206)

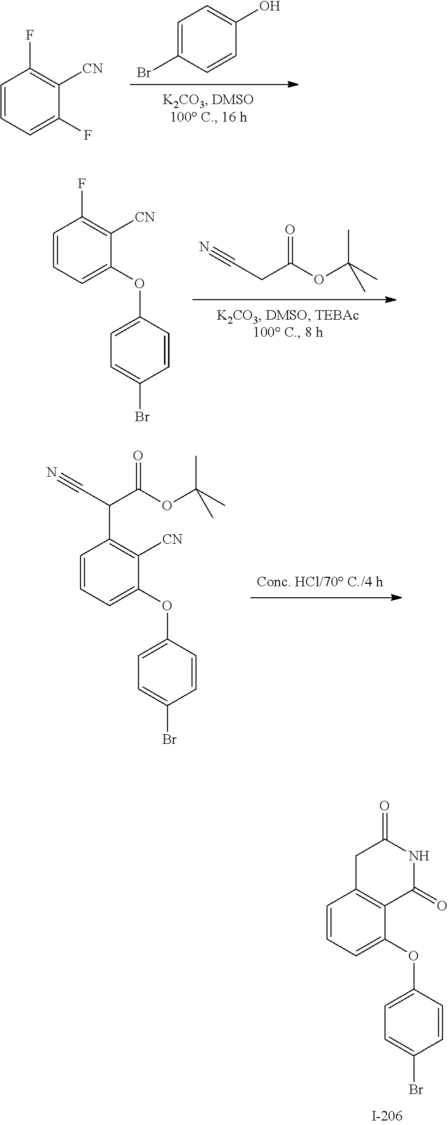

### Step 1: 2-(4-Bromophenoxy)-6-fluorobenzonitrile

A mixture of 2,6-difluorobenzonitrile (3.00 g, 21.566 mmol, 1.00 equiv), 4-bromophenol (4.10 g, 23.723 mmol, 1.10 equiv) and K₂CO₃ (5.96 g, 43.133 mmol, 2.00 equiv) in DMSO (20.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 69% B and concentrated under reduced pressure to afford 2-(4-bromophenoxy)-6-fluorobenzonitrile (3.44 g, 55%) as an off-white solid. ¹H NMR (400 MHz, DMSO-d₆) δ 7.76-7.62 (m, 3H), 7.33-7.18 (m, 3H), 6.82 (d, J=8.6 Hz, 1H); LC/MS (ESI, m/z): [(M−1)]⁻=289.85.

### Step 2: Tert-butyl 2-[3-(4-bromophenoxy)-2-cyanophenyl]-2-cyanoacetate

A mixture of 2-(4-bromophenoxy)-6-fluorobenzonitrile (3.30 g, 11.297 mmol, 1.00 equiv), tert-butyl 2-cyanoacetate (1.59 g, 11.263 mmol, 1.00 equiv) and K₂CO₃ (4.68 g, 33.892 mmol, 3.00 equiv) in DMSO (30.00 mL) was stirred at 100° C. for 8 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 66% B and concentrated under reduced pressure to afford tert-butyl 2-[3-(4-bromophenoxy)-2-cyanophenyl]-2-cyanoacetate (3.5 g, 75%) as an off-white solid. ¹H NMR (400 MHz, DMSO-d₆) δ 7.75 (dd, J=8.6, 7.7 Hz, 1H), 7.70-7.63 (m, 2H), 7.40 (d, J=7.7 Hz, 1H), 7.20-7.15 (m, 2H), 7.10 (dd, J=8.6, 0.9 Hz, 1H), 6.00 (s, 1H), 1.45 (s, 9H); LC/MS (ESI, m/z): [(M−1)]⁻=412.80.

### Step 3: 8-(4-Bromophenoxy)-2,4-dihydroisoquinoline-1,3-dione

A solution of tert-butyl 2-[3-(4-bromophenoxy)-2-cyanophenyl]-2-cyanoacetate (500.00 mg, 1.210 mmol, 1.00 equiv) in conc. HCl (10.00 mL) was stirred for 2 h at 70° C. The mixture was allowed to cool down to room temperature and was filtered. The filter cake was washed with water (3×100 mL) and dried to afford 8-(4-bromophenoxy)-2,4-dihydroisoquinoline-1,3-dione (90 mg, 22%) as an orange solid. ¹H NMR (400 MHz, DMSO-d₆) δ 11.08 (s, 1H), 7.66 (t, J=7.9 Hz, 1H), 7.49 (d, J=8.4 Hz, 2H), 7.26 (d, J=7.7 Hz, 1H), 7.04 (d, J=8.2 Hz, 1H), 6.84 (d, J=8.3 Hz, 2H), 4.07 (s, 2H); LC/MS (ESI, m/z): [(M−1)]⁻=331.80.

### Example 46. Synthesis of 9-Phenoxy-4,5-dihydro-2H-2-benzazepine-1,3-dione (I-209)

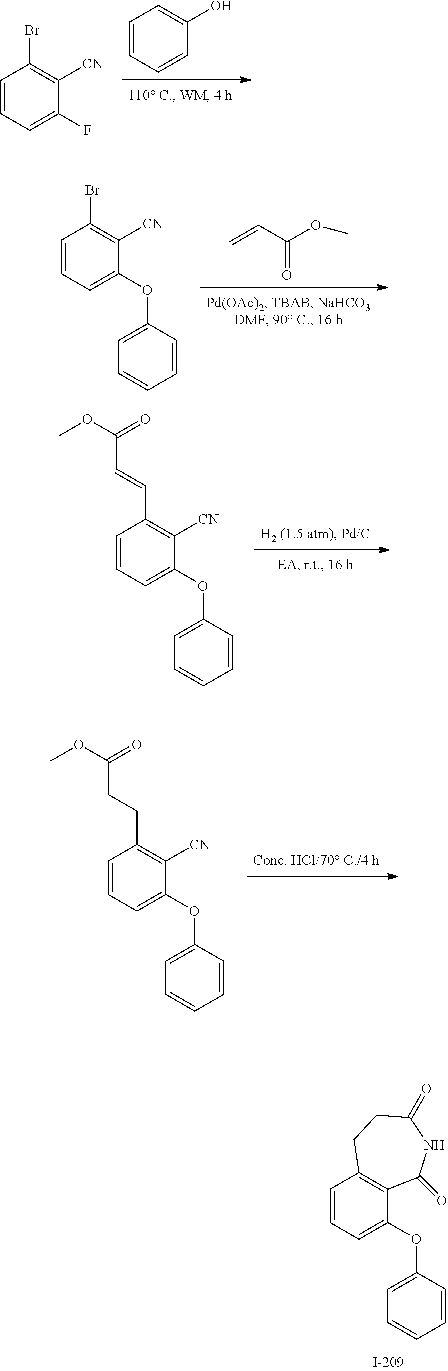

### Step 1: 2-Bromo-6-phenoxybenzonitrile

A mixture of 2-bromo-6-fluorobenzonitrile (3.00 g, 14.999 mmol, 1.00 equiv), phenol (1.55 g, 16.499 mmol, 1.10 equiv) and K₂CO₃ (4.15 g, 29.999 mmol, 2.00 equiv) in DMF (30.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 69% B and concentrated under reduced pressure to afford 2-bromo-6-phenoxybenzonitrile (4.0 g, 97%) as an off-white solid. ¹H NMR (400 MHz, DMSO-d₆) δ 7.63-7.52 (m, 2H), 7.55-7.45 (m, 2H), 7.35-7.27 (m, 1H), 7.27-7.18 (m, 2H), 6.96-6.84 (m, 1H); LC/MS (ESI, m/z): [(M−1)]⁻=271.80.

### Step 2: Methyl (2E)-3-(2-cyano-3-phenoxyphenyl)prop-2-enoate

To a stirred mixture of 2-bromo-6-phenoxybenzonitrile (2.50 g, 9.120 mmol, 1.00 equiv), methyl acrylate (3.93 g, 45.601 mmol, 5 equiv), TBAB (2.94 g, 9.120 mmol, 1 equiv) and NaHCO₃ (1.92 g, 22.800 mmol, 2.5 equiv) in DMF (25.00 mL) was added Pd(OAc)₂ (0.41 g, 1.824 mmol, 0.2 equiv) at room temperature under nitrogen atmosphere. The above mixture was stirred for 16 h at 90° C. The mixture was allowed to cool down to room temperature and was diluted with water (200 mL). The resulting mixture was extracted with EtOAc (3×200 mL). The combined organic layers were washed with brine (100 mL), dried over anhydrous Na₂SO₄. After filtration, the filtrate was concentrated under reduced pressure to afford methyl (2E)-3-(2-cyano-3-phenoxyphenyl)prop-2-enoate (2.4 g, crude) as a brown solid. ¹H NMR (400 MHz, DMSO-d₆) δ 7.88-7.77 (m, 2H), 7.69 (t, J=8.2 Hz, 1H), 7.54-7.45 (m, 2H), 7.34-7.26 (m, 1H), 7.24-7.16 (m, 2H), 7.01-6.92 (m, 2H), 3.79 (s, 3H).

### Step 3: Methyl 3-(2-cyano-3-phenoxyphenyl)propanoate

A mixture of methyl (2E)-3-(2-cyano-3-phenoxyphenyl)prop-2-enoate (2.40 g, 8.593 mmol, 1.00 equiv) in EtOAc (30.00 ml) was stirred at room temperature under hydrogen atmosphere for 16 h. The resulting mixture was filtered. The filter cake was washed with EtOAc (3×100 mL). The filtrate was concentrated under reduced pressure to afford methyl 3-(2-cyano-3-phenoxyphenyl)propanoate (2.40 g, 99%) as a white solid. ¹H NMR (400 MHz, DMSO-d₆) δ 7.57 (dd, J=8.5, 7.7 Hz, 1H), 7.53-7.43 (m, 2H), 7.28 (ddt, J=8.5, 7.0, 1.2 Hz, 1H), 7.23 (dd, J=7.8, 0.9 Hz, 1H), 7.20-7.11 (m, 2H), 6.79 (dd, J=8.5, 0.9 Hz, 1H), 3.62 (s, 3H), 3.09 (t, J=7.6 Hz, 2H), 2.81-2.71 (m, 2H); LC/MS (ESI, m/z): [(M−1)]⁻=282.05.

### Step 4: 9-Phenoxy-4,5-dihydro-2H-2-benzazepine-1,3-dione

To a stirred solution of methyl 3-(2-cyano-3-phenoxyphenyl)propanoate (200.00 mg, 0.711 mmol, 1.00 equiv) in conc. HCl (5.00 mL) was stirred for 2 h at 70° C. The mixture was allowed to cool down to room temperature and was filtered. The filter cake was washed with water (3×100 mL) and dried to afford 9-phenoxy-4,5-dihydro-2H-2-benzazepine-1,3-dione (86.2 mg, 45%) as a white solid. ¹H NMR (400 MHz, DMSO-d₆) δ 12.34 (s, 1H), 7.57 (t, J=8.1 Hz, 1H), 7.47 (t, J=7.7 Hz, 2H), 7.31-7.19 (m, 2H), 7.15 (d, J=8.0 Hz, 2H), 6.78 (d, J=8.4 Hz, 1H), 3.05 (t, J=7.6 Hz, 2H), 2.67 (t, J=7.6 Hz, 2H); LC/MS (ESI, m/z): [(M−1)]⁻=265.90.

### Example 47. Synthesis of 8-[2-(Trifluoromethyl)phenoxy]-2,4-dihydroisoquinoline-1,3-dione (I-210)

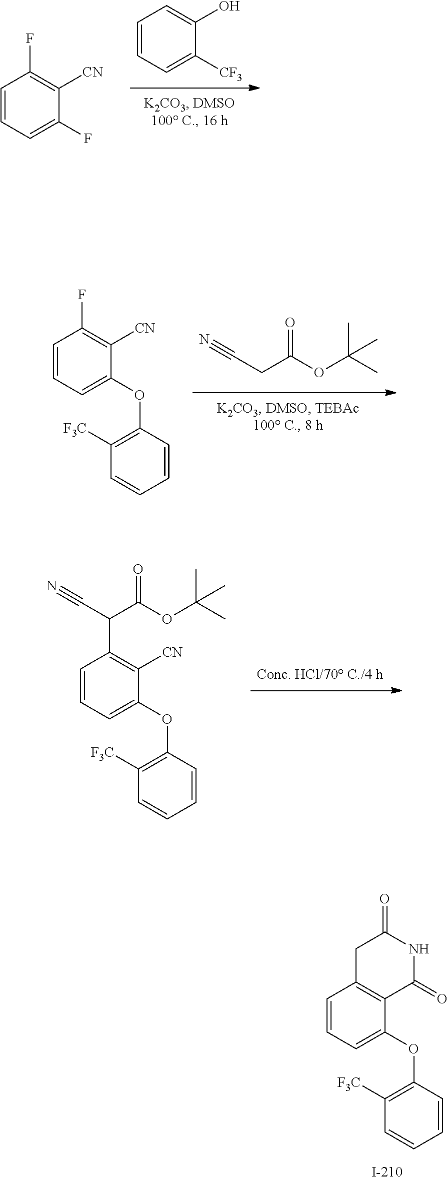

### Step 1: 2-Fluoro-6-[2-(trifluoromethyl)phenoxy]benzonitrile

A mixture of 2,6-difluorobenzonitrile (3.00 g, 21.566 mmol, 1.00 equiv), o-trifluoromethylphenol (3.85 g, 23.723 mmol, 1.10 equiv) and K₂CO₃ (5.96 g, 43.133 mmol, 2.00 equiv) in DMSO (30.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 60% B and concentrated under reduced pressure to afford 2-fluoro-6-[2-(trifluoromethyl)phenoxy]benzonitrile (3.2 g, 53%) as an off-white solid. ¹H NMR (400 MHz, DMSO-d₆) δ 7.90 (dd, J=8.0, 2.1 Hz, 1H), 7.84-7.75 (m, 1H), 7.80-7.70 (m, 1H), 7.52 (t, J=7.7 Hz, 1H), 7.42 (d, J=8.3 Hz, 1H), 7.33 (tt, J=8.6, 1.9 Hz, 1H), 6.84 (d, J=8.6 Hz, 1H); LC/MS (ESI, m/z): [(M−1)]⁻=279.90.

### Step 2: Tert-butyl 2-cyano-2-[2-cyano-3-[2-(trifluoromethyl)phenoxy]phenyl]acetate

A mixture of 2-fluoro-6-[2-(trifluoromethyl)phenoxy]benzonitrile (3.20 g, 11.379 mmol, 1.00 equiv), tert-butyl 2-cyanoacetate (1.61 g, 11.379 mmol, 1 equiv) and K₂CO₃ (4.72 g, 34.152 mmol, 3.00 equiv) in DMSO (30.00 mL) was stirred at 110° C. for 8 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 66% B and concentrated under reduced pressure to afford tert-butyl 2-cyano-2-[2-cyano-3-[2-(trifluoromethyl)phenoxy]phenyl]acetate (3 g, 66%) as an off-white solid. ¹H NMR (400 MHz, DMSO-d₆) δ 7.96-7.87 (m, 1H), 7.84-7.73 (m, 2H), 7.52 (t, J=7.7 Hz, 1H), 7.43 (dd, J=8.2, 5.0 Hz, 1H), 7.34 (d, J=8.4 Hz, 1H), 7.08 (d, J=8.5 Hz, 1H), 6.03 (s, 1H), 1.44 (s, 9H); LC/MS (ESI, m/z): [(M+1)]⁺=403.00.

### Step 3: 8-2-(Trifluoromethyl)phenoxyl-2,4-dihydroisoquinoline-1,3-dione

To a stirred solution of tert-butyl 2-cyano-2-[2-cyano-3-[2-(trifluoromethyl)phenoxy]phenyl]acetate (200.00 mg, 1 equiv) in conc. HCl (5.00 mL) was stirred for 2 h at 70° C. The mixture was allowed to cool down to room temperature and was filtered. The filter cake was washed with water (3×100 mL) and dried to afford 8-[2-(trifluoromethyl)phenoxy]-2,4-dihydroisoquinoline-1,3-dione (51.2 mg, 32%) as an orange solid. ¹H NMR (400 MHz, DMSO-d₆) δ 11.09 (s, 1H), 7.76 (dd, J=7.9, 1.7 Hz, 1H), 7.68 (t, J=7.9 Hz, 1H), 7.59-7.50 (m, 1H), 7.30 (dd, J=7.7, 1.1 Hz, 1H), 7.23 (t, J=7.6 Hz, 1H), 7.01 (dd, J=8.2, 1.2 Hz, 1H), 6.74 (d, J=8.4 Hz, 1H), 4.09 (s, 2H); LC/MS (ESI, m/z): [(M−1)]⁻=319.90.

### Example 48. Synthesis of 8-(2-Isopropylphenoxy)-2,4-dihydroisoquinoline-1,3-dione (I-211)

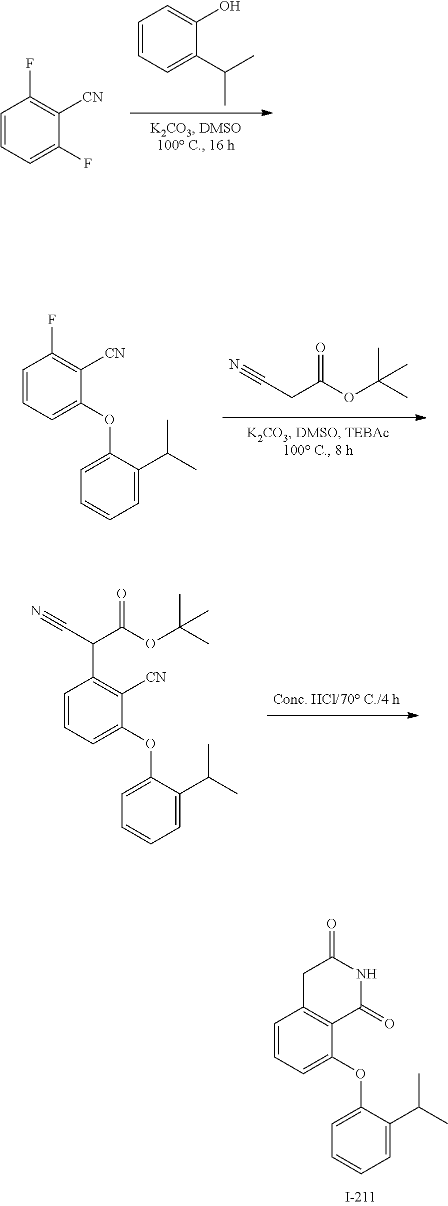

### Step 1: 2-Fluoro-6-(2-isopropylphenoxy)benzonitrile

A mixture of 2,6-difluorobenzonitrile (3.00 g, 21.566 mmol, 1.00 equiv), 2-isopropylphenol (3.23 g, 23.723 mmol, 1.10 equiv) and K₂CO₃ (5.96 g, 43.124 mmol, 2.00 equiv) in DMSO (30.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 60% B and concentrated under reduced pressure to afford 2-fluoro-6-(2-isopropylphenoxy)benzonitrile (3.3 g, 60%) as an off-white solid. ¹H NMR (400 MHz, DMSO-d₆) δ 7.68 (td, J=8.5, 6.8 Hz, 1H), 7.49 (dt, J=7.4, 3.8 Hz, 1H), 7.32 (dd, J=5.9, 3.5 Hz, 2H), 7.21 (t, J=8.8 Hz, 1H), 7.16-7.08 (m, 1H), 6.67-6.52 (m, 1H), 3.05 (hept, J=6.9 Hz, 1H), 1.18 (d, J=6.9 Hz, 6H); LC/MS (ESI, m/z): [(M+1)]⁺=256.05.

### Step 2: Tert-butyl 2-cyano-2-[2-cyano-3-(2-isopropylphenoxy)phenyl]acetate

A mixture of 2-fluoro-6-(2-isopropylphenoxy)benzonitrile (3.30 g, 12.926 mmol, 1.00 equiv), 2-fluoro-6-(2-isopropylphenoxy)benzonitrile (3.30 g, 12.926 mmol, 1.00 equiv) and K₂CO₃ (3.57 g, 25.853 mmol, 2.00 equiv) in DMSO (30.00 mL) was stirred at 100° C. for 16 h. The mixture was allowed to cool down to room temperature and was purified by reverse phase flash with the following conditions Column: WelFlash™ C18-I, 20-40 um, 330 g; Eluent A: water (plus 10 mmol/L FA); Eluent B: ACN; Gradient: 50%-70% B in 20 min; Flow rate: 80 mL/min; Detector: 220/254 nm; desired fractions were collected at 60% B and concentrated under reduced pressure to afford tert-butyl 2-cyano-2-[2-cyano-3-(2-isopropylphenoxy)phenyl]acetate (3.0 g, 2%) as an off-white solid. ¹H NMR (400 MHz, DMSO-d₆) δ 7.69 (td, J=8.2, 3.7 Hz, 1H), 7.49 (q, J=4.4 Hz, 1H), 7.32 (dt, J=8.2, 4.1 Hz, 3H), 7.15-7.02 (m, 1H), 6.83 (dd, J=8.6, 3.8 Hz, 1H), 6.01 (t, J=5.1 Hz, 1H), 3.04 (d, J=12.3 Hz, 1H), 1.46 (t, J=5.0 Hz, 9H), 1.18 (q, J=6.1 Hz, 6H); LC/MS (ESI, m/z): [(M+23)]⁺=339.10.

### Step 3: 8-(2-Isopropylphenoxy)-2,4-dihydroisoquinoline-1,3-dione

To a stirred solution of tert-butyl 2-cyano-2-[2-cyano-3-(2-isopropylphenoxy)phenyl]acetate (200.00 mg, 0.512 mmol, 1.00 equiv) in conc. HCl (5.00 mL) was stirred for 2 h at 70° C. The mixture was allowed to cool down to room temperature and was filtered. The filter cake was washed with water (3×100 mL) and dried to afford 8-(2-isopropylphenoxy)-2,4-dihydroisoquinoline-1,3-dione (61.8 mg, 41%) as an orange solid. ¹H NMR (400 MHz, DMSO-d₆) δ 11.05 (s, 1H), 7.55 (t, J=8.0 Hz, 1H), 7.40 (dd, J=7.3, 2.1 Hz, 1H), 7.24-7.06 (m, 3H), 6.72 (dd, J=7.5, 1.6 Hz, 2H), 4.06 (s, 2H), 3.21 (p, J=6.9 Hz, 1H), 1.21 (d, J=7.0 Hz, 6H); LC/MS (ESI, m/z): [(M−1)]⁻=296.10.

### Example 49. 6-Bromo-1-methyl-3H-quinazoline-2,4-dione (I-189)

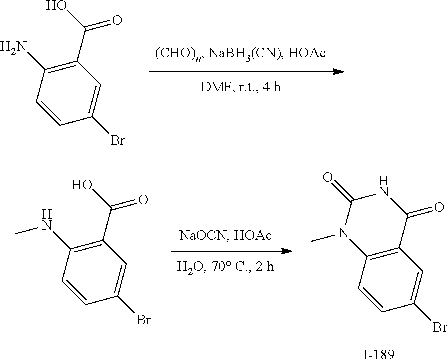

### Step 1: 5-Bromo-2-(methylamino)benzoic acid

To a stirred mixture of 5-bromoanthranilic acid (2.00 g, 9.258 mmol, 1.00 equiv) and (HCHO)ₙ (0.83 g, 27.667 mmol, 2.99 equiv) in DMF (20.00 mL) were added HOAc (5.58 g, 92.919 mmol, 10.04 equiv) and NaBH₃CN (1.76 g, 28.007 mmol, 3.03 equiv) at room temperature. The resulting mixture was stirred for 6 h at room temperature. The resulting mixture was diluted with water (60 mL) and extracted with EtOAc (3×50 mL). The combined organic phase was dried over anhydrous Na₂SO₄. After filtration, the filtrate was concentrated under reduced pressure. The residue was purified by reverse phase Flash chromatography with the following conditions: Column: WelFlash™ C18-I, 20-40 m, 330 g; Eluent A: water (plus 10 mmol/L FA); Eluent B: ACN; Gradient: 30%-50% B in 25 min; Flow rate: 80 mL/min; Detector: 220/254 nm; desired fractions were collected at 42% B and concentrated under reduced pressure to afford 5-bromo-2-(methylamino)benzoic acid (1.8 g, 85%) as a white solid. LC/MS (ESI, m/z): [(M+1)]⁺=230.00, 232.00.

### Step 2: 6-Bromo-1-methyl-3H-quinazoline-2,4-dione

To a stirred solution of 5-bromo-2-(methylamino)benzoic acid (280.00 mg, 1.217 mmol, 1.00 equiv) in AcOH (1.00 mL) and H₂O (16.00 mL) was added NaOCN (112.00 mg, 1.723 mmol, 1.42 equiv) in portions at room temperature. The resulting mixture was stirred for additional 30 min at 40° C. To the above mixture was added NaOH (1.38 g, 0.035 mmol, 0.03 equiv) in portions over 10 min at 40° C. The resulting mixture was stirred for additional 1 h at 70° C. The reaction was monitored by LCMS. The mixture was allowed to cool down to room temperature. The resulting mixture was diluted with water (50 mL). The mixture was acidified to pH 6 with HCl (2 M aq.). The resulting mixture was extracted with EtOAc (30 mL), dried over anhydrous Na₂SO₄. After filtration, the filtrate was concentrated under reduced pressure. The residue was purified by reverse phase Flash chromatography with the following conditions: Column: WelFlash™ C18-I, 20-40 nm, 120 g; Eluent A: water (plus 10 mmol/L NH₄HCO₃); Eluent B: ACN; Gradient: 30%-50% B in 30 min; Flow rate: 60 mL/min; Detector: 220/254 nm; desired fractions were collected at 42% B and concentrated under reduced pressure to afford 6-bromo-1-methyl-3H-quinazoline-2,4-dione (50 mg, 16%) as a yellow solid. ¹H NMR (400 MHz, DMSO-d₆) δ 11.71 (s, 1H), 8.05 (d, J=2.5 Hz, 1H), 7.92 (dd, J=8.9, 2.5 Hz, 1H), 7.41 (d, J=9.0 Hz, 1H), 3.43 (s, 3H). LC/MS (ESI, m/z): [(M+1)]⁺=254.90, 256.90.

### Example 50. N-(1,3-Dioxo-2,4-dihydroisoquinolin-8-yl)benzenesulfonamide (I-213)

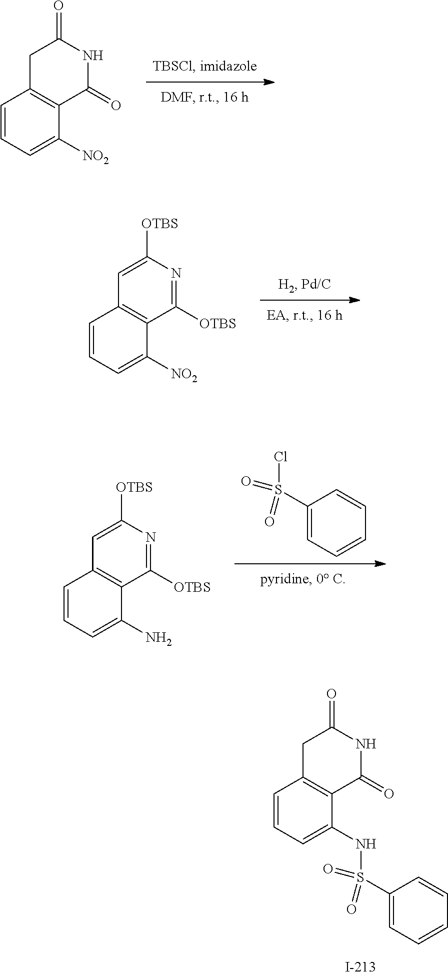 [SMILES: O=C1Cc2cccc([N+](=O)[O-])c2C(=O)N1 ; CC(C)(C)[Si](C)(C)Oc1cc2cccc([N+](=O)[O-])c2c(O[Si](C)(C)C(C)(C)C)n1 ; CC(C)(C)[Si](C)(C)Oc1cc2cccc(N)c2c(O[Si](C)(C)C(C)(C)C)n1 ; O=S(=O)(Cl)c1ccccc1 ; O=C1Cc2cccc(NS(=O)(=O)c3ccccc3)c2C(=O)N1]

### Step 1: 1,3-Bis[(tert-butyldimethylsilyl)oxy]-8-nitroisoquinoline

To a stirred solution of 8-nitro-2,4-dihydroisoquinoline-1,3-dione (200.00 mg, 0.970 mmol, 1.00 equiv) and imidazole (264.18 mg, 3.881 mmol, 4.0 equiv) in DMF (5 mL) was added t-butyldimethylchlorosilane (438.66 mg, 2.910 mmol, 3.00 equiv) in portions at room temperature under nitrogen atmosphere. The resulting mixture was stirred for 16 h at room temperature under nitrogen atmosphere. The reaction was monitored by LCMS. The resulting mixture was diluted with water (20 mL) and extracted with EtOAc (2×20 mL). The combined organic layers was washed with brine (10 mL), dried over anhydrous Na₂SO₄. After filtration, the filtrate was concentrated under reduced pressure. The residue was dissolved in petroleum ether (10 mL). The precipitated solids were collected by filtration and washed with petroleum ether (2×5 mL) to afford 1,3-bis[(tert-butyldimethylsilyl)oxy]-8-nitroisoquinoline as a brown solid. ¹H NMR (400 MHz, DMSO-d₆) δ 7.98 (dd, J=7.5, 1.7 Hz, 1H), 7.83 (t, J=7.8 Hz, 1H), 7.62 (d, J=8.1 Hz, 1H), 6.94 (s, 1H), 0.99 (s, 9H), 0.96 (s, 9H), 0.38 (s, 6H), 0.31 (s, 6H). LC/MS (ESI, m/z): [(M+1)]⁺=435.20.

### Step 2: 1,3-Bis[(tert-butyldimethylsilyl)oxy]isoquinolin-8-amine

To a stirred solution of 1,3-bis[(tert-butyldimethylsilyl)oxy]-8-nitroisoquinoline (330.00 mg, 0.759 mmol, 1.00 equiv) in EtOAc was added Pd/C (10% palladium on activated carbon, 30.00 mg) in portions at room temperature under nitrogen atmosphere. The mixture was hydrogenated at room temperature for 16 h under hydrogen atmosphere using a hydrogen balloon. It was filtered through a Celite pad and the filtrate was concentrated under reduced pressure. This resulted in 1,3-bis[(tert-butyldimethylsilyl)oxy]-8-nitroisoquinoline (242 mg, 35%) as a brown solid. ¹H NMR (400 MHz, DMSO-d₆) δ 7.22 (t, J=7.8 Hz, 1H), 6.76 (d, J=8.0 Hz, 1H), 6.42 (s, 2H), 6.35 (s, 2H), 1.02 (s, 9H), 0.98 (s, 9H), 0.44 (s, 6H), 0.26 (s, 6H). LC/MS (ESI, m/z): [(M+1)]⁺=405.20.

### Step 3: N-(1,3-Dioxo-2,4-dihydroisoquinolin-8-yl)benzenesulfonamide

To a stirred mixture of 1,3-bis[(tert-butyldimethylsilyl)oxy]isoquinolin-8-amine (87.00 mg, 0.215 mmol, 1.00 equiv) in pyridine (4.40 mL) was added benzenesulfonyl chloride (56.95 mg, 0.322 mmol, 1.50 equiv) at 0° C. under air atmosphere. The resulting mixture was stirred for 1 h at 0° C. under air atmosphere. The reaction was monitored by LCMS. The resulting mixture was concentrated under reduced pressure. The residue was purified by reverse phase Flash chromatography with the following conditions: Column: WelFlash™ C18-I, 20-40 nm, 330 g; Eluent A: water (plus 10 mmol/L TFA); Eluent B: ACN; Gradient: 45%-65% B in 30 min; Flow rate: 80 mL/min; Detector: 220/254 nm; desired fractions were collected at 55% B and concentrated under reduced pressure to afford N-(1,3-dioxo-2,4-dihydroisoquinolin-8-yl)benzenesulfonamide (23 mg, 34%) as a yellow solid. ¹H NMR (400 MHz, Chloroform-d) δ 11.43 (s, 1H), 8.10 (s, 1H), 7.97-7.91 (m, 2H), 7.74-7.67 (m, 1H), 7.61-7.54 (m, 1H), 7.51-7.47 (m, 3H), 6.93 (dq, J=7.6, 1.1 Hz, 1H), 3.99 (s, 2H). LC/MS (ESI, m/z): [(M+1)]⁺=316.95.

### Example 51. 8-Phenoxy-2,4-dihydro-2,7-naphthyridine-1,3-dione (I-214)

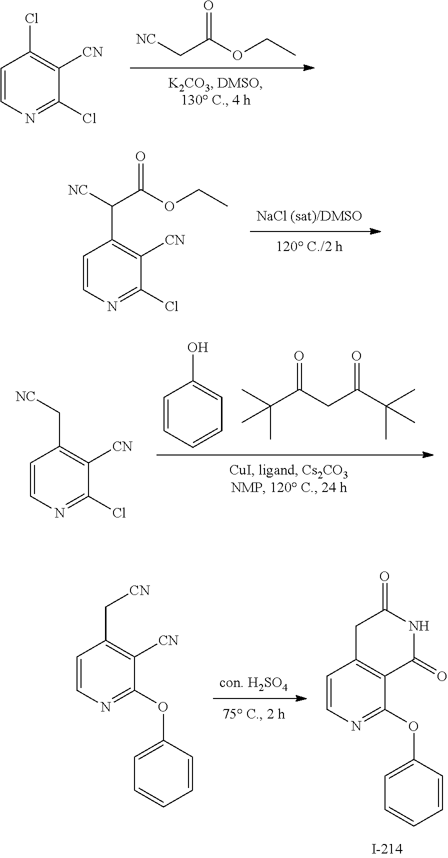

### Step 1: Ethyl 2-(2-chloro-3-cyanopyridin-4-yl)-2-cyanoacetate

To a stirred mixture of 2,4-dichloropyridine-3-carbonitrile (4.00 g, 23.121 mmol, 1.00 equiv) and ethyl cyanoacetate (2.63 g, 23.250 mmol, 1.01 equiv) in DMSO (80.00 mL) was added K₂CO₃ (6.42 g, 46.474 mmol, 2.01 equiv) at room temperature under air atmosphere. The resulting mixture was stirred for 4 h at 130° C. under nitrogen atmosphere. The reaction was monitored by LCMS. The mixture was allowed to cool down to room temperature. The resulting mixture was diluted with water (240 mL) and extracted with EtOAc (3×100 mL). The combined organic layers was dried over anhydrous Na₂SO₄. After filtration, the filtrate was concentrated under reduced pressure. The residue was purified by reverse phase Flash chromatography with the following conditions: Column: WelFlash™ C18-I, 20-40 nm, 330 g; Eluent A: water (plus 10 mmol/L FA); Eluent B: ACN; Gradient: 16%-36% B in 30 min; Flow rate: 80 mL/min; Detector: 220/254 nm; desired fractions were collected at 27% B and concentrated under reduced pressure to afford ethyl 2-(2-chloro-3-cyanopyridin-4-yl)-2-cyanoacetate (4.19 g, 73%) as a yellow solid. LC/MS (ESI, m z): [(M+1)]⁺=250.00, 252.00.

### Step 2: 2-Chloro-4-(cyanomethyl)pyridine-3-carbonitrile

To a stirred solution of ethyl 2-(2-chloro-3-cyanopyridin-4-yl)-2-cyanoacetate (4.19 g, 16.783 mmol, 1.00 equiv) in DMSO (10 mL) was added sat. aq. NaCl (10 mL) at room temperature under air atmosphere. The resulting mixture was stirred for 2 h at 120° C. under air atmosphere. The reaction was monitored by LCMS. The mixture was allowed to cool down to room temperature. The resulting mixture was diluted with water (20 mL) and extracted with EtOAc (3×15 mL). The combined organic layers was dried over anhydrous Na₂SO₄. After filtration, the filtrate was concentrated under reduced pressure. The residue was purified by reverse phase Flash chromatography with the following conditions: Column: WelFlash™ C18-I, 20-40 nm, 330 g; Eluent A: water (plus 10 mmol/L FA); Eluent B: ACN; Gradient: 25%-45% B in 25 min; Flow rate: 80 mL/min; Detector: 220/254 nm; desired fractions were collected at 42% B and concentrated under reduced pressure to afford 2-chloro-4-(cyanomethyl)pyridine-3-carbonitrile (2.66 g, 89%) as a yellow solid. ¹H NMR (400 MHz, DMSO-d₆) δ 8.73 (d, J=5.2 Hz, 1H), 7.72 (dt, J=5.2, 0.8 Hz, 1H), 4.45 (s, 2H). LC/MS (ESI, m/z): [(M−1)]⁻=175.95, 177.95.

### Step 3: 4-(Cyanomethyl)-2-phenoxypyridine-3-carbonitrile

To a stirred mixture of 2-chloro-4-(cyanomethyl)pyridine-3-carbonitrile (400.00 mg, 2.252 mmol, 1.00 equiv) and phenol (233.60 mg, 2.482 mmol, 1.10 equiv) in NMP (8.00 mL) were added CuCl (40.00 mg, 0.404 mmol, 0.18 equiv), 2,2,6,6-tetramethylheptane-3,5-dione (80.00 mg, 0.434 mmol, 0.19 equiv) and Cs₂CO₃ (1472.00 mg, 4.518 mmol, 2.01 equiv) at room temperature under air atmosphere. The resulting mixture was stirred for 24 h at 120° C. under nitrogen atmosphere. The reaction was monitored by LCMS. The mixture was allowed to cool down to room temperature. The resulting mixture was filtered. The filter cake was washed with MeOH (3×20 mL). The filtrate was concentrated under reduced pressure. The residue was purified by reverse phase Flash chromatography with the following conditions: Column: WelFlash™ C18-I, 20-40 nm, 120 g; Eluent A: water (plus 10 mmol/L TFA); Eluent B: ACN; Gradient: 37%-57% B in 25 min; Flow rate: 60 mL/min; Detector: 220/254 nm; desired fractions were collected at 52% B and concentrated under reduced pressure to afford 4-(cyanomethyl)-2-phenoxypyridine-3-carbonitrile (46.9 mg, 9%) as a yellow solid. ¹H NMR (400 MHz, DMSO-d₆) δ 8.41 (d, J=5.2 Hz, 1H), 7.51-7.45 (m, 2H), 7.39 (d, J=5.2 Hz, 1H), 7.34-7.28 (m, 1H), 7.28-7.23 (m, 2H), 4.44 (s, 2H). LC/MS (ESI, m/z): [(M−1)]⁻=233.95.

### Step 4: 8-Phenoxy-2,4-dihydro-2,7-naphthyridine-1,3-dione

A mixture of 4-(cyanomethyl)-2-phenoxypyridine-3-carbonitrile (46.90 mg, 0.199 mmol, 1.00 equiv) and con. HCl (1.00 mL) was stirred for 2 h at 75° C. under nitrogen atmosphere. The reaction was monitored by LCMS. The mixture was allowed to cool down to room temperature. The resulting mixture was concentrated under reduced pressure. The residue was purified by pre-HPLC with the following conditions: Column: Atlantis HILIC Column 19×150 mm, 5 um; Eluent A: water (plus 0.1% FA); Eluent B: ACN; Gradient: 26%-63% B in 8 min; Flow rate: 60 mL/min; Detector: 220/254 nm; desired fractions were concentrated under reduced pressure to afford 8-phenoxy-2,4-dihydro-2,7-naphthyridine-1,3-dione (2 mg, 4%) as a green solid. ¹H NMR (400 MHz, DMSO-d₆) δ 11.24 (s, 1H), 8.20 (s, 0.6H), 7.73 (s, 0.3H), 7.50-7.34 (m, 2H), 7.23 (t, J=8.0 Hz, 1H), 7.11-7.05 (m, 2.6H), 6.91 (s, 0.3H), 5.58 (s, 0.3H), 4.07 (d, J=6.7 Hz, 2H). LC/MS (ESI, m/z): [(M+1)]⁺=255.05.

### Example 52. 8-(Benzylamino)-2,4-dihydroisoquinoline-1,3-dione (I-215)

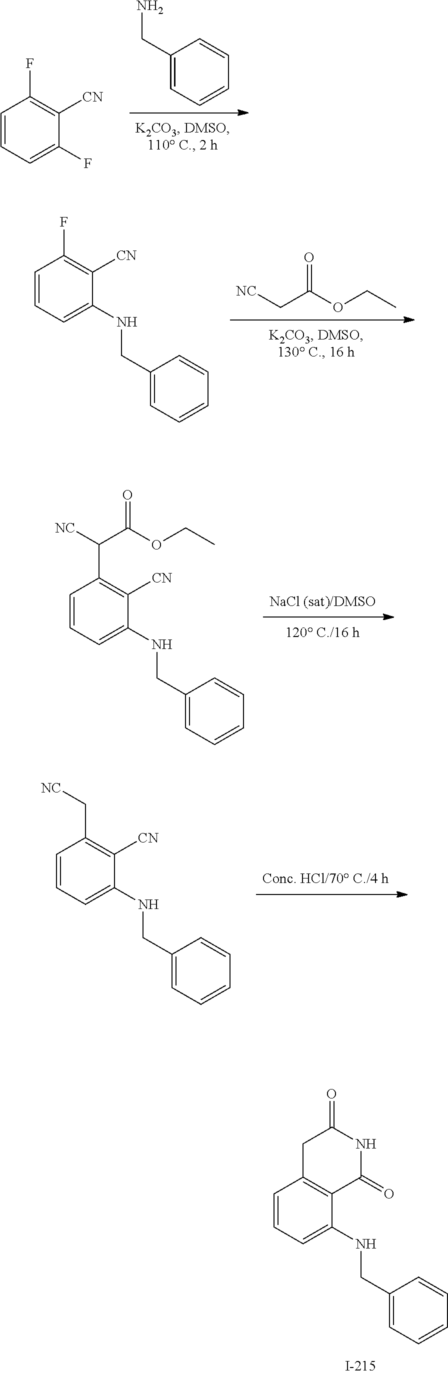

### Step 1: 2-(Benzylamino)-6-fluorobenzonitrile

To a stirred solution of 2,6-difluorobenzonitrile (1.00 g, 7.189 mmol, 1.00 equiv) and benzylamine (0.77 g, 7.189 mmol, 1.00 equiv) in DMSO (15 mL) was added K₂CO₃ (2.98 g, 21.562 mmol, 3.00 equiv) in portions at room temperature under air atmosphere. The resulting mixture was stirred for 16 h at 110° C. under air atmosphere. The reaction was monitored by LCMS. The mixture was allowed to cool down to room temperature. The resulting mixture was diluted with water (80 mL) and extracted with EtOAc (2×50 mL). The combined organic layers were washed with brine (2×30 mL), dried over anhydrous Na₂SO₄. After filtration, the filtrate was concentrated under reduced pressure. This resulted in 2-(benzylamino)-6-fluorobenzonitrile (1.95 g, crude) as a light yellow solid. The crude product was used in the next step directly without further purification. LC/MS (ESI, m/z): [(M−1)]⁻=225.15.

### Step 2: 2-(Benzylamino)-6-(cyanomethyl)benzonitrile

To a stirred solution of 2-(benzylamino)-6-fluorobenzonitrile (800.00 mg, 3.536 mmol, 1.00 equiv) and ethyl cyanoacetate (439.96 mg, 3.889 mmol, 1.10 equiv) in DMSO (5 mL) was added K₂CO₃ (977.35 mg, 7.072 mmol, 2.00 equiv) in portions at room temperature under air atmosphere. The resulting mixture was stirred for overnight at 120° C. under air atmosphere. The reaction was monitored by LCMS. The mixture was allowed to cool down to room temperature. The resulting mixture was diluted with water (20 mL). The mixture was acidified to pH=4 with HCl (2 M aq.). The resulting mixture was extracted with EtOAc (2×20 mL). The combined organic layers was washed with brine (20 mL), dried over anhydrous Na₂SO₄. After filtration, the filtrate was concentrated under reduced pressure. The residue was purified by reverse flash chromatography with the following conditions: column, C¹⁸ silica gel; mobile phase, ACN in water, 25% to 45% gradient in 10 min; detector, UV 254 nm. This resulted in ethyl 2-[3-(benzylamino)-2-cyanophenyl]-2-cyanoacetate (150 mg, 13%) as a light brown solid. LC/MS (ESI, m/z): [(M+1)]⁺=320.10.

### Step 3: 2-(Benzylamino)-6-(cyanomethyl)benzonitrile

To a stirred solution of ethyl 2-[3-(benzylamino)-2-cyanophenyl]-2-cyanoacetate (60.00 mg, 0.188 mmol, 1.00 equiv) in DMSO (1 mL) was added sat. aq. NaCl (1 mL) at room temperature under air atmosphere. The resulting mixture was stirred for overnight at 100° C. under air atmosphere. The reaction was monitored by LCMS. The mixture was allowed to cool down to room temperature. The resulting mixture was diluted with water (10 mL) and extracted with EtOAc (2×10 mL). The combined organic layers was washed with brine (10 mL), dried over anhydrous Na₂SO₄. After filtration, the filtrate was concentrated under reduced pressure. The residue was purified by reverse flash chromatography with the following conditions: column, C¹⁸ silica gel; mobile phase, ACN in water, 35% to 55% gradient in 10 min; detector, UV 254 nm. This resulted in 2-(benzylamino)-6-(cyanomethyl)benzonitrile (40 mg, 86%) as a brown solid. LC/MS (ESI, m/z): [(M+1)]⁺=248.20.

### Step 4: 8-(Benzylamino)-2,4-dihydroisoquinoline-1,3-dione

A solution of 2-(benzylamino)-6-(cyanomethyl)benzonitrile (45.00 mg, 0.182 mmol, 1.00 equiv) in conc. HCl (1.50 mL) was stirred for 2 h at 75° C. under nitrogen atmosphere. The reaction was monitored by LCMS. The mixture was allowed to cool down to room temperature. The resulting mixture was concentrated under reduced pressure. The residue was purified by reverse phase Flash chromatography with the following conditions: Column: WelFlash™ C18-I, 20-40 nm, 120 g; Eluent A: water (plus 10 mmol/L FA); Eluent B: ACN; Gradient: 40%-60% B in 25 min; Flow rate: 60 mL/min; Detector: 220/254 nm; desired fractions were collected at 52% B and concentrated under reduced pressure to afford 8-(benzylamino)-2,4-dihydroisoquinoline-1,3-dione (30 mg, 62%) as a yellow solid. ¹H NMR (400 MHz, Chloroform-d) δ 8.95 (s, 1H), 7.94 (s, 1H), 7.40-7.35 (m, 4H), 7.34-7.29 (m, 2H), 6.58 (d, J=8.6 Hz, 1H), 6.48 (dd, J=7.3, 1.2 Hz, 1H), 4.52 (d, J=5.6 Hz, 2H), 3.97 (s, 2H). LC/MS (ESI, m/z): [(M−1)]⁻=264.95.

### Example 53. 5-Methyl-8-phenoxy-2,4-dihydroisoquinoline-1,3-dione (I-216)

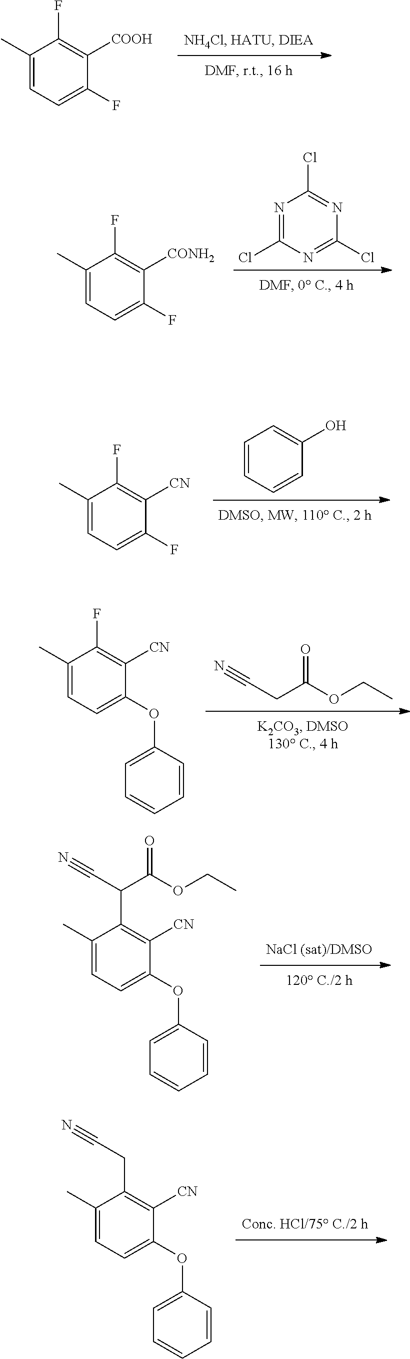

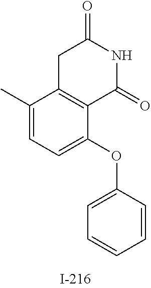 [SMILES: Cc1ccc(Oc2ccccc2)c2c1CC(=O)NC2=O]

### Step 1: 2,6-Difluoro-3-methylbenzamide

To a stirred mixture of 2,6-difluoro-3-methylbenzoic acid (5.00 g, 29.048 mmol, 1.00 equiv) and DIEA (18.77 g, 145.238 mmol, 5.00 equiv) in DMF (300.00 mL) were added HATU (16.57 g, 43.579 mmol, 1.50 equiv) and NH₄Cl (7.77 g, 145.238 mmol, 5.00 equiv) in portions at room temperature under air atmosphere. The resulting mixture was stirred for 16 h at room temperature under air atmosphere. The reaction was monitored by LCMS. The resulting mixture was diluted with water (900 mL) and extracted with EtOAc (3×300 mL). The combined organic layers was dried over anhydrous Na₂SO₄. After filtration, the filtrate was concentrated under reduced pressure to afford 2,6-difluoro-3-methylbenzamide (4.3818 g, crude) as a yellow solid. LC/MS (ESI, m/z): [(M+1)]⁺=172.05.

### Step 2: 2,6-Difluoro-3-methylbenzonitrile

To a stirred solution of 2,6-difluoro-3-methylbenzamide (4.38 g, 25.592 mmol, 1.00 equiv) in DMF (22.00 mL) was added cyanuric chloride (5.19 g, 28.145 mmol, 1.10 equiv) at 0° C. under air atmosphere. The resulting mixture was stirred for 4 h at 0° C. under air atmosphere. The resulting mixture was diluted with water (66 mL) and extracted with EtOAc (3×40 mL). The combined organic phase was dried over anhydrous Na₂SO₄. After filtration, the filtrate was concentrated under reduced pressure to afford the title compound (2.49 g, 64%) as a yellow solid. ¹H NMR (400 MHz, DMSO-d₆) δ 7.75 (tdd, J=8.8, 6.6, 0.8 Hz, 1H), 7.33 (td, J=8.8, 1.3 Hz, 1H), 2.26 (s, 3H).

### Step 3: 2-Fluoro-3-methyl-6-phenoxybenzonitrile

To a stirred mixture of 2,6-difluoro-3-methylbenzonitrile (1.00 g, 6.530 mmol, 1.00 equiv) and phenol (0.61 g, 6.482 mmol, 0.99 equiv) in DMSO (15.00 mL) was added K₂CO₃ (1.80 g, 13.024 mmol, 1.99 equiv) in portions at room temperature under air atmosphere. The resulting mixture was stirred for 2 h at 110° C. under air atmosphere with microwave radiation. The mixture was allowed to cool down to room temperature. The resulting mixture was diluted with water (40 mL) and extracted with EtOAc (2×30 mL). The combined organic phase was dried over anhydrous Na₂SO₄. After filtration, the filtrate was concentrated under reduced pressure. The residue was purified by reverse phase Flash chromatography with the following conditions: Column: WelFlash™ C18-I, 20-40 nm 330 g; Eluent A: water (plus 10 mmol/L TFA); Eluent B: ACN; Gradient: 45%-65% B in 30 min; Flow rate: 80 mL/min; Detector: 220/254 nm; desired fractions were collected at 59% B and concentrated under reduced pressure to afford the title compound (0.61 g, 41%) as a yellow solid. ¹H NMR (400 MHz, DMSO-d₆) δ 7.79 (t, J=7.8 Hz, 1H), 7.46-7.33 (m, 3H), 7.14 (d, J=7.3 Hz, 1H), 6.91 (d, J=8.4 Hz, 2H), 2.08 (s, 3H). LC/MS (ESI, m/z): [(M+1)]⁺=228.05.

### Step 4: Ethyl 2-cyano-2-(2-cyano-6-methyl-3-phenoxyphenyl)acetate

To a stirred mixture of 2-fluoro-3-methyl-6-phenoxybenzonitrile (0.61 g, 2.684 mmol, 1.00 equiv) and ethyl cyanoacetate (0.31 g, 2.741 mmol, 1.02 equiv) in DMSO (12.00 mL) was added K₂CO₃ (0.75 g, 5.427 mmol, 2.02 equiv) at room temperature under air atmosphere. The resulting mixture was stirred for 4 h at 130° C. under nitrogen atmosphere. The mixture was allowed to cool down to room temperature. The resulting mixture was diluted with water (36 mL) and acidified to pH 5 with HCl (2 M aq.). The resulting mixture was extracted with EtOAc (3×20 mL). The combined organic phase was dried over anhydrous Na₂SO₄. After filtration, the filtrate was concentrated under reduced pressure to afford the title compound (1.14 g, crude) as a yellow solid. LC/MS (ESI, m/z): [(M−1)]⁻=318.90.

### Step 5: 2-(Cyanomethyl)-3-methyl-6-phenoxybenzonitrile

A stirred solution of ethyl 2-cyano-2-(2-cyano-6-methyl-3-phenoxyphenyl)acetate (1.14 g, 3.559 mmol, 1.00 equiv) in DMSO (10 mL) and brine (2 mL) was stirred for 2 h at 120° C. under air atmosphere. The mixture was allowed to cool down to room temperature. The resulting mixture was diluted with water (50 mL) and extracted with EtOAc (3×30 mL). The combined organic phase was dried over anhydrous Na₂SO₄. After filtration, the filtrate was concentrated under reduced pressure. The residue was purified by silica gel column chromatography, eluted with petroleum ether/EtOAc (3:1) to afford 2-(cyanomethyl)-3-methyl-6-phenoxybenzonitrile (0.57 g, 65%) as a yellow solid. ¹H NMR (400 MHz, DMSO-d₆) δ 7.76 (dd, J=8.0, 0.9 Hz, 1H), 7.50 (d, J=8.0 Hz, 1H), 7.42-7.33 (m, 2H), 7.14-7.08 (m, 1H), 6.89-6.82 (m, 2H), 4.29 (s, 2H), 2.11 (s, 3H). LC/MS (ESI, m/z): [(M−1)]⁻=246.95.

### Step 6: 5-Methyl-8-phenoxy-2,4-dihydroisoquinoline-1,3-dione

A solution of 2-(cyanomethyl)-3-methyl-6-phenoxybenzonitrile (0.57 g, 2.296 mmol, 1.00 equiv) in conc. HCl (16.00 mL) was stirred for 2 h at 75° C. under nitrogen atmosphere. The mixture was allowed to cool down to room temperature. The resulting mixture was concentrated under reduced pressure. The residue was purified by reverse phase Flash chromatography with the following conditions: Column: WelFlash™ C18-I, 20-40 nm 120 g; Eluent A: water (plus 10 mmol/L FA); Eluent B: ACN; Gradient: 30%-50% B in 25 min; Flow rate: 60 mL/min; Detector: 220/254 nm; desired fractions were collected at 42% B and concentrated under reduced pressure to afford the title compound (0.21 g, 34%) as a yellow solid. ¹H NMR (400 MHz, DMSO-d₆) δ 10.96 (s, 1H), 7.60 (d, J=7.9 Hz, 1H), 7.32-7.20 (m, 3H), 6.95 (t, J=7.3 Hz, 1H), 6.71-6.68 (m, 2H), 4.02 (s, 2H), 2.07 (s, 3H). LC/MS (ESI, m/z): [(M+1)]⁺=268.05.

### Example 54. 8-(2-Chlorophenoxy)-2,4-dihydroisoquinoline-1,3-dione (I-201)

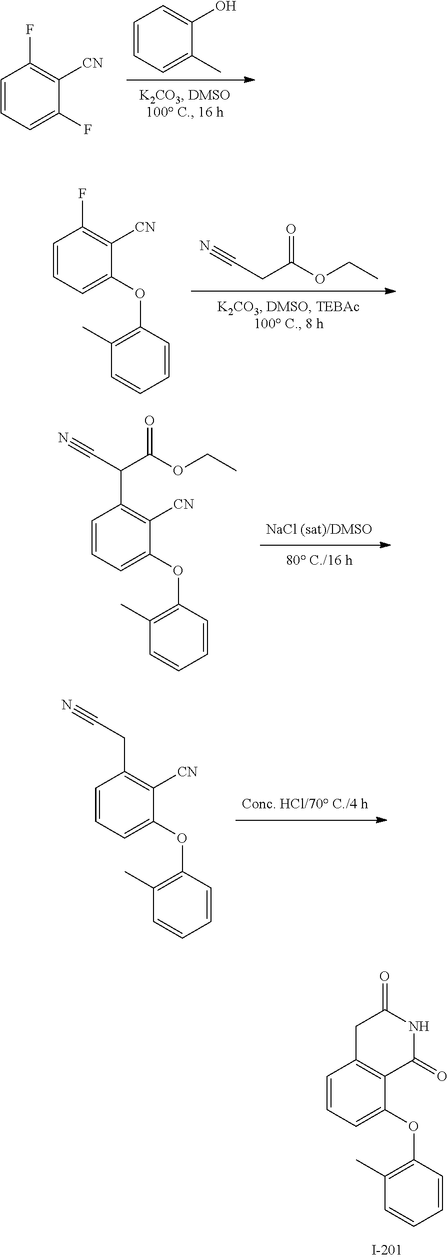

### Step 1: 2-Fluoro-6-(2-methylphenoxy)benzonitrile

To a stirred mixture of 2,6-difluorobenzonitrile (3.00 g, 21.566 mmol, 1.00 equiv) and o-cresol (2.57 g, 23.723 mmol, 1.10 equiv) in DMF (30.00 mL) was added K₂CO₃ (5.96 g, 43.133 mmol, 2.00 equiv) in portions at room temperature under air atmosphere. The resulting mixture was stirred for 16 h at 100° C. under air atmosphere. The mixture was allowed to cool down to room temperature and was diluted with water (90 mL). The resulting mixture was extracted with EtOAc (3×40 mL) and dried over anhydrous Na₂SO₄. After filtration, the filtrate was concentrated under reduced pressure. The residue was purified by reverse phase Flash chromatography with the following conditions: Column: WelFlash™ C18-I, 20-40 um, 330 g; Eluent A: water (plus 10 mmol/L FA); Eluent B: ACN; Gradient: 47%-67% B in 30 min; Flow rate: 80 mL/min; Detector: 220/254 nm; The desired fractions were collected at 65% B and concentrated under reduced pressure to afford the title compound (3.24 g, 66%) as a yellow solid. LC/MS (ESI, m/z): [(M−1)]⁻=226.05.

### Step 2: Ethyl 2-[2-cyano-3-(2-methylphenoxy)phenyl]-2-isocyanoacetate

To a stirred mixture of 2-fluoro-6-(2-methylphenoxy)benzonitrile (3.24 g, 14.258 mmol, 1.00 equiv) and ethyl cyanoacetate (1.61 g, 14.258 mmol, 1.00 equiv) in DMSO (25.00 mL) was added K₂CO₃ (3.94 g, 28.516 mmol, 2.00 equiv) in portions at room temperature under air atmosphere. The resulting mixture was stirred for 16 h at 120° C. under nitrogen atmosphere. The mixture was allowed to cool down to room temperature and diluted with water (75 mL). The mixture was acidified to pH 5 with 2 NHCl (aq.). The resulting mixture was extracted with EtOAc (3×50 mL). The combined organic phase was dried over anhydrous Na₂SO₄. After filtration, the filtrate was concentrated under reduced pressure to afford the title compound (5.42 g, crude) as a yellow solid. LC/MS (ESI, m/z): [(M+18)]⁺=338.15.

### Step 3: 2-(Isocyanomethyl)-6-(2-methylphenoxy)benzonitrile

A stirred mixture of ethyl 2-[2-cyano-3-(2-methylphenoxy)phenyl]-2-isocyanoacetate (2.70 g, 8.428 mmol, 1.00 equiv) in brine (1.59 mL) and DMSO (3.17 mL) was stirred for 2 h at 120° C. under air atmosphere. The mixture was allowed to cool down to room temperature. The resulting mixture was diluted with water (30 mL) and was extracted with EtOAc (3×30 mL). The combined organic phase was dried over anhydrous Na₂SO₄. After filtration, the filtrate was concentrated under reduced pressure. The residue was purified by silica gel column chromatography, eluted with petroleum ether/EtOAc (2:1) to afford the title compound (1.06 g, 51%) as a yellow solid. ¹H NMR (400 MHz, DMSO-d₆) δ 7.65 (dd, J=8.6, 7.7 Hz, 1H), 7.41 (dd, J=7.5, 1.8 Hz, 1H), 7.36-7.20 (m, 2H), 7.25 (td, J=7.4, 1.4 Hz, 1H), 7.10 (dd, J=8.0, 1.3 Hz, 1H), 6.70 (d, J=8.6 Hz, 1H), 4.33 (s, 2H), 2.16 (s, 3H). LC/MS (ESI, m/z): [(M+1)]⁺=249.20.

### Step 4: 8-(2-Methylphenoxy)-2,4-dihydroisoquinoline-1,3-dione

A stirred mixture of 2-(isocyanomethyl)-6-(2-methylphenoxy)benzonitrile (500.00 mg, 2.014 mmol, 1.00 equiv) in conc. HCl (14.00 mL) was stirred for 2 h at 75° C. under nitrogen atmosphere. The mixture was allowed to cool down to room temperature. The precipitated solids were collected by filtration and washed with water (3×50 mL) and dried to afford the title compound (180 mg, 33%) as a white solid. ¹H NMR (400 MHz, DMSO-d₆) δ 11.04 (s, 1H), 7.55 (t, J=8.0 Hz, 1H), 7.31 (dt, J=7.5, 1.4 Hz, 1H), 7.20-7.04 (m, 3H), 6.75-6.70 (m, 2H), 4.06 (s, 2H), 2.20 (s, 3H). LC/MS (ESI, m/z): [(M+1)]⁺=268.05.

### Example 55. 8-(2-Chlorophenoxy)-2,4-dihydroisoquinoline-1,3-dione (I-202)

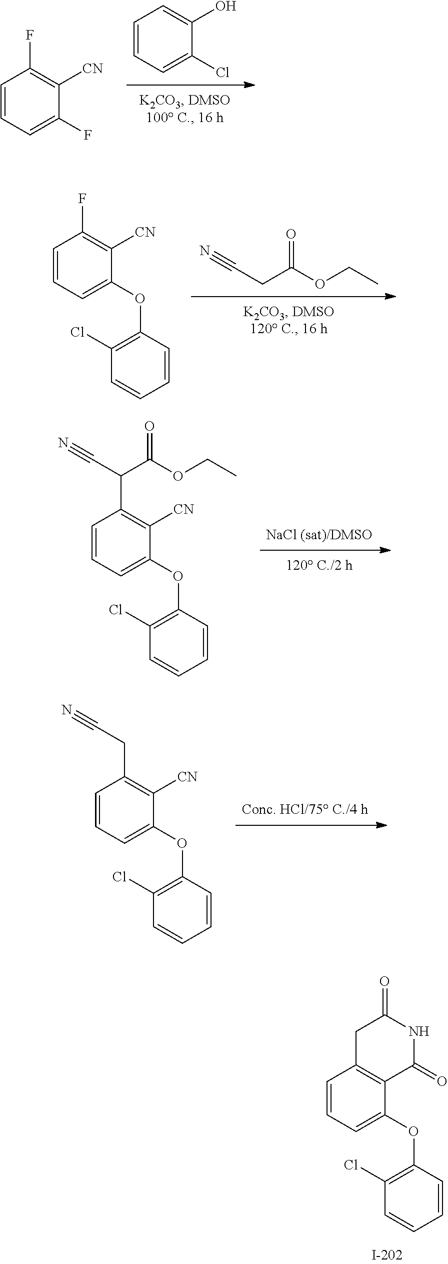

### Step 1: 2-(2-Chlorophenoxy)-6-fluorobenzonitrile

To a stirred mixture of 2,6-difluorobenzonitrile (2.00 g, 14.378 mmol, 1.00 equiv) and 2-chlorophenol (2.03 g, 15.815 mmol, 1.1 equiv) in DMF (20.00 mL) was added K₂CO₃ (3.97 g, 28.755 mmol, 2 equiv) in portions at room temperature under air atmosphere. The resulting mixture was stirred for 16 h at 100° C. under air atmosphere. The mixture was allowed to cool down to room temperature. The resulting mixture was diluted with water (60 mL) and extracted with EtOAc (3×30 mL). The combined organic phase was dried over anhydrous Na₂SO₄. After filtration, the filtrate was concentrated under reduced pressure. The residue was purified by reverse phase Flash chromatography with the following conditions: Column: WelFlash™ C18-I, 20-40 um, 330 g; Eluent A: water (plus 10 mmol/L FA); Eluent B: ACN; Gradient: 40%-60% B in 30 min; Flow rate: 80 mL/min; Detector: 220/254 nm; desired fractions were collected at 55% B and concentrated under reduced pressure to afford the title compound (2.4 g, 67%) as a yellow solid. ¹H NMR (400 MHz, DMSO-d₆) δ 7.75-7.65 (m, 2H), 7.53-7.37 (m, 3H), 7.30-7.23 (m, 1H), 6.64-6.57 (m, 1H). LC/MS (ESI, m/z): [(M−1)]⁻=245.95, 247.95.

### Step 2: Ethyl 2-[3-(2-chlorophenoxy)-2-cyanophenyl]-2-isocyanoacetate

To a stirred mixture of 2-(2-chlorophenoxy)-6-fluorobenzonitrile (1.00 g, 4.038 mmol, 1.00 equiv) in DMSO (7.50 mL) were added ethyl cyanoacetate (456.76 mg, 4.038 mmol, 1.00 equiv) and K₂CO₃ (1116.14 mg, 8.076 mmol, 2.00 equiv) in portions at room temperature under air atmosphere. The resulting mixture was stirred for 16 h at 120° C. under nitrogen atmosphere. The mixture was allowed to cool down to room temperature. The resulting mixture was diluted with water (30 mL), acidified to pH 5 with 2 NHCl (aq.) and extracted with EtOAc (3×15 mL). The combined organic phase was dried over anhydrous Na₂SO₄. After filtration, the filtrate was concentrated under reduced pressure to afford the title compound (1.75 g, crude) as a yellow solid. ¹H NMR (400 MHz, DMSO-d₆) δ 7.76-7.66 (m, 2H), 7.49 (ddd, J=8.1, 7.2, 1.5 Hz, 1H), 7.44-7.36 (m, 3H), 6.86 (dd, J=8.6, 0.8 Hz, 1H), 6.13 (s, 1H), 4.29 (q, J=7.1 Hz, 2H), 1.24 (t, J=7.1 Hz, 3H). LC/MS (ESI, m/z): [(M−1)]⁻=338.90, 340.90.

### Step 3: 2-(2-Chlorophenoxy)-6-(cyanomethyl)benzonitrile

A stirred mixture of ethyl 2-[3-(2-chlorophenoxy)-2-cyanophenyl]-2-isocyanoacetate (800.00 mg, 2.348 mmol, 1.00 equiv) in DMSO (0.90 mL) and brine (0.45 mL) was stirred for 2 h at 120° C. under air atmosphere. The mixture was allowed to cool down to room temperature. The resulting mixture was diluted with water (30 mL). The resulting mixture was extracted with EtOAc (3×20 mL), dried over anhydrous Na₂SO₄. After filtration, the filtrate was concentrated under reduced pressure. The residue was purified by silica gel column chromatography, eluted with Petroleum ether/EtOAc (2:1) to afford 2-(2-chlorophenoxy)-6-(isocyanomethyl)benzonitrile (311 mg, 49.30%) as a yellow solid. ¹H NMR (400 MHz, DMSO-d₆) δ 7.72-7.63 (m, 2H), 7.53-7.44 (m, 1H), 7.42-7.33 (m, 3H), 6.75 (dd, J=8.6, 0.8 Hz, 1H), 4.35 (s, 2H). LC/MS (ESI, m/z): [(M−1)]⁻=266.90, 268.90.

### Step 4: 8-(2-Chlorophenoxy)-2,4-dihydroisoquinoline-1,3-dione

A stirred mixture of 2-(2-chlorophenoxy)-6-(isocyanomethyl)benzonitrile (0.15 g, 0.558 mmol, 1.00 equiv) in conc. HCl (3.11 mL) was stirred for 4 h at 75° C. under nitrogen atmosphere. The mixture was allowed to cool down to room temperature. The precipitated solids were collected by filtration, washed with water (3×50 mL) and dried to afford the title compound (0.13 g, 83%) as a white solid. ¹H NMR (400 MHz, DMSO-d₆) δ 11.08 (s, 1H), 7.64-7.56 (m, 2H), 7.28 (ddd, J=8.1, 7.4, 1.6 Hz, 1H), 7.21 (dd, J=7.7, 1.0 Hz, 1H), 7.15 (td, J=7.7, 1.5 Hz, 1H), 6.88-6.82 (m, 2H), 4.07 (s, 2H). LC/MS (ESI, m/z): [(M−1)]⁻=285.90, 287.90.

### Example 56. 8-Phenoxy-2,4-dihydro-2,6-naphthyridine-1,3-dione (I-217)

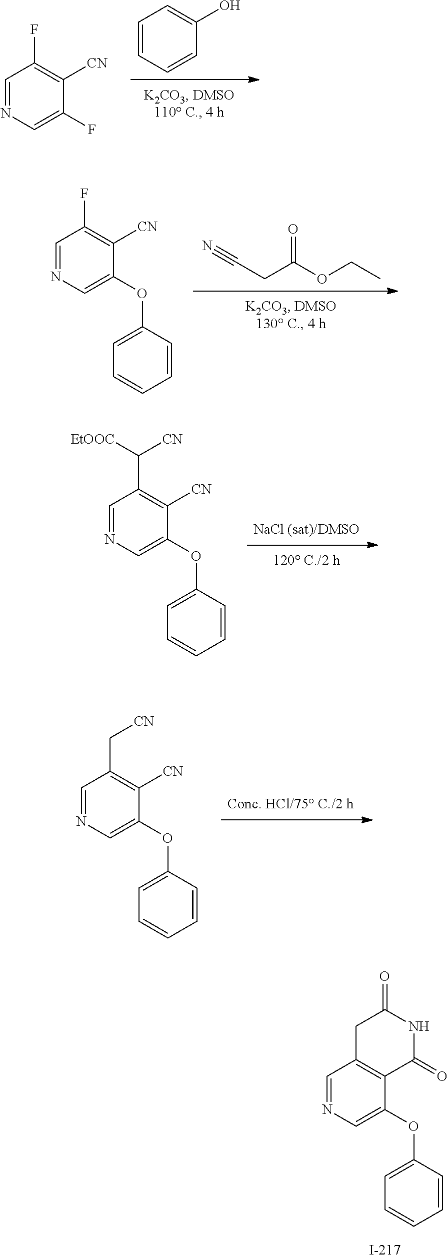

### Step 1: 3-Fluoro-5-phenoxypyridine-4-carbonitrile

To a stirred mixture of 3,5-difluoropyridine-4-carbonitrile (1.00 g, 7.138 mmol, 1.00 equiv) and phenol (740.00 mg, 7.863 mmol, 1.10 equiv) in DMSO (6.00 mL) was added K₂CO₃ (1.97 g, 14.276 mmol, 2.00 equiv) at room temperature under air atmosphere. The resulting mixture was stirred for 4 h at 110° C. under nitrogen atmosphere. The reaction was monitored by LCMS. The mixture was allowed to cool down to room temperature. The resulting mixture was diluted with water (40 mL) and extracted with EtOAc (3×30 mL). The combined organic layers was dried over anhydrous Na₂SO₄. After filtration, the filtrate was concentrated under reduced pressure. The residue was purified by reverse phase Flash chromatography with the following conditions: Column: WelFlash™ C18-I, 20-40 nm, 330 g; Eluent A: water (plus 10 mmol/L FA); Eluent B: ACN; Gradient: 41%-61% B in 25 min; Flow rate: 80 mL/min; Detector: 220/254 nm; desired fractions were collected at 56% B and concentrated under reduced pressure to afford 3-fluoro-5-phenoxypyridine-4-carbonitrile (1.171 g, 77%) as a yellow solid. ¹H NMR (400 MHz, DMSO-d₆) δ 8.69 (s, 1H), 8.21 (s, 1H), 7.56-7.48 (m, 2H), 7.37-7.27 (m, 3H). LC/MS (ESI, m/z): [(M+1)]⁺=215.05.

### Step 2: Ethyl 2-cyano-2-(4-cyano-5-phenoxypyridin-3-yl)acetate

To a stirred mixture of 3-fluoro-5-phenoxypyridine-4-carbonitrile (1.17 g, 5.462 mmol, 1.00 equiv) and ethyl cyanoacetate (0.62 g, 5.481 mmol, 1.00 equiv) in DMSO (6.00 mL) was added K₂CO₃ (1.50 g, 10.853 mmol, 1.99 equiv) at room temperature under air atmosphere. The resulting mixture was stirred for 4 h at 130° C. under nitrogen atmosphere. The reaction was monitored by LCMS. The mixture was allowed to cool down to room temperature. The resulting mixture was diluted with water (30 mL). The mixture was acidified to pH 5 with HCl (2 M aq.). The resulting mixture was extracted with EtOAc (3×30 mL). The combined organic layers was dried over anhydrous Na₂SO₄. After filtration, the filtrate was concentrated under reduced pressure to afford ethyl 2-cyano-2-(4-cyano-5-phenoxypyridin-3-yl)acetate (1.5 g, crude) as yellow oil. LC/MS (ESI, m/z): [(M−1)]⁻=305.95.

### Step 3: 3-(Cyanomethyl)-5-phenoxypyridine-4-carbonitrile

A solution of ethyl 2-cyano-2-(4-cyano-5-phenoxypyridin-3-yl)acetate (1.50 g, 4.881 mmol, 1.00 equiv) in DMSO (1.80 mL) and con. aq. NaCl (1.00 mL) was stirred for 2 h at 120° C. under air atmosphere. The reaction was monitored by LCMS. The mixture was allowed to cool down to room temperature. The resulting mixture was diluted with water (50 mL) and extracted with EtOAc (5×30 mL). The combined organic layers was dried over anhydrous Na₂SO₄. After filtration, the filtrate was concentrated under reduced pressure. The residue was purified by reverse phase Flash chromatography with the following conditions: Column: WelFlash™ C18-I, 20-40 nm, 330 g; Eluent A: water (plus 10 mmol/L FA); Eluent B: ACN; Gradient: 36%-56% B in 25 min; Flow rate: 80 mL/min; Detector: 220/254 nm; desired fractions were collected at 49% B and concentrated under reduced pressure to afford 3-(cyanomethyl)-5-phenoxypyridine-4-carbonitrile (0.63 g, 55%) as a yellow solid. ¹H NMR (400 MHz, DMSO-d₆) δ 8.62 (s, 1H), 8.34 (s, 1H), 7.58-7.46 (m, 2H), 7.39-7.30 (m, 1H), 7.30-7.23 (m, 2H), 4.39 (s, 2H). LC/MS (ESI, m/z): [(M+1)]⁺=236.05.

### Step 4: 8-Phenoxy-2,4-dihydro-2,6-naphthyridine-1,3-dione

A solution of 3-(cyanomethyl)-5-phenoxypyridine-4-carbonitrile (300.00 mg, 1.275 mmol, 1.00 equiv) in con. HCl (8.00 mL) was stirred for 2 h at 75° C. under nitrogen atmosphere. The reaction was monitored by LCMS. The mixture was allowed to cool down to room temperature. The resulting mixture was concentrated under reduced pressure. The residue was dissolved in DMF (10 mL). The precipitated solids were collected by filtration, washed with water (7×10 mL) and dried to afford 8-phenoxy-2,4-dihydro-2,6-naphthyridine-1,3-dione (8 mg, 2%) as a yellow solid. H NMR (400 MHz, DMSO-d₆) δ 11.36 (s, 1H), 8.53 (s, 1H), 8.29 (s, 1H), 7.46-7.28 (m, 2H), 7.14 (dt, J=15.0, 7.4 Hz, 1H), 7.02-6.91 (m, 2H), 4.09 (s, 2H). LC/MS (ESI, m/z): [(M+1)]⁺=255.05.

### Example 57. 4-Phenoxy-6,8-dihydro-1,6-naphthyridine-5,7-dione

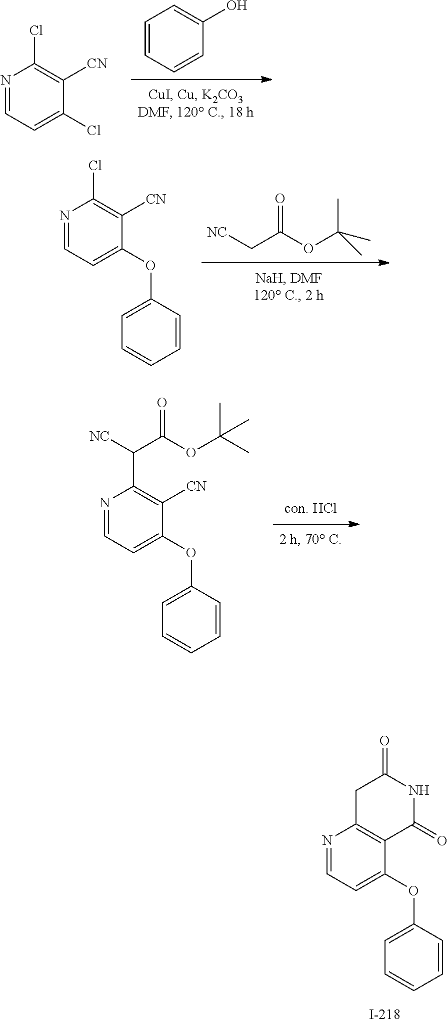

### Step 1: 2-Chloro-4-phenoxypyridine-3-carbonitrile

To a stirred mixture of 2,4-dichloropyridine-3-carbonitrile (2.00 g, 11.561 mmol, 1.00 equiv) and phenol (1.31 g, 13.941 mmol, 1.21 equiv) in DMF (30.00 mL) were added Cu (0.22 g, 3.525 mmol, 0.30 equiv), K₂CO₃ (3.20 g, 23.154 mmol, 2.00 equiv) and CuI (0.22 g, 1.155 mmol, 0.10 equiv) at room temperature under air atmosphere. The resulting mixture was stirred for 18 h at 120° C. under nitrogen atmosphere. The reaction was monitored by LCMS. The mixture was allowed to cool down to room temperature. The resulting mixture was diluted with water (100 mL) and extracted with EtOAc (3×50 mL). The combined organic layers was dried over anhydrous Na₂SO₄. After filtration, the filtrate was concentrated under reduced pressure. The residue was purified by reverse phase Flash chromatography with the following conditions: Column: WelFlash™C18-I, 20-40 nm, 330 g; Eluent A: water (plus 10 mmol/L FA); Eluent B: ACN; Gradient: 46%-66% B in 25 min; Flow rate: 80 mL/min; Detector: 220/254 nm; desired fractions were collected at 42% B and concentrated under reduced pressure to afford 2-chloro-4-phenoxypyridine-3-carbonitrile (2.24 g, 84%) as a yellow solid. ¹H NMR (400 MHz, DMSO-d₆) δ 8.46 (d, J=6.0 Hz, 1H), 7.62-7.53 (m, 2H), 7.45-7.39 (m, 1H), 7.38-7.32 (m, 2H), 6.82 (d, J=6.0 Hz, 1H).

### Step 2: Tert-butyl 2-cyano-2-(3-cyano-4-phenoxypyridin-2-yl)acetate

To a stirred solution of tert-butyl 2-cyanoacetate (122.60 mg, 0.868 mmol, 2.00 equiv) in DMSO (2.00 mL) was added NaH (60% dispersion in mineral oil, 20.86 mg, 0.869 mmol, 2.00 equiv) at room temperature under air atmosphere. The resulting mixture was stirred for 1 h at room temperature under nitrogen atmosphere. To the mixture was added 2-chloro-4-phenoxypyridine-3-carbonitrile (100.00 mg, 0.434 mmol, 1.00 equiv) at room temperature under nitrogen atmosphere. The resulting mixture was stirred for 2 h at 120° C. under nitrogen atmosphere. The reaction was monitored by LCMS. The mixture was allowed to cool down to room temperature. The resulting mixture was diluted with water (20 mL) and extracted with EtOAc (3×20 mL). The combined organic layers was dried over anhydrous Na₂SO₄. After filtration, the filtrate was concentrated under reduced pressure. The residue was purified by reverse phase Flash chromatography with the following conditions: Column: WelFlash™ C18-I, 20-40 nm, 120 g; Eluent A: water (plus 10 mmol/L NH₄HCO₃); Eluent B: ACN; Gradient: 33%-53% B in 25 min; Flow rate: 60 mL/min; Detector: 220/254 nm; desired fractions were collected at 46% B and concentrated under reduced pressure to afford tert-butyl 2-cyano-2-(3-cyano-4-phenoxypyridin-2-yl)acetate (11.8 mg, 8%) as a yellow solid. ¹H NMR (400 MHz, DMSO-d₆) δ 8.19 (d, J=7.2 Hz, 1H), 7.76-7.64 (m, 1H), 7.63-7.53 (m, 2H), 7.47-7.39 (m, 1H), 7.38-7.31 (m, 2H), 6.19 (d, J=7.3 Hz, 1H), 1.51 (s, 9H). LC/MS (ESI, m/z): [(M−1)]⁻=334.00.

### Step 3: 4-Phenoxy-6,8-dihydro-1,6-naphthyridine-5,7-dione

A solution of tert-butyl 2-cyano-2-(3-cyano-4-phenoxypyridin-2-yl)acetate (45.50 mg, 0.136 mmol, 1.00 equiv) in con. HCl (1.00 mL) was stirred for 30 min at room temperature under air atmosphere. The resulting mixture was stirred for additional 2 h at 70° C. under air atmosphere. The reaction was monitored by LCMS. The mixture was allowed to cool down to room temperature and concentrated under reduced pressure. The residue was purified by pre-HPLC with the following conditions: Column: Atlantis HILIC Column 19×150 mm, 5 um; Eluent A: water (plus 0.05% FA); Eluent B: ACN; Gradient: 10%-40% B in 7 min; Flow rate: 60 mL/min; Detector: 220/254 nm; desired fractions were concentrated under reduced pressure to afford 4-phenoxy-6,8-dihydro-1,6-naphthyridine-5,7-dione (5.4 mg, 16%) as a yellow solid. ¹H NMR (400 MHz, DMSO-d₆) δ 12.31 (br, 1H), 10.19 (br, 1H), 7.77 (d, J=7.2 Hz, 1H), 7.56-7.48 (m, 3H), 7.41-7.30 (m, 1H), 7.27-7.12 (m, 2H), 5.54 (d, J=7.2 Hz, 1H). LC/MS (ESI, m/z): [(M+1)]⁺=255.05.

### Example 58. 8-(2-Methylphenoxy)-2H-isoquinoline-1,3,4-trione (I-219)

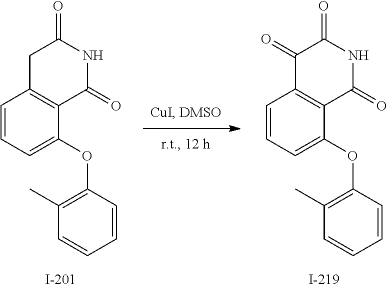 [SMILES: Cc1ccccc1Oc1cccc2c1C(=O)NC(=O)C2 ; Cc1ccccc1Oc1cccc2c1C(=O)NC(=O)C2=O]

To a stirred mixture of 8-(2-methylphenoxy)-2,4-dihydroisoquinoline-1,3-dione (20.00 mg, 0.075 mmol, 1.00 equiv) in DMSO (1.00 mL) was added CuI (6.00 mg, 0.032 mmol, 0.42 equiv) at room temperature under air atmosphere. The resulting mixture was stirred for 12 h at room temperature under air atmosphere. The reaction was monitored by LCMS. The resulting mixture was purified by pre-HPLC with the following conditions: Column: Atlantis HILIC Column 19×150 mm, 5 um; Eluent A: water (plus 0.1% FA); Eluent B: ACN; Gradient: 25%-50% B in 7 min; Flow rate: 60 mL/min; Detector: 220/254 nm; desired fractions were concentrated under reduced pressure to afford 8-(2-methylphenoxy)-2H-isoquinoline-1,3,4-trione (8 mg, 38%) as a yellow solid. ¹H NMR (400 MHz, DMSO-d₆) δ 11.74 (s, 1H), 7.88-7.75 (m, 2H), 7.36 (dt, J=7.0, 1.5 Hz, 1H), 7.25-7.18 (m, 2H), 7.12 (td, J=7.4, 1.3 Hz, 1H), 6.79 (dd, J=8.1, 1.3 Hz, 1H), 2.22 (s, 3H). LC/MS (ESI, m/z): [(M+1)]⁺=282.05.

All of the U.S. patents, U.S. patent application publications, U.S. patent applications, foreign patents, foreign patent applications, and non-patent publications referred to in this specification are incorporated herein by reference in their entireties.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of formula I′: 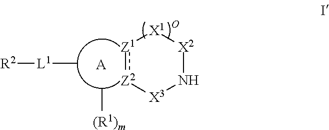
or a pharmaceutically acceptable salt thereof, wherein:
X¹ is —CR₂—, —O—, —NR—, —CF₂—, 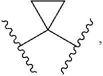 [SMILES: *C1(*)CC1]
C(O)—, —C(S)—, or 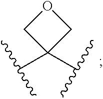 [SMILES: *C1(*)COC1]
X² and X³ are independently —CR₂—, —C(O)—, —C(S)—, —CR₂C(O)—, or 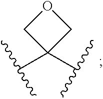 [SMILES: *C1(*)COC1]
Z¹ and Z² are independently a carbon atom or a nitrogen atom;
Ring A is a fused ring selected from benzo, a 4-6 membered saturated or partially unsaturated carbocyclic or heterocyclic ring having 1-3 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —S—, —C(O)—, —C(S)—, —CR₂—, —CRF—, —CF₂—, —NR—, or —S(O)₂—;
each R¹ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃; or
two R¹ groups are optionally taken together to form an optionally substituted 5-8 membered partially unsaturated or aryl fused ring having 0-2 heteroatoms independently selected from nitrogen, oxygen, or sulfur;
each R⁴ is independently selected from an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each R is independently selected from hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same carbon or nitrogen are optionally taken together with their intervening atoms to form an optionally substituted 4-7 membered saturated, partially unsaturated, or heteroaryl ring having 0-3 heteroatoms, in addition to the carbon or nitrogen, independently selected from nitrogen, oxygen, and sulfur;
R² is selected 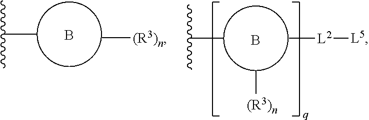
or hydrogen;
Ring B is phenyl, a 4-10 membered saturated or partially unsaturated mono- or bicyclic carbocyclic or heterocyclic ring having 1-3 heteroatoms independently selected from nitrogen, oxygen, and sulfur, or a 5-6 membered heteroaryl ring having 1-4 heteroatoms independently selected from nitrogen, oxygen, and sulfur, wherein Ring B is further optionally substituted with 1-2 oxo groups;
each R³ is independently selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃;
L² is a covalent bond or a bivalent, saturated or unsaturated, straight or branched C₁₋₂₀ hydrocarbon chain, wherein 0-6 methylene units of L are independently replaced by —C(D)(H)—, —C(D)₂-, —CRF—, —CF₂—, -Cy-, —O—, —N(R)—, —Si(R)₂—, —Si(OH)(R)—, —Si(OH)₂—, —P(O)(OR)—, —P(O)(R)—, —P(O)(NR₂)—, —S—, —OC(O)—, —C(O)O—, —C(O)—, —S(O)—, —S(O)₂—, —N(R)S(O)₂—, —S(O)₂N(R)—, —N(R)C(O)—, —C(O)N(R)—, —OC(O)N(R)—, —N(R)C(O)O—, 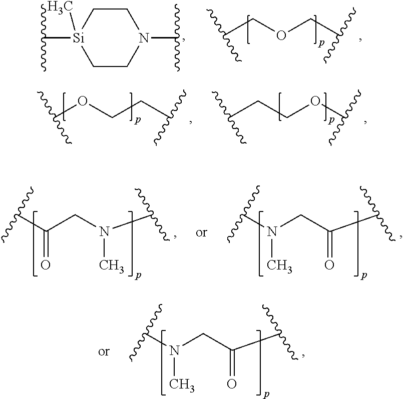
wherein:
each -Cy- is independently an optionally substituted bivalent ring selected from phenylenyl, an 8-10 membered bicyclic arylenyl, a 4-7 membered saturated or partially unsaturated carbocyclylenyl, a 4-11 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-11 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
each p is independently 0, 1, 2, 3, 4, 5, 6, 7, 8, 9, or 10;
R⁵ is selected from hydrogen, deuterium, R⁴, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, and —SiR₃; [symbol]
is a single or double bond;
m is 0, 1, 2, 3 or 4;
n is 0, 1, 2, 3 or 4;
o is 0, 1, or 2;
each p is independently 0, 1, 2, 3, 4, 5, 6, 7, 8, 9, or 10; and
q is 0 or 1.

2. The compound of claim 1, wherein said compound is any one of formulae: 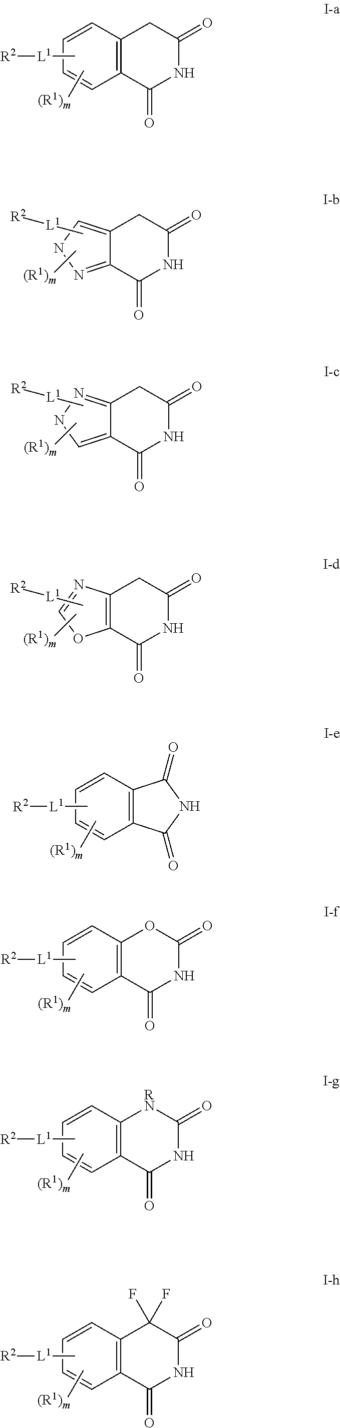 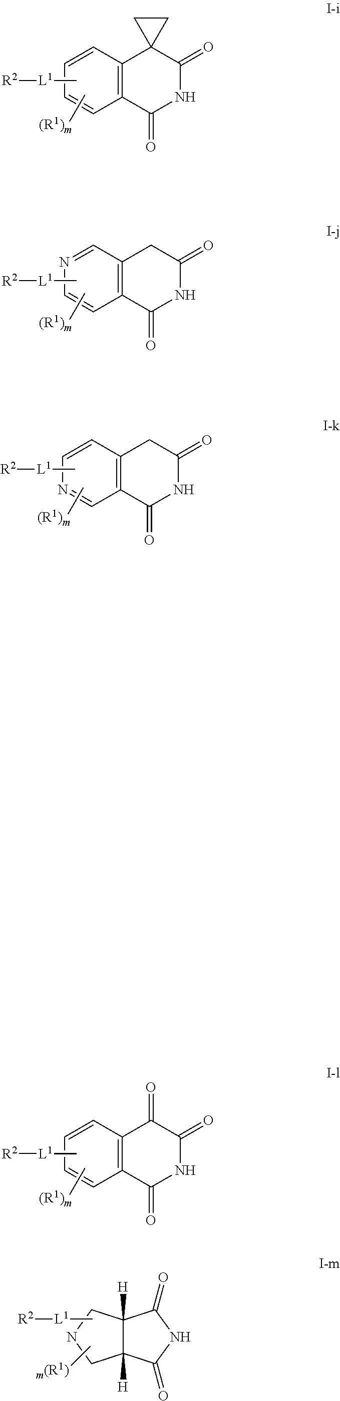
or a pharmaceutically acceptable salt thereof.

3. The compound of any either claim 1 or claim 2, wherein each R¹ is independently selected from hydrogen, halogen, —CN, —NO₂, —OR, —SR, —NR₂, —S(O)₂R, —S(O)₂NR₂, —S(O)R, —CFR₂, —CF₂R, —CF₃, —CR₂(OR), —CR₂(NR₂), —C(O)R, —C(O)OR, —C(O)NR₂, —C(O)N(R)OR, —OC(O)R, —OC(O)NR₂, —C(S)NR₂, —N(R)C(O)OR, —N(R)C(O)R, —N(R)C(O)NR₂, —N(R)S(O)₂R, —OP(O)R₂, —OP(O)(OR)₂, —OP(O)(OR)NR₂, —OP(O)(NR₂)₂, —Si(OR)R₂, and —SiR₃.

4. The compound of any one of claims 1-3, wherein R² is 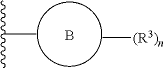
or hydrogen.

5. The compound of any one of claims 1-4, wherein said compound is selected from those depicted in Table 1 of the specification, or a pharmaceutically acceptable salt thereof.

6. A pharmaceutical composition comprising a compound according to any one of the preceding claims, or a pharmaceutically acceptable salt thereof, and a pharmaceutically acceptable carrier, adjuvant, or vehicle.

7. A method of inhibiting CRBN in a biological sample comprising contacting the sample with the compound of any one of claims 1-5, or a pharmaceutically acceptable salt thereof.

8. A method of binding to and altering the specificity of a cereblon complex to induce the ubiquitination and degradation of a complex-associated protein selected from the group listed in paragraph [00174] in a biological sample, comprising contacting the sample with the compound of any one of claims 1-5, or a pharmaceutically acceptable salt thereof.

9. A method of treating an CRBN-mediated disorder, disease, or condition in a patient comprising administering to said patient the pharmaceutical composition of claim 6.

10. The method of claim 9, wherein the disorder is selected from proliferative disorders, neurological disorders and disorders associated with transplantation.

11. The method of claim 10, wherein the disorder is a proliferative disorder.

12. The method of claim 11, wherein the proliferative disorder is a hematological cancer.

13. The method of claim 11, wherein the proliferative disorder is a leukemia.

14. The method of claim 13, wherein the leukemia is selected from the group consisting of acute leukemia, acute lymphoblastic leukemia (ALL), chronic lymphocytic leukemia (CLL), acute myelogenous leukemia, acute myeloid leukemia (AML), adult acute basophilic leukemia, adult acute eosinophilic leukemia, adult acute megakaryoblastic leukemia, adult acute minimally differentiated myeloid leukemia, adult acute monoblastic leukemia, adult acute monocytic leukemia, adult acute myeloblastic leukemia with maturation, adult acute myeloblastic leukemia without maturation, adult acute myeloid leukemia with abnormalities, adult acute myelomonocytic leukemia, adult erythroleukemia, adult pure erythroid leukemia, secondary acute myeloid leukemia, untreated adult acute myeloid leukemia, adult acute myeloid leukemia in remission, adult acute promyelocytic leukemia with PML-RARA, alkylating agent-related acute myeloid leukemia, prolymphocytic leukemia, and chronic myelomonocytic leukemia.

15. The method of claim 11, wherein the proliferative disorder is a lymphoma.

16. The method of claim 15, wherein the lymphoma is selected from the group consisting of adult grade III lymphomatoid granulomatosis, adult nasal type extranodal NK/T-cell lymphoma, anaplastic large cell lymphoma, angioimmunoblastic T-cell lymphoma, cutaneous B-Cell non-Hodgkin lymphoma, extranodal marginal zone lymphoma of mucosa-associated lymphoid tissue, hepatosplenic T-cell lymphoma, intraocular lymphoma, lymphomatous involvement of non-cutaneous extranodal site, mature T-cell and NK-cell non-Hodgkin lymphoma, nodal marginal zone lymphoma, post-transplant lymphoproliferative disorder, recurrent adult Burkitt lymphoma, recurrent adult diffuse large cell lymphoma, recurrent adult diffuse mixed cell lymphoma, recurrent adult diffuse small cleaved cell lymphoma, recurrent adult grade III lymphomatoid granulomatosis, recurrent adult immunoblastic lymphoma, recurrent adult lymphoblastic lymphoma, recurrent adult T-cell leukemia/lymphoma, recurrent cutaneous T-cell non-Hodgkin lymphoma, recurrent grade 1 follicular lymphoma, recurrent grade 2 follicular lymphoma, recurrent grade 3 follicular lymphoma, recurrent mantle cell lymphoma, recurrent marginal zone lymphoma, recurrent mycosis fungoides and Sezary syndrome, recurrent small lymphocytic lymphoma, refractory chronic lymphocytic leukemia, refractory hairy cell leukemia, Richter syndrome, small intestinal lymphoma, splenic marginal zone lymphoma, T-cell large granular lymphocyte leukemia, testicular lymphoma, Waldenstrom macroglobulinemia, adult T-cell leukemia-lymphoma, peripheral T-cell lymphoma, B-cell lymphoma, Hodgkin's disease, cutaneous T-cell lymphoma, diffuse large B-cell lymphoma, MALT lymphoma, mantle cell lymphoma, non-Hodgkins lymphoma, central nervous system lymphoma, refractory primary-cutaneous large B-cell lymphoma (Leg-type), relapsed or refractory chronic lymphocytic leukemia, refractory anemia, refractory anemia with excess blasts, refractory anemia with ringed sideroblasts, refractory cytopenia with multilineage dysplasia, and secondary myelodysplastic syndromes.

17. The method of claim 10, wherein the disorder is a neurological disorder.

18. The method of claim 17, wherein the neurological disorder is Alzheimer's disease.

19. The method of claim 10, wherein the disorder is a disorder associated with transplantation.

20. The method of claim 19, wherein the disorder associated with transplantation is graft-versus-host disease.
